(19) United States
(12) Patent Application Publication
(10) Pub. No.: US 2024/0368610 A1
(43) Pub. Date: Nov. 7, 2024

(54) INCREASING GENE EDITING AND SITE-DIRECTED INTEGRATION EVENTS UTILIZING MEIOTIC AND GERMLINE PROMOTERS

(71) Applicant: Monsanto Technology LLC, St. Louis, MO (US)

(72) Inventors: Matthew J. BAUER, Manchester, MO (US); Lisa KANIZAY, Chesterfield, MO (US); Jonathan LAMB, Wildwood, MO (US); Matthew S. MARENGO, Wildwood, MO (US); Brent O'BRIEN, St. Charles, MO (US)

(73) Assignee: Monsanto Technology LLC, St. Louis, MO (US)

(21) Appl. No.: 18/624,980

(22) Filed: Apr. 2, 2024

Related U.S. Application Data
(62) Division of application No. 17/470,784, filed on Sep. 9, 2021, now Pat. No. 11,981,900.
(60) Provisional application No. 63/076,705, filed on Sep. 10, 2020.

(51) Int. Cl. C12N 15/82; C12N 9/22; C12N 15/11

(52) U.S. Cl. CPC C12N 15/8213; C12N 9/22; C12N 15/11; C12N 2310/20

(57) ABSTRACT

This disclosure provides methods and compositions for increasing genome editing and site-directed integration events utilizing guided endonucleases and meiotic cell-preferred, egg cell-preferred or embryo tissue-preferred promoters.

### CROSS-REFERENCE TO RELATED APPLICATIONS

This application is a continuation of U.S. application Ser. No. 17/470,784, filed Sep. 9, 2021, which claims the benefit of U.S. Provisional Patent Application No. 63/076,705, filed Sep. 10, 2020, each of which is hereby incorporated by reference in its entirety.

### FIELD

The present disclosure relates to compositions and methods related to expressing guided nucleases and guide nucleic acids in egg cells and embryo tissues in plants.

### REFERENCE TO AN ELECTRONIC SEQUENCE LISTING

The contents of the electronic sequence listing (777052068201SEQLIST.xml; Size: 217,655 bytes; and Date of Creation: Mar. 30, 2024) is herein incorporated by reference in its entirety.

### BACKGROUND

CRISPR (Clustered Regularly Interspaced Short Palindromic Repeats) nucleases (e.g., Cas12a, CasX, Cas9) are proteins guided by guide RNAs to a target nucleic acid molecule, where the nuclease can cleave one or two strands of a target nucleic acid molecule.

### SUMMARY

In one aspect, this disclosure provides a plant comprising (a) a first nucleic acid sequence encoding a guided nuclease operably linked to a heterologous egg cell-preferred or embryo tissue-preferred promoter; and (b) a second nucleic acid sequence encoding at least one guide nucleic acid operably linked to a heterologous second promoter, where the at least one guide nucleic acid is capable of forming a complex with the guided nuclease and hybridizing to a target sequence within a genome of the plant and wherein the complex induces modification of the target sequence. In some embodiments, the guided nuclease is a CRISPR effector protein. In some embodiments, the modification is a staggered cut within a double-stranded DNA molecule of the genome. In some embodiments, the modification is an insertion of a transgene. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the egg cell-preferred promoter is selected from the group consisting of an EA1 promoter and an ES4 promoter. In some embodiments, the egg cell-preferred promoter comprises a nucleic acid sequence at least 90% identical to a nucleic acid sequence selected from the group consisting of SEQ ID NOs: 2-3, 21-38, 41-45, and 65-82 or a functional fragment thereof. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the plant. In some embodiments, the first nucleic acid sequence, the second nucleic acid sequence, or both, are stably integrated into a genome of a haploid inducer line. Several embodiments relate to a seed produced by the plant. In some embodiments, the seed comprises at least one mutation in a gene of interest comprising the target sequence as compared to a seed from a control plant of the same variety that lacks the first nucleic acid sequence or second nucleic acid sequence

In one aspect, this disclosure provides a plant comprising (a) a first nucleic acid sequence encoding a guided nuclease operably linked to a heterologous meiotic cell-preferred promoter; and (b) a second nucleic acid sequence encoding at least one guide nucleic acid operably linked to a heterologous second promoter, where the at least one guide nucleic acid is capable of forming a complex with the guided nuclease and hybridizing to a target sequence within a genome of the plant and wherein the complex induces modification of the target sequence. In some embodiments, the guided nuclease is a CRISPR effector protein. In some embodiments, the modification is a staggered cut within a double-stranded DNA molecule of the genome. In some embodiments, the modification is an insertion of a transgene. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the meiotic cell-preferred promoter is selected from the group consisting of a DMC1 promoter, a Mps1 promoter, and an Adf1 promoter. In some embodiments, the meiotic cell-preferred promoter comprises a nucleic acid sequence at least 90% identical to a nucleic acid sequence selected from the group consisting of SEQ ID NOs: 4-6 and 83-85 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the plant. In some embodiments, the first nucleic acid sequence, the second nucleic acid sequence, or both, are stably integrated into a genome of a haploid inducer line.

In one aspect, this disclosure provides a method of editing a genome of a plant comprising: (a) introducing to a plant cell: (i) a first nucleic acid sequence encoding a guided nuclease operably linked to a heterologous egg cell-preferred promoter; and (ii) a second nucleic acid sequence encoding at least one guide nucleic acid operably linked to a heterologous second promoter, where the at least one guide nucleic acid is capable of forming a complex with the guided nuclease and hybridizing to a target sequence within the genome; and (b) regenerating at least one plant from the plant cell of step (a), where the guided nuclease and at least one guide nucleic acid form a ribonucleoprotein within at least one egg cell of the plant, and where the ribonucleoprotein generates at least one modification within the target sequence in the at least one egg cell. In some embodiments, the guided nuclease is a CRISPR effector protein. In some embodiments, the modification is a staggered cut within a double-stranded DNA molecule of the genome. In some embodiments, the modification is an insertion of a transgene. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the egg cell-preferred promoter is selected from the group consisting of an EA1 promoter and an ES4 promoter. In some embodiments, the egg cell-preferred promoter comprises a nucleic acid sequence at least 90% identical to a nucleic acid sequence selected from the group consisting of SEQ ID NOs: 2-3, 21-38, 41-45, and 65-82 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, an ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the plant. In some embodiments, the first nucleic acid sequence, the second nucleic acid sequence, or both, are stably integrated into a genome of a haploid inducer line.

In one aspect, this disclosure provides a method of editing a genome of a plant comprising: (a) introducing to a plant cell: (i) a first nucleic acid sequence encoding a guided nuclease operably linked to a heterologous meiotic cell-preferred promoter; and (ii) a second nucleic acid sequence encoding at least one guide nucleic acid operably linked to a heterologous second promoter, where the at least one guide nucleic acid is capable of forming a complex with the guided nuclease and hybridizing to a target sequence within the genome; and (b) regenerating at least one plant from the plant cell of step (a), where the guided nuclease and at least one guide nucleic acid form a ribonucleoprotein within at least one meiotic cell of the plant, and where the ribonucleoprotein generates at least one modification within the target sequence in the at least one meiotic cell. In some embodiments, the guided nuclease is a CRISPR effector protein. In some embodiments, the modification is a staggered cut within a double-stranded DNA molecule of the genome. In some embodiments, the modification is an insertion of a transgene. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the meiotic cell-preferred promoter is selected from the group consisting of a DMC1 promoter, a Mps1 promoter, and an Adf1 promoter. In some embodiments, the meiotic cell-preferred promoter comprises a nucleic acid sequence at least 90% identical to a nucleic acid sequence selected from the group consisting of SEQ ID NOs: 4-6 and 83-85 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the plant. In some embodiments, the meiotic cell is from a haploid inducer line.

In one aspect, this disclosure provides a method of editing a genome of a plant cell comprising: (a) crossing a first plant with a second plant, where the first plant comprises a first nucleic acid sequence encoding a guided nuclease operably linked to a heterologous embryo tissue-preferred promoter, and where the second plant comprises a second nucleic acid sequence encoding at least one guide nucleic acid operably linked to a heterologous second promoter, where the at least one guide nucleic acid is capable of forming a complex with the guided nuclease and hybridizing to a target sequence within the genome; and (b) obtaining at least one embryo from the crossing of step (a), where the guided nuclease and the at least one guide nucleic acid form a ribonucleoprotein within the at least one embryo, and where the ribonucleoprotein generates at least one modification within the target sequence in the at least one embryo. In some embodiments, the guided nuclease is a CRISPR effector protein. In some embodiments, the modification is a staggered cut within a double-stranded DNA molecule of the genome. In some embodiments, the modification is an insertion of a transgene. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the first plant. In some embodiments, the first plant is a hybrid inducer.

In one aspect, this disclosure provides a method of editing a genome of a plant cell comprising: (a) crossing a first plant with a second plant, where the first plant comprises a first nucleic acid sequence encoding a guided nuclease operably linked to a heterologous meiotic-cell preferred promoter, and where the second plant comprises a second nucleic acid sequence encoding at least one guide nucleic acid operably linked to a heterologous second promoter, where the at least one guide nucleic acid is capable of forming a complex with the guided nuclease and hybridizing to a target sequence within the genome; and (b) obtaining at least one embryo from the crossing of step (a), where the guided nuclease and the at least one guide nucleic acid form a ribonucleoprotein within the at least one meiotic cell, and where the ribonucleoprotein generates at least one modification within the target sequence in the at least one meiotic cell. In some embodiments, the guided nuclease is a CRISPR effector protein. In some embodiments, the modification is a staggered cut within a double-stranded DNA molecule of the genome. In some embodiments, the modification is an insertion of a transgene. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the meiotic cell-preferred promoter is selected from the group consisting of a DMC1 promoter, a Mps1 promoter, and an Adf1 promoter. In some embodiments, the meiotic cell-preferred promoter comprises a nucleic acid sequence at least 90% identical to a nucleic acid sequence selected from the group consisting of SEQ ID NOs: 4-6 and 83-85 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the plant. In some embodiments, the first nucleic acid sequence, the second nucleic acid sequence, or both, are stably integrated into a genome of a haploid inducer line.

In one aspect, this disclosure provides a method of editing a genome of a plant comprising: (a) introducing to a plant cell: (i) a first nucleic acid sequence encoding a guided nuclease operably linked to a heterologous embryo tissue-preferred promoter; and (ii) a second nucleic acid sequence encoding at least one guide nucleic acid operably linked to a heterologous second promoter, where the at least one guide nucleic acid is capable of forming a complex with the guided nuclease and hybridizing to a target sequence within the genome; (b) regenerating at least one plant from the plant cell of step (a); and (c) fertilizing the at least one plant to create at least one embryo, where the guided nuclease and at least one guide nucleic acid form a ribonucleoprotein within the at least one embryo from step (c), and where the ribonucleoprotein generates at least one modification within the target sequence in the at least one embryo. In some embodiments, the guided nuclease is a CRISPR effector protein. In some embodiments, the modification is a staggered cut within a double-stranded DNA molecule of the genome. In some embodiments, the modification is an insertion of a transgene. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the first plant. In some embodiments, the first plant is a hybrid inducer.

In one aspect, this disclosure provides a method of generating a site-directed integration in a plant comprising: (a) introducing to a plant cell: (i) a first nucleic acid sequence encoding a guided nuclease operably linked to a heterologous egg cell-preferred promoter; (ii) a second nucleic acid sequence encoding one or more guide nucleic acids operably linked to a heterologous second promoter, where the one or more guide nucleic acids are (A) capable of forming a complex with the guided nuclease and hybridizing to a target sequence within a genome of the plant; and (B) capable of hybridizing to a first site and a second site flanking a nucleic acid sequence encoding a gene of interest; and (iii) a third nucleic acid sequence encoding the gene of interest; and (b) regenerating at least one plant from the plant cell of step (a); where the guided nuclease and at least one guide RNA form a ribonucleoprotein within at least one egg cell of the plant, where the ribonucleoprotein generates a double-stranded break within the target sequence, the first site, and the second site, and where the gene of interest is integrated into the target site in the at least one egg cell. In some embodiments, the guided nuclease is a CRISPR effector protein. In some embodiments, the modification is a staggered cut within a double-stranded DNA molecule of the genome. In some embodiments, the modification is an insertion of a transgene. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the egg cell-preferred promoter is selected from the group consisting of an EA1 promoter and an ES4 promoter. In some embodiments, the egg cell-preferred promoter comprises a nucleic acid sequence at least 90% identical to a nucleic acid sequence selected from the group consisting of SEQ ID NOs: 2-3, 21-38, 41-45, and 65-82 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, an ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the plant. In some embodiments, the first nucleic acid sequence, the second nucleic acid sequence, or both, are stably integrated into a genome of a haploid inducer line.

In one aspect, this disclosure provides a method of generating a site-directed integration in a plant comprising: (a) introducing to a plant cell: (i) a first nucleic acid sequence encoding a guided nuclease operably linked to a heterologous embryo tissue-preferred promoter; (ii) a second nucleic acid sequence encoding one or more guide nucleic acids operably linked to a heterologous second promoter, where the one or more guide nucleic acids are (A) capable of forming a complex with the guided nuclease and hybridizing to a target sequence within a genome of the plant; and (B) capable of hybridizing to a first site and a second site flanking a nucleic acid sequence encoding a gene of interest; (iii) a third nucleic acid sequence encoding the gene of interest; (b) regenerating at least one plant from the plant cell of step (a); and (c) fertilizing the at least one plant from step (b) to create at least one embryo; where the guided nuclease and at least one guide RNA form a ribonucleoprotein within at least one embryo, where the ribonucleoprotein generates a double-stranded break within the target sequence, the first site, and the second site, and where the gene of interest is integrated into the target site in the at least one embryo. In some embodiments, the guided nuclease is a CRISPR effector protein. In some embodiments, the modification is a staggered cut within a double-stranded DNA molecule of the genome. In some embodiments, the modification is an insertion of a transgene. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the first plant. In some embodiments, the first plant is a hybrid inducer.

In one aspect, this disclosure provides a recombinant DNA construct comprising (a) a first nucleic acid sequence encoding a guided nuclease operably linked to a heterologous egg cell-preferred promoter, meiotic cell preferred promoter or embryo tissue-preferred promoter; and (b) a second nucleic acid sequence encoding at least one guide nucleic acid operably linked to a heterologous second promoter, where the at least one guide nucleic acid is capable of hybridizing to a target sequence within a genome of a plant. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the egg cell-preferred promoter is selected from the group consisting of an EA1 promoter and an ES4 promoter. In some embodiments, the egg cell-preferred promoter comprises a nucleic acid sequence at least 90% identical to a nucleic acid sequence selected from the group consisting of SEQ ID NOs: 2-3, 21-38, 41-45, and 65-82 or a functional fragment thereof. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the meiotic cell-preferred promoter is selected from the group consisting of a DMC1 promoter, a Mps1 promoter, and an Adf1 promoter. In some embodiments, the meiotic cell-preferred promoter comprises a nucleic acid sequence at least 90% identical to a nucleic acid sequence selected from the group consisting of SEQ ID NOs: 4-6 and 83-85 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first nucleic acid sequence, the second nucleic acid sequence, or both, are stably integrated into a genome of the plant. In some embodiments, the first nucleic acid sequence, the second nucleic acid sequence, or both, are stably integrated into a genome of a haploid inducer line. In some embodiments, the recombinant DNA construct is integrated into the genome of a haploid inducer line.

Several embodiments relate to a recombinant DNA construct comprising (a) a first nucleic acid sequence encoding a DNA modification enzyme operably linked to one or more one or more TALE binding sites and a minimal promoter; and (b) a second nucleic acid sequence encoding a TALE operably linked to a egg cell-preferred promoter, meiotic cell-preferred promoter or embryo tissue-preferred promoter, wherein the minimal promoter does not drive expression of the DNA modification enzyme in the absence of TALE binding to the to one or more one or more TALE binding sites. In some embodiments, the recombinant DNA construct further comprises a third nucleic acid sequence encoding a guide nucleic acid operably linked to a third promoter. In some embodiments, the DNA modification enzyme is a guided nuclease. In some embodiments, the guided nuclease is a CRISPR effector protein. In some embodiments, the first nucleic acid sequence encoding a DNA modification enzyme and the minimal promoter are operably linked 1, 2, 3, 4, 5, 6, 7, 8, 9, 10 or more TALE binding sites. In some embodiments, the CRISPR effector protein is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for expression in a plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the egg cell-preferred promoter is selected from the group consisting of an EA1 promoter and an ES4 promoter. In some embodiments, the egg cell-preferred promoter comprises a nucleic acid sequence at least 90% identical to a nucleic acid sequence selected from the group consisting of SEQ ID NOs: 2-3, 21-38, 41-45, and 65-82 or a functional fragment thereof. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the meiotic cell-preferred promoter is selected from the group consisting of a DMC1 promoter, a Mps1 promoter, and an Adf1 promoter. In some embodiments, the meiotic cell-preferred promoter comprises a nucleic acid sequence at least 90% identical to a nucleic acid sequence selected from the group consisting of SEQ ID NOs: 4-6 and 83-85 or a functional fragment thereof. In some embodiments, the third promoter is a Pol III promoter. In some embodiments, the third promoter is selected from the group consisting of a tissue-preferred promoter, a tissue-specific promoter, an inducible promoter, and a constitutive promoter. In some embodiments, the third promoter is a meiotic cell-preferred, an egg cell-preferred or an embryo tissue-preferred promoter. In some embodiments, the third promoter is meiotic cell-specific, an egg cell-specific or an embryo tissue-specific promoter. In some embodiments, the third promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the third promoter comprises a nucleic acid sequence at least 90% identical to a nucleic acid sequence selected from the group consisting of SEQ ID NOs: 1-6, 21-45 and 65-88 or a functional fragment thereof. In some embodiments, the third promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the third nucleic acid sequence encoding at least one guide nucleic acid is operably linked to one or more self-cleaving ribozymes. In some embodiments, the first nucleic acid sequence, the second nucleic acid sequence, and/or the third nucleic acid sequence, are stably integrated into a genome of the plant. In some embodiments the plant is from a haploid inducer line. In some embodiments the guide nucleic acid is provided to the plant by bombardment. Several embodiments relate to a plant comprising the recombinant DNA construct. Several embodiments relate to a seed produced by a plant comprising the recombinant DNA construct. In some embodiments, the recombinant DNA construct is integrated into the genome of a haploid inducer line.

Several embodiments relate to a recombinant DNA construct comprising (a) a first nucleic acid sequence encoding at least one guide nucleic acid operably linked to one or more one or more TALE binding sites and a minimal promoter; and (b) a second nucleic acid sequence encoding a TALE operably linked to an egg cell-preferred promoter, a meiotic cell-preferred promoter or an embryo tissue-preferred promoter, wherein the minimal promoter does not drive expression of the guide nucleic acid in the absence of TALE binding to the to one or more one or more TALE binding sites. In some embodiments, the recombinant DNA construct further comprises a third nucleic acid sequence encoding a guided nuclease operably linked to a third promoter. In some embodiments, the guided nuclease is a CRISPR effector protein. In some embodiments, the first nucleic acid sequence encoding guide nucleic acid and the minimal promoter are operably linked 1, 2, 3, 4, 5, 6, 7, 8, 9, 10 or more TALE binding sites. In some embodiments, the CRISPR effector protein is selected from the group consisting of Cas9, Cas12a (e.g., LbCas12a, FnCas12a) and CasX. In some embodiments, the third nucleic acid sequence comprises a nucleic acid sequence at least 90% identical to SEQ ID NO: 7. In some embodiments, the third nucleic acid sequence is codon-optimized for expression in a plant. In some embodiments, the third nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the egg cell-preferred promoter is selected from the group consisting of an EA1 promoter and an ES4 promoter. In some embodiments, the egg cell-preferred promoter comprises a nucleic acid sequence at least 90% identical to a nucleic acid sequence selected from the group consisting of SEQ ID NOs: 2-3, 21-38, 41-45, and 65-82 or a functional fragment thereof. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the meiotic cell-preferred promoter is selected from the group consisting of a DMC1 promoter, a Mps1 promoter, and an Adf1 promoter. In some embodiments, the meiotic cell-preferred promoter comprises a nucleic acid sequence at least 90% identical to a nucleic acid sequence selected from the group consisting of SEQ ID NOs: 4-6 and 83-85 or a functional fragment thereof. In some embodiments, the third promoter is selected from the group consisting of a tissue-preferred promoter, a tissue-specific promoter, an inducible promoter, and a constitutive promoter. In some embodiments, the third promoter is a meiotic cell-preferred, an egg cell-preferred or an embryo tissue-preferred promoter. In some embodiments, the third promoter is meiotic cell-specific, an egg cell-specific or an embryo tissue-specific promoter. In some embodiments, the third promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the third promoter comprises a nucleic acid sequence at least 90% identical to a nucleic acid sequence selected from the group consisting of SEQ ID NOs: 1-6, 21-45 and 65-88 or a functional fragment thereof. In some embodiments, the third promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the first nucleic acid sequence encoding the at least one guide nucleic acid is operably linked to one or more self-cleaving ribozymes. In some embodiments, the first nucleic acid sequence, the second nucleic acid sequence, and/or the third nucleic acid sequence, are stably integrated into a genome of the plant. In some embodiments the plant is from a haploid inducer line. Several embodiments relate to a plant comprising the recombinant DNA construct. Several embodiments relate to a seed produced by a plant comprising the recombinant DNA construct. In some embodiments, the recombinant DNA construct is integrated into the genome of a haploid inducer line.

Several embodiments relate to a recombinant DNA construct comprising (a) a first nucleic acid sequence encoding a guided nuclease; (b) a second nucleic acid sequence encoding a first promoter; and (c) a third nucleic acid sequence encoding DNA modification enzyme operably linked to an egg cell-preferred promoter, a meiotic cell-preferred promoter or an embryo tissue-preferred promoter, wherein the third nucleic acid is positioned between the first nucleic acid and the second nucleic acid, and wherein the third nucleic acid comprises a first target site for the DNA modification enzyme at the 5′ end and a second target site for the DNA modification enzyme at the 5′ end. In some embodiments, the recombinant DNA construct further comprises a fourth nucleic acid sequence encoding one or more guide nucleic acids operably linked to a third promoter. In some embodiments, the DNA modification enzyme is a recombinase. In some embodiments, the first and second target sites are Lox sites. In some embodiments, the DNA modification enzyme is an endonuclease. In some embodiments, the DNA modification enzyme is a CRISPR effector protein. In some embodiments, the guided nuclease is a CRISPR effector protein. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the first promoter is a meiotic cell-preferred, an egg cell-preferred or an embryo tissue-preferred promoter. In some embodiments, the first promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the first promoter comprises a nucleic acid sequence at least 90% identical to a nucleic acid sequence selected from the group consisting of SEQ ID NOs: 1-6, 21-45 and 65-88 or a functional fragment thereof. In some embodiments, the first promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the egg cell-preferred promoter is selected from the group consisting of an EA1 promoter and an ES4 promoter. In some embodiments, the egg cell-preferred promoter comprises a nucleic acid sequence at least 90% identical to a nucleic acid sequence selected from the group consisting of SEQ ID NOs: 2-3, 21-38, 41-45, and 65-82 or a functional fragment thereof. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the meiotic cell-preferred promoter is selected from the group consisting of a DMC1 promoter, a Mps1 promoter, and an Adf1 promoter. In some embodiments, the meiotic cell-preferred promoter comprises a nucleic acid sequence at least 90% identical to a nucleic acid sequence selected from the group consisting of SEQ ID NOs: 4-6 and 83-85 or a functional fragment thereof. In some embodiments the third promoter is a Pol III promoter. Several embodiments relate to a plant comprising the recombinant DNA construct. Several embodiments relate to a seed produced by a plant comprising the recombinant DNA construct. In some embodiments the plant is from a haploid inducer line. In some embodiments, the recombinant DNA construct is integrated into the genome of a haploid inducer line.

In several embodiments, high levels of egg, embryo, and/or meiotic tissue specific expression of a DNA modification enzyme such as a guided nuclease (e.g., a CRISPR/Cas system), is achieved by providing to a plant cell: 1) an expression construct comprising a promoter as described in Table 1 operably linked to a sequence encoding a CRISPR effector protein, such as a dCas12a or dCas9 fused to a transcription activator; 2) an expression construct comprising one or more target sites operably linked to a minimal promoter and a sequence encoding the DNA modification enzyme; and 3) an expression construct encoding a guide RNA that hybridizes with the one or more target sites; and generating a plant therefrom. In some embodiments, 1, 2, 3, 4, 5, 6, 7, 8, 9, 10 or more target sites are operably linked to the minimal promoter. In some embodiments, the plant cell is from a haploid inducer line. In some embodiments, a female plant is generated that expresses high levels of the DNA modification enzyme in egg, embryo, and/or meiotic tissue. In some embodiments, the female plant is outcrossed and a population of R1 plants comprising unique edits are identified.

In several embodiments, high levels of egg, embryo, and/or meiotic tissue specific expression of a DNA modification enzyme such as a guided nuclease (e.g., a CRISPR/Cas system), is achieved by providing to a plant cell: 1) an expression construct comprising a promoter as described in Table 1 operably linked to a sequence encoding a TALE and 2) and an expression construct comprising one or more TALE binding sites (TB) operably linked to a minimal promoter and a sequence encoding the DNA modification enzyme and generating a plant therefrom. In some embodiments, an expression construct encoding one or more guide nucleic acids is further provided. In some embodiments, 1, 2, 3, 4, 5, 6, 7, 8, 9, 10 or more TBs are operably linked to the minimal promoter. In some embodiments, the plant cell is from a haploid inducer line. In some embodiments, a female plant is generated that expresses high levels of the DNA modification enzyme in egg, embryo, and/or meiotic tissue. In some embodiments, the female plant is outcrossed and a population of R1 plants comprising unique edits are identified.

Several embodiments relate to a method of generating two or more progeny plants with unique edits from a single transformed plant cell, the method comprising: (a) introducing to the plant cell: a first nucleic acid sequence encoding a guided nuclease operably linked to a heterologous meiosis-preferred promoter; and a second nucleic acid sequence encoding at least one guide nucleic acid operably linked to a heterologous second promoter, wherein the at least one guide nucleic acid is capable of hybridizing to a target sequence within the genome; and (b) regenerating a first plant from the plant cell of step (a), wherein the guided nuclease and at least one guide nucleic acid form a ribonucleoprotein within at least one meiotic cell of the first plant, and wherein the ribonucleoprotein generates at least one modification within the target sequence in the at least one meiotic cell; (c) pollinating the first plant of step (b); (d) germinating two or more seeds produced from step (c) to produced two or more progeny plants with unique edits. In some embodiments, the modification is a staggered cut within a double-stranded DNA molecule of the genome. In some embodiments, the target sequence comprises genic DNA. In some embodiments, the target sequence comprises intergenic DNA. In some embodiments, the target sequence is within a gene of interest. In some embodiments, the gene of interest encodes a protein or a non-protein-coding RNA. In some embodiments, the gene of interest encodes a non-protein-coding RNA is selected from the group consisting of a microRNA, a small interfering RNA (siRNA), a trans-acting siRNA, or a precursor thereof. In some embodiments, the guided nuclease is a CRISPR effector protein. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the meiotic cell-preferred promoter is selected from the group consisting of a DMC1 promoter, a Mps1 promoter, and an Adf1 promoter. In some embodiments, the meiotic cell-preferred promoter comprises a nucleic acid sequence at least 90% identical to a nucleic acid sequence selected from the group consisting of SEQ ID NOs: 4-6 and 83-85 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first plant is self-pollinated. In some embodiments the first plant is outcrossed. In some embodiments, the plant cell is from a haploid inducer line. In some embodiments, the progeny plants are haploid. In some embodiments, the method further comprises screening the haploid progeny for modifications at the target site. In some embodiments, the method further comprises inducing doubling of the genome of the haploid plants.

Several embodiments relate to a method of generating two or more progeny plants with unique edits from a single transformed plant cell, the method comprising: (a) introducing to the plant cell: a first nucleic acid sequence encoding a guided nuclease operably linked to a heterologous first promoter; and a second nucleic acid sequence encoding at least one guide nucleic acid operably linked to a heterologous meiosis-preferred promoter, wherein the at least one guide nucleic acid is capable of hybridizing to a target sequence within the genome; and (b) regenerating a first plant from the plant cell of step (a), wherein the guided nuclease and at least one guide nucleic acid form a ribonucleoprotein within at least one meiotic cell of the first plant, and wherein the ribonucleoprotein generates at least one modification within the target sequence in the at least one meiotic cell; (c) pollinating the first plant of step (b); (d) germinating two or more seeds produced from step (c) to produced two or more progeny plants with unique edits. In some embodiments, the modification is a staggered cut within a double-stranded DNA molecule of the genome. In some embodiments, the target sequence comprises genic DNA. In some embodiments, the target sequence comprises intergenic DNA. In some embodiments, the target sequence is within a gene of interest. In some embodiments, the gene of interest encodes a protein or a non-protein-coding RNA. In some embodiments, the gene of interest encodes a non-protein-coding RNA is selected from the group consisting of a microRNA, a small interfering RNA (siRNA), a trans-acting siRNA, or a precursor thereof. In some embodiments, the guided nuclease is a CRISPR effector protein. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the meiotic cell-preferred promoter is selected from the group consisting of a DMC1 promoter, a Mps1 promoter, and an Adf1 promoter. In some embodiments, the meiotic cell-preferred promoter comprises a nucleic acid sequence at least 90% identical to a nucleic acid sequence selected from the group consisting of SEQ ID NOs: 4-6 and 83-85 or a functional fragment thereof. In some embodiments, the first promoter is selected from the group consisting of a tissue-preferred promoter, a tissue-specific promoter, an inducible promoter, and a constitutive promoter. In some embodiments, the first promoter is a meiotic cell-preferred, an egg cell-preferred or an embryo tissue-preferred promoter. In some embodiments, the first promoter is meiotic cell-specific, an egg cell-specific or an embryo tissue-specific promoter. In some embodiments, the first promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the first promoter comprises a nucleic acid sequence at least 90% identical to a nucleic acid sequence selected from the group consisting of SEQ ID NOs: 1-6, 21-45 and 65-88 or a functional fragment thereof. In some embodiments, the first promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first plant is self-pollinated. In some embodiments the first plant is outcrossed. In some embodiments, the plant cell is from a haploid inducer line. In some embodiments, the progeny plants are haploid. In some embodiments, the method further comprises screening the haploid progeny for modifications at the target site. In some embodiments, the method further comprises inducing doubling of the genome of the haploid plants.

A method of generating two or more progeny plants with unique edits from a single transformed plant cell, the method comprising: (a) introducing to the plant cell: a first nucleic acid sequence encoding a guided nuclease operably linked to a heterologous egg cell-preferred promoter; and a second nucleic acid sequence encoding at least one guide nucleic acid operably linked to a heterologous second promoter, wherein the at least one guide nucleic acid is capable of hybridizing to a target sequence within the genome; and (b) regenerating a first plant from the plant cell of step (a), wherein the guided nuclease and at least one guide nucleic acid form a ribonucleoprotein within at least one egg cell of the first plant, and wherein the ribonucleoprotein generates at least modification within the target sequence in the at least one egg cell; (c) pollinating the first plant of step (b); (d) germinating two or more seeds produced from step (c) to produced two or more progeny plants with unique edits. In some embodiments, the modification is a staggered cut within a double-stranded DNA molecule of the genome. In some embodiments, the target sequence comprises genic DNA. In some embodiments, the target sequence comprises intergenic DNA. In some embodiments, the target sequence is within a gene of interest. In some embodiments, the gene of interest encodes a protein or a non-protein-coding RNA. In some embodiments, the gene of interest encodes a non-protein-coding RNA is selected from the group consisting of a microRNA, a small interfering RNA (siRNA), a trans-acting siRNA, or a precursor thereof. In some embodiments, the guided nuclease is a CRISPR effector protein. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the egg cell-preferred promoter is selected from the group consisting of an EA1 promoter and an ES4 promoter. In some embodiments, the egg cell-preferred promoter comprises a nucleic acid sequence at least 90% identical to a nucleic acid sequence selected from the group consisting of SEQ ID NOs: 2-3, 21-38, 41-45, and 65-82 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, an ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first plant is self-pollinated. In some embodiments the first plant is outcrossed. In some embodiments, the plant cell is from a haploid inducer line. In some embodiments, the progeny plants are haploid. In some embodiments, the method further comprises screening the haploid progeny for modifications at the target site. In some embodiments, the method further comprises inducing doubling of the genome of the haploid plants.

A method of generating two or more progeny plants with unique edits from a single transformed plant cell, the method comprising: (a) introducing to the plant cell: a first nucleic acid sequence encoding a guided nuclease operably linked to a heterologous first promoter; and a second nucleic acid sequence encoding at least one guide nucleic acid operably linked to a heterologous egg cell-preferred promoter, wherein the at least one guide nucleic acid is capable of hybridizing to a target sequence within the genome; and (b) regenerating a first plant from the plant cell of step (a), wherein the guided nuclease and at least one guide nucleic acid form a ribonucleoprotein within at least one egg cell of the first plant, and wherein the ribonucleoprotein generates at least one modification within the target sequence in the at least one egg cell; (c) pollinating the first plant of step (b); (d) germinating two or more seeds produced from step (c) to produced two or more progeny plants with unique edits. In some embodiments, the modification is a staggered cut within a double-stranded DNA molecule of the genome. In some embodiments, the target sequence comprises genic DNA. In some embodiments, the target sequence comprises intergenic DNA. In some embodiments, the target sequence is within a gene of interest. In some embodiments, the gene of interest encodes a protein or a non-protein-coding RNA. In some embodiments, the gene of interest encodes a non-protein-coding RNA is selected from the group consisting of a microRNA, a small interfering RNA (siRNA), a trans-acting siRNA, or a precursor thereof. In some embodiments, the guided nuclease is a CRISPR effector protein. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the egg cell-preferred promoter is selected from the group consisting of an EA1 promoter and an ES4 promoter. In some embodiments, the egg cell-preferred promoter comprises a nucleic acid sequence at least 90% identical to a nucleic acid sequence selected from the group consisting of SEQ ID NOs: 2-3, 21-38, 41-45, and 65-82 or a functional fragment thereof. In some embodiments, the first promoter is selected from the group consisting of a tissue-preferred promoter, a tissue-specific promoter, an inducible promoter, and a constitutive promoter. In some embodiments, the first promoter is a meiotic cell-preferred, an egg cell-preferred or an embryo tissue-preferred promoter. In some embodiments, the first promoter is meiotic cell-specific, an egg cell-specific or an embryo tissue-specific promoter. In some embodiments, the first promoter is selected from the group consisting of a DSUL1 promoter, an EA1 promoter, an ES4 promoter, a DMC1 promoter, a Mps1 promoter, an Adf1 promoter and an EAL1 promoter. In some embodiments, the first promoter comprises a nucleic acid sequence at least 90% identical to a nucleic acid sequence selected from the group consisting of SEQ ID NOs: 1-6, 21-45 and 65-88 or a functional fragment thereof. In some embodiments, the first promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first plant is self-pollinated. In some embodiments the first plant is outcrossed. In some embodiments, the plant cell is from a haploid inducer line. In some embodiments, the progeny plants are haploid. In some embodiments, the method further comprises screening the haploid progeny for modifications at the target site. In some embodiments, the method further comprises inducing doubling of the genome of the haploid plants.

A method of generating two or more progeny plants with unique edits from a single transformed plant cell, the method comprising: (a) introducing to the plant cell: a first nucleic acid sequence encoding a guided nuclease operably linked to a heterologous embryonic cell-preferred promoter; and a second nucleic acid sequence encoding at least one guide nucleic acid operably linked to a heterologous second promoter, wherein the at least one guide nucleic acid is capable of hybridizing to a target sequence within the genome; and (b) regenerating a first plant from the plant cell of step (a), (c) pollinating the first plant of step (b), wherein the guided nuclease and at least one guide nucleic acid form a ribonucleoprotein within embryonic cells, and wherein the ribonucleoprotein generates at least one modification within the target sequence in the embryonic cells; (d) germinating two or more seeds produced from step (c) to produced two or more progeny plants with unique edits. In some embodiments, the modification is a staggered cut within a double-stranded DNA molecule of the genome. In some embodiments, the target sequence comprises genic DNA. In some embodiments, the target sequence comprises intergenic DNA. In some embodiments, the target sequence is within a gene of interest. In some embodiments, the gene of interest encodes a protein or a non-protein-coding RNA. In some embodiments, the gene of interest encodes a non-protein-coding RNA is selected from the group consisting of a microRNA, a small interfering RNA (siRNA), a trans-acting siRNA, or a precursor thereof. In some embodiments, the guided nuclease is a CRISPR effector protein. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the second promoter is a Pol III promoter. In some embodiments, the second promoter is selected from the group consisting of a tissue-preferred promoter, a tissue-specific promoter, an inducible promoter, and a constitutive promoter. In some embodiments, the second promoter is a meiotic cell-preferred, an egg cell-preferred or an embryo tissue-preferred promoter. In some embodiments, the second promoter is meiotic cell-specific, an egg cell-specific or an embryo tissue-specific promoter. In some embodiments, the second promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the second promoter comprises a nucleic acid sequence at least 90% identical to a nucleic acid sequence selected from the group consisting of SEQ ID NOs: 1-6, 21-45 and 65-88 or a functional fragment thereof. In some embodiments, the second promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first plant is self-pollinated. In some embodiments the first plant is outcrossed. In some embodiments, the plant cell is from a haploid inducer line. In some embodiments, the progeny plants are haploid. In some embodiments, the method further comprises screening the haploid progeny for modifications at the target site. In some embodiments, the method further comprises inducing doubling of the genome of the haploid plants.

A method of generating two or more progeny plants with unique edits from a single transformed plant cell, the method comprising: (a) introducing to the plant cell: a first nucleic acid sequence encoding a guided nuclease operably linked to a heterologous first promoter; and a second nucleic acid sequence encoding at least one guide nucleic acid operably linked to a heterologous embryonic cell-preferred promoter, wherein the at least one guide nucleic acid is capable of hybridizing to a target sequence within the genome; and (b) regenerating a first plant from the plant cell of step (a), (c) pollinating the first plant of step (b), wherein the guided nuclease and at least one guide nucleic acid form a ribonucleoprotein within embryonic cells, and wherein the ribonucleoprotein generates at least one modification within the target sequence in the embryonic cells; (d) germinating two or more seeds produced from step (c) to produced two or more progeny plants with unique edits. In some embodiments, the modification is a staggered cut within a double-stranded DNA molecule of the genome. In some embodiments, the target sequence comprises genic DNA. In some embodiments, the target sequence comprises intergenic DNA. In some embodiments, the target sequence is within a gene of interest. In some embodiments, the gene of interest encodes a protein or a non-protein-coding RNA. In some embodiments, the gene of interest encodes a non-protein-coding RNA is selected from the group consisting of a microRNA, a small interfering RNA (siRNA), a trans-acting siRNA, or a precursor thereof. In some embodiments, the guided nuclease is a CRISPR effector protein. In some embodiments, the guided nuclease is selected from the group consisting of Cas9, Cas12a (e.g., LbCas12a, FnCas12a) and CasX. In some embodiments, the first nucleic acid sequence comprises a nucleic acid sequence at least 90% identical to SEQ ID NO: 7. In some embodiments, the first nucleic acid sequence is codon-optimized for the plant. In some embodiments, the first nucleic acid sequence encodes at least one nuclear localization signal. In some embodiments, the at least one nuclear localization signal comprises a nucleic acid sequence selected from the group consisting of SEQ ID NOs: 8 and 9. In some embodiments, the embryo tissue-preferred promoter is selected from the group consisting of a DSUL1 promoter, an EA1 promoter, an ES4 promoter, and an EAL1 promoter. In some embodiments, the embryo tissue-preferred promoter comprises a nucleic acid sequence at least 90% identical to a nucleic acid sequence selected from the group consisting of SEQ ID NOs: 1-3, 29-40, 43-45 and 86-88 or a functional fragment thereof. In some embodiments, the first promoter is selected from the group consisting of a tissue-preferred promoter, a tissue-specific promoter, an inducible promoter, and a constitutive promoter. In some embodiments, the first promoter is a meiotic cell-preferred, an egg cell-preferred or an embryo tissue-preferred promoter. In some embodiments, the first promoter is meiotic cell-specific, an egg cell-specific or an embryo tissue-specific promoter. In some embodiments, the first promoter is selected from the group consisting of a DSUL1 promoter, an EA1 promoter, a ES4 promoter, a DMC1 promoter, a Mps1 promoter, an Adf1 promoter and an EAL1 promoter. In some embodiments, the first promoter comprises a nucleic acid sequence at least 90% identical to a nucleic acid sequence selected from the group consisting of SEQ ID NOs: 1-6, 21-45 and 65-88 or a functional fragment thereof. In some embodiments, the first promoter is a constitutive promoter selected from the group consisting of a CaMV 35S promoter, an Actin promoter, a Rab15 promoter, and a Ubiquitin promoter. In some embodiments, the at least one guide nucleic acid comprises at least one guide RNA. In some embodiments, the second nucleic acid sequence encoding at least one guide nucleic acid is operably linked to one or more self-cleaving ribozymes. In some embodiments, the first plant is self-pollinated. In some embodiments the first plant is outcrossed. In some embodiments, the plant cell is from a haploid inducer line. In some embodiments, the progeny plants are haploid. In some embodiments, the method further comprises screening the haploid progeny for modifications at the target site. In some embodiments, the method further comprises inducing doubling of the genome of the haploid plants.

### FIGURE LEGEND

FIG. 1. Editing in reciprocal F1 plants with reproductive promoters. Transgenic R1 lines were used as either the female or male to generate reciprocal F1s. The black bars represent percent of events with an active LbCas12a when provided by the female or male parent, as indicated by new edits being present in F1 plants. The grey bars are the percent of F1 individuals containing new edits. The light grey bars are the number of unique edits found in the F1s.

FIG. 2: Depicts a schematic illustrating vectors designed for TALE induced expression of LbCas12a. 35S(-46) is the 35S minimal promoter. TB indicates the TALE Binding site.

FIG. 3. RNA expression of Cas12a and TALE in corn leaf protoplasts.

FIG. 4: Schematic illustration of T-DNA vectors designed for TALE-induced Meiocyte/Embryo/egg-cell preferred expression of LbCas12a in planta. LB indicates Left Border. RB indicates Right Border. P indicates promoter. 35S(-46) is the 35S minimal promoter. TB is the TALE Binding site.

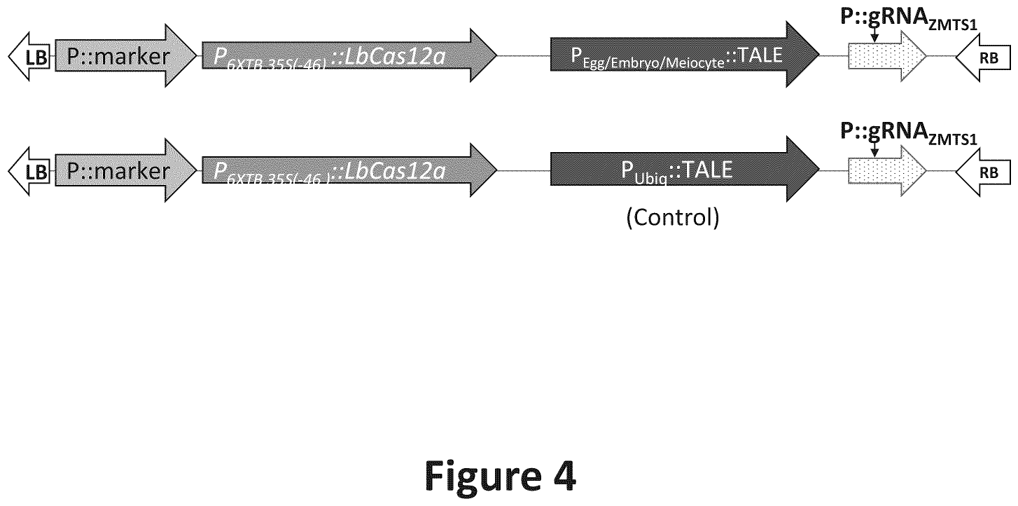

FIG. 5: Schematic illustration of T-DNA vectors designed for Meiocyte/Embryo/egg-cell preferred expression of Cas12a driven by a strong constitutive promoter. P indicates Promoter. Cre is for Cre recombinase. Arrowheads represent directionality.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of this disclosure includes, unless otherwise indicated, conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et. al. eds., (1987)); Plant Breeding Methodology (N. F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

As used herein, terms in the singular and the singular forms “a,” “an,” and “the,” for example, include plural referents unless the content clearly dictates otherwise.

Any composition, nucleic acid molecule, polypeptide, cell, plant, etc. provided herein is specifically envisioned for use with any method provided herein.

Several embodiments described herein relate to compositions and methods for expressing a DNA modification enzyme, for example a guided nuclease, preferentially in the egg, meiotic, and/or embryonic cells of a plant. In some embodiments, compositions and methods are provided for preferential expression of components of a CRISPR/Cas editing system in egg, meiotic, and/or embryonic cells of a plant. Several embodiments relate to compositions and methods for producing offspring with unique edits from a parent comprising an expression cassette that preferentially provides a DNA modification enzyme, for example a guided nuclease, in egg, meiotic, and/or embryonic cells. In some embodiments, a female parent plant is provided that preferentially expresses a DNA modification enzyme, for example a guided nuclease, preferentially in egg, meiotic, and/or embryonic cells. In some embodiments, a male parent plant is provided that preferentially expresses a DNA modification enzyme, for example a guided nuclease, preferentially in egg, meiotic, and/or embryonic cells. In some embodiments, a population of seeds wherein 2 or more of the seeds comprise unique edits is provided wherein the population of seeds is produced from a parent expressing a DNA modification enzyme, for example a guided nuclease, preferentially in egg, meiotic, and/or embryonic cells. Nonlimiting examples of expression elements useful in the composition and methods described herein are provided in Table 1.

As used herein, an “egg cell” refers to a haploid egg cell produced by the female gametophyte of a plant. Upon fertilization by a haploid pollen cell, a diploid zygote is formed, which gives rise to an embryo. As used herein, “embryo tissue” refers to diploid tissue comprising precursor tissues for leaf, stem, and root tissue, as well as one or more cotyledons. Embryo tissue is eventually incorporated into a seed. Once the embryo begins to germinate, a seedling or plantlet is generated. As used herein, meiosis refers to a process of cell division in sexually-reproducing organisms that produces gametes. Meiosis involves two rounds of cell division that ultimately result in four haploid cells. A meiotic cell refers to a cell undergoing meiosis.

In an aspect, this disclosure provides a recombinant DNA construct comprising a first nucleic acid sequence encoding a guided nuclease capable of generating a staggered cut in a double-stranded DNA molecule operably linked to a heterologous egg cell-preferred promoter; and (b) a second nucleic acid sequence encoding at least one guide nucleic acid operably linked to a heterologous second promoter, where the at least one guide nucleic acid is capable of hybridizing to a target sequence within a genome of a plant. In an aspect, this disclosure provides a recombinant DNA construct comprising a first nucleic acid sequence encoding a guided nuclease capable of generating a staggered cut in a double-stranded DNA molecule operably linked to a heterologous embryo tissue-preferred promoter; and (b) a second nucleic acid sequence encoding at least one guide nucleic acid operably linked to a heterologous second promoter, where the at least one guide nucleic acid is capable of hybridizing to a target sequence within a genome of a plant. In an aspect, this disclosure provides a recombinant DNA construct comprising a first nucleic acid sequence encoding a guided nuclease capable of generating a staggered cut in a double-stranded DNA molecule operably linked to a heterologous meiosis-preferred promoter; and (b) a second nucleic acid sequence encoding at least one guide nucleic acid operably linked to a heterologous second promoter, where the at least one guide nucleic acid is capable of hybridizing to a target sequence within a genome of a plant cell.

In an aspect, this disclosure provides a recombinant DNA construct comprising a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to an egg cell-preferred promoter, where the at least one guide nucleic acid is capable of hybridizing to a target sequence within a genome of a plant. In an aspect, this disclosure provides a recombinant DNA construct comprising a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to an embryo tissue-preferred promoter, where the at least one guide nucleic acid is capable of hybridizing to a target sequence within a genome of a plant. In an aspect, this disclosure provides a recombinant DNA construct comprising a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to an meiotic-preferred promoter, where the at least one guide nucleic acid is capable of hybridizing to a target sequence within a genome of a plant cell.

In an aspect, this disclosure provides a plant comprising a recombinant DNA construct comprising a first nucleic acid sequence encoding a guided nuclease capable of generating a staggered cut in a double-stranded DNA molecule operably linked to a heterologous egg cell-preferred promoter; and (b) a second nucleic acid sequence encoding at least one guide nucleic acid operably linked to a heterologous second promoter, where the at least one guide nucleic acid is capable of hybridizing to a target sequence within a genome of the plant. In an aspect, this disclosure provides a plant comprising a recombinant DNA construct comprising a first nucleic acid sequence encoding a guided nuclease capable of generating a staggered cut in a double-stranded DNA molecule operably linked to a heterologous embryo tissue-preferred promoter; and (b) a second nucleic acid sequence encoding at least one guide nucleic acid operably linked to a heterologous second promoter, where the at least one guide nucleic acid is capable of hybridizing to a target sequence within a genome of the plant. In an aspect, this disclosure provides a plant comprising a recombinant DNA construct comprising a first nucleic acid sequence encoding a guided nuclease capable of generating a staggered cut in a double-stranded DNA molecule operably linked to a heterologous meiosis-preferred promoter; and (b) a second nucleic acid sequence encoding at least one guide nucleic acid operably linked to a heterologous second promoter, where the at least one guide nucleic acid is capable of hybridizing to a target sequence within a genome of the plant.

In an aspect, this disclosure provides a plant comprising a recombinant DNA construct comprising a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to an egg cell-preferred promoter, where the at least one guide nucleic acid is capable of hybridizing to a target sequence within a genome of the plant. In an aspect, this disclosure provides a plant comprising a recombinant DNA construct comprising a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to an embryo tissue-preferred promoter, where the at least one guide nucleic acid is capable of hybridizing to a target sequence within a genome of the plant. In an aspect, this disclosure provides a plant comprising a recombinant DNA construct comprising a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to an meiosis-preferred promoter, where the at least one guide nucleic acid is capable of hybridizing to a target sequence within a genome of the plant.

In an aspect, this disclosure provides a seed of any plant provided herein.

### Nucleic Acids and Amino Acids

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise deoxyribonucleotides, ribonucleotides, or combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded.

As used herein, the term “recombinant” in reference to a nucleic acid (DNA or RNA) molecule, protein, construct, vector, etc., refers to a nucleic acid or amino acid molecule or sequence that is man-made and not normally found in nature, and/or is present in a context in which it is not normally found in nature, including a nucleic acid molecule (DNA or RNA) molecule, protein, construct, etc., comprising a combination of polynucleotide or protein sequences that would not naturally occur contiguously or in close proximity together without human intervention, and/or a polynucleotide molecule, protein, construct, etc., comprising at least two polynucleotide or protein sequences that are heterologous with respect to each other.

In one aspect, methods and compositions provided herein comprise a vector. As used herein, the term “vector” refers to a DNA molecule used as a vehicle to carry exogenous genetic material into a cell.

In an aspect, one or more polynucleotide sequences from a vector are stably integrated into a genome of a plant. In an aspect, one or more polynucleotide sequences from a vector are stably integrated into a genome of a plant cell.

In an aspect, a first nucleic acid sequence and a second nucleic acid sequence are provided in a single vector. In another aspect, a first nucleic acid sequence is provided in a first vector, and a second nucleic acid sequence is provided in a second vector.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Without being limiting, nucleic acids can be detected using hybridization. Hybridization between nucleic acids is discussed in detail in Sambrook et. al. (1989, Molecular Cloning: A Laboratory Manual, 2nd Ed., Cold Spring Harbor Laboratory Press, Cold Spring Harbor, NY).

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

The terms “percent sequence complementarity” or “percent complementarity” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

For optimal alignment of sequences to calculate their percent identity, various pair-wise or multiple sequence alignment algorithms and programs are known in the art, such as ClustalW or Basic Local Alignment Search Tool (BLAST®), etc., that can be used to compare the sequence identity or similarity between two or more nucleotide or protein sequences. Although other alignment and comparison methods are known in the art, the alignment and percent identity between two sequences (including the percent identity ranges described above) can be as determined by the ClustalW algorithm, see, e.g., Chenna R. et. al., “Multiple sequence alignment with the Clustal series of programs,” Nucleic Acids Research 31: 3497-3500 (2003); Thompson J D et. al., “Clustal W: Improving the sensitivity of progressive multiple sequence alignment through sequence weighting, position-specific gap penalties and weight matrix choice,” Nucleic Acids Research 22: 4673-4680 (1994); Larkin M A et. al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

As used herein, a first nucleic acid molecule can “hybridize” a second nucleic acid molecule via non-covalent interactions (e.g., Watson-Crick base-pairing) in a sequence-specific, antiparallel manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymine (T), adenine (A) pairing with uracil (U), and guanine (G) pairing with cytosine (C). In addition, it is also known in the art that for hybridization between two RNA molecules (e.g., dsRNA), guanine base pairs with uracil. For example, G/U base-pairing is partially responsible for the degeneracy (i.e., redundancy) of the genetic code in the context of tRNA anti-codon base-pairing with codons in mRNA. In the context of this disclosure, a guanine of a protein-binding segment (dsRNA duplex) of a subject DNA-targeting RNA molecule is considered complementary to an uracil, and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et. al.). Typically, the length for a hybridizable nucleic acid is at least 10 nucleotides. Illustrative minimum lengths for a hybridizable nucleic acid are: at least 15 nucleotides; at least 18 nucleotides; at least 20 nucleotides; at least 22 nucleotides; at least 25 nucleotides; and at least 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST® programs (basic local alignment search tools) and PowerBLAST programs known in the art (see Altschul et. al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Generating Edits

Several embodiments described herein relate to compositions and methods for producing heritable edits in the genome of a progeny plant by expressing a DNA modification enzyme, for example a guided nuclease, preferentially in egg, meiotic, and/or embryonic cells of a parent plant. In some embodiments, the parent plant expressing the DNA modification enzyme is female. In some embodiments, the parent plant expressing the DNA modification enzyme is male.

As used herein, the term “genome editing” or “editing” refers to any modification of a nucleotide sequence in a site-specific manner. In the present disclosure genome editing techniques include the use of DNA modification enzymes, such as endonucleases, recombinases, transposases, deaminases, methylases, helicases and any combination thereof. In an aspect, a “modification” comprises the hydrolytic deamination of cytidine or deoxycytidine to uridine or deoxyuridine, respectively. In some embodiments, a sequence-specific editing system comprises an adenine deaminase. In an aspect, a “modification” comprises the hydrolytic deamination of adenine or adenosine. In an aspect, a “modification” comprises the hydrolytic deamination of adenosine or deoxyadenosine to inosine or deoxyinosine, respectively. In an aspect, a “modification” comprises the insertion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In some embodiments, a “modification” comprises the insertion of one or more transgenes. In another aspect, a “modification” comprises the deletion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In a further aspect, a “modification” comprises the inversion of at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In still another aspect, a “modification” comprises the substitution of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In still another aspect, a “modification” comprises the duplication of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In some embodiments, a “modification” comprises the substitution of an “A” for a “C”, “G” or “T” in a nucleic acid sequence. In some embodiments, a “modification” comprises the substitution of an “C” for a “A”, “G” or “T” in a nucleic acid sequence. In some embodiments, a “modification” comprises the substitution of an “G” for a “A”, “C” or “T” in a nucleic acid sequence. In some embodiments, a “modification” comprises the substitution of an “T” for a “A”, “C” or “G” in a nucleic acid sequence. In some embodiments, a “modification” comprises the substitution of an “C” for a “U” in a nucleic acid sequence. In some embodiments, a “modification” comprises the substitution of an “G” for a “A” in a nucleic acid sequence. In some embodiments, a “modification” comprises the substitution of an “A” for a “G” in a nucleic acid sequence. In some embodiments, a “modification” comprises the substitution of an “T” for a “C” in a nucleic acid sequence.

In an aspect, this disclosure provides a method of editing a genome of a plant comprising: (a) introducing to a plant cell: (i) a first nucleic acid sequence encoding a guided nuclease capable of generating a staggered cut in a double-stranded DNA molecule operably linked to a heterologous meiosis-preferred promoter; and (b) a second nucleic acid sequence encoding at least one guide nucleic acid operably linked to a heterologous second promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one meiotic cell of the plant, and where the ribonucleoprotein generates at least one double-stranded break within the target sequence in the at least one meiotic cell.

In an aspect, this disclosure provides a method of editing a genome of a plant comprising: (a) introducing to a plant cell: (i) a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to a heterologous meiosis-preferred promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one meiotic cell of the plant, and where the ribonucleoprotein generates at least one double-stranded break within the target sequence in the at least one meiotic cell.

In an aspect, this disclosure provides a method of editing a genome of a plant comprising: (a) introducing to a plant cell: (i) a first nucleic acid sequence encoding a guided nuclease capable of generating a staggered cut in a double-stranded DNA molecule operably linked to a heterologous egg cell-preferred promoter; and (b) a second nucleic acid sequence encoding at least one guide nucleic acid operably linked to a heterologous second promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one egg cell of the plant, and where the ribonucleoprotein generates at least one double-stranded break within the target sequence in the at least one egg cell.

In an aspect, this disclosure provides a method of editing a genome of a plant comprising: (a) introducing to a plant cell: (i) a first nucleic acid sequence encoding a guided nuclease capable of generating a staggered cut in a double-stranded DNA molecule operably linked to a heterologous promoter; and (b) a second nucleic acid sequence encoding at least one guide nucleic acid operably linked to an egg cell-preferred promoter, where the at least one guide nucleic acid is capable of hybridizing to a target sequence within the genome; and (b) regenerating at least one plant from the plant cell of step (a), where the guided nuclease and at least one guide nucleic acid form a ribonucleoprotein within at least one egg cell of the plant, and where the ribonucleoprotein generates at least one double-stranded break within the target sequence in the at least one egg cell.

In an aspect, this disclosure provides a method of editing a genome of a plant cell comprising: (a) crossing a first plant with a second plant, where the first plant comprises a first nucleic acid sequence encoding a guided nuclease capable of generating a staggered cut in a double-stranded DNA molecule operably linked to a heterologous embryo tissue-preferred promoter, and where the second plant comprises a second nucleic acid sequence encoding at least one guide nucleic acid operably linked to a heterologous second promoter, wherein the at least one guide nucleic acid is capable of hybridizing to a target sequence within the genome; and (b) obtaining at least one embryo from the crossing of step (a), where the guided nuclease and the at least one guide nucleic acid form a ribonucleoprotein within the at least one embryo, and where the ribonucleoprotein generates at least one double-stranded break within the target sequence in the at least one embryo.

In an aspect, this disclosure provides a method of editing a genome of a plant cell comprising: (a) crossing a first plant with a second plant, where the first plant comprises a first nucleic acid sequence encoding a guided nuclease capable of generating a staggered cut in a double-stranded DNA molecule operably linked to a heterologous promoter, and where the second plant comprises a second nucleic acid sequence encoding at least one guide nucleic acid operably linked to an embryo tissue-preferred promoter, wherein the at least one guide nucleic acid is capable of hybridizing to a target sequence within the genome; and (b) obtaining at least one embryo from the crossing of step (a), where the guided nuclease and the at least one guide nucleic acid form a ribonucleoprotein within the at least one embryo, and where the ribonucleoprotein generates at least one double-stranded break within the target sequence in the at least one embryo.

In an aspect, this disclosure provides a method of editing a genome of a plant comprising: (a) introducing to a plant cell: (i) a first nucleic acid sequence encoding a guided nuclease capable of generating a staggered cut in a double-stranded DNA molecule operably linked to a heterologous embryo tissue-preferred promoter; and (ii) a second nucleic acid sequence encoding at least one guide nucleic acid operably linked to a heterologous second promoter, where the at least one guide nucleic acid is capable of hybridizing to a target sequence within the genome; (b) regenerating at least one plant from the plant cell of step (a); and (c) fertilizing the at least one plant to create at least one embryo, where the guided nuclease and at least one guide nucleic acid form a ribonucleoprotein within the at least one embryo from step (c), and where the ribonucleoprotein generates at least one double-stranded break within the target sequence in the at least one embryo.

In an aspect, this disclosure provides a method of editing a genome of a plant comprising: (a) introducing to a plant cell: (i) a first nucleic acid sequence encoding a guided nuclease capable of generating a staggered cut in a double-stranded DNA molecule operably linked to a heterologous promoter; and (ii) a second nucleic acid sequence encoding at least one guide nucleic acid operably linked to an embryo tissue-preferred promoter, where the at least one guide nucleic acid is capable of hybridizing to a target sequence within the genome; (b) regenerating at least one plant from the plant cell of step (a); and (c) fertilizing the at least one plant to create at least one embryo, where the guided nuclease and at least one guide nucleic acid form a ribonucleoprotein within the at least one embryo from step (c), and where the ribonucleoprotein generates at least one double-stranded break within the target sequence in the at least one embryo.

In an aspect, this disclosure provides a method of generating a site-directed integration in a plant comprising: (a) introducing to a plant cell: (i) a first nucleic acid sequence encoding a guided nuclease capable of generating a staggered cut in a double-stranded DNA molecule operably linked to a heterologous meiosis-preferred promoter; and (ii) a second nucleic acid sequence encoding one or more guide nucleic acids operably linked to a heterologous second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); where the guided nuclease and at least one guide RNA form a ribonucleoprotein within at least one meiotic cell of the plant, where the ribonucleoprotein generates a double-stranded break within the target sequence, the first site, and the second site, and where the gene of interest is integrated into the target site in the at least one egg cell.

In an aspect, this disclosure provides a method of generating a site-directed integration in a plant comprising: (a) introducing to a plant cell: (i) a first nucleic acid sequence encoding a guided nuclease capable of generating a staggered cut in a double-stranded DNA molecule operably linked to a heterologous promoter; and (ii) a second nucleic acid sequence encoding one or more guide nucleic acids operably linked to a meiosis-preferred second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); where the guided nuclease and at least one guide RNA form a ribonucleoprotein within at least one meiotic cell of the plant, where the ribonucleoprotein generates a double-stranded break within the target sequence, the first site, and the second site, and where the gene of interest is integrated into the target site in the at least one meiotic cell.

In an aspect, this disclosure provides a method of generating a site-directed integration in a plant comprising: (a) introducing to a plant cell: (i) a first nucleic acid sequence encoding a guided nuclease capable of generating a staggered cut in a double-stranded DNA molecule operably linked to a heterologous egg cell-preferred promoter; and (ii) a second nucleic acid sequence encoding one or more guide nucleic acids operably linked to a heterologous second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); where the guided nuclease and at least one guide RNA form a ribonucleoprotein within at least one egg cell of the plant, where the ribonucleoprotein generates a double-stranded break within the target sequence, the first site, and the second site, and where the gene of interest is integrated into the target site in the at least one egg cell.

In an aspect, this disclosure provides a method of generating a site-directed integration in a plant comprising: (a) introducing to a plant cell: (i) a first nucleic acid sequence encoding a guided nuclease capable of generating a staggered cut in a double-stranded DNA molecule operably linked to a heterologous promoter; and (ii) a second nucleic acid sequence encoding one or more guide nucleic acids operably linked to an egg cell-preferred second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); where the guided nuclease and at least one guide RNA form a ribonucleoprotein within at least one egg cell of the plant, where the ribonucleoprotein generates a double-stranded break within the target sequence, the first site, and the second site, and where the gene of interest is integrated into the target site in the at least one egg cell.

In an aspect, this disclosure provides a method of generating a site-directed integration in a plant comprising: (a) introducing to a plant cell: (i) a first nucleic acid sequence encoding a guided nuclease capable of generating a staggered cut in a double-stranded DNA molecule operably linked to a heterologous embryo tissue-preferred promoter; (ii) a second nucleic acid sequence encoding one or more guide nucleic acids operably linked to a heterologous second promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); and (c) fertilizing the at least one plant from step (b) to create at least one embryo; where the guided nuclease and at least one guide RNA form a ribonucleoprotein within at least one embryo, where the ribonucleoprotein generates a double-stranded break within the target sequence, the first site, and the second site, and where the gene of interest is integrated into the target site in the at least one embryo.

In an aspect, this disclosure provides a method of generating a site-directed integration in a plant comprising: (a) introducing to a plant cell: (i) a first nucleic acid sequence encoding a guided nuclease capable of generating a staggered cut in a double-stranded DNA molecule operably linked to a heterologous promoter; (ii) a second nucleic acid sequence encoding one or more guide nucleic acids operably linked to an embryo tissue-preferred promoter, wherein the one or more guide nucleic acids are (A) capable of hybridizing to a target sequence within a genome of the plant; and (B) capable of hybridizing to a first site and a second site flanking a nucleic acid sequence encoding a gene of interest; and (iii) a third nucleic acid sequence encoding the gene of interest; (b) regenerating at least one plant from the plant cell of step (a); and (c) fertilizing the at least one plant from step (b) to create at least one embryo; where the guided nuclease and at least one guide RNA form a ribonucleoprotein within at least one embryo, where the ribonucleoprotein generates a double-stranded break within the target sequence, the first site, and the second site, and where the gene of interest is integrated into the target site in the at least one embryo.

### Regulatory Elements

Regulatory elements such as promoters, leaders (also known as 5′ UTRs), enhancers, introns, and transcription termination regions (or 3′ UTRs) play an integral part in the overall expression of genes in living cells. The term “regulatory element,” as used herein, refers to a DNA molecule having gene-regulatory activity. The term “gene-regulatory activity,” as used herein, refers to the ability to affect the expression of an operably linked transcribable DNA molecule, for instance by affecting the transcription and/or translation of the operably linked transcribable DNA molecule. Regulatory elements, such as promoters, leaders, enhancers, introns and 3′ UTRs that function in plants are useful for modifying plant phenotypes through genetic engineering. Regulatory elements may be characterized by their gene expression pattern, e.g., positive and/or negative effects such as constitutive expression or temporal, spatial, developmental, tissue, environmental, physiological, pathological, cell cycle, and/or chemically responsive expression, and any combination thereof, as well as by quantitative or qualitative indications. As used herein, a “gene expression pattern” is any pattern of transcription of an operably linked DNA molecule into a transcribed RNA molecule. The transcribed RNA molecule may be translated to produce a protein molecule or may provide an antisense or other regulatory RNA molecule, such as a double-stranded RNA (dsRNA), a transfer RNA (tRNA), a ribosomal RNA (rRNA), a microRNA (miRNA), a small interfering RNA (siRNA), and the like. As used herein, the term “protein expression” is any pattern of translation of a transcribed RNA molecule into a protein molecule. Protein expression may be characterized by its temporal, spatial, developmental, or morphological qualities, as well as by quantitative or qualitative indications.

As commonly understood in the art, the term “promoter” refers to a DNA sequence that contains an RNA polymerase binding site, transcription start site, and/or TATA box and assists or promotes the transcription and expression of an associated transcribable polynucleotide sequence and/or gene (or transgene). A promoter may be initially isolated from the 5′ untranslated region (5′ UTR) of a genomic copy of a gene. A promoter can be synthetically produced, varied or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein. A promoter can be classified according to a variety of criteria relating to the pattern of expression of an associated coding or transcribable sequence or gene (including a transgene) operably linked to the promoter, such as constitutive, developmental, tissue-specific, cell cycle-specific, inducible, etc.

In some embodiments, a promoter is operably linked 5′ to a leader sequence. As used herein, the term “leader” refers to a DNA molecule isolated from the untranslated 5′ region (5′ UTR) a gene and defined generally as a nucleotide segment between the transcription start site (TSS) and the protein coding sequence start site. Alternately, leaders may be synthetically produced or manipulated DNA elements. A leader can be used as a 5′ regulatory element for modulating expression of an operably linked transcribable DNA molecule. Leader molecules may be used with a heterologous promoter or with their native promoter.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous.

Promoters that express within a specific tissue(s) of an organism, with no expression in other tissues, are referred to as “tissue-specific” promoters. Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of a plant, but with lower levels of expression in other tissue(s) of the plant. In another aspect, a promoter provided herein is a tissue-specific promoter. In a further aspect, a promoter provided herein is a tissue-preferred promoter. In an aspect, a tissue-preferred promoter comprises a tissue-specific promoter.

Promoters that express within a meiotic cell(s) of an organism, with no expression in non-meiotic cells, are referred to as “meiotic cell-specific” or “meiosis-specific” promoters. Promoters that drive enhanced expression in meiotic cells of an organism relative to other cells of the organism are referred to as “meiotic cell-preferred” or “meiosis-preferred” promoters. Thus, a “meiotic cell-preferred” or “meiosis-preferred” promoter causes relatively higher or preferential expression in cells of a plant undergoing meiosis, but with lower levels of expression in other cell(s) of the plant. In another aspect, a promoter provided herein is a meiosis-specific promoter. In a further aspect, a promoter provided herein is a meiosis-preferred promoter. In an aspect, a meiosis-preferred promoter comprises a meiosis-specific promoter. Promoters that express in a cell cycle dependent manner, are referred to as “cell cycle-specific” promoters. In another aspect, a promoter provided herein is a cell cycle-specific promoter. In a further aspect, a promoter provided herein is a cell cycle-preferred promoter. In an aspect, a cell cycle-preferred promoter comprises a cell cycle-specific promoter.

Determination of promoter activity can be performed using any method standard in the art. For example, without being limiting, a promoter of interest can be used to drive expression of a fluorophore or other reporting molecule, and the concentration of the expressed molecule can be used to determine promoter activity in different cell or tissue types.

Several embodiments described herein relate to expression of a DNA modification enzyme, for example a guided nuclease, preferentially in egg, meiotic, and/or embryonic cells of a plant. Nonlimiting examples of expression elements useful in the composition and methods described herein are provided in Table 1.

TABLE 1
Plant Egg, Embryo, and Meiotic Cell Promoters

| | | | Promoter | 3′UTR |
|---|---|---|---|---|
| | | | and leader | SEQ |
| | | Expected | SEQ | ID |
| Source | Gene name/ID | Expression | ID NO: | NO: |
| Corn | ZmDSUL 1 | Embryo | 1 | — |
| Corn | ZmEA1 | Egg/Embryo | 2 | — |
| Corn | ZmES4 | Egg/Embryo | 3 | — |
| Corn | ZmDMC1 | Meiocyte | 4 | — |
| Corn | ZmMps1 | Meiocyte | 5 | — |
| Corn | ZmAdf1 | Meiocyte | 6 | — |
| Corn | GRMZM2G141762 | Ovary | 21 | — |
| | | (including Egg) | | |
| Corn | A1ZM025370 | Egg | 22 | — |
| Corn | GRMZM2G119150 | Ovary | 23 | — |
| | | (including Egg) | | |
| Corn | Zm.AC185611 | Egg and zygote | 24 | 46 |
| Corn | GRMZM2G025133 | Zygote and egg | 25 | 47 |
| Corn | GRMZM2G103251 | Zygote | 26 | 48 |
| Corn | P-Zm.EAL1:1 | Egg | 27 | — |
| | GRMZM2G456746 | | | |
| Corn | P-ZmES2/3 | Egg and zygote | 28 | — |
| | GRMZM2G128301 | | | |
| Corn | GRMZM2G466856 | Egg and | 29 | 49 |
| | | Embryo | | |
| Corn | GRMZM2G075386 | Egg and | 30 | 50 |
| | | Embryo | | |
| Corn | GRMZM2G466848 | Egg and | 31 | 51 |
| | | Embryo | | |
| Corn | GRMZM2G047842 | Egg and | 32 | 52 |
| | | Embryo | | |
| Corn | GRMZM2G025720 | Egg and | 33 | 53 |
| | | Embryo | | |
| Corn | GRMZM2G083190 | Egg and | 34 | 54 |
| | | Embryo | | |
| Corn | GRMZM2G536120 | Egg and | 35 | 55 |
| | | Embryo | | |
| Corn | GRMZM2G125162 | Egg and | 36 | 56 |
| | | Embryo | | |
| Corn | AC211413.4_FG001 | Egg and | 37 | 57 |
| | | Embryo | | |
| Corn | GRMZM2G457612 | Egg and | 38 | — |
| | | Embryo | | |
| Corn | GRMZM2G328205 | Embryo | 39 | 58 |
| Corn | GRMZM2G337139 | Embryo | 40 | 59 |
| Corn | AC215302.3_FG001 | Egg specific | 41 | 60 |
| Corn | GRMZM2G417287 | Egg specific | 42 | 61 |
| Corn | GRMZM2G103251 | Egg and | 43 | 62 |
| | | Embryo | | |
| Corn | GRMZM2G035685 | Egg and | 44 | 63 |
| | | Embryo | | |
| Corn | GRMZM2G150827 | Egg and | 45 | 64 |
| | | Embryo | | |
| Soy | Glyma.05G128300 | Ovule | 65 | 70 |
| Soy | Glyma.03G037900 | Ovule | 66 | 71 |
| Soy | Glyma.04G248800 | Ovule | 67 | 72 |
| Soy | Glyma.04G090700 | Ovule | 68 | 73 |
| Soy | Glyma.07G050200 | Ovule | 69 | 74 |
| Arabidopsis | Yao | Egg | 75 | 89 |
| | AT4G05410 | | | |
| Arabidopsis | EC1.1 | Egg | 76 | 90 |
| | AT1G76750 | | | |
| Arabidopsis | DD45 | Egg | 77 | 91 |
| | AT2G21740 | | | |
| Arabidopsis | DD33 | Egg | 78 | 92 |
| | AT2G20070 | | | |
| Arabidopsis | AtP5p | Egg | 79 | 93 |
| | AT1G71470 | | | |
| Arabidopsis | EC1.3 | Egg | 80 | 94 |
| | AT2G21750 | | | |
| Arabidopsis | EC1.4 | Egg | 81 | 95 |
| | AT4G39340 | | | |
| Arabidopsis | EC1.5 | Egg | 82 | 96 |
| | AT5G64720 | | | |
| Arabidopsis | CDC45 | Meiotic | 83 | 97 |
| | AT3G25100 | | | |
| Arabidopsis | MGE1p | Meiotic | 84 | 98 |
| | AT4G40020 | | | |
| Arabidopsis | MGE2p | Meiotic | 85 | 99 |
| | AT4G20900 | | | |
| Arabidopsis | WOX2 | Early embryo | 86 | 100 |
| | AT5G59340 | | | |
| Arabidopsis | F17L21.26 | Early embryo | 87 | 101 |
| | AT1G27470 | | | |
| Arabidopsis | POLA3 | Early embryo | 88 | 102 |
| | AT5G41880 | | (Promoter + | |
| | | | 5′UTR | |
| | | | intron + | |
| | | | leader) | |

In one embodiment, fragments of a promoter sequence disclosed in Table 1 are provided. Promoter fragments may comprise egg, embryo, and/or meiotic expression activity, as described above, and may be useful alone or in combination with other promoters and promoter fragments, such as in constructing chimeric promoters, or in combination with other expression elements and expression element fragments. In some embodiments, fragments of a promoter are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule having promoter activity as disclosed herein. In some embodiments, fragments of a promoter are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, at least about 1000, at least about 1050, at least about 1100, or at least about 1150 contiguous nucleotides, of a DNA sequence comprising a TATA box and having at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, at least about 99 percent identity, or at least about 100 percent identity to SEQ ID NO. 1-6, 21-45, 65-69 or 75-88, having promoter activity as disclosed herein. Methods for producing such fragments from a starting promoter molecule are well known in the art.

In an aspect, a meiosis cell-preferred or meiosis-preferred promoter comprises a DMC1 promoter. In an aspect, a meiotic cell-preferred or meiosis-preferred promoter comprises an Mps1 promoter. In an aspect, a meiotic cell-preferred or meiosis-preferred promoter comprises an Adf1 promoter. In an aspect, a meiotic cell-preferred or meiosis-preferred promoter comprises a promoter selected from the group consisting of a DMC1 promoter, a Mps1 promoter, and an Adf1 promoter.

In an aspect, an expression element described in Table 1 is operably linked to a nucleic acid encoding a DNA modification enzyme, such as a guided nuclease. In an aspect, an expression element described in Table 1 is operably linked to a nucleic acid encoding a Cas9 nuclease. In an aspect, an expression element described in Table 1 is operably linked to a nucleic acid encoding a Cas12a nuclease. In an aspect, an expression element described in Table 1 is operably linked to a nucleic acid encoding a CasX nuclease. In an aspect, an expression element described in Table 1 is operably linked to a nucleic acid encoding a guide nucleic acid. In an aspect, an expression element described in Table 1 is operably linked to a nucleic acid encoding a guide RNA. In an aspect, an expression element described in Table 1 is operably linked to a nucleic acid encoding a single-guide RNA. In some embodiments, the guide RNA acid is flanked by self-cleaving ribozymes. In an aspect, an expression element described in Table 1 is operably linked to a nucleic acid encoding a recombinase (e.g., Cre recombinase). In an aspect, an expression element described in Table 1 is operably linked to a nucleic acid encoding a TALE.

In an aspect, a DMC1 promoter is operably linked to a nucleic acid encoding a guided nuclease. In an aspect, a DMC1 promoter is operably linked to a nucleic acid encoding a Cas9 nuclease. In an aspect, a DMC1 promoter is operably linked to a nucleic acid encoding a Cas12a nuclease. In an aspect, a DMC1 promoter is operably linked to a nucleic acid encoding a CasX nuclease. In an aspect, a DMC1 promoter is operably linked to a nucleic acid encoding a guide nucleic acid. In an aspect, a DMC1 promoter is operably linked to a nucleic acid encoding a guide RNA. In an aspect, a DMC1 promoter is operably linked to a nucleic acid encoding a single-guide RNA. In some embodiments, the guide RNA acid is flanked by self-cleaving ribozymes. In an aspect, a DMC1 promoter is operably linked to a nucleic acid encoding a recombinase (e.g., Cre recombinase). In an aspect, a DMC1 promoter is operably linked to a nucleic acid encoding a TALE.

In an aspect, a Mps1 promoter is operably linked to a nucleic acid encoding a guided nuclease. In an aspect, a Mps1 promoter is operably linked to a nucleic acid encoding a Cas9 nuclease. In an aspect, a Mps1 promoter is operably linked to a nucleic acid encoding a Cas12a nuclease. In an aspect, a Mps1 promoter is operably linked to a nucleic acid encoding a CasX nuclease. In an aspect, a Mps1 promoter is operably linked to a nucleic acid encoding a guide nucleic acid. In an aspect, a Mps1 promoter is operably linked to a nucleic acid encoding a guide RNA. In an aspect, a Mps1 promoter is operably linked to a nucleic acid encoding a single-guide RNA. In some embodiments, the guide RNA acid is flanked by self-cleaving ribozymes. In an aspect, a Mps1 promoter is operably linked to a nucleic acid encoding a recombinase (e.g., Cre recombinase). In an aspect, a Mps1 promoter is operably linked to a nucleic acid encoding a TALE.

In an aspect, an Adf1 promoter is operably linked to a nucleic acid encoding a guided nuclease. In an aspect, an Adf1 promoter is operably linked to a nucleic acid encoding a Cas9 nuclease. In an aspect, an Adf1 promoter is operably linked to a nucleic acid encoding a Cas12a nuclease. In an aspect, an Adf1 promoter is operably linked to a nucleic acid encoding a CasX nuclease. In an aspect, an Adf1 promoter is operably linked to a nucleic acid encoding a guide nucleic acid. In an aspect, an Adf1 promoter is operably linked to a nucleic acid encoding a guide RNA. In an aspect, an Adf1 promoter is operably linked to a nucleic acid encoding a single-guide RNA. In some embodiments, the guide RNA acid is flanked by self-cleaving ribozymes. In an aspect, an Adf1 promoter is operably linked to a nucleic acid encoding a recombinase (e.g., Cre recombinase). In an aspect, an Adf1 promoter is operably linked to a nucleic acid encoding a TALE.

In an aspect, a DMC1 promoter comprises a nucleic acid sequence at least 80% identical to SEQ ID NO: 4, or a functional fragment thereof. In an aspect, a DMC1 promoter comprises a nucleic acid sequence at least 85% identical to SEQ ID NO: 4, or a functional fragment thereof. In an aspect, a DMC1 promoter comprises a nucleic acid sequence at least 90% identical to SEQ ID NO: 4, or a functional fragment thereof. In an aspect, a DMC1 promoter comprises a nucleic acid sequence at least 95% identical to SEQ ID NO: 4, or a functional fragment thereof. In an aspect, a DMC1 promoter comprises a nucleic acid sequence at least 96% identical to SEQ ID NO: 4, or a functional fragment thereof. In an aspect, a DMC1 promoter comprises a nucleic acid sequence at least 97% identical to SEQ ID NO: 4, or a functional fragment thereof. In an aspect, a DMC1 promoter comprises a nucleic acid sequence at least 98% identical to SEQ ID NO: 4, or a functional fragment thereof. In an aspect, a DMC1 promoter comprises a nucleic acid sequence at least 99% identical to SEQ ID NO: 4, or a functional fragment thereof. In an aspect, a DMC1 promoter comprises a nucleic acid sequence 100% identical to SEQ ID NO: 4, or a functional fragment thereof.

In an aspect, a Mps1 promoter comprises a nucleic acid sequence at least 80% identical to SEQ ID NO: 5, or a functional fragment thereof. In an aspect, a Mps1 promoter comprises a nucleic acid sequence at least 85% identical to SEQ ID NO: 5, or a functional fragment thereof. In an aspect, a Mps1 promoter comprises a nucleic acid sequence at least 90% identical to SEQ ID NO: 5, or a functional fragment thereof. In an aspect, a Mps1 promoter comprises a nucleic acid sequence at least 95% identical to SEQ ID NO: 5, or a functional fragment thereof. In an aspect, a Mps1 promoter comprises a nucleic acid sequence at least 96% identical to SEQ ID NO: 5, or a functional fragment thereof. In an aspect, a Mps1 promoter comprises a nucleic acid sequence at least 97% identical to SEQ ID NO: 5, or a functional fragment thereof. In an aspect, a Mps1 promoter comprises a nucleic acid sequence at least 98% identical to SEQ ID NO: 5, or a functional fragment thereof. In an aspect, a Mps1 promoter comprises a nucleic acid sequence at least 99% identical to SEQ ID NO:5, or a functional fragment thereof. In an aspect, a Mps1 promoter comprises a nucleic acid sequence 100% identical to SEQ ID NO: 5, or a functional fragment thereof.

In an aspect, an Adf1 promoter comprises a nucleic acid sequence at least 80% identical to SEQ ID NO: 6, or a functional fragment thereof. In an aspect, an Adf1 promoter comprises a nucleic acid sequence at least 85% identical to SEQ ID NO: 6, or a functional fragment thereof. In an aspect, an Adf1 promoter comprises a nucleic acid sequence at least 90% identical to SEQ ID NO: 6, or a functional fragment thereof. In an aspect, an Adf1 promoter comprises a nucleic acid sequence at least 95% identical to SEQ ID NO: 6, or a functional fragment thereof. In an aspect, an Adf1 promoter comprises a nucleic acid sequence at least 96% identical to SEQ ID NO: 6, or a functional fragment thereof. In an aspect, an Adf1 promoter comprises a nucleic acid sequence at least 97% identical to SEQ ID NO: 6, or a functional fragment thereof. In an aspect, an Adf1 promoter comprises a nucleic acid sequence at least 98% identical to SEQ ID NO: 6, or a functional fragment thereof. In an aspect, an Adf1 promoter comprises a nucleic acid sequence at least 99% identical to SEQ ID NO: 6, or a functional fragment thereof. In an aspect, an Adf1 promoter comprises a nucleic acid sequence 100% identical to SEQ ID NO: 6, or a functional fragment thereof.

In an aspect, a meiocyte-preferred promoter comprises a nucleic acid sequence at least 80% identical to a nucleic acid sequence selected from the group consisting of SEQ ID NOs: 4-6, 83-85 or a functional fragment thereof. In an aspect, a meiocyte-preferred promoter comprises a nucleic acid sequence at least 85% identical to a nucleic acid sequence selected from the group consisting of SEQ ID NOs: 4-6, 83-85 or a functional fragment thereof. In an aspect, a meiocyte-preferred promoter comprises a nucleic acid sequence at least 90% identical to a nucleic acid sequence selected from the group consisting of SEQ ID NOs: 4-6, 83-85 or a functional fragment thereof. In an aspect, a meiocyte-preferred promoter comprises a nucleic acid sequence at least 95% identical to a nucleic acid sequence selected from the group consisting of SEQ ID NOs: 4-6, 83-85 or a functional fragment thereof. In an aspect, a meiocyte-preferred promoter comprises a nucleic acid sequence at least 96% identical to a nucleic acid sequence selected from the group consisting of SEQ ID NOs: 4-6, 83-85 or a functional fragment thereof. In an aspect, a meiocyte-preferred promoter comprises a nucleic acid sequence at least 97% identical to a nucleic acid sequence selected from the group consisting of SEQ ID NOs: 4-6, 83-85 or a functional fragment thereof. In an aspect, a meiocyte-preferred promoter comprises a nucleic acid sequence at least 98% identical to a nucleic acid sequence selected from the group consisting of SEQ ID NOs: 4-6, 83-85 or a functional fragment thereof. In an aspect, a meiocyte-preferred promoter comprises a nucleic acid sequence at least 99% identical to a nucleic acid sequence selected from the group consisting of SEQ ID NOs: 4-6, 83-85 or a functional fragment thereof. In an aspect, a meiocyte-preferred promoter comprises a nucleic acid sequence 100% identical to a nucleic acid sequence selected from the group consisting of SEQ ID NOs: 4-6, 83-85 or a functional fragment thereof.

As used herein, an “egg cell-preferred promoter” refers to a promoter that exhibits higher or preferential expression in an egg cell as compared to other cell or tissue types of the plant. Egg cell-preferred promoters can exhibit expression in nearby cells such as, without being limiting, synergids, antipodal cells, central cells, integument cells, stigma cells, and style cells. An egg cell-preferred promoter can also exhibit expression in other ovary cells, for example the ovule. An egg cell-preferred promoter can also exhibit expression in other plant tissues, such as, without being limiting, pollen cells, root cells, embryo cells, stem cells, meristem cells, floral cells, and leaf cells, as long as the egg cell-preferred promoter exhibits higher or preferential expression in an egg cell.

As used herein, an “egg cell-specific promoter” refers to a promoter that exhibits expression exclusively in an egg cell. In an aspect, an egg cell-preferred promoter comprises an egg cell-specific promoter.

As used herein, an “ovule tissue preferred promoter” refers to a promoter that exhibits higher or preferential expression in at least one or all of the ovule tissue as compared to other cell or tissue types of the plant. In seed plants, the ovule is the structure that gives rise to and contains the female reproductive cells. As used herein, the ovule is initially composed of unreduced tissue that gives rise to the haploid tissue of the female gametophyte. The female gametophyte further develops into the “mature egg sac”, comprised of four unique cell types: one egg cell, a central cell, two synergids and three or more antipodal cells. As used herein, the ovule preferred promoter can exhibit expression in pre or post pollinated ovule. An ovule preferred promoter can also exhibit expression in other ovary cells.

As used herein, an “ovule tissue-specific promoter” refers to a promoter that exhibits expression exclusively in ovule. In an aspect, an ovule tissue-preferred promoter comprises an ovule tissue-specific promoter.

As used herein, an “embryo tissue-preferred promoter” refers to a promoter that exhibits higher or preferential expression in embryo tissue as compared to other cell or tissue types of the plant. Embryo tissue-preferred promoters can exhibit expression in nearby cells such as, without being limiting, endosperm cells, cotyledon cells, and seed coat cells. An embryo tissue-preferred promoter can also exhibit expression in other plant tissues, such as, without being limiting, pollen cells, root cells, egg cells, stem cells, meristem cells, floral cells, and leaf cells, as long as the embryo tissue-preferred promoter exhibits higher or preferential expression in embryo tissue.

As used herein, an “embryo tissue-specific promoter” refers to a promoter that exhibits expression exclusively in embryo tissue. In an aspect, an embryo tissue-preferred promoter comprises an embryo tissue-specific promoter.

As used herein, an “zygote cell-preferred promoter” refers to a promoter that exhibits higher or preferential expression in zygotes as compared to other cell or tissue types of the plant. Upon fertilization of an egg cell by a haploid pollen cell, a diploid zygote is formed, which gives rise to an embryo. A zygote cell-preferred promoter can also exhibit expression in other plant cells, such as, without being limiting, pollen cells, egg cells, stem cells, meristem cells, endosperm cells, cotyledon cells, floral cells, leaf cells and embryo tissue as long as the zygote tissue-preferred promoter exhibits higher or preferential expression in the zygote.

As used herein, an “zygote cell-specific promoter” refers to a promoter that exhibits expression exclusively in the zygote. In an aspect, a zygote cell-preferred promoter comprises a zygote cell-specific promoter.

It will be appreciated that the same promoter can be both egg cell-preferred, zygote cell-preferred and embryo-tissue preferred, as a fertilized egg upon pollination will develop into a zygote which will give rise to embryo tissue.

In an aspect, an embryo tissue-preferred or embryo tissue-specific promoter comprises a DSUL1 promoter. In an aspect, an egg cell-preferred or embryo tissue-preferred promoter comprises an EA1 promoter. In an aspect, an egg cell-preferred or embryo tissue-preferred promoter comprises an ES4 promoter. In an aspect, an egg cell-preferred or embryo tissue-preferred promoter comprises an EAL1 promoter. In an aspect, an egg cell-preferred or embryo tissue-preferred promoter comprises a promoter selected from the group consisting of a DSUL1 promoter, an EA1 promoter, an ES4 promoter, and an EAL1 promoter.

In an aspect, a DSUL1 promoter is operably linked to a nucleic acid encoding a guided nuclease. In an aspect, a DSUL1 promoter is operably linked to a nucleic acid encoding a Cas9 nuclease. In an aspect, a DSUL1 promoter is operably linked to a nucleic acid encoding a Cas12a nuclease. In an aspect, a DSUL1 promoter is operably linked to a nucleic acid encoding a CasX nuclease. In an aspect, a DSUL1 promoter is operably linked to a nucleic acid encoding a guide nucleic acid. In an aspect, a DSUL1 promoter is operably linked to a nucleic acid encoding a guide RNA. In an aspect, a DSUL1 promoter is operably linked to a nucleic acid encoding a single-guide RNA. In some embodiments, the guide RNA acid is flanked by self-cleaving ribozymes. In an aspect, a DSUL1 promoter is operably linked to a nucleic acid encoding a recombinase (e.g., Cre recombinase). In an aspect, a DSUL1 promoter is operably linked to a nucleic acid encoding a TALE.

In an aspect, an EA1 promoter is operably linked to a nucleic acid encoding a guided nuclease. In an aspect, an EA1 promoter is operably linked to a nucleic acid encoding a Cas9 nuclease. In an aspect, an EA1 promoter is operably linked to a nucleic acid encoding a Cas12a nuclease. In an aspect, an EA1 promoter is operably linked to a nucleic acid encoding a CasX nuclease. In an aspect, an EA1 promoter is operably linked to a nucleic acid encoding a guide nucleic acid. In an aspect, an EA1 promoter is operably linked to a nucleic acid encoding a guide RNA. In an aspect, an EA1 promoter is operably linked to a nucleic acid encoding a single-guide RNA. In some embodiments, the guide RNA acid is flanked by self-cleaving ribozymes. In an aspect, an EA1 promoter is operably linked to a nucleic acid encoding a recombinase (e.g., Cre recombinase). In an aspect, an EA1 promoter is operably linked to a nucleic acid encoding a TALE.

In an aspect, an ES4 promoter is operably linked to a nucleic acid encoding a guided nuclease. In an aspect, an ES4 promoter is operably linked to a nucleic acid encoding a Cas9 nuclease. In an aspect, an ES4 promoter is operably linked to a nucleic acid encoding a Cas12a nuclease. In an aspect, an ES4 promoter is operably linked to a nucleic acid encoding a CasX nuclease. In an aspect, an ES4 promoter is operably linked to a nucleic acid encoding a guide nucleic acid. In an aspect, an ES4 promoter is operably linked to a nucleic acid encoding a guide RNA. In an aspect, an ES4 promoter is operably linked to a nucleic acid encoding a single-guide RNA. In some embodiments, the guide RNA acid is flanked by self-cleaving ribozymes. In an aspect, an ES4 promoter is operably linked to a nucleic acid encoding a recombinase (e.g., Cre recombinase). In an aspect, an ES4 promoter is operably linked to a nucleic acid encoding a TALE.

In an aspect, an EAL1 promoter is operably linked to a nucleic acid encoding a guided nuclease. In an aspect, an EAL1 promoter is operably linked to a nucleic acid encoding a Cas9 nuclease. In an aspect, an EAL1 promoter is operably linked to a nucleic acid encoding a Cas12a nuclease. In an aspect, an EAL1 promoter is operably linked to a nucleic acid encoding a CasX nuclease. In an aspect, an EAL1 promoter is operably linked to a nucleic acid encoding a guide nucleic acid. In an aspect, an EAL1 promoter is operably linked to a nucleic acid encoding a guide RNA. In an aspect, an EAL1 promoter is operably linked to a nucleic acid encoding a single-guide RNA. In some embodiments, the guide RNA acid is flanked by self-cleaving ribozymes. In an aspect, an EAL1 promoter is operably linked to a nucleic acid encoding a recombinase (e.g., Cre recombinase). In an aspect, an EAL1 promoter is operably linked to a nucleic acid encoding a TALE.

In an aspect, a DSUL1 promoter comprises a nucleic acid sequence at least 80% identical to SEQ ID NO: 1, or a functional fragment thereof. In an aspect, a DSUL1 promoter comprises a nucleic acid sequence at least 85% identical to SEQ ID NO: 1, or a functional fragment thereof. In an aspect, a DSUL1 promoter comprises a nucleic acid sequence at least 90% identical to SEQ ID NO: 1, or a functional fragment thereof. In an aspect, a DSUL1 promoter comprises a nucleic acid sequence at least 95% identical to SEQ ID NO: 1, or a functional fragment thereof. In an aspect, a DSUL1 promoter comprises a nucleic acid sequence at least 96% identical to SEQ ID NO: 1, or a functional fragment thereof. In an aspect, a DSUL1 promoter comprises a nucleic acid sequence at least 97% identical to SEQ ID NO: 1, or a functional fragment thereof. In an aspect, a DSUL1 promoter comprises a nucleic acid sequence at least 98% identical to SEQ ID NO: 1, or a functional fragment thereof. In an aspect, a DSUL1 promoter comprises a nucleic acid sequence at least 99% identical to SEQ ID NO: 1, or a functional fragment thereof. In an aspect, a DSUL1 promoter comprises a nucleic acid sequence 100% identical to SEQ ID NO: 1, or a functional fragment thereof.

In an aspect, an EA1 promoter comprises a nucleic acid sequence at least 80% identical to SEQ ID NO: 2, or a functional fragment thereof. In an aspect, an EA1 promoter comprises a nucleic acid sequence at least 85% identical to SEQ ID NO: 2, or a functional fragment thereof. In an aspect, an EA1 promoter comprises a nucleic acid sequence at least 90% identical to SEQ ID NO: 2, or a functional fragment thereof. In an aspect, an EA1 promoter comprises a nucleic acid sequence at least 95% identical to SEQ ID NO: 2, or a functional fragment thereof. In an aspect, an EA1 promoter comprises a nucleic acid sequence at least 96% identical to SEQ ID NO: 2, or a functional fragment thereof. In an aspect, an EA1 promoter comprises a nucleic acid sequence at least 97% identical to SEQ ID NO: 2, or a functional fragment thereof. In an aspect, an EA1 promoter comprises a nucleic acid sequence at least 98% identical to SEQ ID NO: 2, or a functional fragment thereof. In an aspect, an EA1 promoter comprises a nucleic acid sequence at least 99% identical to SEQ ID NO: 2, or a functional fragment thereof. In an aspect, an EA1 promoter comprises a nucleic acid sequence 100% identical to SEQ ID NO: 2, or a functional fragment thereof.

In an aspect, an ES4 promoter comprises a nucleic acid sequence at least 80% identical to SEQ ID NO: 3, or a functional fragment thereof. In an aspect, an ES4 promoter comprises a nucleic acid sequence at least 85% identical to SEQ ID NO: 3, or a functional fragment thereof. In an aspect, an ES4 promoter comprises a nucleic acid sequence at least 90% identical to SEQ ID NO: 3, or a functional fragment thereof. In an aspect, an ES4 promoter comprises a nucleic acid sequence at least 95% identical to SEQ ID NO: 3, or a functional fragment thereof. In an aspect, an ES4 promoter comprises a nucleic acid sequence at least 96% identical to SEQ ID NO: 3, or a functional fragment thereof. In an aspect, an ES4 promoter comprises a nucleic acid sequence at least 97% identical to SEQ ID NO: 3, or a functional fragment thereof. In an aspect, an ES4 promoter comprises a nucleic acid sequence at least 98% identical to SEQ ID NO: 3, or a functional fragment thereof. In an aspect, an ES4 promoter comprises a nucleic acid sequence at least 99% identical to SEQ ID NO: 3, or a functional fragment thereof. In an aspect, an ES4 promoter comprises a nucleic acid sequence 100% identical to SEQ ID NO: 3, or a functional fragment thereof.

In an aspect, an egg cell-preferred promoter, ovule tissue-preferred promoter, zygote cell-preferred or an embryo tissue-preferred promoter comprises a nucleic acid sequence at least 80% identical to a nucleic acid sequence selected from the group consisting of SEQ ID NOs: 1-3, 21-45, 65-69, 75-82, 86-88 or a functional fragment thereof. In an aspect, an egg cell-preferred promoter or an embryo tissue-preferred promoter comprises a nucleic acid sequence at least 85% identical to a nucleic acid sequence selected from the group consisting of SEQ ID NOs: 1-3, 21-45, 65-69, 75-82, 86-88 or a functional fragment thereof. In an aspect, an egg cell-preferred promoter or an embryo tissue-preferred promoter comprises a nucleic acid sequence at least 90% identical to a nucleic acid sequence selected from the group consisting of SEQ ID NOs: 1-3, 21-45, 65-69, 75-82, 86-88 or a functional fragment thereof. In an aspect, an egg cell-preferred promoter or an embryo tissue-preferred promoter comprises a nucleic acid sequence at least 95% identical to a nucleic acid sequence selected from the group consisting of SEQ ID NOs: 1-3, 21-45, 65-69, 75-82, 86-88 or a functional fragment thereof. In an aspect, an egg cell-preferred promoter or an embryo tissue-preferred promoter comprises a nucleic acid sequence at least 96% identical to a nucleic acid sequence selected from the group consisting of SEQ ID NOs: 1-3, 21-45, 65-69, 75-82, 86-88 or a functional fragment thereof. In an aspect, an egg cell-preferred promoter or an embryo tissue-preferred promoter comprises a nucleic acid sequence at least 97% identical to a nucleic acid sequence selected from the group consisting of SEQ ID NOs: 1-3, 21-45, 65-69, 75-82, 86-88 or a functional fragment thereof. In an aspect, an egg cell-preferred promoter or an embryo tissue-preferred promoter comprises a nucleic acid sequence at least 98% identical to a nucleic acid sequence selected from the group consisting of SEQ ID NOs: 1-3, 21-45, 65-69, 75-82, 86-88 or a functional fragment thereof. In an aspect, an egg cell-preferred promoter or an embryo tissue-preferred promoter comprises a nucleic acid sequence at least 99% identical to a nucleic acid sequence selected from the group consisting of SEQ ID NOs: 1-3, 21-45, 65-69, 75-82, 86-88 or a functional fragment thereof. In an aspect, an egg cell-preferred promoter or an embryo tissue-preferred promoter comprises a nucleic acid sequence 100% identical to a nucleic acid sequence selected from the group consisting of SEQ ID NOs: 1-3, 21-45, 65-69, 75-82, 86-88 or a functional fragment thereof.

It is appreciated in the art that a fragment of a promoter sequence can function to drive transcription of an operably linked nucleic acid molecule. For example, without being limiting, if a 1000 bp promoter is truncated to 500 bp, and the 500 bp fragment is capable of driving transcription, the 500 bp fragment is referred to as a “functional fragment.”

In an aspect, an egg cell-preferred promoter is operably linked to a nucleic acid sequence encoding a DNA modification enzyme, such as a guided nuclease. In an aspect, an egg cell-preferred promoter is operably linked to a nucleic acid sequence encoding a Cas9 nuclease. In an aspect, an egg cell-preferred promoter is operably linked to a nucleic acid sequence encoding a Cas12a nuclease. In an aspect, an egg cell-preferred promoter is operably linked to a nucleic acid sequence encoding a CasX nuclease. In an aspect, an egg cell-preferred promoter is operably linked to a nucleic acid sequence encoding a guide nucleic acid. In an aspect, an egg cell-preferred promoter is operably linked to a nucleic acid sequence encoding a guide RNA. In an aspect, an egg cell-preferred promoter is operably linked to a nucleic acid sequence encoding a single-guide RNA. In some embodiments, the guide RNA acid is flanked by self-cleaving ribozymes. In an aspect, an egg cell-preferred promoter is operably linked to a nucleic acid sequence encoding a recombinase (e.g., Cre recombinase). In an aspect, an egg cell-preferred promoter is operably linked to a nucleic acid sequence encoding a TALE.

In an aspect, an egg cell-specific promoter is operably linked to a nucleic acid sequence encoding a DNA modification enzyme, such as a guided nuclease. In an aspect, an egg cell-specific promoter is operably linked to a nucleic acid sequence encoding a Cas9 nuclease. In an aspect, an egg cell-specific promoter is operably linked to a nucleic acid sequence encoding a Cas12a nuclease. In an aspect, an egg cell-specific promoter is operably linked to a nucleic acid sequence encoding a CasX nuclease. In an aspect, an egg cell-specific promoter is operably linked to a nucleic acid sequence encoding a guide nucleic acid. In an aspect, an egg cell-specific promoter is operably linked to a nucleic acid sequence encoding a guide RNA. In an aspect, an egg cell-specific promoter is operably linked to a nucleic acid sequence encoding a single-guide RNA. In some embodiments, the guide RNA acid is flanked by self-cleaving ribozymes. In an aspect, an egg cell-specific promoter is operably linked to a nucleic acid sequence encoding a recombinase (e.g., Cre recombinase). In an aspect, an egg cell-specific promoter is operably linked to a nucleic acid sequence encoding a TALE.

In an aspect, an embryo tissue-preferred promoter is operably linked to a nucleic acid sequence encoding a DNA modification enzyme, such as a guided nuclease. In an aspect, an embryo tissue-preferred promoter is operably linked to a nucleic acid sequence encoding a Cas9 nuclease. In an aspect, an embryo tissue-preferred promoter is operably linked to a nucleic acid sequence encoding a Cas12a nuclease. In an aspect, an embryo tissue-preferred promoter is operably linked to a nucleic acid sequence encoding a CasX nuclease. In an aspect, an embryo tissue-preferred promoter is operably linked to a nucleic acid sequence encoding a guide nucleic acid. In an aspect, an embryo tissue-preferred promoter is operably linked to a nucleic acid sequence encoding a guide RNA. In an aspect, an embryo tissue-preferred promoter is operably linked to a nucleic acid sequence encoding a single-guide RNA. In some embodiments, the guide RNA acid is flanked by self-cleaving ribozymes. In an aspect, an embryo tissue-preferred promoter is operably linked to a nucleic acid sequence encoding a recombinase (e.g., Cre recombinase). In an aspect, an embryo tissue-preferred promoter is operably linked to a nucleic acid sequence encoding a TALE.

In an aspect, an embryo tissue-specific promoter is operably linked to a nucleic acid sequence encoding a DNA modification enzyme, such as a guided nuclease. In an aspect, an embryo tissue-specific promoter is operably linked to a nucleic acid sequence encoding a Cas9 nuclease. In an aspect, an embryo tissue-specific promoter is operably linked to a nucleic acid sequence encoding a Cas12a nuclease. In an aspect, an embryo tissue-specific promoter is operably linked to a nucleic acid sequence encoding a CasX nuclease. In an aspect, an embryo tissue-specific promoter is operably linked to a nucleic acid sequence encoding a guide nucleic acid. In an aspect, an embryo tissue-specific promoter is operably linked to a nucleic acid sequence encoding a guide RNA. In an aspect, an embryo tissue-specific promoter is operably linked to a nucleic acid sequence encoding a single-guide RNA. In some embodiments, the guide RNA acid is flanked by self-cleaving ribozymes. In an aspect, an embryo tissue-specific promoter is operably linked to a nucleic acid sequence encoding a recombinase (e.g., Cre recombinase). In an aspect, an embryo tissue-specific promoter is operably linked to a nucleic acid sequence encoding a TALE.

Several embodiments described herein relate to methods and compositions for providing egg, embryo, and/or meiotic plant tissue preferred or specific expression of a DNA modification enzyme, such as a guided nuclease (e.g., a CRISPR/Cas system), from a constitutive promoter. In some embodiments, a transcribable polynucleotide encoding a DNA modification enzyme, such as a guided nuclease (e.g., a CRISPR/Cas system) is operably linked to a constitutive promoter by excision of a intervening polynucleotide sequence preferentially or specifically in egg, embryo, and/or meiotic plant tissue. In some embodiments, the intervening sequence is excised by a recombinase that is preferentially or selectively expressed in egg, embryo, and/or meiotic plant tissue. In some embodiments, the intervening sequence is excised by Cre-mediated excision of an intervening Cre expression cassette, where Cre is preferentially or selectively expressed in egg, embryo, and/or meiotic plant tissue.

Promoters that drive expression in all or most tissues of the plant are referred to as “constitutive” promoters. Promoters that drive expression during certain periods or stages of development are referred to as “developmental” promoters. An “inducible” promoter is a promoter that initiates transcription in response to an environmental stimulus such as heat, cold, drought, light, or other stimuli, such as wounding or chemical application. A promoter can also be classified in terms of its origin, such as being heterologous, homologous, chimeric, synthetic, etc.

As used herein, the term “heterologous” in reference to a promoter is a promoter sequence having a different origin relative to its associated transcribable DNA sequence, coding sequence or gene (or transgene), and/or not naturally occurring in the plant species to be transformed. The term “heterologous” can refer more broadly to a combination of two or more DNA molecules or sequences, such as a promoter and an associated transcribable DNA sequence, coding sequence or gene, when such a combination is man-made and not normally found in nature.

In an aspect, a promoter provided herein is a constitutive promoter. In still another aspect, a promoter provided herein is an inducible promoter. In an aspect, a promoter provided herein is selected from the group consisting of a constitutive promoter, a tissue-specific promoter, a tissue-preferred promoter, and an inducible promoter.

RNA polymerase III (Pol III) promoters can be used to drive the expression of non-protein coding RNA molecules, such as guide RNAs. In an aspect, a promoter provided herein is a Pol III promoter. In another aspect, a Pol III promoter provided herein is operably linked to a nucleic acid molecule encoding a non-protein coding RNA. In yet another aspect, a Pol III promoter provided herein is operably linked to a nucleic acid molecule encoding a guide nucleic acid. In still another aspect, a Pol III promoter provided herein is operably linked to a nucleic acid molecule encoding a single-guide RNA. In a further aspect, a Pol III promoter provided herein is operably linked to a nucleic acid molecule encoding a CRISPR RNA (crRNA). In another aspect, a Pol III promoter provided herein is operably linked to a nucleic acid molecule encoding a tracer RNA (tracrRNA). In some embodiments, a nucleic acid molecule encoding a non-protein coding RNA (e.g., a gRNA, a single-guide RNA, a crRNA, a tracrRNA, etc.) is operably linked to a Pol III promoter by excision of a intervening polynucleotide sequence preferentially or specifically in egg, embryo, and/or meiotic plant tissue. In some embodiments, the intervening sequence is excised by a recombinase that is preferentially or selectively expressed in egg, embryo, and/or meiotic plant tissue. In some embodiments, the intervening sequence is excised by Cre-mediated excision of an intervening Cre expression cassette, where Cre is preferentially or selectively expressed in egg, embryo, and/or meiotic plant tissue.

Non-limiting examples of Pol III promoters include a U6 promoter, an H1 promoter, a 5S promoter, an Adenovirus 2 (Ad2) VAI promoter, a tRNA promoter, and a 7SK promoter. See, for example, Schramm and Hernandez, 2002, Genes & Development, 16:2593-2620, which is incorporated by reference herein in its entirety. In an aspect, a Pol III promoter provided herein is selected from the group consisting of a U6 promoter, an H1 promoter, a 5S promoter, an Adenovirus 2 (Ad2) VAI promoter, a tRNA promoter, and a 7SK promoter. In another aspect, a guide RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a single-guide RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a CRISPR RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a tracer RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter.

In an aspect, a promoter provided herein is a Dahlia Mosaic Virus (DaMV) promoter. In another aspect, a promoter provided herein is a U6 promoter. In another aspect, a promoter provided herein is an actin promoter. In an aspect, a promoter provided herein is a Cauliflower Mosaic Virus (CaMV) 35S promoter. In an aspect, a promoter provided herein is a ubiquitin promoter.

In an aspect, a constitutive promoter is selected from the group consisting of a CaMV 35S promoter, an actin promoter, and a ubiquitin promoter.

Examples describing a promoter that can be used herein include without limitation U.S. Pat. No. 6,437,217 (maize RS81 promoter), U.S. Pat. No. 5,641,876 (rice actin promoter), U.S. Pat. No. 6,426,446 (maize RS324 promoter), U.S. Pat. No. 6,429,362 (maize PR-1 promoter), U.S. Pat. No. 6,232,526 (maize A3 promoter), U.S. Pat. No. 6,177,611 (constitutive maize promoters), U.S. Pat. Nos. 5,322,938, 5,352,605, 5,359,142 and 5,530,196 (35S promoter), U.S. Pat. No. 6,433,252 (maize L3 oleosin promoter), U.S. Pat. No. 6,429,357 (rice actin 2 promoter as well as a rice actin 2 intron), U.S. Pat. No. 5,837,848 (root specific promoter), U.S. Pat. No. 6,294,714 (light inducible promoters), U.S. Pat. No. 6,140,078 (salt inducible promoters), U.S. Pat. No. 6,252,138 (pathogen inducible promoters), U.S. Pat. No. 6,175,060 (phosphorus deficiency inducible promoters), U.S. Pat. No. 6,635,806 (gamma-coixin promoter), and U.S. patent application Ser. No. 09/757,089 (maize chloroplast aldolase promoter). Additional promoters that can find use are a nopaline synthase (NOS) promoter (Ebert et. al., 1987), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et. al., Plant Molecular Biology (1987) 9: 315-324), the CaMV 35S promoter (Odell et. al., Nature (1985) 313: 810-812), the figwort mosaic virus 35S-promoter (U.S. Pat. Nos. 6,051,753; 5,378,619), the sucrose synthase promoter (Yang and Russell, Proceedings of the National Academy of Sciences, USA (1990) 87: 4144-4148), the R gene complex promoter (Chandler et. al., Plant Cell (1989) 1: 1175-1183), and the chlorophyll a/b binding protein gene promoter, PC1SV (U.S. Pat. No. 5,850,019), and AGRtu.nos (GenBank Accession V00087; Depicker et. al., Journal of Molecular and Applied Genetics (1982) 1: 561-573; Bevan et. al., 1983) promoters.

Promoter hybrids can also be used and constructed to enhance transcriptional activity (see U.S. Pat. No. 5,106,739), or to combine desired transcriptional activity, inducibility and tissue specificity or developmental specificity. Promoters that function in plants include but are not limited to promoters that are inducible, viral, synthetic, constitutive, temporally regulated, spatially regulated, and spatio-temporally regulated. Other promoters that are tissue-enhanced, tissue-specific, or developmentally regulated are also known in the art and envisioned to have utility in the practice of this disclosure.

In an aspect, a constitutive promoter is operably linked to a nucleic acid sequence encoding a guided nuclease by Cre-mediated excision of an intervening Cre expression cassette, where Cre is preferentially or selectively expressed in egg, embryo, and/or meiotic plant tissue. In an aspect, a constitutive promoter is operably linked to a nucleic acid sequence encoding a Cas9 nuclease by Cre-mediated excision of an intervening Cre expression cassette, where Cre is preferentially or selectively expressed in egg, embryo, and/or meiotic plant tissue. In an aspect, a constitutive promoter is operably linked to a nucleic acid sequence encoding a Cas12a nuclease by Cre-mediated excision of an intervening Cre expression cassette, where Cre is preferentially or selectively expressed in egg, embryo, and/or meiotic plant tissue. In an aspect, a constitutive promoter is operably linked to a nucleic acid sequence encoding a CasX nuclease by Cre-mediated excision of an intervening Cre expression cassette, where Cre is preferentially or selectively expressed in egg, embryo, and/or meiotic plant tissue. In an aspect, a constitutive promoter is operably linked to a nucleic acid sequence encoding a guide nucleic acid by Cre-mediated excision of an intervening Cre expression cassette, where Cre is preferentially or selectively expressed in egg, embryo, and/or meiotic plant tissue. In an aspect, a constitutive promoter is operably linked to a nucleic acid sequence encoding a guide RNA by Cre-mediated excision of an intervening Cre expression cassette, where Cre is preferentially or selectively expressed in egg, embryo, and/or meiotic plant tissue. In an aspect, a constitutive promoter is operably linked to a nucleic acid sequence encoding a single-guide RNA by Cre-mediated excision of an intervening Cre expression cassette, where Cre is preferentially or selectively expressed in egg, embryo, and/or meiotic plant tissue.

In an aspect, an inducible promoter is operably linked to a nucleic acid sequence encoding a guided nuclease. In an aspect, an inducible promoter is operably linked to a nucleic acid sequence encoding a Cas9 nuclease. In an aspect, an inducible promoter is operably linked to a nucleic acid sequence encoding a Cas12a nuclease. In an aspect, an inducible promoter is operably linked to a nucleic acid sequence encoding a CasX nuclease. In an aspect, an inducible promoter is operably linked to a nucleic acid sequence encoding a guide nucleic acid. In an aspect, an inducible promoter is operably linked to a nucleic acid sequence encoding a guide RNA. In an aspect, an inducible promoter is operably linked to a nucleic acid sequence encoding a single-guide RNA. In some embodiments, the guide RNA acid is flanked by self-cleaving ribozymes. In an aspect, an inducible promoter is operably linked to a nucleic acid sequence encoding a recombinase (e.g., Cre recombinase). In an aspect, an inducible promoter is operably linked to a nucleic acid sequence encoding a TALE.

In an aspect, a developmental promoter is operably linked to a nucleic acid sequence encoding a guided nuclease. In an aspect, a developmental promoter is operably linked to a nucleic acid sequence encoding a Cas9 nuclease. In an aspect, a developmental promoter is operably linked to a nucleic acid sequence encoding a Cas12a nuclease. In an aspect, a developmental promoter is operably linked to a nucleic acid sequence encoding a CasX nuclease. In an aspect, a developmental promoter is operably linked to a nucleic acid sequence encoding a guide nucleic acid. In an aspect, a developmental promoter is operably linked to a nucleic acid sequence encoding a guide RNA. In an aspect, a developmental promoter is operably linked to a nucleic acid sequence encoding a single-guide RNA. In some embodiments, the guide RNA acid is flanked by self-cleaving ribozymes. In an aspect, a developmental promoter is operably linked to a nucleic acid sequence encoding a recombinase (e.g., Cre recombinase). In an aspect, a developmental promoter is operably linked to a nucleic acid sequence encoding a TALE.

As used herein the term “leader” refers to a nucleotide segment between the transcription start site (TSS) and protein coding sequence start site of a gene. It is isolated from the untranslated 5′ region of the genomic copy of a gene. Leaders can be used as 5′ regulatory elements to regulate the expression of operably linked transcribable polynucleotide molecules. The leader molecule can be used with a heterologous promoter or with its native promoter.

As used herein, the term “3′ transcription termination molecule” or “3′ UTR” refers to a DNA sequence that is used during transcription to produce the 3′ untranslated region (3′ UTR) of an mRNA molecule. The 3′ untranslated region of an mRNA molecule may be generated by specific cleavage and 3′ polyadenylation, a.k.a. polyA tail. A 3′ UTR may be operably linked to and located downstream of a transcribable polynucleotide molecule and may include polynucleotides that provide a polyadenylation signal and other regulatory signals capable of affecting transcription, mRNA processing, or gene expression. PolyA tails are thought to function in mRNA stability and in initiation of translation. Examples of 3′ transcription termination molecules in the art are the nopaline synthase 3′ region (see, Fraley, et. al., Proc. Natl. Acad. Sci. USA, 80:4803-4807 (1983)); wheat hsp17 3′ region; pea rubisco small subunit 3′ region; cotton E6 3′ region (U.S. Pat. No. 6,096,950); 3′ regions disclosed in WO0011200A2; and the coixin 3′ UTR (U.S. Pat. No. 6,635,806). 3′ UTRs typically find beneficial use for the recombinant expression of specific genes. 3′UTRs can be used as 3′regulatory elements to regulate the expression of operably linked transcribable polynucleotide molecules. 3′UTRs can be used as 3′ regulatory elements to regulate the tissue/cell preferred expression of operably linked transcribable polynucleotide molecules. 3′ UTRs can be used with a heterologous promoter or with its native promoter. Non-limiting examples of 3′ UTRs useful in practicing the various embodiments described here include SEQ ID NOs 46-64, 70-74, 89-102

### DNA Modification Enzymes

Several embodiments relate to compositions and methods for preferential or specific expression of one or more components of a genome editing system in egg, embryo, and/or meiotic plant tissue. Several embodiments relate to a gene regulatory element as described in Table 1 operably linked to a heterologous transcribable DNA molecule encoding one or more components of a genome editing system. Genome editing systems may be used to introduce one or more insertions, deletions, substitutions, base modifications, translocations, or inversions to a genome of a host cell. In some embodiments, a gene regulatory element as described in Table 1 is operably linked to a heterologous transcribable DNA molecule encoding a sequence-specific DNA modification enzyme, such as a CRISPR-Cas effector protein, a zinc finger protein, or a transcription activator (TAL) protein. In some embodiments, the sequence-specific DNA modification enzyme maybe a fusion protein. In some embodiments, the sequence-specific DNA modification enzyme maybe a guided nuclease.

Guided nucleases are nucleases that form a complex (e.g., a ribonucleoprotein) with a guide nucleic acid molecule (e.g., a guide RNA), which then guides the complex to a target site within a target sequence. One non-limiting example of guided nucleases are CRISPR nucleases.

CRISPR (Clustered Regularly Interspaced Short Palindromic Repeats) nucleases (e.g., Cas9, CasX, Cas12a (also referred to as Cpf1), CasY) are proteins found in bacteria that are guided by guide RNAs (“gRNAs”) to a target nucleic acid molecule, where the endonuclease can then cleave one or two strands the target nucleic acid molecule. Although the origins of CRISPR nucleases are bacterial, many CRISPR nucleases have been shown to function in eukaryotic cells.

While not being limited by any particular scientific theory, a CRISPR nuclease forms a complex with a guide RNA (gRNA), which hybridizes with a complementary target site, thereby guiding the CRISPR nuclease to the target site. In class II CRISPR-Cas systems, CRISPR arrays, including spacers, are transcribed during encounters with recognized invasive DNA and are processed into small interfering CRISPR RNAs (crRNAs). The crRNA comprises a repeat sequence and a spacer sequence which is complementary to a specific protospacer sequence in an invading pathogen. The spacer sequence can be designed to be complementary to target sequences in a eukaryotic genome.

In some embodiments, a gene regulatory element as described herein is operably linked to a heterologous transcribable DNA molecule encoding a CRISPR-Cas effector protein. In some embodiments, the CRISPR-Cas effector protein is selected from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. Examples of CRISPR-Cas effector proteins include, but are not limited to, Cas9, C2c1, C2c3, C2c4, C2c5, C2c8, C2c9, C2c10, Cas12a (also referred to as Cpf1), Cas12b, Cas12c, Cas12d, Cas12e, Cas12h, Cas12i, Cas12g, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), Csf5, Cas14a, Cas14b, and Cas14c effector protein. In some embodiments, a gene regulatory element as described herein is operably linked to a CRISPR-Cas effector protein comprising a mutation in its nuclease active site (e.g., RuvC, HNH, and/or NUC domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation. In some embodiments, a gene regulatory element as described herein is operably linked to a CRISPR-Cas effector protein having a mutation in its nuclease active site to generate a nickase activity operably linked to a reverse transcriptase enzyme.

CRISPR effector proteins associate with their respective crRNAs in their active forms. CasX, similar to the class II endonuclease Cas9, requires another non-coding RNA component, referred to as a trans-activating crRNA (tracrRNA), to have functional activity. Nucleic acid molecules provided herein can combine a crRNA and a tracrRNA into one nucleic acid molecule in what is herein referred to as a “single guide RNA” (sgRNA). Cas12a does not require a tracrRNA to be guided to a target site; a crRNA alone is sufficient for Cas12a. The gRNA guides the active CRISPR nuclease complex to a target site, where the CRISPR nuclease can cleave the target site.

When a CRISPR effector protein and a guide RNA form a complex, the whole system is called a “ribonucleoprotein.” Ribonucleoproteins provided herein can also comprise additional nucleic acids or proteins.

In an aspect, a CRISPR effector protein and a guide nucleic acid form a ribonucleoprotein in an egg cell. In another aspect, a CRISPR effector protein and a guide nucleic acid form a ribonucleoprotein in embryo tissue. In an aspect, a Cas9 nuclease and a guide nucleic acid form a ribonucleoprotein in an egg cell. In another aspect, a Cas9 nuclease and a guide nucleic acid form a ribonucleoprotein in embryo tissue. In an aspect, a Cas12a nuclease and a guide nucleic acid form a ribonucleoprotein in an egg cell. In another aspect, a Cas12a nuclease and a guide nucleic acid form a ribonucleoprotein in embryo tissue. In an aspect, a CasX nuclease and a guide nucleic acid form a ribonucleoprotein in an egg cell. In another aspect, a CasX nuclease and a guide nucleic acid form a ribonucleoprotein in embryo tissue. In an aspect, a CRISPR effector protein and a guide RNA form a ribonucleoprotein in an egg cell. In another aspect, a CRISPR effector protein and a guide RNA form a ribonucleoprotein in embryo tissue. In an aspect, a Cas9 nuclease and a guide RNA form a ribonucleoprotein in an egg cell. In another aspect, a Cas9 nuclease and a guide RNA form a ribonucleoprotein in embryo tissue. In an aspect, a Cas12a nuclease and a guide RNA form a ribonucleoprotein in an egg cell. In another aspect, a Cas12a nuclease and a guide RNA form a ribonucleoprotein in embryo tissue. In an aspect, a CasX nuclease and a guide RNA form a ribonucleoprotein in an egg cell. In another aspect, a CasX nuclease and a guide RNA form a ribonucleoprotein in embryo tissue. In an aspect, a guided nuclease and a single-guide RNA form a ribonucleoprotein in an egg cell. In another aspect, a guided nuclease and a single-guide RNA form a ribonucleoprotein in embryo tissue. In another aspect, a CasX nuclease and a single-guide RNA form a ribonucleoprotein in embryo tissue. In another aspect, a Cas9 nuclease and a single-guide RNA form a ribonucleoprotein in embryo tissue.

In an aspect, a ribonucleoprotein generates at least one double-stranded break within a target site in an egg cell. In an aspect, a ribonucleoprotein generates at least one double-stranded break within a target site in embryo tissue. In an aspect, a ribonucleoprotein generates at least one single-stranded break within a target site in an egg cell. In an aspect, a ribonucleoprotein generates at least one single-stranded break within a target site in embryo tissue.

A prerequisite for cleavage of the target site by a CRISPR ribonucleoprotein is the presence of a conserved Protospacer Adjacent Motif (PAM) near the target site. Depending on the CRISPR nuclease, cleavage can occur within a certain number of nucleotides (e.g., between 18-23 nucleotides for Cas12a) from the PAM site. PAM sites are only required for type I and type II CRISPR associated proteins, and different CRISPR endonucleases recognize different PAM sites. Without being limiting, Cas12a can recognize at least the following PAM sites: TTTN, and YTN; and CasX can recognize at least the following PAM sites: TTCN, TTCA, and TTC (where T is thymine; C is cytosine; A is adenine; Y is thymine or cytosine; and N is thymine, cytosine, guanine, or adenine).

Cas12a is an RNA-guided nuclease of a class II, type V CRISPR/Cas system. Cas12a nucleases generate staggered cuts when cleaving a double-stranded DNA molecule. Staggered cuts of double-stranded DNA produce a single-stranded DNA overhang of at least one nucleotide. This is in contrast to a blunt-end cut (such as those generated by Cas9), which does not produce a single-stranded DNA overhang when cutting double-stranded DNA.

In an aspect, a Cas12a nuclease provided herein is a Lachnospiraceae bacterium Cas12a (LbCas12a) nuclease. In another aspect, a Cas12a nuclease provided herein is a Francisella novicida Cas12a (FnCas12a) nuclease. In an aspect, a Cas12a nuclease is selected from the group consisting of LbCas12a and FnCas12a.

In an aspect, a Cas12a nuclease, or a nucleic acid encoding a Cas12a nuclease, is derived from a bacteria genus selected from the group consisting of Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Acidaminococcus, Peregrinibacteria, Butyrivibrio, Parcubacteria, Smithella, Candidatus, Moraxella, and Leptospira.

In an aspect, a Cas12a nuclease is encoded by a polynucleotide comprising a sequence at least 80% identical to a polynucleotide selected from the group consisting of SEQ ID NO: 7. In another aspect, a Cas12a nuclease is encoded by a polynucleotide comprising a sequence at least 85% identical to a polynucleotide selected from the group consisting of SEQ ID NO: 7. In another aspect, a Cas12a nuclease is encoded by a polynucleotide comprising a sequence at least 90% identical to a polynucleotide selected from the group consisting of SEQ ID NO: 7. In another aspect, a Cas12a nuclease is encoded by a polynucleotide comprising a sequence at least 95% identical to a polynucleotide selected from the group consisting of SEQ ID NO: 7. In another aspect, a Cas12a nuclease is encoded by a polynucleotide comprising a sequence at least 96% identical to a polynucleotide selected from the group consisting of SEQ ID NO: 7. In another aspect, a Cas12a nuclease is encoded by a polynucleotide comprising a sequence at least 97% identical to a polynucleotide selected from the group consisting of SEQ ID NO: 7. In another aspect, a Cas12a nuclease is encoded by a polynucleotide comprising a sequence at least 98% identical to a polynucleotide selected from the group consisting of SEQ ID NO: 7. In another aspect, a Cas12a nuclease is encoded by a polynucleotide comprising a sequence at least 99% identical to a polynucleotide selected from the group consisting of SEQ ID NO: 7. In another aspect, a Cas12a nuclease is encoded by a polynucleotide comprising a sequence 100% identical to a polynucleotide selected from the group consisting of SEQ ID NO: 7.

CasX is a type of class II CRISPR-Cas nuclease that has been identified in the bacterial phyla Deltaproteobacteria and Planctomycetes. Similar to Cas12a, CasX nucleases generate staggered cuts when cleaving a double-stranded DNA molecule. However, unlike Cas12a, CasX nucleases require a crRNA and a tracrRNA, or a single-guide RNA, in order to target and cleave a target nucleic acid.

In an aspect, a CasX nuclease provided herein is a CasX nuclease from the phylum Deltaproteobacteria. In another aspect, a CasX nuclease provided herein is a CasX nuclease from the phylum Planctomycetes. Without being limiting, additional suitable CasX nucleases are those set forth in WO 2019/084148, which is incorporated by reference herein in its entirety.

In an aspect, a guided nuclease capable of generating a staggered cut in a double-stranded DNA molecule is selected from the group consisting of Cas12a and CasX. In an aspect, a guided nuclease is selected from the group consisting of Cas12a and CasX.

In an aspect, a guided nuclease is a RNA-guided nuclease. In another aspect, a guided nuclease is a CRISPR nuclease. In another aspect, a guided nuclease is a Cas12a nuclease. In another aspect, a guided nuclease is a CasX nuclease.

As used herein, a “nuclear localization signal” (NLS) refers to an amino acid sequence that “tags” a protein for import into the nucleus of a cell. In an aspect, a nucleic acid molecule provided herein encodes a nuclear localization signal. In another aspect, a nucleic acid molecule provided herein encodes two or more nuclear localization signals.

In an aspect, a CRISPR effector protein provided herein comprises a nuclear localization signal. In an aspect, a Cas9 effector protein provided herein comprises a nuclear localization signal. In an aspect, a nuclear localization signal is positioned on the N-terminal end of a Cas12a nuclease. In a further aspect, a nuclear localization signal is positioned on the C-terminal end of a Cas9 effector protein. In yet another aspect, a nuclear localization signal is positioned on both the N-terminal end and the C-terminal end of a Cas9 effector protein.

In an aspect, a Cas12a effector protein provided herein comprises a nuclear localization signal. In an aspect, a nuclear localization signal is positioned on the N-terminal end of a Cas12a effector protein. In a further aspect, a nuclear localization signal is positioned on the C-terminal end of a Cas12a effector protein. In yet another aspect, a nuclear localization signal is positioned on both the N-terminal end and the C-terminal end of a Cas12a effector protein.

In an aspect, a CasX effector protein provided herein comprises a nuclear localization signal. In an aspect, a nuclear localization signal is positioned on the N-terminal end of a CasX effector protein. In a further aspect, a nuclear localization signal is positioned on the C-terminal end of a CasX effector protein. In yet another aspect, a nuclear localization signal is positioned on both the N-terminal end and the C-terminal end of a CasX effector protein.

In an aspect, a ribonucleoprotein comprises at least one nuclear localization signal. In another aspect, a ribonucleoprotein comprises at least two nuclear localization signals. In an aspect, a nuclear localization signal provided herein is encoded by SEQ ID NO: 8 or 9.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www[dot]kazusa[dot]or[dot]jp[forwards slash]codon and these tables can be adapted in a number of ways. See Nakamura et. al., 2000, Nucl. Acids Res. 28:292. Computer algorithms for codon optimizing a particular sequence for expression in a particular plant cell are also available, such as Gene Forge (Aptagen; Jacobus, PA), are also available.

As used herein, “codon optimization” refers to a process of modifying a nucleic acid sequence for enhanced expression in a plant cell of interest by replacing at least one codon (e.g., at least 1, 2, 3, 4, 5, 10, 15, 20, 25, 50, or more codons) of a sequence with codons that are more frequently or most frequently used in the genes of the plant cell while maintaining the original amino acid sequence (e.g., introducing silent mutations).

In an aspect, one or more codons (e.g., 1, 2, 3, 4, 5, 10, 15, 20, 25, 50, or more, or all codons) in a sequence encoding a guided nuclease correspond to the most frequently used codon for a particular amino acid. In another aspect, one or more codons (e.g., 1, 2, 3, 4, 5, 10, 15, 20, 25, 50, or more, or all codons) in a sequence encoding a Cas9 effector protein, a Cas12a effector protein or a CasX effector protein correspond to the most frequently used codon for a particular amino acid. As to codon usage in plants, reference is made to Campbell and Gowri, 1990, Plant Physiol., 92: 1-11; and Murray et. al., 1989, Nucleic Acids Res., 17:477-98, each of which is incorporated herein by reference in their entireties.

In an aspect, a nucleic acid molecule encodes a guided nuclease that is codon optimized for a plant. In an aspect, a nucleic acid molecule encodes a Cas9 effector protein that is codon optimized for a plant. In an aspect, a nucleic acid molecule encodes a Cas12a effector protein that is codon optimized for a plant. In an aspect, a nucleic acid molecule encodes a CasX effector protein that is codon optimized for a plant.

In another aspect, a nucleic acid molecule provided herein encodes a guided nuclease that is codon optimized for a plant cell. In another aspect, a nucleic acid molecule provided herein encodes a guided nuclease that is codon optimized for a monocotyledonous plant species. In another aspect, a nucleic acid molecule provided herein encodes a guided nuclease that is codon optimized for a dicotyledonous plant species. In a further aspect, a nucleic acid molecule provided herein encodes a guided nuclease that is codon optimized for a gymnosperm plant species. In a further aspect, a nucleic acid molecule provided herein encodes a guided nuclease that is codon optimized for an angiosperm plant species. In a further aspect, a nucleic acid molecule provided herein encodes a guided nuclease that is codon optimized for a corn cell. In a further aspect, a nucleic acid molecule provided herein encodes a guided nuclease that is codon optimized for a soybean cell. In a further aspect, a nucleic acid molecule provided herein encodes a guided nuclease that is codon optimized for a rice cell. In a further aspect, a nucleic acid molecule provided herein encodes a guided nuclease that is codon optimized for a wheat cell. In a further aspect, a nucleic acid molecule provided herein encodes a guided nuclease that is codon optimized for a cotton cell. In a further aspect, a nucleic acid molecule provided herein encodes a guided nuclease that is codon optimized for a sorghum cell. In a further aspect, a nucleic acid molecule provided herein encodes a guided nuclease that is codon optimized for an alfalfa cell. In a further aspect, a nucleic acid molecule provided herein encodes a guided nuclease that is codon optimized for a sugarcane cell. In a further aspect, a nucleic acid molecule provided herein encodes a guided nuclease that is codon optimized for an Arabidopsis cell. In a further aspect, a nucleic acid molecule provided herein encodes a guided nuclease that is codon optimized for a tomato cell. In a further aspect, a nucleic acid molecule provided herein encodes a guided nuclease that is codon optimized for a cucumber cell. In a further aspect, a nucleic acid molecule provided herein encodes a guided nuclease that is codon optimized for a potato cell. In a further aspect, a nucleic acid molecule provided herein encodes a guided nuclease that is codon optimized for an onion cell.

In another aspect, a nucleic acid molecule provided herein encodes a Cas12a effector protein that is codon optimized for a plant cell. In another aspect, a nucleic acid molecule provided herein encodes a Cas12a effector protein that is codon optimized for a monocotyledonous plant species. In another aspect, a nucleic acid molecule provided herein encodes a Cas12a effector protein that is codon optimized for a dicotyledonous plant species. In a further aspect, a nucleic acid molecule provided herein encodes a Cas12a effector protein that is codon optimized for a gymnosperm plant species. In a further aspect, a nucleic acid molecule provided herein encodes a Cas12a effector protein that is codon optimized for an angiosperm plant species. In a further aspect, a nucleic acid molecule provided herein encodes a Cas12a effector protein that is codon optimized for a corn cell. In a further aspect, a nucleic acid molecule provided herein encodes a Cas12a effector protein that is codon optimized for a soybean cell. In a further aspect, a nucleic acid molecule provided herein encodes a Cas12a effector protein that is codon optimized for a rice cell. In a further aspect, a nucleic acid molecule provided herein encodes a Cas12a effector protein that is codon optimized for a wheat cell. In a further aspect, a nucleic acid molecule provided herein encodes a Cas12a effector protein that is codon optimized for a cotton cell. In a further aspect, a nucleic acid molecule provided herein encodes a Cas12a effector protein that is codon optimized for a sorghum cell. In a further aspect, a nucleic acid molecule provided herein encodes a Cas12a effector protein that is codon optimized for an alfalfa cell. In a further aspect, a nucleic acid molecule provided herein encodes a Cas12a effector protein that is codon optimized for a sugar cane cell. In a further aspect, a nucleic acid molecule provided herein encodes a Cas12a effector protein that is codon optimized for an Arabidopsis cell. In a further aspect, a nucleic acid molecule provided herein encodes a Cas12a effector protein that is codon optimized for a tomato cell. In a further aspect, a nucleic acid molecule provided herein encodes a Cas12a effector protein that is codon optimized for a cucumber cell. In a further aspect, a nucleic acid molecule provided herein encodes a Cas12a effector protein that is codon optimized for a potato cell. In a further aspect, a nucleic acid molecule provided herein encodes a Cas12a effector protein that is codon optimized for an onion cell.

In another aspect, a nucleic acid molecule provided herein encodes a CasX effector protein that is codon optimized for a plant cell. In another aspect, a nucleic acid molecule provided herein encodes a CasX effector protein that is codon optimized for a monocotyledonous plant species. In another aspect, a nucleic acid molecule provided herein encodes a CasX effector protein that is codon optimized for a dicotyledonous plant species. In a further aspect, a nucleic acid molecule provided herein encodes a CasX effector protein that is codon optimized for a gymnosperm plant species. In a further aspect, a nucleic acid molecule provided herein encodes a CasX effector protein that is codon optimized for an angiosperm plant species. In a further aspect, a nucleic acid molecule provided herein encodes a CasX effector protein that is codon optimized for a corn cell. In a further aspect, a nucleic acid molecule provided herein encodes a CasX effector protein that is codon optimized for a soybean cell. In a further aspect, a nucleic acid molecule provided herein encodes a CasX effector protein that is codon optimized for a rice cell. In a further aspect, a nucleic acid molecule provided herein encodes a CasX effector protein that is codon optimized for a wheat cell. In a further aspect, a nucleic acid molecule provided herein encodes a CasX effector protein that is codon optimized for a cotton cell. In a further aspect, a nucleic acid molecule provided herein encodes a CasX effector protein that is codon optimized for a sorghum cell. In a further aspect, a nucleic acid molecule provided herein encodes a CasX effector protein that is codon optimized for an alfalfa cell. In a further aspect, a nucleic acid molecule provided herein encodes a CasX effector protein that is codon optimized for a sugar cane cell. In a further aspect, a nucleic acid molecule provided herein encodes a CasX effector protein that is codon optimized for an Arabidopsis cell. In a further aspect, a nucleic acid molecule provided herein encodes a CasX effector protein that is codon optimized for a tomato cell. In a further aspect, a nucleic acid molecule provided herein encodes a CasX effector protein that is codon optimized for a cucumber cell. In a further aspect, a nucleic acid molecule provided herein encodes a CasX effector protein that is codon optimized for a potato cell. In a further aspect, a nucleic acid molecule provided herein encodes a CasX effector protein that is codon optimized for an onion cell.

### Guide Nucleic Acids

As used herein, a “guide nucleic acid” refers to a nucleic acid that forms a ribonucleoprotein (e.g., a complex) with a CRISPR effector protein (e.g., without being limiting, Cas9, Cas12a, CasX) and then guides the ribonucleoprotein to a specific sequence in a target nucleic acid molecule, where the guide nucleic acid and the target nucleic acid molecule share complementary sequences. In an aspect, a ribonucleoprotein provided herein comprises at least one guide nucleic acid.

In an aspect, a guide nucleic acid comprises DNA. In another aspect, a guide nucleic acid comprises RNA. In an aspect, a guide nucleic acid comprises DNA, RNA, or a combination thereof. In an aspect, a guide nucleic acid is single-stranded. In another aspect, a guide nucleic acid is at least partially double-stranded.

When a guide nucleic acid comprises RNA, it can be referred to as a “guide RNA.” In another aspect, a guide nucleic acid comprises DNA and RNA. In another aspect, a guide RNA is single-stranded. In another aspect, a guide RNA is double-stranded. In a further aspect, a guide RNA is partially double-stranded.

In an aspect, a guide nucleic acid comprises a guide RNA. In another aspect, a guide nucleic acid comprises at least one guide RNA. In another aspect, a guide nucleic acid comprises at least two guide RNAs. In another aspect, a guide nucleic acid comprises at least three guide RNAs. In another aspect, a guide nucleic acid comprises at least five guide RNAs. In another aspect, a guide nucleic acid comprises at least ten guide RNAs.

In another aspect, a guide nucleic acid comprises at least 10 nucleotides. In another aspect, a guide nucleic acid comprises at least 11 nucleotides. In another aspect, a guide nucleic acid comprises at least 12 nucleotides. In another aspect, a guide nucleic acid comprises at least 13 nucleotides. In another aspect, a guide nucleic acid comprises at least 14 nucleotides. In another aspect, a guide nucleic acid comprises at least 15 nucleotides. In another aspect, a guide nucleic acid comprises at least 16 nucleotides. In another aspect, a guide nucleic acid comprises at least 17 nucleotides. In another aspect, a guide nucleic acid comprises at least 18 nucleotides. In another aspect, a guide nucleic acid comprises at least 19 nucleotides. In another aspect, a guide nucleic acid comprises at least 20 nucleotides. In another aspect, a guide nucleic acid comprises at least 21 nucleotides. In another aspect, a guide nucleic acid comprises at least 22 nucleotides. In another aspect, a guide nucleic acid comprises at least 23 nucleotides. In another aspect, a guide nucleic acid comprises at least 24 nucleotides. In another aspect, a guide nucleic acid comprises at least 25 nucleotides. In another aspect, a guide nucleic acid comprises at least 26 nucleotides. In another aspect, a guide nucleic acid comprises at least 27 nucleotides. In another aspect, a guide nucleic acid comprises at least 28 nucleotides. In another aspect, a guide nucleic acid comprises at least 30 nucleotides. In another aspect, a guide nucleic acid comprises at least 35 nucleotides. In another aspect, a guide nucleic acid comprises at least 40 nucleotides. In another aspect, a guide nucleic acid comprises at least 45 nucleotides. In another aspect, a guide nucleic acid comprises at least 50 nucleotides.

In another aspect, a guide nucleic acid comprises between 10 nucleotides and 50 nucleotides. In another aspect, a guide nucleic acid comprises between 10 nucleotides and 40 nucleotides. In another aspect, a guide nucleic acid comprises between 10 nucleotides and 30 nucleotides. In another aspect, a guide nucleic acid comprises between 10 nucleotides and 20 nucleotides. In another aspect, a guide nucleic acid comprises between 16 nucleotides and 28 nucleotides. In another aspect, a guide nucleic acid comprises between 16 nucleotides and 25 nucleotides. In another aspect, a guide nucleic acid comprises between 16 nucleotides and 20 nucleotides.

In an aspect, a guide nucleic acid comprises at least 70% sequence complementarity to a target site. In an aspect, a guide nucleic acid comprises at least 75% sequence complementarity to a target site. In an aspect, a guide nucleic acid comprises at least 80% sequence complementarity to a target site. In an aspect, a guide nucleic acid comprises at least 85% sequence complementarity to a target site. In an aspect, a guide nucleic acid comprises at least 90% sequence complementarity to a target site. In an aspect, a guide nucleic acid comprises at least 91% sequence complementarity to a target site. In an aspect, a guide nucleic acid comprises at least 92% sequence complementarity to a target site. In an aspect, a guide nucleic acid comprises at least 93% sequence complementarity to a target site. In an aspect, a guide nucleic acid comprises at least 94% sequence complementarity to a target site. In an aspect, a guide nucleic acid comprises at least 95% sequence complementarity to a target site. In an aspect, a guide nucleic acid comprises at least 96% sequence complementarity to a target site. In an aspect, a guide nucleic acid comprises at least 97% sequence complementarity to a target site. In an aspect, a guide nucleic acid comprises at least 98% sequence complementarity to a target site. In an aspect, a guide nucleic acid comprises at least 99% sequence complementarity to a target site. In an aspect, a guide nucleic acid comprises 100% sequence complementarity to a target site. In another aspect, a guide nucleic acid comprises between 70% and 100% sequence complementarity to a target site. In another aspect, a guide nucleic acid comprises between 80% and 100% sequence complementarity to a target site. In another aspect, a guide nucleic acid comprises between 90% and 100% sequence complementarity to a target site. In an aspect, a guide nucleic acid is capable of hybridizing to a target site.

As noted above, some guided nucleases, such as CasX and Cas9, require another non-coding RNA component, referred to as a trans-activating crRNA (tracrRNA), to have functional activity. Guide nucleic acid molecules provided herein can combine a crRNA and a tracrRNA into one nucleic acid molecule in what is herein referred to as a “single guide RNA” (sgRNA). The gRNA guides the active CasX complex to a target site within a target sequence, where CasX can cleave the target site. In other embodiments, the crRNA and tracrRNA are provided as separate nucleic acid molecules. In an aspect, a guide nucleic acid comprises a crRNA. In another aspect, a guide nucleic acid comprises a tracrRNA. In a further aspect, a guide nucleic acid comprises a sgRNA.

### Target Sites

As used herein, a “target sequence” refers to a selected sequence or region of a DNA molecule in which a modification (e.g., cleavage, deamination, site-directed integration) is desired. A target sequence comprises a target site.

As used herein, a “target site” refers to the portion of a target sequence that is modified (e.g., cleaved) by a CRISPR effector protein. In contrast to a non-target nucleic acid (e.g., non-target ssDNA) or non-target region, a target site comprises significant complementarity to a guide nucleic acid or a guide RNA.

In an aspect, a target site is 100% complementary to a guide nucleic acid. In another aspect, a target site is 99% complementary to a guide nucleic acid. In another aspect, a target site is 98% complementary to a guide nucleic acid. In another aspect, a target site is 97% complementary to a guide nucleic acid. In another aspect, a target site is 96% complementary to a guide nucleic acid. In another aspect, a target site is 95% complementary to a guide nucleic acid. In another aspect, a target site is 94% complementary to a guide nucleic acid. In another aspect, a target site is 93% complementary to a guide nucleic acid. In another aspect, a target site is 92% complementary to a guide nucleic acid. In another aspect, a target site is 91% complementary to a guide nucleic acid. In another aspect, a target site is 90% complementary to a guide nucleic acid. In another aspect, a target site is 85% complementary to a guide nucleic acid. In another aspect, a target site is 80% complementary to a guide nucleic acid.

In an aspect, a target site comprises at least one PAM site. In an aspect, a target site is adjacent to a nucleic acid sequence that comprises at least one PAM site. In another aspect, a target site is within 5 nucleotides of at least one PAM site. In a further aspect, a target site is within 10 nucleotides of at least one PAM site. In another aspect, a target site is within 15 nucleotides of at least one PAM site. In another aspect, a target site is within 20 nucleotides of at least one PAM site. In another aspect, a target site is within 25 nucleotides of at least one PAM site. In another aspect, a target site is within 30 nucleotides of at least one PAM site.

In an aspect, a target site is positioned within genic DNA. In another aspect, a target site is positioned within a gene. In another aspect, a target site is positioned within a gene of interest. In another aspect, a target site is positioned within an exon of a gene. In another aspect, a target site is positioned within an intron of a gene. In another aspect, a target site is positioned within 5′-UTR of a gene. In another aspect, a target site is positioned within a 3′-UTR of a gene. In another aspect, a target site is positioned within intergenic DNA.

In an aspect, a target DNA molecule is single-stranded. In another aspect, a target DNA molecule is double-stranded.

In an aspect, a target sequence comprises genomic DNA. In an aspect, a target sequence is positioned within a nuclear genome. In an aspect, a target sequence comprises chromosomal DNA. In an aspect, a target sequence comprises plasmid DNA. In an aspect, a target sequence is positioned within a plasmid. In an aspect, a target sequence comprises mitochondrial DNA. In an aspect, a target sequence is positioned within a mitochondrial genome. In an aspect, a target sequence comprises plastid DNA. In an aspect, a target sequence is positioned within a plastid genome. In an aspect, a target sequence comprises chloroplast DNA. In an aspect, a target sequence is positioned within a chloroplast genome. In an aspect, a target sequence is positioned within a genome selected from the group consisting of a nuclear genome, a mitochondrial genome, and a plastid genome.

In an aspect, a target sequence comprises genic DNA. As used herein, “genic DNA” refers to DNA that encodes one or more genes. In another aspect, a target sequence comprises intergenic DNA. In contrast to genic DNA, “intergenic DNA” comprises noncoding DNA, and lacks DNA encoding a gene. In an aspect, intergenic DNA is positioned between two genes.

In an aspect, a target sequence encodes a gene. As used herein, a “gene” refers to a polynucleotide that can produce a functional unit (e.g., without being limiting, for example, a protein, or a non-coding RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a non-protein-coding RNA molecule or a precursor thereof. In another aspect, a gene encodes a protein. In some embodiments, the target sequence is selected from the group consisting of: a promoter, an enhancer sequence, a leader sequence, a transcriptional start site, a transcriptional stop site, a polyadenylation site, an exon, an intron, a splice site, a 5′-UTR, a 3′-UTR, a protein coding sequence, a non-protein-coding sequence, a miRNA, a pre-miRNA and a miRNA binding site.

Non-limiting examples of a non-protein-coding RNA molecule include a microRNA (miRNA), a miRNA precursor (pre-miRNA), a small interfering RNA (siRNA), a small RNA (18 to 26 nucleotides in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single guide RNA (sgRNA). In an aspect, a non-protein-coding RNA molecule comprises a miRNA. In an aspect, a non-protein-coding RNA molecule comprises a siRNA. In an aspect, a non-protein-coding RNA molecule comprises a ta-siRNA. In an aspect, a non-protein-coding RNA molecule is selected from the group consisting of a miRNA, a siRNA, and a ta-siRNA.

As used herein, a “gene of interest” refers to a polynucleotide sequence encoding a protein or a non-protein-coding RNA molecule that is to be integrated into a target sequence, or, alternatively, an endogenous polynucleotide sequence encoding a protein or a non-protein-coding RNA molecule that is to be edited by a ribonucleoprotein. In an aspect, a gene of interest encodes a protein. In another aspect, a gene of interest encodes a non-protein-coding RNA molecule. In an aspect, a gene of interest is exogenous to a targeted DNA molecule. In an aspect, a gene of interest replaces an endogenous gene in a targeted DNA molecule.

### Mutations

In an aspect, a ribonucleoprotein or method provided herein generates at least one mutation in a target sequence of an egg, embryo, and/or meiotic cell.

In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to an egg cell-preferred promoter or a second nucleic acid encoding at least one guide nucleic acid operably linked to a heterologous second promoter. In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to an embryo tissue-preferred promoter or a second nucleic acid encoding at least one guide nucleic acid operably linked to a heterologous second promoter.

In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a heterologous promoter or a second nucleic acid encoding at least one guide nucleic acid operably linked to an egg cell-preferred promoter. In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a heterologous promoter or a second nucleic acid encoding at least one guide nucleic acid operably linked to an embryo tissue-preferred promoter.

As used herein, a “mutation” refers to a non-naturally occurring alteration to a nucleic acid or amino acid sequence as compared to a naturally occurring reference nucleic acid or amino acid sequence from the same organism. It will be appreciated that, when identifying a mutation, the reference sequence should be from the same nucleic acid (e.g, gene, non-coding RNA) or amino acid (e.g, protein). In determining if a difference between two sequences comprises a mutation, it will be appreciated in the art that the comparison should not be made between homologous sequences of two different species or between homologous sequences of two different varieties of a single species. Rather, the comparison should be made between the edited (e.g., mutated) sequence and the endogenous, non-edited (e.g., “wildtype”) sequence of the same organism.

Several types of mutations are known in the art. In an aspect, a mutation comprises an insertion. An “insertion” refers to the addition of one or more nucleotides or amino acids to a given polynucleotide or amino acid sequence, respectively, as compared to an endogenous reference polynucleotide or amino acid sequence. In another aspect, a mutation comprises a deletion. A “deletion” refers to the removal of one or more nucleotides or amino acids to a given polynucleotide or amino acid sequence, respectively, as compared to an endogenous reference polynucleotide or amino acid sequence. In another aspect, a mutation comprises a substitution. A “substitution” refers to the replacement of one or more nucleotides or amino acids to a given polynucleotide or amino acid sequence, respectively, as compared to an endogenous reference polynucleotide or amino acid sequence. In another aspect, a mutation comprises an inversion. An “inversion” refers to when a segment of a polynucleotide or amino acid sequence is reversed end-to-end. In an aspect, a mutation provided herein comprises a mutation selected from the group consisting of an insertion, a deletion, a substitution, and an inversion.

In an aspect, a plant or seed comprises at least one mutation in a gene of interest, where the at least one mutation results in the deletion of one or more amino acids from a protein encoded by the gene of interest as compared to a wildtype protein.

In an aspect, a plant or seed comprises at least one mutation in a gene of interest, where the at least one mutation results in the substitution of one or more amino acids within a protein encoded by the gene of interest as compared to a wildtype protein.

In an aspect, a plant or seed comprises at least one mutation in a gene of interest, where the at least one mutation results in the insertion of one or more amino acids within a protein encoded by the gene of interest as compared to a wildtype protein.

Mutations in coding regions of genes (e.g., exonic mutations) can result in a truncated protein or polypeptide when a mutated messenger RNA (mRNA) is translated into a protein or polypeptide. In an aspect, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a seed or plant comprises at least one mutation, where the at least one mutation results in the introduction of a premature stop codon in a messenger RNA encoded by the gene of interest as compared to a wildtype messenger RNA.

In an aspect, a mutation provided herein comprises a null mutation. As used herein, a “null mutation” refers to a mutation that confers a complete loss-of-function for a protein encoded by a gene comprising the mutation, or, alternatively, a mutation that confers a complete loss-of-function for a small RNA encoded by a genomic locus. A null mutation can cause lack of mRNA transcript production, a lack of small RNA transcript production, a lack of protein function, or a combination thereof.

A mutation provided herein can be positioned in any part of an endogenous gene. In an aspect, a mutation provided herein is positioned within an exon of an endogenous gene. In another aspect, a mutation provided herein is positioned within an intron of an endogenous gene. In a further aspect, a mutation provided herein is positioned within a 5′-untranslated region of an endogenous gene. In still another aspect, a mutation provided herein is positioned within a 3′-untranslated region of an endogenous gene. In yet another aspect, a mutation provided herein is positioned within a promoter of an endogenous gene.

In an aspect, a mutation is positioned at a splice site within a gene. A mutation at a splice site can interfere with the splicing of exons during mRNA processing. If one or more nucleotides are inserted, deleted, or substituted at a splice site, splicing can be perturbed. Perturbed splicing can result in unspliced introns, missing exons, or both, from a mature mRNA sequence. Typically, although not always, a “GU” sequence is required at the 5′ end of an intron and a “AG” sequence is required at the 3′ end of an intron for proper splicing. If either of these splice sites are mutated, splicing perturbations can occur.

In an aspect, a seed or plant comprises at least one mutation, where the at least one mutation comprises the deletion of one or more splice sites from a gene of interest. In another aspect, a seed or plant comprises at least one mutation, where the at least one mutation is positioned within one or more splice sites from a gene of interest.

In an aspect, a mutation comprises a site-directed integration. In an aspect, a site-directed integration comprises the insertion of all or part of a desired sequence into a target sequence.

As used herein, “site-directed integration” refers to all, or a portion, of a desired sequence (e.g., an exogenous gene, an edited endogenous gene) being inserted or integrated at a desired site or locus within the plant genome (e.g., target sequence). As used herein, a “desired sequence” refers to a DNA molecule comprising a nucleic acid sequence that is to be integrated into a genome of a plant or plant cell. The desired sequence can comprise a transgene or construct. In an aspect, a nucleic acid molecule comprising a desired sequence comprises one or two homology arms flanking the desired sequence to promote the targeted insertion event through homologous recombination and/or homology-directed repair.

In an aspect, a method provided herein comprises site-directed integration of a desired sequence into a target sequence.

Any site or locus within the genome of a plant can be chosen for site-directed integration of a transgene or construct of the present disclosure. In an aspect, a target sequence is positioned within a B, or supernumerary, chromosome.

For site-directed integration, a double-strand break (DSB) or nick may first be made at a target sequence via a guided nuclease or ribonucleoprotein provided herein. In the presence of a desired sequence, the DSB or nick can then be repaired by homologous recombination (HR) between the homology arm(s) of the desired sequence and the target sequence, or by non-homologous end joining (NHEJ), resulting in site-directed integration of all or part of the desired sequence into the target sequence to create the targeted insertion event at the site of the DSB or nick.

In an aspect, site-directed integration comprises the use of NHEJ repair mechanisms endogenous to a cell. In another aspect, site-directed integration comprises the use of HR repair mechanisms endogenous to a cell.

In an aspect, repair of a double-stranded break generates at least one mutation in a gene of interest as compared to a control plant of the same line or variety.

In an aspect, a mutation comprises the integration of at least 5 contiguous nucleotides of a desired sequence into a target sequence. In an aspect, a mutation comprises the integration of at least 10 contiguous nucleotides of a desired sequence molecule into a target sequence. In an aspect, a mutation comprises the integration of at least 15 contiguous nucleotides of a desired sequence into a target sequence. In an aspect, a mutation comprises the integration of at least 20 contiguous nucleotides of a desired sequence into a target sequence. In an aspect, a mutation comprises the integration of at least 25 contiguous nucleotides of a desired sequence into a target sequence. In an aspect, a mutation comprises the integration of at least 50 contiguous nucleotides of a desired sequence into a target sequence. In an aspect, a mutation comprises the integration of at least 100 contiguous nucleotides of a desired sequence into a target sequence. In an aspect, a mutation comprises the integration of at least 250 contiguous nucleotides of a desired sequence into a target sequence. In an aspect, a mutation comprises the integration of at least 500 contiguous nucleotides of a desired sequence into a target sequence. In an aspect, a mutation comprises the integration of at least 1000 contiguous nucleotides of a desired sequence into a target sequence. In an aspect, a mutation comprises the integration of at least 2000 contiguous nucleotides of a desired sequence into a target sequence.

In an aspect, a mutation comprises the integration of between 5 contiguous nucleotides and 3500 contiguous nucleotides of a desired sequence into a target sequence. In an aspect, a mutation comprises the integration of between 5 contiguous nucleotides and 2500 contiguous nucleotides of a desired sequence into a target sequence. In an aspect, a mutation comprises the integration of between 5 contiguous nucleotides and 1500 contiguous nucleotides of a desired sequence into a target sequence. In an aspect, a mutation comprises the integration of between 5 contiguous nucleotides and 750 contiguous nucleotides of a desired sequence into a target sequence. In an aspect, a mutation comprises the integration of between 5 contiguous nucleotides and 500 contiguous nucleotides of a desired sequence into a target sequence. In an aspect, a mutation comprises the integration of between 5 contiguous nucleotides and 250 contiguous nucleotides of a desired sequence into a target sequence. In an aspect, a mutation comprises the integration of between 5 contiguous nucleotides and 150 contiguous nucleotides of a desired sequence into a target sequence. In an aspect, a mutation comprises the integration of between 25 contiguous nucleotides and 2500 contiguous nucleotides of a desired sequence into a target sequence. In an aspect, a mutation comprises the integration of between 25 contiguous nucleotides and 1500 contiguous nucleotides of a desired sequence into a target sequence. In an aspect, a mutation comprises the integration of between 25 contiguous nucleotides and 750 contiguous nucleotides of a desired sequence into a target sequence. In an aspect, a mutation comprises the integration of between 50 contiguous nucleotides and 2500 contiguous nucleotides of a desired sequence into a target sequence. In an aspect, a mutation comprises the integration of between 50 contiguous nucleotides and 1500 contiguous nucleotides of a desired sequence into a target sequence. In an aspect, a mutation comprises the integration of between 50 contiguous nucleotides and 750 contiguous nucleotides of a desired sequence into a target sequence. In an aspect, a mutation comprises the integration of between 100 contiguous nucleotides and 2500 contiguous nucleotides of a desired sequence into a target Sequence. In an aspect, a mutation comprises the integration of between 100 contiguous nucleotides and 1500 contiguous nucleotides of a desired sequence into a target Sequence. In an aspect, a mutation comprises the integration of between 100 contiguous nucleotides and 750 contiguous nucleotides of a desired sequence into a target Sequence.

In an aspect, a method provided herein comprises detecting an edit or a mutation in a target sequence. The screening and selection of mutagenized or edited plants or plant cells can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the above-referenced techniques are known in the art.

### Recombinases

Several embodiments described herein relate to methods and compositions for preferably or specifically inducing site-specific recombination in egg, embryo, and/or meiotic plant tissue. Several embodiments described herein relate to methods and compositions for providing egg, embryo, and/or meiotic plant tissue preferred or specific expression of a recombinase. Several embodiments described herein relate to methods and compositions for preferably or specifically expressing a DNA modification enzyme, such as a guided nuclease (e.g., a CRISPR/Cas system), in egg, embryo, and/or meiotic plant tissue by inducing site-specific recombination to operably linking a polynucleotide encoding a DNA modification enzyme to a constitutive promoter. In some embodiments, a transcribable polynucleotide encoding a DNA modification enzyme, such as a guided nuclease (e.g., a CRISPR/Cas system) is operably linked to a constitutive promoter by recombinase-mediated excision of a intervening polynucleotide sequence preferentially or specifically in egg, embryo, and/or meiotic plant tissue.

Site-specific recombination occurs when DNA strand exchange takes place between DNA segments possessing at least some sequence homology with each other. Site-specific recombinases are able to recognize and bind to “recombination sites,” which are short, specific DNA sequences which are cleaved by the recombinase, allowing the exchange of DNA strands, which is followed by strand repair. Typically, each recombinase protein binds to a specific, and unique, recombination site. As used herein, a “recombinase” refers to an enzyme that is capable of catalyzing site-specific recombination events within DNA. Recombinases are capable of excising DNA, inserting DNA, inverting DNA, translocating DNA, and/or exchanging DNA.

In an aspect, this disclosure provides methods and compositions for specifically or preferentially providing a recombinase in egg, embryo, and/or meiotic plant tissue. In an aspect, this disclosure provides a nucleic acid sequence encoding a recombinase operably linked to a promoter as described in Table 1. In an aspect, a recombinant nucleic acid construct comprises a sequence encoding at least one recombinase operably linked to a promoter as described in Table 1. In an aspect, a recombinant nucleic acid construct comprising a sequence encoding at least one recombinase operably linked to a promoter as described in Table 1 is provided to a plant cell in combination with a recombinant nucleic acid construct comprising a polynucleotide encoding a DNA modification enzyme, an intervening sequence flanked by recombination sites, and a constitutive promoter, wherein excision of the intervening sequence operably links polynucleotide encoding a DNA modification enzyme to the constitutive promoter preferentially in egg, embryo, and/or meiotic tissue.

In an aspect, a recombinase is a tyrosine recombinase. In an aspect, a tyrosine recombinase is selected from the group consisting of a Cre recombinase and a Flp recombinase.

In an aspect, a recombinase is Cre recombinase. Cre-lox is a site-specific recombination system derived from the bacteriophage P1. Cre-lox can be used to invert a nucleic acid sequence, delete a nucleic acid sequence, or translocate a nucleic acid sequence. In this system, Cre recombinase recombines a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete.

In an aspect, a recombinase is Flippase (Flp). The Flp-FRT site-specific recombination system comes from the 2p plasmid from the baker's yeast Saccharomyces cerevisiae and is similar to the Cre-lox system. Flp is capable of inducing recombination between flippase recognition target (FRT) sites. FRT sites comprise 34 nucleotides. Flp binds to the “arms” of the FRT sites (one arm is in reverse orientation) and cleaves the FRT site at either end of an intervening nucleic acid sequence. After cleavage, Flp recombines nucleic acid sequences between two FRT sites.

In an aspect, a recombination site is a lox site. In an aspect, a lox site is selected from the group consisting of a loxP site, a lox 2272 site, a loxN site, a lox 511 site, a lox 5171 site, a lox71 site, a lox66 site, a loxLTR site, an M2 site, an M3 site, an M7 site, and an M11 site. In an aspect, a recombination site is an FRT site.

### TALE

Several embodiments provided herein relate to the use of TALE activators to preferentially express a DNA modification enzyme, such as a guided nuclease (e.g., a CRISPR/Cas system), in egg, embryo, and/or meiotic plant tissue. In several embodiments, high levels of egg, embryo, and/or meiotic tissue specific expression of a DNA modification enzyme such as a guided nuclease (e.g., a CRISPR/Cas system), by providing to a plant cell: 1) an expression construct comprising a promoter as described in Table 1 operably linked to a sequence encoding a TALE and 2) and an expression construct comprising one or more TALE binding sites (TB) operably linked to a minimal promoter and a sequence encoding the DNA modification enzyme and generating a plant therefrom. In some embodiments, an expression construct encoding one or more guide nucleic acids is further provided. In some embodiments, levels of egg, embryo, and/or meiotic tissue specific expression of a DNA modification enzyme can be modulated by altering the number of TBs.

As used herein, “TALE protein” refers to a transcription activator-like effector (TALE) protein or a homolog thereof. TALE proteins were originally identified as a virulence factor from the phytopathogenic bacterial genera Xanthomonas or Ralstonia. These proteins are secreted by the phytopathogenic bacteria to alter transcription of host genes in plant cells. TALE proteins bind DNA in the nucleus, via a domain of DNA-binding repeats, where they act as transcriptional activators thereby contributing to virulence. The TALE moves to the nucleus, where it recognizes and binds to a specific DNA sequence in the regulatory region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs. As used herein, a “TALE binding site” (TBS) refers to a specific DNA sequence that is recognized and bound by the “TALE DNA-binding domain” of the TALE protein.

### Plants

Any plant or plant cell can be used with the methods and compositions provided herein. In an aspect, a plant is selected from the group consisting of a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, and a potato plant. In an aspect, a plant is an angiosperm. In an aspect, a plant is a gymnosperm. In an aspect, a plant is a monocotyledonous plant. In an aspect, a plant is a dicotyledonous plant. In an aspect, a plant is a plant of a family selected from the group consisting of Alliaceae, Anacardiaceae, Apiaceae, Arecaceae, Asteraceae, Brassicaceae, Caesalpiniaceae, Cucurbitaceae, Ericaceae, Fabaceae, Juglandaceae, Malvaceae, Mimosaceae, Moraceae, Musaceae, Orchidaceae, Papilionaceae, Pinaceae, Poaceae, Rosaceae, Rutaceae, Rubiaceae, and Solanaceae.

In an aspect, a plant cell is selected from the group consisting of a corn cell, a rice cell, a sorghum cell, a wheat cell, an alfalfa cell, a barley cell, a millet cell, a rye cell, a sugarcane cell, a cotton cell, a soybean cell, a canola cell, a tomato cell, an onion cell, a cucumber cell, an Arabidopsis cell, and a potato cell. In an aspect, a plant cell is an angiosperm plant cell. In an aspect, a plant cell is a gymnosperm plant cell. In an aspect, a plant cell is a monocotyledonous plant cell. In an aspect, a plant cell is a dicotyledonous plant cell. In an aspect, a plant cell is a plant cell of a family selected from the group consisting of Alliaceae, Anacardiaceae, Apiaceae, Arecaceae, Asteraceae, Brassicaceae, Caesalpiniaceae, Cucurbitaceae, Ericaceae, Fabaceae, Juglandaceae, Malvaceae, Mimosaceae, Moraceae, Musaceae, Orchidaceae, Papilionaceae, Pinaceae, Poaceae, Rosaceae, Rutaceae, Rubiaceae, and Solanaceae.

As used herein, a “variety” refers to a group of plants within a species (e.g., without being limiting Zea mays) that share certain genetic traits that separate them from other possible varieties within that species. Varieties can be inbreds or hybrids, though commercial plants are often hybrids to take advantage of hybrid vigor. Individuals within a hybrid cultivar are homogeneous, nearly genetically identical, with most loci in the heterozygous state.

As used herein, the term “inbred” means a line that has been bred for genetic homogeneity. In an aspect, a seed provided herein is an inbred seed. In an aspect, a plant provided herein is an inbred plant.

As used herein, the term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines. In an aspect, a seed provided herein is a hybrid seed. In an aspect, a plant provided herein is a hybrid plant.

### Transformation

Methods can involve transient transformation or stable integration of any nucleic acid molecule into any plant or plant cell provided herein.

As used herein, “stable integration” or “stably integrated” refers to a transfer of DNA into genomic DNA of a targeted cell or plant that allows the targeted cell or plant to pass the transferred DNA to the next generation of the transformed organism. Stable transformation requires the integration of transferred DNA within the reproductive cell(s) of the transformed organism. As used herein, “transiently transformed” or “transient transformation” refers to a transfer of DNA into a cell that is not transferred to the next generation of the transformed organism. In a transient transformation the transformed DNA does not typically integrate into the transformed cell's genomic DNA. In one aspect, a method stably transforms a plant cell or plant with one or more nucleic acid molecules provided herein. In another aspect, a method transiently transforms a plant cell or plant with one or more nucleic acid molecules provided herein.

In an aspect, a nucleic acid molecule encoding a guided nuclease is stably integrated into a genome of a plant. In an aspect, a nucleic acid molecule encoding a Cas12a nuclease is stably integrated into a genome of a plant. In an aspect, a nucleic acid molecule encoding a CasX nuclease is stably integrated into a genome of a plant. In an aspect, a nucleic acid molecule encoding a guide nucleic acid is stably integrated into a genome of a plant. In an aspect, a nucleic acid molecule encoding a guide RNA is stably integrated into a genome of a plant. In an aspect, a nucleic acid molecule encoding a single-guide RNA is stably integrated into a genome of a plant.

Numerous methods for transforming cells with a recombinant nucleic acid molecule or construct are known in the art, which can be used according to methods of the present application. Any suitable method or technique for transformation of a cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants.

In an aspect, a method comprises providing a cell with a nucleic acid molecule via Agrobacterium-mediated transformation. In an aspect, a method comprises providing a cell with a nucleic acid molecule via polyethylene glycol-mediated transformation. In an aspect, a method comprises providing a cell with a nucleic acid molecule via biolistic transformation. In an aspect, a method comprises providing a cell with a nucleic acid molecule via liposome-mediated transfection. In an aspect, a method comprises providing a cell with a nucleic acid molecule via viral transduction. In an aspect, a method comprises providing a cell with a nucleic acid molecule via use of one or more delivery particles. In an aspect, a method comprises providing a cell with a nucleic acid molecule via microinjection. In an aspect, a method comprises providing a cell with a nucleic acid molecule via electroporation.

In an aspect, a nucleic acid molecule is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

Other methods for transformation, such as vacuum infiltration, pressure, sonication, and silicon carbide fiber agitation, are also known in the art and envisioned for use with any method provided herein.

Methods of transforming cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell with any of the nucleic acid molecules provided herein.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

Delivery vehicles, vectors, particles, nanoparticles, formulations and components thereof for expression of one or more elements of a nucleic acid molecule are as used in WO 2014/093622. In an aspect, a method of providing a nucleic acid molecule or a protein to a cell comprises delivery via a delivery particle. In an aspect, a method of providing a nucleic acid molecule to a plant cell or plant comprises delivery via a delivery vesicle. In an aspect, a delivery vesicle is selected from the group consisting of an exosome and a liposome. In an aspect, a method of providing a nucleic acid molecule to a plant cell or plant comprises delivery via a viral vector. In an aspect, a viral vector is selected from the group consisting of an adenovirus vector, a lentivirus vector, and an adeno-associated viral vector. In another aspect, a method providing a nucleic acid molecule to a plant cell or plant comprises delivery via a nanoparticle. In an aspect, a method providing a nucleic acid molecule to a plant cell or plant comprises microinjection. In an aspect, a method providing a nucleic acid molecule to a plant cell or plant comprises polycations. In an aspect, a method providing a nucleic acid molecule to a plant cell or plant comprises a cationic oligopeptide.

In an aspect, a delivery particle is selected from the group consisting of an exosome, an adenovirus vector, a lentivirus vector, an adeno-associated viral vector, a nanoparticle, a polycation, and a cationic oligopeptide. In an aspect, a method provided herein comprises the use of one or more delivery particles. In another aspect, a method provided herein comprises the use of two or more delivery particles. In another aspect, a method provided herein comprises the use of three or more delivery particles.

Suitable agents to facilitate transfer of nucleic acids into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning includes (a) surfactants, (b) organic solvents, aqueous solutions, or aqueous mixtures of organic solvents, (c) oxidizing agents, (e) acids, (f) bases, (g) oils, (h) enzymes, or combinations thereof.

Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e. g., plant-sourced oils, crop oils (such as those listed in the 9^{th} Compendium of Herbicide Adjuvants, publicly available on line at www(dot)herbicide(dot)adjuvants(dot)com) can be used, e. g., paraffinic oils, polyol fatty acid esters, or oils with short-chain molecules modified with amides or polyamines such as polyethyleneimine or N-pyrrolidine.

Examples of useful surfactants include sodium or lithium salts of fatty acids (such as tallow or tallowamines or phospholipids) and organosilicone surfactants. Other useful surfactants include organosilicone surfactants including nonionic organosilicone surfactants, e. g., trisiloxane ethoxylate surfactants or a silicone polyether copolymer such as a copolymer of polyalkylene oxide modified heptamethyl trisiloxane and allyloxypolypropylene glycol methylether (commercially available as Silwet® L-77).

Useful physical agents can include (a) abrasives such as carborundum, corundum, sand, calcite, pumice, garnet, and the like, (b) nanoparticles such as carbon nanotubes or (c) a physical force. Carbon nanotubes are disclosed by Kam et. al. (2004) Am. Chem. Soc, 126 (22):6850-6851, Liu et. al. (2009) Nano Lett, 9(3): 1007-1010, and Khodakovskaya et. al. (2009) ACS Nano, 3(10):3221-3227. Physical force agents can include heating, chilling, the application of positive pressure, or ultrasound treatment. Embodiments of the method can optionally include an incubation step, a neutralization step (e.g., to neutralize an acid, base, or oxidizing agent, or to inactivate an enzyme), a rinsing step, or combinations thereof. The methods of the invention can further include the application of other agents which will have enhanced effect due to the silencing of certain genes. For example, when a polynucleotide is designed to regulate genes that provide herbicide resistance, the subsequent application of the herbicide can have a dramatic effect on herbicide efficacy.

Agents for laboratory conditioning of a plant cell to permeation by polynucleotides include, e.g., application of a chemical agent, enzymatic treatment, heating or chilling, treatment with positive or negative pressure, or ultrasound treatment. Agents for conditioning plants in a field include chemical agents such as surfactants and salts.

In an aspect, a transformed or transfected cell is a plant cell. Recipient plant cell or explant targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a guard cell, a trichome cell, a root hair cell, a storage root cell, or a tuber cell. In another aspect, this disclosure provides a protoplast. In another aspect, this disclosure provides a plant callus cell. Any cell from which a fertile plant can be regenerated is contemplated as a useful recipient cell for practice of this disclosure. Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference. Transformed explants, cells or tissues can be subjected to additional culturing steps, such as callus induction, selection, regeneration, etc., as known in the art. Transformed cells, tissues or explants containing a recombinant DNA insertion can be grown, developed or regenerated into transgenic plants in culture, plugs or soil according to methods known in the art. In one aspect, this disclosure provides plant cells that are not reproductive material and do not mediate the natural reproduction of the plant. In another aspect, this disclosure also provides plant cells that are reproductive material and mediate the natural reproduction of the plant. In another aspect, this disclosure provides plant cells that cannot maintain themselves via photosynthesis. In another aspect, this disclosure provides somatic plant cells. Somatic cells, contrary to germline cells, do not mediate plant reproduction. In one aspect, this disclosure provides a non-reproductive plant cell.

### Use of Haploids/Haploid Induction Lines

Several embodiments relate to the use of the methods and compositions as described herein in combination with haploid induction techniques. As used herein, a “haploid” cell or nucleus comprises a single set of unpaired chromosomes (x). In contrast, a “diploid” cell or nucleus comprises two complete sets of chromosomes (2x) that are capable of homologous pairing. As used herein, a “haploid plant” describes a sporophyte comprising a plurality of cells comprising a haploid nuclear genome. A haploid plant provided herein can be a maternal haploid plant, meaning it has lost its paternal nuclear genome while retaining its maternal nuclear genome. Alternatively, a haploid plant provided herein can be a paternal haploid plant, meaning it has lost its maternal nuclear genome while retaining its paternal nuclear genome. Typically, maternal mitochondria and plastid (e.g., chloroplast) genomes are retained in both maternal and paternal haploid plants.

In some embodiments, a ‘doubled haploid (DH)’ method is used to rapidly produce homozygous plants. Progenies of DH plants are genetically homogeneous material, allowing breeders to evaluate their traits of interest on genetically fixed material at an early stage of the breeding cycle, thus increasing breeding efficiency (see Gilles L M et. al. Curr Biol. 2017 Oct. 23; 27(20):R1095-R1097. The DH technology relies on two main steps: (1) a haploid induction system to generate haploid embryos or plantlets, and (2) a chromosome doubling step to restore diploidy of these plantlets.

“Haploid induction (HI)” is a phenomena in some plants characterized by loss of the parental inducer chromosomes during embryo development. As used herein, a “haploid induction (HI) plant” is a plant capable of inducing haploidization in a progeny plant by eliminating one set of chromosomes. Maternal haploid induction is triggered by the pollinator (male) parent. Paternal haploid induction is triggered by the female parent.

Haploid inducer lines are routinely used in plant breeding especially for maize. A number of known haploid-inducing maize lines exist including but not limited to: stock 6, MHI (Moldovian Haploid Inducer), indeterminate gametophyte (ig 1) mutation, KEMS, RWK, ZEM, ZMS, KMS. Haploid inducer lines have also been created in Arabidopsis thaliana, Brassica juncea and maize by the use of engineered centromeric histone 3 (CENH3) variants (See Ravi and Chan. 2010. Nature. 464:615-6190). In some aspects, the haploid induction lines described here include the Maize Stock 6 line, maize plants harboring mutations in the ig-1 locus, MHI inducer lines, KEMS inducer lines, RWK inducer lines, ZEM inducer lines, ZMS inducer lines, KMS inducer lines. In some aspects the Haploid inducer line comprises a modified MATRILINEAL/NOT LIKE DAD/ZmPHOSPHOLIPASE-A1 (MATL/NLD/ZmPLA1) gene. In some aspects, the haploid inducer line described herein comprises a modified CEN H3 variant.

The benefits offered by haploid induction systems to crop breeding programs are diverse, as DH technology can be used in conjunction with several different molecular techniques to overcome various constraints to crop improvement. One example is the use of haploid induction systems to expand the application of genome editing technologies to crops. The genome editing component (for e.g., the guided nuclease) could be introduced into the Haploid Inducer line which is then crossed to a non-inducer maize line. The haploid progeny is then screened for nuclease-induced mutations and genome doubling is subsequently induced to produce diploid, editing component-free, genome-edited cultivars. This methodology has been described in detail in US20190169596 (Application U.S. Ser. No. 16/275,200) and is incorporated herein by reference in its entirety.

The present disclosure will now be described with reference to the following examples. It should be appreciated that these examples are not intended to limit the scope of the claims to the present disclosure; but are rather intended to be examples of certain embodiments. Any variations in the exemplified methods that occur to the skilled artisan are intended to fall within the scope of the present disclosure.

### EXAMPLES

### Example 1. Expression of Cas12a in Meiotic Egg Cells or Embryo Tissue to Generate Germinal Mutations or Targeted Integration of Template DNA

Several Agrobacterium T-DNA vectors were generated to preferentially express Cas12a in corn egg cells and/or corn embryo cells undergoing meiosis (meiocytes). See Table 2.

TABLE 2
Cassettes designed to express Cas12a preferentially, or solely, in corn egg
cells and/or corn embryo cells and/or cells undergoing meiosis.

| | | | Promoter |
|---|---|---|---|
| Construct | Promoter::LbCas12a | Expression | SEQ ID NO. |
| 1 | ZmDSUL1::LbCas12a | Embryo | 1 |
| 2 | ZmEA1::LbCas12a | Egg/Embryo | 2 |
| 3 | ZmES4::LbCas12a | Egg/Embryo | 3 |
| 4 | ZmDMC1::LbCas12a | Meiocyte | 4 |
| 5 | ZmMps1::LbCas12a | Meiocyte | 5 |
| 6 | ZmAdf1::LbCas12a | Meiocyte | 6 |

The plant codon optimized LbCas12a sequence (SEQ ID NO: 7) in these cassettes was flanked by NLS sequences at the 5′ and 3′ ends (SEQ ID:8 and SEQ ID:9) and operably linked to a transcription terminator sequence from a rice Lipid transfer protein (LTP) gene (described in US20200080096 as SEQ ID NO:8). Each vector also contained an expression cassette encoding a Cas12a gRNA targeting a unique corn genomic site (ZmTS1) under the control of the Pol III ZmU6 promoter (SEQ ID NO: 10); an expression cassette flanked by ZmTS1 target sites, where the cassette comprised a constitutive promoter operably linked to a Gene of Interest (GOI); and an expression cassette for a selectable marker conferring resistance to the herbicide glyphosate. Corn 01DKD2 cultivar embryos were transformed with the vectors described above by agrobacterium-mediated transformation and R0 plants were regenerated from the transformed corn cells. DNA was extracted from leaf samples from 59-153 R0 seedlings generated from each construct. The genomic target site was sequenced and analyzed for the presence of targeted mutations. A Taqman based assay was also performed to identify the copy number of the Cas12a carrying construct. For all reproductive promoters except ZmDSUL, no mutations were detected at ZmTS1 site in the R0 generation. The ZmDSUL:LbCas12a (Construct 1), showed about a 32% target site mutation rate in the R0 seedlings (See Table 3). Lack of mutations at the ZmTS1 target site in the newly transformed (or R0) plants is expected where LbCas12a expression is confined to the reproductive tissue.

20 R0 lines from each transformed construct were grown to maturity and at least one ear from each transformed corn plant was self-pollinated. 10 R1 lines were selected and up to 16 seedlings per line were germinated, screened for mutations in ZmTS1 and the mutation rates (cutting rates) were calculated. Taqman assays were also performed to determine the presence and copy number of the LbCas12a expression cassette. The overall target site mutation rate among all lines generated from Constructs 2-5 ranged from about a 1.1% to 14%. The average mutation rates are shown in Table 3. 4 R1 seedlings expressing ZmDSUL::LbCas12a (Construct 1) were tested and no mutations were observed. Co-expression of Cas12a and its cognitive gRNA in cells undergoing meiosis are expected to generate a double stranded break at the ZmTS1 target site and subsequent imperfect DNA repair generates unique mutations in egg cells and embryos created by the pollination.

TABLE 3
Average mutation rates observed at
ZmTS1 target site in R0 and R1 plants

| | Promoter | | | | | |
|---|---|---|---|---|---|---|
| | ZmDMC1 | ZmMps1 | ZmAdf1 | ZmES4 | ZmEA1 | DSUL1 |
| Construct | 4 | 5 | 6 | 3 | 2 | 1 |
| Expression | Meiotic | Meiotic | Meiotic | Egg/ | Egg/ | Embryo |
| | | | | Embryo | Embryo | |
| R0 Events | 153 | 110 | 153 | 81 | 115 | 59 |
| tested | | | | | | |
| R0 Cutting | 0% | 0% | 0% | 0% | 0% | 32% |
| Rate | | | | | | |
| (Avg.) | | | | | | |
| R1 Cutting | 7.69% | 2.67% | 1.10% | 14.04% | 3.60% | 0.00% |
| (Avg.) | | | | | | |

Mutation rates in R1 plants generated from individual R0 lines can vary as shown in Tables 4-8. For example, in the highest cutting promoter, ZmES4, some lines with single copy Cpf1 cassette showed 0% mutation rate, while another line showed about an 11% mutation rate. The highest mutation rate observed was 46%, which originated from an R0 line that had two copies of ZmES4:LbCas12a. In the ZmES4:Cas12a plants, consistently among all lines tested, R0 lines with 2 copies of Cas12a had a higher target mutation rate than one copy R0 lines (see Table 7).

TABLE 4
Mutation rates in R1 seedlings from pZmDMC1::LbCas12a
transformed Corn plants.

| | Number | Number | Number | % mutations | Cas12a |
|---|---|---|---|---|---|
| | of R1 | Cas12a+ | Cas12a+ | in Cas12a+ | Copy |
| R0 Event | plants | plants | Mutation+ | plants | number |
| ZM_S22307337 | 13 | 10 | 0 | 0.00% | 1 |
| ZM_S22307366 | 12 | 8 | 0 | 0.00% | 1 |
| ZM_S22307385 | 15 | 11 | 0 | 0.00% | 1 |
| ZM_S22307402 | 16 | 14 | 0 | 0.00% | 1 |
| ZM_S22307410 | 16 | 14 | 3 | 21.43% | 1 |
| ZM_S22307411 | 16 | 13 | 1 | 7.69% | 1 |
| ZM_S22307475 | 14 | 11 | 1 | 9.09% | 1 |
| ZM_S22307488 | 12 | 11 | 1 | 9.09% | 1 |
| ZM_S22307495 | 15 | 12 | 1 | 8.33% | 2 |

TABLE 5
Mutation rates in R1 seedlings from pZmMPS4::LbCas12a
transformed Corn plants.

| | Number | Number | Number | % Mutant in | Cas12a |
|---|---|---|---|---|---|
| | of R1 | Cas12a+ | Cas12a+ | Cas12a+ | copy |
| R0 Event | plants | plants | Mutation+ | plants | number |
| ZM_S22324677 | 15 | 12 | 1 | 8.33% | 2 |
| ZM_S22324740 | 16 | 12 | 0 | 0.00% | 1 |
| ZM_S22324702 | 13 | 8 | 0 | 0.00% | 1 |
| ZM_S22324681 | 16 | 9 | 0 | 0.00% | 1 |
| ZM_S22324728 | 16 | 14 | 0 | 0.00% | 2 |
| ZM_S22324698 | 15 | 11 | 0 | 0.00% | 1 |
| ZM_S22324652 | 14 | 10 | 1 | 10.00% | 2 |
| ZM_S22324654 | 16 | 9 | 1 | 11.11% | 1 |
| ZM_S22324675 | 15 | 14 | 1 | 7.14% | 1 |
| ZM_S22324723 | 13 | 10 | 0 | 0.00% | 1 |

TABLE 6
Mutation rates in R1 seedlings from pZmAdf1::LbCas12a
transformed Corn plants.

| | Number | Number | Number | % mutant in | Cas12a |
|---|---|---|---|---|---|
| | of R1 | Cas12a+ | Cas12a+ | Cas12a+ | copy |
| R0 Event | plants | plants | Mutation+ | plants | number |
| ZM_S22309320 | 16 | 12 | 1 | 8.33% | 2 |
| ZM_S22309333 | 11 | 8 | 0 | 0.00% | 1 |
| ZM_S22309363 | 14 | 9 | 0 | 0.00% | 1 |
| ZM_S22309387 | 16 | 13 | 0 | 0.00% | 1 |
| ZM_S22309393 | 15 | 9 | 0 | 0.00% | 1 |
| ZM_S22309410 | 14 | 9 | 0 | 0.00% | 1 |
| ZM_S22309419 | 12 | 10 | 0 | 0.00% | 1 |
| ZM_S22309422 | 14 | 10 | 0 | 0.00% | 1 |
| ZM_S22309465 | 14 | 11 | 0 | 0.00% | 1 |

TABLE 7
Mutation rates in R1 seedlings from pZmES4::LbCas12a
transformed Corn plants.

| | Number | Number | Number | % mutant in | LbCas12a |
|---|---|---|---|---|---|
| | of R1 | Cas12a+ | Cas12a + | Cas12a+ | Copy |
| R0 Event | plants | plants | Mutation+ | plants | Number |
| ZM_S22321323 | 16 | 13 | 6 | 46.15% | 2 |
| ZM_S22321283 | 15 | 9 | 0 | 0.00% | 1 |
| ZM_S22321299 | 15 | 11 | 4 | 36.36% | 2 |
| ZM_S22321291 | 15 | 8 | 1 | 12.50% | 2 |
| ZM_S22321261 | 15 | 11 | 0 | 0.00% | 1 |
| ZM_S22321307 | 13 | 12 | 3 | 25.00% | 2 |
| ZM_S22321298 | 16 | 12 | 1 | 8.33% | 1 |
| ZM_S22321335 | 13 | 9 | 3 | 33.33% | 2 |
| ZM_S22321326 | 10 | 9 | 1 | 11.11% | 1 |
| ZM_S22321262 | 14 | 13 | 1 | 7.69% | 1 |

TABLE 8
Mutation rates in R1 seedlings from pZmEA1::LbCas12a
transformed Corn plants.

| | Number | Number | Number | % Mutations | Cas12a |
|---|---|---|---|---|---|
| | of R1 | Cas12+a | Cas12a+ | in Cas12a+ | copy |
| R0 Event | plants | plants | Mutation+ | plants | number |
| ZM_S22317328 | 12 | 9 | 1 | 11.11% | 1 |
| ZM_S22317003 | 14 | 10 | 1 | 10.00% | 1 |
| ZM_S22317338 | 14 | 10 | 2 | 20.00% | 1 |
| ZM_S22317300 | 15 | 10 | 0 | 0.00% | 1 |
| ZM_S22317305 | 13 | 9 | 0 | 0.00% | 1 |
| ZM_S22316987 | 16 | 12 | 1 | 8.33% | 1 |
| ZM_S22317322 | 15 | 10 | 0 | 0.00% | 1 |
| ZM_S22317345 | 16 | 11 | 0 | 0.00% | 1 |
| ZM_S22316989 | 13 | 11 | 0 | 0.00% | 1 |
| ZM_S22317296 | 10 | 8 | 0 | 0.00% | 1 |

TABLE 9
Mutation rates in R1 seedlings from pZmEDSUL1::LbCas12a
transformed Corn plants.

| | Number | Number | Number | % mutations | Cas12a |
|---|---|---|---|---|---|
| | of R1 | Cas12a+ | Cas12a+ | in Cas12a+ | copy |
| R0 Event | plants | plants | Mutation+ | plants | number |
| ZM_S22307430 | 13 | 10 | 0 | 0.00% | 1 |
| ZM_S22307433 | 12 | 8 | 0 | 0.00% | 1 |
| ZM_S22307451 | 14 | 10 | 0 | 0.00% | 1 |
| ZM_S22307456 | 15 | 7 | 0 | 0.00% | 1 |

To increase confidence in the targeted mutation rate, three R0 lines were selected from ZmES4:LbCas12a transformants. For the selected lines, 152, 112, and 86 R1 seedlings were screened. The mutation rates were 74.2%, 49.4%, and 8.3%, respectively. The lines with 74.2% and 49.4% observed mutation rates were R1 seeds from a R0 line that had 2 copies of ZmES4:Cas12a.

Unique targeted mutations in R1 plants: Two R0 lines (ZM_S22321326 and ZM_S22321323) from ZmES4::LbCas12a, which is expected to selectively express LbCas12a in egg and synergid cells, were analyzed to assess the types of mutations created. Five mutant plants were produced from the ZM_S22321326 line. The sequencing results from the ZmTS1 gRNA target loci indicated that all five plants had unique mutations at the target site. 49 mutant seedlings were analyzed from the ZM_S22321323 R0 line which had two copies of ZmES4:LbCas12a. Out of 49 mutant seedlings, 56 target site mutations were identified. This suggests that some plants contained heterologous mutations. The 49 plants had a total of 23 unique mutations.

TABLE 10
Type and number of unique mutations observed in R1 plants
from ZmES4::LbCas12a R0 ZM_S22321326 event.

| | ZM_S22321326 | |
|---|---|---|
| | | # of |
| Mutation | Mutation Type | Mutations |
| 1 | 1 bp deletion at nucleotide 16 of the target site | 1 |
| 2 | 9 bp deletion at nucleotide 16 of the target site | 1 |
| 3 | 8 bp deletion at nucleotide 17 of the target site | 1 |
| 4 | 7 bp deletion at nucleotide 15 of the target site | 1 |
| 5 | 2 bp deletion at nucleotide 23 of the target site | 1 |
| | Total | 5 |

TABLE 11
Type and number of unique mutations observed in R1 plants
from ZmES4::LbCas12a R0 ZM_S22321323 event.

| | ZM_S22321323 | |
|---|---|---|
| | | # of |
| Mutation | Mutation Type | Mutations |
| 1 | 8 bp deletion at nucleotide 17 of the target site | 9 |
| 2 | 10 bp deletion at nucleotide 15 of the target site | 6 |
| 3 | 7 bp deletion at nucleotide 15 of the target site | 4 |
| 4 | 2 bp deletion at nucleotide 15 of the target site | 2 |
| 5 | 12 bp deletion at nucleotide 16 of the target site | 1 |
| 6 | 35 bp deletion at nucleotide 2 of the target site | 2 |
| 7 | 240 bp deletion at nucleotide 15 of the target site | 1 |
| 8 | 229 bp deletion at nucleotide 15 of the target site | 2 |
| 9 | 9 bp deletion at nucleotide 16 of the target site | 10 |
| 10 | 7 bp deletion at nucleotide 20 of the target site | 1 |
| 11 | 13 bp deletion at nucleotide 15 of the target site | 2 |
| 12 | 10 bp deletion at nucleotide 13 of the target site | 2 |
| 13 | 1nucletide substitution at position 15 of the target | 1 |
| | site and a 12 bp deletion starting at position | |
| | 16 of the target site | |
| 14 | 14 bp deletion at nucleotide 14 of the target site | 2 |
| 15 | 10 bp deletion at nucleotide 16 of the target site | 1 |
| 16 | 11 bp deletion at nucleotide 10 of the target site | 1 |
| 17 | 1 bp deletion at nucleotide 1 of the target site | 1 |
| 18 | 6 bp deletion at nucleotide 15 of the target site | 1 |
| 19 | 17 bp deletion at nucleotide 13 of the target site | 1 |
| 20 | 13 bp deletion at nucleotide 15 of the target site | 3 |
| 21 | 1 nucleotide substitution at position 8 | 1 |
| | of the target site | |
| 22 | 8 bp deletion at nucleotide 14 of the target site | 1 |
| 23 | 8 bp deletion at nucleotide 18 of the target site | 1 |
| | Total | 56 |

### Testing for Site Directed Integration of Template DNA in Plants Expressing Cas12a.

In addition to the Cas12a and gRNA expression cassette, each vector also contained an expression cassette for a gene of interest (GOI) that was flanked by the ZmTS1 gRNA target sequences. Expression of Cas12a in reproductive tissues is expected to create double stranded breaks on both sides of the GOI cassette releasing it from the T-DNA. This released DNA could serve as a donor for targeted insertion at the genomic ZmTS1 target site. When the CRISPR-Cpf1 complex cuts the target site within the genome, the non-homologous end joining (NHEJ) DNA repair pathway could insert the donor GOI cassette into the genomic target site. This form of SDI is also known as trans-fragment targeting (TFT). To test for SDI by TFT, flank PCR assays similar to those described in WO2019084148 were used to identify putative targeted insertions. Primers were designed to PCR amplify the expected insertion flanking sequence. Four separate PCR reactions were carried out: a left flank PCR and a right flank PCR for potential inserts that were positioned in the sense orientation, and a left flank PCR and a right flank PCR for inserts that were positioned in the antisense direction. In the initial screen of R0 lines, two plants showed flank positive PCRs. Both were identified in ZmES4:LbCas12a line and both produced only one flank PCR product. While one flank positive plant was in a line with an established mutation rate (11.11%), the other flank positive plant was in a line that had 0% mutation rate.

Taken together, the data shows that reproductive editing can be achieved when LbCas12a is expressed under the control of the promoter DMC1, Mps1 or Adf1 that preferentially or solely express in cells undergoing meiosis. Reproductive editing can also be achieved when LbCas12a is expressed by the Egg or Embryo expressing promoters like ZmES4 and ZmEA1. Additionally, the data demonstrates that a single R0 plant can produce many R1 offspring each with unique target site edits. This suggests that these promoters can be used to drive the expression of nucleases so as to increase the frequency of unique edits produced per transformed plants.

### Germinal Mutations are Heritable to the F1 Generation

Two R1 individuals from the ZmES4::LbCas12a R0 event ZM_S22321323 were grown to maturity and cross pollinated with a wild type 01DKD2 tester. R1 plant-1 comprised a 7 bp deletion at nucleotide 15 of the target site (see mutation 3 in Table 11) and R1 plant-2 comprised a 9 bp deletion at nucleotide 16 of the target site (see mutation 9 in Table 11). 32 seedlings for each of the F1 lines were planted and screened for inheritance of mutations in ZmTS1 target site. 11 of the 32 F1 seedlings from Plant-1 cross germinated and 6 of them inherited the R1 event specific mutation. 5 of the 32 seedlings from plant-2 cross germinated and 3 of them inherited the R1 event specific mutation.

### Example 2. Expression of Cas12a in Cells Undergoing Meiosis to Generate Germinal Mutations

Several constructs are generated to preferentially express Cas12a in corn cells undergoing meiosis, in corn egg cells and/or corn embryos. See Table 12.

TABLE 12
Cassettes designed to express Cas12a preferentially, or solely, in
cells undergoing meiosis, corn egg cells and/or in corn embryos.

| | | | Promoter |
|---|---|---|---|
| Construct | Promoter::LbCas12a | Expression | SEQ ID NO. |
| 7 (Control) | ZmUbqM1::LbCas12a | Constitutive | 11 |
| 8 | ZmDSUL1::LbCas12a | Embryo | 1 |
| 9 | ZmEA1::LbCas12a | Egg/Embryo | 2 |
| 10 | ZmES4::LbCas12a | Egg/Embryo | 3 |
| 11 | ZmDMC1::LbCas12a | Meiotic | 4 |
| 12 | ZmMps1::LbCas12a | Meiotic | 5 |
| 13 | ZmAdf1::LbCas12a | Meiotic | 6 |

The plant codon optimized LbCas12a sequence (SEQ ID NO: 7) in the expression cassettes described in Table 12 is flanked by NLS sequences at the 5′ and 3′ ends (SEQ ID:8 and SEQ ID:9). Each construct described in Table 12 is introduced simultaneously with a construct (“gRNA construct”) encoding a gRNA complementary to a target site under the control of the Pol III ZmU6 promoter (SEQ ID NO: 10) into corn cells using biolistic transformation methods routinely used in the art. Alternatively, the constructs described in Table 12 can be transformed either biolistically or through an Agrobacterium T-DNA vector into cells containing a gRNA construct. The resulting transformed corn cells comprise one of constructs 7-13, as well as the gRNA construct. Corn plants are regenerated from the transformed corn cells and grown to maturity. At least one ear from each transformed corn plant is pollinated. Seed resulting from the pollination is screened for mutations in the target site and the number and types of mutations produced using constructs 8-13 is compared to the transformed corn plants produced using construct 7. Co-expression of Cas12a from constructs 8-13 and its cognitive guide RNA from the gRNA construct is expected to result in double stranded breaks in the genomic DNA at the target site, with subsequent DNA repair generating one or more unique mutations.

### Example 3. Expression of gRNA in Egg or Embryo Tissue with a Constitutively-Expressed Cas12a to Generate Germinal Mutations

Several constructs are generated to preferentially express a guide RNA (gRNA) complementary to a target site under the control of a Pol II promoter in meiotic cells, corn egg cells and/or corn embryos. See Table 3. Following transcription, Pol-II products are rapidly modified with a 5′ cap and poly-A tail and exported from the nucleus. These modifications and altered localization could prevent efficient use of gRNA. To optimize the gRNA availability and performance, self-cleaving ribozymes are incorporated into the gRNA cassette design. It has been reported that self-cleaving ribozymes facilitate cleavage/processing of the gRNA transcript from Pol II expressed transcripts to produce the precise guide molecule (see Wang et. al., 2018, J. of Integrative Plant Biol, 60:8, 626-631).

TABLE 13
Constructs designed to express a gRNA preferentially, or solely,
in corn egg cells and/or corn embryos.

| Construct | Promoter::gRNA | Promoter SEQ ID NO. |
|---|---|---|
| 14 (Control) | ZmUbqM1::ribozyme-gRNA- | 11 |
| | ribozyme | |
| 15 | ZmDSUL1::ribozyme-gRNA- | 1 |
| | ribozyme | |
| 16 | ZmEA1::ribozyme-gRNA- | 2 |
| | ribozyme | |
| 17 | ZmES4::ribozyme-gRNA- | 3 |
| | ribozyme | |
| 18 | ZmDMC1::ribozyme-gRNA- | 4 |
| | ribozyme | |
| 19 | ZmMps1::ribozyme-gRNA- | 5 |
| | ribozyme | |
| 20 | ZmAdf1::ribozyme-gRNA- | 6 |
| | ribozyme | |

The constructs described in Table 13 are stably introduced into corn cells using transformation methods routinely used in the art. Additionally, a construct (“Cas12a construct”) comprising a plant codon optimized nucleic acid sequence encoding a Cas12a protein (SEQ ID NO: 1) flanked by NLS sequences at the 5′ and 3′ ends (SEQ ID:2 and SEQ ID:3) and under the control of a ubiquitous ZmUbgM1 promoter (SEQ ID NO: 11) is co-introduced with each construct provided in Table 13. The resulting transformed corn cells comprise one of constructs 14-20, as well as the Cas12a construct. Corn plants are regenerated from the transformed corn cells and grown to maturity. At least one ear from each transformed corn plant is pollinated. Seed resulting from the pollination is screened for mutations in the target site, and the number and type of mutations produced using constructs 15-20 is compared to the transformed corn plants produced using construct 14. Selective expression of gRNA is expected to generate one or more unique mutations in each meiotic cell, egg cell, or in each embryo created by the pollination.

### Example 4. Expression of Cas12a and gRNA as a Single Transcript in Cells Undergoing Meiosis, Egg Cells or Embryo Tissue to Generate Germinal Mutations

Several constructs are generated to preferentially express LbCas12a and a guide RNA (gRNA) complementary to a target site as a single transcript in cells undergoing meiosis, corn egg cells and/or corn embryos. See Table 14.

TABLE 14
Constructs designed to express a LbCas12a and a gRNA
flanked by self-cleaving ribozymes in a single transcript
in meiotic cells, corn egg cells and/or corn embryos.

| | | Promoter |
|---|---|---|
| Construct | Promoter::gRNA | SEQ ID NO. |
| 21 (Control) | ZmUbqM1::LbCas12a-ribozyme-gRNA- | 11 |
| | ribozyme | |
| 22 | ZmDSUL1::LbCas12a-ribozyme-gRNA- | 1 |
| | ribozyme | |
| 23 | ZmEA1::LbCas12a-ribozyme-gRNA- | 2 |
| | ribozyme | |
| 24 | ZmES4::LbCas12a-ribozyme-gRNA- | 3 |
| | ribozyme | |
| 25 | ZmDMC1::LbCas12a-ribozyme-gRNA- | 4 |
| | ribozyme | |
| 26 | ZmMps1::LbCas12a-ribozyme-gRNA- | 5 |
| | ribozyme | |
| 27 | ZmAdf1::LbCas12a-ribozyme-gRNA- | 6 |
| | ribozyme | |

Each construct described in Table 14 is stably introduced into corn cells using biolistic transformation methods or agrobacterium transformation methods routinely used in the art. The resulting transformed corn cells comprise one of constructs 21-27. Corn plants are regenerated from the transformed corn cells and grown to maturity. At least one ear from each transformed corn plant is pollinated. The LbCas12a and gRNA are transcribed as part of a single transcript in cells where the promoter expresses. Subsequently, ribozyme mediated cleavage occurs releasing the gRNA segments. LbCas12a protein transcribed from the transcript forms ribonucleoproteins (RNPs) with the gRNAs. The RNPs will generate a double-stranded break at the target site and subsequent repair will generate one or more unique mutations in each meiotic cell, egg cell, or in each embryo created by the pollination. Seed resulting from the pollination is screened for mutations in the target site and the number and type of mutations produced using constructs 22-27 is compared to the transformed corn plants produced using construct 21.

### Example 5. Generating Mutations Via Crossing

Transgenic corn plants comprising one of Constructs 8-13 (see Example 2, Table 2) are generated and grown to flowering stage. An additional transgenic corn plant comprising the gRNA construct of Example 2 is also generated and grown to flowering stage. The corn plants comprising one of Constructs 8-13 are crossed with the corn pant comprising the gRNA construct, generating progeny corn plants comprising Cas12a and the gRNA being expressed in the resulting embryos.

Alternatively, transgenic corn plants comprising one of Constructs 15-20 (see Example 3, Table 13) are generated and grown to flowering stage. An additional transgenic corn plant comprising the Cas12a construct of Example 3 is also generated and grown to flowering stage. The corn plants comprising one of Constructs 15-20 are crossed with the corn pant comprising the Cas12a construct, generating progeny corn plants comprising Cas12a and the gRNA being expressed in the resulting embryos.

Co-expression of Cas12a and the gRNA generate a double-stranded break within the target site, thereby generating a unique mutation in each cell where the both components of the CRISPR system are expressed. Resulting embryos, or plants arising from the resulting embryos, are screened to identify mutations in the target site.

### Example 6. Expression of Cas12a During Meiosis, in Egg Cells or Embryo Tissue of Reciprocal F1s to Generate Different Germinal Mutations

R1 seeds containing T-DNA vectors that preferentially express Cas12a in corn meiotic tissue, egg and/or corn embryo cells as described in Example 1 were planted. See Table 15 for a list of constructs.

TABLE 15
Cassettes to express Cas12a preferentially, or solely, in corn
zygotes or embryos.

| | | Promoter |
|---|---|---|
| | Promoter::LbCas12a::terminator | SEQ ID |
| Construct | (3′UTR) | NO. |
| 6 | ZmAdf1::LbCas12a::OsLTP_{Term} | 6 |
| 5 | ZmMps1::LbCas12a::OsLTP_{Term} | 5 |
| 4 | ZmDMC1::LbCas12a::OsLTP_{Term} | 4 |
| 3 | ZmES4::LbCas12a::OsLTP_{Term} | 3 |

R1 individuals from each transformed construct representing 4-10 independent events per construct were grown to maturity and reciprocal crosses were attempted with all individuals. F1 seeds were recovered from 6-29 crosses per direction per construct. Up to 72 seedlings from each F1 ear were germinated, screened for mutations in the ZmTS1 target site and the mutation rates (cutting rates) were calculated. See Table 16.

TABLE 16
Total events and samples screened in F1 reciprocal cross progeny

| Promoter | | | |
|---|---|---|---|
| driving | | | Total F1 |
| Cas12a | Event parent | Total events | samples |
| ZmAdf1 | female | 14 | 508 |
| | Male | 10 | 85 |
| ZmDMC1 | female | 6 | 94 |
| | Male | 4 | 21 |
| ZmMps1 | female | 6 | 169 |
| | Male | 5 | 43 |
| ZmES4 | female | 8 | 652 |
| | Male | 7 | 194 |

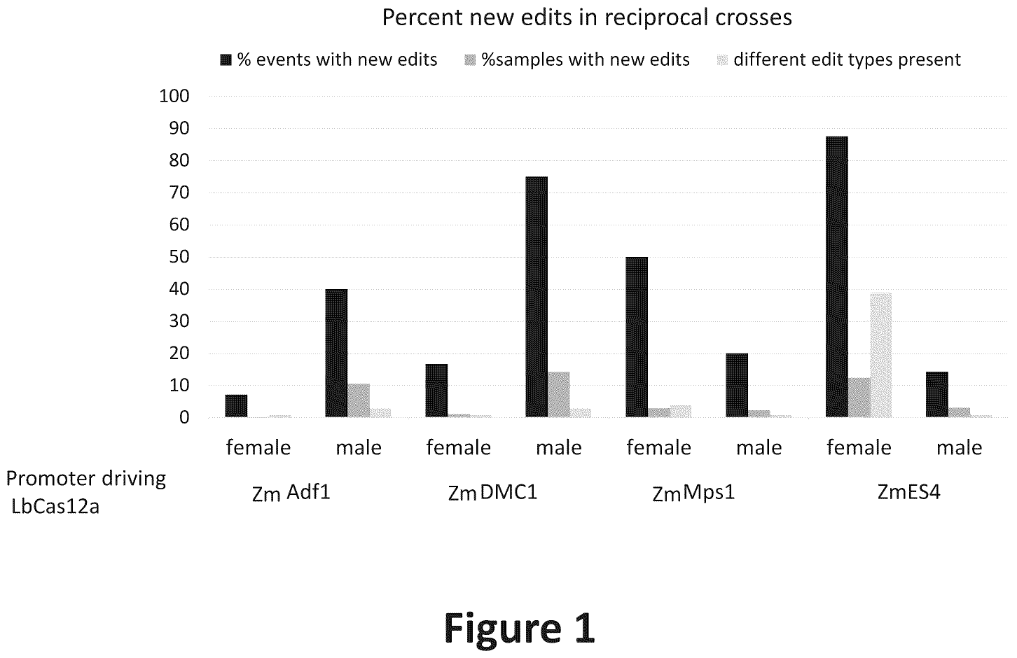

Taqman assays were performed to determine the copy number of the LbCas12a expression cassette. The overall target site mutation rate among all F1s ranged from 0 to 14%, and showed directional expression (FIG. 1 and Table 16).

F1 mutation rate was compared to the mutation rate from the event parent (R1) in order to determine how many new edits (present in F1 and not in the R1 parent) were generated. These data are summarized in FIG. 1. The ZmES4:LbCas12a construct showed the highest rate of cutting from the female (average of 12% of samples containing new edits). ZmDMC1:LbCas12a showed the highest rate of cutting from the male (average of 14% of samples containing new edits).

ZmES4:LbCas12a not only produced a high percentage of F1 plants with new edits when carried by the female parent, it also produced a high number of unique edits in the F1 plants (FIG. 1, Table 17). When carried by the female parent, ZmES4:LbCas12a produced 39 different mutation types as detected in F1 plants. The other promoters tested produced a maximum of 4 unique edits types in 1 progeny (see FIG. 1).

The new edits observed in the ZmES4:LbCas12a F1 plants were highly abundant. Sequencing of the target site showed 40%-100% of the sequencing reads containing the mutant alleles, indicating these edits are made early in embryo/zygote development and are thus fixed in the plant (Table 17). Edits that become fixed in the plant will be present in that plant's germline and thus inherited in its progeny.

ZmES4 retains high embryo/zygote expression generation after generation, and many independent events show functional activity of LbCas12a. Nearly every edited plant in each event contains a unique edit (Table 17). Moreover, all but one ZmES4 event tested produced new edits, with mutation rates per event of up to 20% (Table 17).

TABLE 17
ZmES4::LbCas12a editing summary in F1s by event.

| | | | | | | | | % | | | |
|---|---|---|---|---|---|---|---|---|---|---|---|
| | | | | Total | Total | | % | samples | Different | | |
| | | | Total | samples | samples | % | samples | with | edits^{&} in | | |
| | | Total | samples | with | with new | samples | with | new | total | | Different |
| | Event | Samples | with | new** | heritable | with | new | heritable | edited | Different | heritable |
| Event | parent | (Cas12a+) | edits* | edits | edits^{$} | edits | edits | edits | samples | new edits | edits |
| ZM_S22 | female | 55 | 26 | 11 | 11 | 47.27 | 20.00 | 20.00 | 12 | 9 | 9 |
| 321294 | male | 12 | 0 | 0 | 0 | 0.00 | 0.00 | 0.00 | 0 | 0 | 0 |
| ZM_S22 | female | 16 | 6 | 0 | 0 | 37.50 | 0.00 | 0.00 | 1 | 0 | 0 |
| 321298 | male | 0 | 0 | 0 | 0 | 0.00 | 0.00 | 0.00 | 0 | 0 | 0 |
| ZM_S22 | female | 39 | 26 | 4 | 4 | 66.67 | 10.26 | 10.26 | 10 | 4 | 4 |
| 321299 | male | 13 | 4 | 0 | 0 | 30.77 | 0.00 | 0.00 | 1 | 0 | 0 |
| ZM_S22 | female | 162 | 100 | 25 | 25 | 61.73 | 15.43 | 15.43 | 20 | 16 | 16 |
| 321304 | male | 38 | 7 | 0 | 0 | 18.42 | 0.00 | 0.00 | 2 | 0 | 0 |
| ZM_S22 | female | 72 | 29 | 2 | 2 | 40.28 | 2.78 | 2.78 | 4 | 2 | 2 |
| 321307 | male | 27 | 2 | 0 | 0 | 7.41 | 0.00 | 0.00 | 1 | 0 | 0 |
| ZM_S22 | female | 182 | 114 | 24 | 24 | 62.64 | 13.19 | 13.19 | 22 | 15 | 15 |
| 321323 | male | 70 | 36 | 6 | 6 | 51.43 | 8.57 | 8.57 | 6 | 1 | 1 |
| ZM_S22 | female | 92 | 63 | 17 | 17 | 68.48 | 18.48 | 18.48 | 19 | 14 | 14 |
| 321324 | male | 4 | 0 | 0 | 0 | 0.00 | 0.00 | 0.00 | 0 | 0 | 0 |
| ZM_S22 | female | 34 | 2 | 2 | 2 | 5.88 | 5.88 | 5.88 | 2 | 2 | 2 |
| 321326 | male | 30 | 0 | 0 | 0 | 0.00 | 0.00 | 0.00 | 0 | 0 | 0 |

*Total samples with edits includes samples with both new edits and edits that were inherited.
**New edits are defined as an edit found in the F1 that was not detected in the R1 parent.
^{$}Heritable edits are defined as edits that detected by sequencing with at least 40% of total sequencing reads from a sa,ple mapping to the edit.
^{&}Different edits refers to the number of unique edits identified.

### Example 7. Expression of Cas12a in Corn Egg Cells, Meiotic Cells or Embryos to Enable Editing from a Haploid Induction Line to a Target Genome

Vectors are generated to preferentially express Cas12a in zygote, embryo, egg and/or meiotic cells of a haploid induction (HI) line, which generates haploids when crossed to another line. Non-limiting examples of promoters and regulatory sequences useful to drive expression embryo, egg and/or meiotic cells are provided in Table 1. An expression cassette is provided in which a plant codon optimized LbCas12a sequence is flanked by NLS sequences at the 5′ and 3′ ends and operably linked to a promoter as described in Table 1. Each vector may also contain an expression cassette encoding one or more Cas12a gRNAs targeting unique corn genomic target sites; and, optionally, an expression cassette encoding a selectable marker conferring resistance to the herbicide glyphosate. The vector may either be transformed directly into the haploid induction line, or events containing the vector may be generated in a different germplasm, which is then crossed into the haploid induction line. The resulting “editing inducer” will contain the haploid induction trait as well as the expression cassette encoding Cas12a and, optionally, a gRNA directed to the target site. In some embodiments, the gRNA could be delivered to the haploid induction line or WT germplasm separately from the cassette expressing Cas12a.

Editing induced by a DNA modification enzyme, such as a guided nuclease (e.g., a CRISPR/Cas system,) expressed from the haploid inducer genome is limited by the efficiency of editing that can occur in the short period after fertilization but before elimination of the genome contributed by the haploid inducer. In systems where haploid induction is contributed by the female parent, having strong expression of the genome editing components from the maternal genome just before or shortly after fertilization is a desired characteristic. Thus, identifying and using promoters that express at high levels in the maternal tissue (e.g., in egg and embryo tissue), such as the ZmES4 promoter (see Example 6) is highly desired.

An inducer line carrying an event comprising the expression construct(s) described above (editing inducer) is crossed as a female to a male wild type germplasm. After pollen from the male wild type parent contacts the female haploid inducer parent, there will be a period of time (shortly before/during, and/or after fertilization) during which the editing machinery (e.g., a CRISPR/Cas system) expressed from the inducer parent will contact the genome inherited from the male parent, and induce a modification. In certain embodiments, genome editing occurs while the progeny of the induction cross is in the zygote phase of its life cycle. In certain embodiments, genome editing requires a longer period of time, potentially spanning several rounds of mitotic divisions in the tissues of the progeny plant.

Following the editing of the wild type genome, the inducer genome and the expression cassettes encoding the editing machinery (e.g., Cas12a and gRNA), will be lost from the cells in a certain frequency of progeny via one of the mechanisms of genome elimination that is characteristic of maternal haploid induction lines, thus yielding a corresponding frequency of haploid progeny plants containing edits in the male genome but no expression cassettes encoding the editing machinery. Haploid progeny can be identified using any method known in the art including sequencing of genomic DNA, genotyping with molecular markers, and phenotyping with plant or seed markers. The haploid progeny can be screened for editing at the target site(s) by any method known in the art, including sequencing of the target site. Edited haploids are selected and then doubled using any method known in the art, such as colchine treatment. The doubled haploid is homozygous for the edited allele and can be utilized in a breeding pipeline.

A single editing inducer line could be used to create a wide variety of unique edits across multiple germplasms, alleviating germplasm dependent transformation constraints. Further, an inducer line expressing the editing machinery (e.g., Cas12a and gRNA) in the egg, embryo, and/or meiotic tissue can produce a wide array of unique edits, eliminating the need to create multiple transgenic events to identify a particular edit or generate a range of edited alleles.

### Example 8: TALE Activators can Drive Robust LbCas12a Expression in Corn Protoplasts

This example describes the use of TALEs to induce robust and specific expression of a transcribable polynucleotide in egg, embryo, and/or meiotic plant cells.

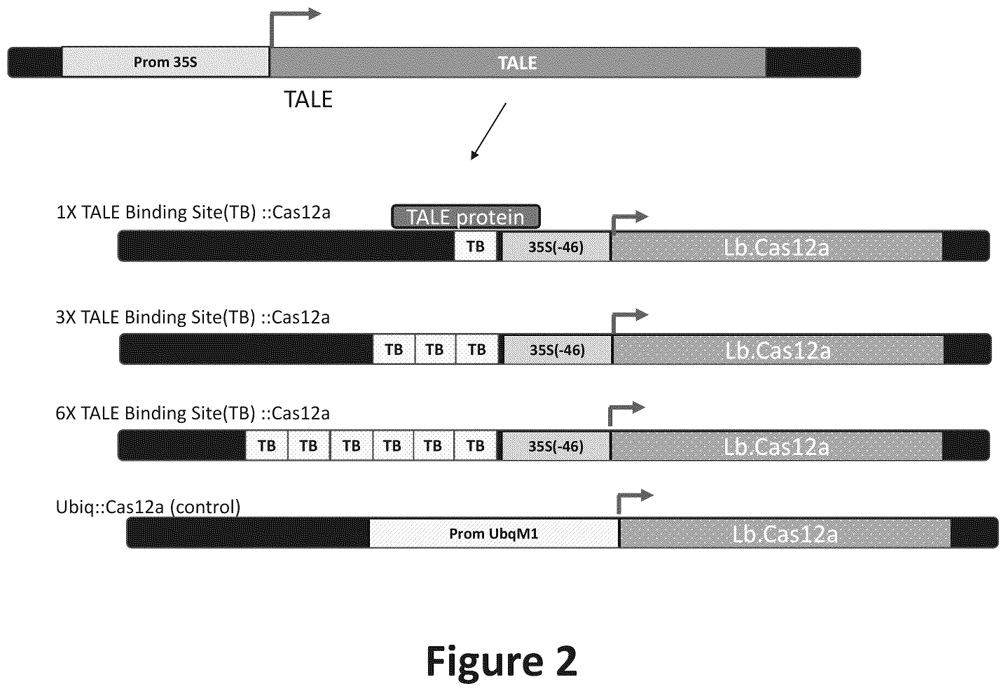

Transcription Activator-Like Effectors (TALEs) are transcription factors that comprise a C terminal activation domain and can activate/increase the expression of an operably linked transcribable polynucleotide once TALEs bind to the TALE binding site at or near the promoter. To test if a TALE could boost expression of Cas12a, several cassettes were designed that employed the minimal 35S(-46) promoter operably linked to a Zea mays DNAK intron and placed upstream of a polynucleotide sequence encoding Cas12a (See FIG. 2). The minimal 35S(-46) promoter has the TATA box, but lacks the transcription factor binding sites that will induce gene expression. Three expression constructs were generated, each comprising the 35S(-46):Lb.Cas12a cassette with one to six TALE protein binding sites (TB) (SEQ ID NO 15) placed upstream of the minimal 35S(-46) promoter (see Table 7). As a control, a polynucleotide sequence encoding LbCas12a was operably linked to the constitutive Ubiquitin promoter. Finally, a separate expression construct comprising a polynucleotide sequence (SEQ ID NO 16) encoding the TALE protein with the C terminal activation domain (SEQ ID NO 17) operably linked to the full 35S promoter was also generated.

TABLE 18
Cassettes designed to express Cas12a preferentially, or solely, in the
presence of TALE activator protein. TB = Tale Binding site.

| | | | Regulatory |
|---|---|---|---|
| Construct | Cassette | Details | Seq |
| 28 | 1XTB:35S(−46): | At most one TALE | 12 |
| | LbCas12a | protein will bind | |
| | | upstream of the 35S(−46): | |
| | | Lb.Cas12a | |
| 29 | 3XTB:35S(−46): | At most three TALE | 13 |
| | LbCas12a | proteins will | |
| | | bind upstream of the 35S(−46): | |
| | | Lb.Cas12a | |
| 30 | 6XTB:35S(−46): | At most six TALE | 14 |
| | LbCas12a | proteins will bind | |
| | | upstream of the 35S(−46): | |
| | | Lb.Cas12a | |
| 31 | Ubiquitin: | TALE proteins are not | 11 |
| (control) | LbCas12a | expected to bind/drive | |
| | | the expression of the | |
| | | Ubiquitin promoter | |

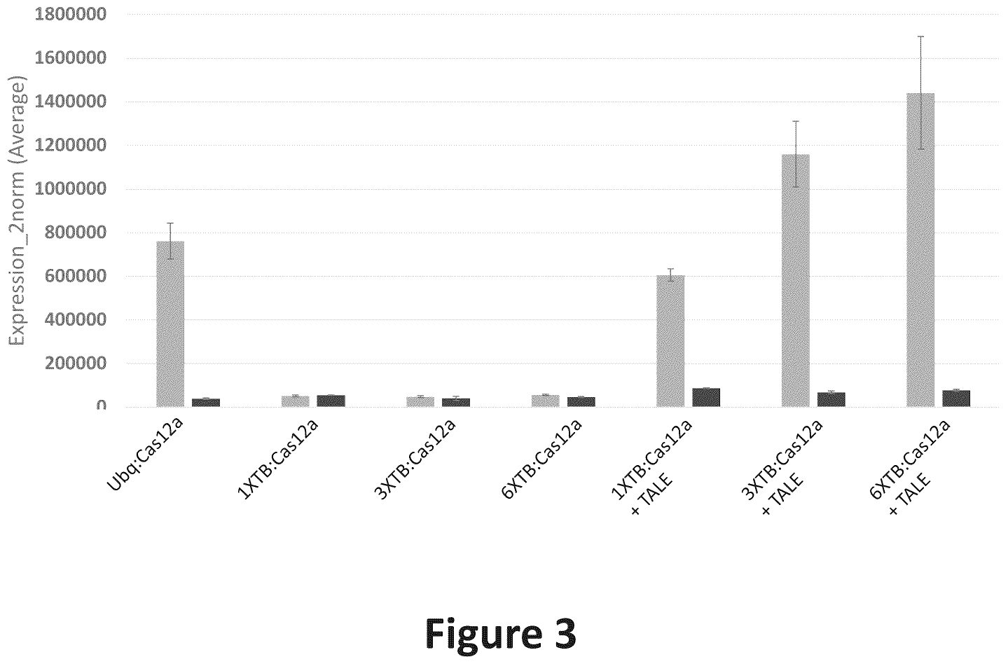

The constructs described in Table 18 were transfected into corn leaf protoplasts with and without the TALE expressing construct. After 18-24 hours, RNA was isolated and the expression of Cas12a and TALE was quantified using TaqMan assays (See FIG. 3).

The data showed that the 1 XTB:Cas12a, 3XTB:Cas12a and 6XTB:Cas12a constructs did not express well as the Ubq:Cas12a control in the absence of TALE expression (see FIG. 3). However, in the presence of TALE expression, high expression of Cas12a was observed, and expression increased with the number of TALE binding sites. The 3XTB:Cas12a and 6XTB:Cas12a constructs showed higher Cas12a expression than the Ubq:Cas12a control (see FIG. 3). This data suggests that not only can a TALE protein induce high expression of a gene operably linked to a TALE binding site, but that expression can be modulated depending on how many of the TALE binding sites are included.

### Example 9: TALE Induced Meiocyte/Embryo/Egg-Cell Preferred Expression of LbCas12a

A potential downside of tissue/cell preferred promoters is that they tend to not be robustly expressed. This example describes constructs that have been generated to overcome this limitation and induce robust expression of a transcribable polynucleotide, such as Cas12a, in a tissue/cell preferred manner.

Several constructs are generated for robust, TALE-induced Cas12a expression preferentially in egg, embryo and/or meiotic cells. Constructs are generated comprising a plant codon optimized LbCas12a coding sequence flanked by NLS sequences at the 5′ and 3′ ends and operably linked to the OsLTP transcription terminator sequence and the minimal 35S(-46) promoter with 1, 3, or 6 TALE binding sites. Expression constructs are also generated comprising a TALE coding sequence (SEQ ID NO 16) operably linked to a promoter that preferentially or solely expresses in egg, embryo and/or meiotic cells. Non-limiting examples of promoters and regulatory sequences to drive preferential egg, embryo and/or meiotic cell expression are provided in Table 1. An expression cassette comprising a TALE coding sequence operably linked to a constitutive Ubiquitin promoter is generated as a control. Corn 01DKD2 cultivar embryos are transformed with a vector(s) comprising the expression cassettes as described above and an expression cassette encoding a Cas12a gRNA complementary to a unique corn genomic target site (ZmTS1) under the control of a plant Pol III promoter and an expression cassette for a selectable marker conferring resistance to the herbicide glyphosate by agrobacterium-mediated transformation and R0 plants are generated from the transformed corn cells. Several R0 lines from each transformed construct are grown to maturity and at least one ear from each transformed corn plant are pollinated. Several R1 lines are selected, seedlings are germinated and screened for LbCas12a induced edits in the target site and the editing rates are calculated. It is anticipated that when plants comprising the Cas12a, gRNA and TALE expression vectors described above reach the reproductive stage, TALE expressed preferentially in the meiocytes, egg cells, and/or embryo cells will bind to TALE protein binding sites upstream of the 35S(-46):Lb.Cas12a and induce robust expression preferentially in meiocytes, egg cells, and/or embryo cells. The R1 plants generated from the transformed R0 lines are expected to exhibit a significant number of unique mutations at the ZmTS1 target site.

### Example 10: Tissue Preferred High Expression of CRISPR/Cas12a Editing System Components

This example describes the design of a vector to enable Meiocyte/Embryo/egg-cell preferred expression of Cas12a that is driven by a strong constitutive promoter.

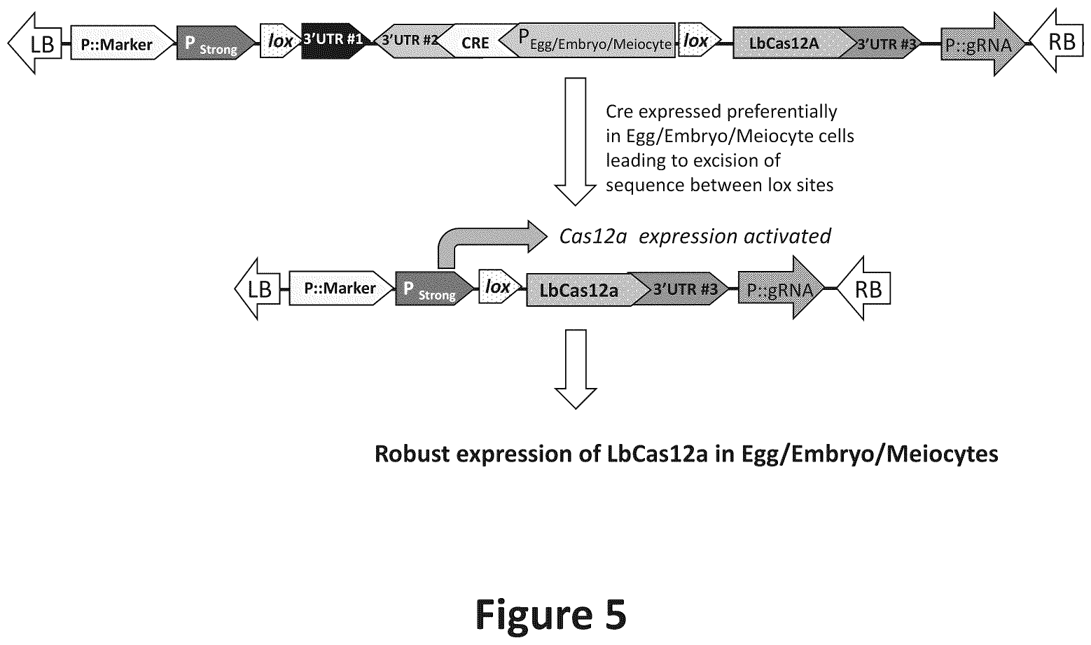

A recombinant Agrobacterium T-DNA construct is produced that comprises in 5′ to 3′ order, a left Border (LB) sequence; a selectable marker cassette in forward orientation; a sequence encoding a strong expression promoter in forward orientation; a first lox site, a first 3′UTR (UnTranslated Region) in forward orientation; a CRE recombinase cassette comprising a second 3′UTR in reverse orientation, a sequence encoding the CRE recombinase in reverse orientation operably linked to a promoter that preferentially or solely expresses in meiocytes, egg cells, and/or embryo cells; a second lox site in the same orientation as the first; a sequence encoding a plant codon optimized LbCas12a nuclease in forward orientation and operably linked to a third 3′UTR also in the forward orientation; and optionally an LbCas12a guide RNA expression cassette. The recombinant T-DNA construct is illustrated in FIG. 5. Non limiting examples of a strong promoters include: the 35S promoter from Cauliflower mosaic virus (SEQ ID NO:18), a promoter from Citrus Yellow mosaic virus (SEQ ID NO:19), Ubiquitin promoter from Sorghum bicolor (SEQ ID NO 20), and Zea mays Ubiquitin promoter (SEQ ID NO:11).

The vector described above comprises an expression cassette comprising a sequence encoding CRISPR/Cas editing system components and a constitutive, strong/high-expressing promoter that is interrupted by an expression cassette that preferentially expresses Cre recombinase in egg cells and/or embryo cells and/or cells undergoing meiosis. The Cre expression cassette is flanked by lox sites in the same orientation, such that preferential expression of Cre in egg, embryo and/or meiotic cells will cause excision of the sequence between the lox sites and operably linking the sequence encoding CRISPR/Cas editing system components with the constitutive, strong/high-expressing promoter. The excised sequence includes the Cre expression cassette and the first 3′UTR sequence that is intended to terminate any unintended transcription that might be initiated by the constitutive, strong/high-expressing promoter.

The vector is introduced to plant cells via Agrobacterium-mediated transformation. Once Cre recombinase is expressed in egg, embryo and/or meiotic cells, it mediates the excision of the sequence between the lox sites enabling high expression of Cas12a only in the tissue where Cre is expressed.

1.-21. (canceled)

22. A recombinant DNA construct comprising (a) a first nucleic acid sequence encoding a CRISPR effector protein operably linked to one or more TALE binding sites and a minimal promoter; and (b) a second nucleic acid sequence encoding a TALE operably linked to an egg cell-preferred promoter, meiotic cell-preferred promoter or embryo tissue-preferred promoter, wherein the minimal promoter does not drive expression of the DNA modification enzyme in the absence of TALE binding to the to one or more one or more TALE binding sites.

23. The recombinant DNA construct of claim 22, further comprising a third nucleic acid sequence encoding a guide nucleic acid operably linked to a third promoter.

24. The recombinant DNA construct of claim 22, wherein the one or more TALE binding sites consists of 1, 2, 3, 4, 5, 6, 7, 8, 9, 10 or more TALE binding sites.

25. The recombinant DNA construct of claim 22, wherein the second nucleic acid sequence encoding a TALE is operably linked to a heterologous egg cell-preferred promoter selected from the group consisting of: an EA1 promoter and an ES4 promoter.

26. The recombinant DNA construct of claim 22, wherein the second nucleic acid sequence encoding a TALE is operably linked to a heterologous egg cell-preferred promoter comprising a nucleic acid sequence that is at least 85%, at least 90%, at least 95%, at least 98%, at least 99%, or 100% identical to a nucleic acid sequence selected from the group consisting of SEQ ID NOs: 2-3, 21-38, 41-45, and 65-82 or a functional fragment thereof.

27. The recombinant DNA construct of claim 22, wherein the second nucleic acid sequence encoding a TALE is operably linked to an embryo tissue-preferred promoter selected from the group consisting of: a DSUL1 promoter, an EA1 promoter, an ES4 promoter, and an EAL1 promoter.

28. The recombinant DNA construct of claim 22, wherein the second nucleic acid sequence encoding a TALE is operably linked to an embryo tissue-preferred promoter comprising a nucleic acid sequence that is at least 85%, at least 90%, at least 95%, at least 98%, at least 99%, or 100% identical to a nucleic acid sequence selected from the group consisting of SEQ ID NOs: 1-3, 29-40, 43-45 and 86-88 or a functional fragment thereof.

29. The recombinant DNA construct of claim 22, wherein the second nucleic acid sequence encoding a TALE is operably linked to a meiotic cell-preferred promoter selected from the group consisting of a DMC1 promoter, a Mps1 promoter, and an Adf1 promoter.

30. The recombinant DNA construct of claim 22, wherein the second nucleic acid sequence encoding a TALE is operably linked to a meiotic cell-preferred promoter comprising a nucleic acid sequence at least 90% identical to a nucleic acid sequence selected from the group consisting of SEQ ID NOs: 4-6 and 83-85 or a functional fragment thereof.

31. The recombinant DNA construct of claim 22, wherein the CRISPR effector protein is selected from the group consisting of Cas9, Cas12a, Cas12b and CasX.

32. The recombinant DNA construct of claim 22, wherein the minimal promoter is a 35S(-46) promoter.

33. A plant comprising in its genome the recombinant DNA construct of claim 22.

34.-59. (canceled)


SEQUENCE LISTING (102 sequences)

SEQ ID NO: 1 - DNA, 1568 bp, Zea mays
  regulatory 1..1568: note = promoter - Promoter from ZmDSUL1; regulatory_class = promoter
  source 1..1568: mol_type = unassigned DNA
tttgctcacaggtgactcagtagatgacgatcatgatgcgctttaggtcatatatttgaagttcatcactgacctcatcaaggaacatgtttctagtgaccttggtgttagggtgttcttatggggtcgtaggtcatgctaccatcactgtcgatcgaggcattgtaggaatcgttcagtcaagtcattgaccgcccttcactcaatcagtcattgaccgcccatcactcaaccaggtcacgagtacctgacttatgcatgggacaatacttcctttactaggtgcgataaggcactatatggcaatggttgtcttcatgggggtaggctcgatgtcactctgaagtcacaaccctaggcgtgataccatactttttttgtgcaatagatagagcaactcgtaggtcatggatcttagagttccaagggcccttaaggggggtctaggcgatcaagaggactcaaggagaatgagagcgtcttggagtgtcctaggggccttgagtgccctctgtttcacttgttctaacaaatggttgtggctgggatagtcttacatgtctacggaggcccgagaccagatcctctgatatgttaggcttgtatcttagctagagtcgaggttagtcgcggatcagacaattccttctatcgaggttctttgaaaggcatcctatcaactcattagggtatgatctactcccgttgttaataaacgtattttataacacaacactcacatcatgaattgtatttaaactcaaccagaacttcagcatctcctcaccagtatgaatcatcacatattcatcaacgatgttttcagagtccatgcctctaaataaatttattataaaaaatattctataactaatctaataatacttattttatattaaaaaattagtacttatataactttagtcaaagttcaaactgtttgaggaagccaacattatactttttatggatggttgaagtatacagttttctataaactttgtcaaattatgtatggtaacttaacttagaaaataacaagaaataatggagagatatcacatttggaaacggcgtagtattccacttggaacacttttattttaagatgcaaaaaaaaatggctggtggagctaaaatattttttatttgagccgagacattctttttttgtgaactacttcatctgcgcagcggcagacttcgcgtgcgtattaatgtttatactgtgttgaactttagatacataaaccgataaacgcatgcaaattaaaagttgttcttgctgccacgcctatcggttgagttgcatgactttcaggccaacacactgcaagcaaaatggaattggagatttactacgagagcgagtggtcaagtattcatgactagccggctgcctcgtcttcttctcgaatggaatggctcccacttgctgcaaacggcccgccggagacgcttcgttaccccactaatccctggaaaggagtttacatggacagtgggagcctatataagcacgttcatggcgagacactacatccgagaaggacgatcaggcttcaggc

SEQ ID NO: 2 - DNA, 2000 bp, Zea mays
  regulatory 1..2000: note = promoter - Promoter from ZmEA1; regulatory_class = promoter
  source 1..2000: mol_type = unassigned DNA
cacgtccacgtgcaccaaggaggagaggacactactgtcgccacttttattttactagtagtcttccagagaccgcccaggcgcgcgcccaaccatgcgccccttcgactgcgaccctaccctagagctagccagcaaacatacgaacagcagctaatccgccgtctttgtggaggactgcaaagtaaagctgcgggttcgcaaggaacgccgcgcgggatataacctgcccggcgacgccgggtgcaaccattgtcaccaagggacaaacagatgacacagctggacccaccacgaaggacactatggccgccgccaccgccaccgccgccaccgccagtagtgagaacactgctcccatgatcttgaaggagagtgatgaatcaaacaagtcgtcaagcgcgctcgctctctggtatagggttggtactattgatagacaccgcgtggaatttctgcgacagagaagtcatctatttgtaggtcggccagaggcggctgctggccttctatgggggtgcgttgcatgcgtgcgtaattgctgccgtctcacgtacggcgaagtgcgcgtgctggggaaaacgaatcggcctgggaactccaagaggcgttgctccgcctccgactctgagtgcatagccaagtactgcagctactgcccgctccgcccgatgcgccgatcaacacccatttcgcaaagtgtagccggccccatcaaatgcagaagtcgactcgtcgattcggagtgagaacgatcattttccgacaatactaatccaaaggcaggctcatggtggtacatgtccaagtccaatcttctaatgggtatagttaggttatttaaaataataccctgagttctgtcactttcttcatttttaatactaatccaagctgccacgacggattggtgggtagtggacgagtagtatcggcaaaaaaaaaaatactacttttgccggtgaaatttaattactactctacataattagcaaatgaaattaatcacctcttatgcacgtatgtgttttaatttccatcagttcacttttagggctgatttggtggtcagtgatcccgagaggatccatgagaggaatccccttgatattcaaaattgaattgtaaggggatttctctcccatgaatcatcttggaatctctgatcaccaaatcagcccttaaaatgaaaaagaaatttatattatccggaagctttggaaaatatactcataatttcgtatataaaccaaaagcattattaaaccttgagtcaaatgcatgtaaccttgaggggttacctaatcctagtgtgccaaagcatgtaaccttgaggggtcggttccttaggaaggcgccgtaaggcaagggtaaaaccaacgttctacccgaggtaaaaccacacgcccttgtgagccccagattcacctggatgtcttgtatttacggcaccgggctgcgggggccaatgcaccatcatttaagctatgtcgaccgctggcctttatagcatggcctcgaggtgactcgtagttaatgaaattaatttaattactactctacataattagcagatgaaattaattacctcttatgcacgttctcactactaccaagcaacaattcagcttctgcatttcgctaccgttctcttcaacttcaatgcgctcgactgatcgcgcacattgcgaagctgtctcttcgtcgtcgcctcccatcgtgatttgagactggggagcaaatgcgcacggcatgcatcgcaatgcacgcgatgaagctgagcagccgcctggccaacctcgatccggcgctgcagcctactacaaatagatgttcaattaacacaacacgcagcgaccgctgtccagtgtccattcattccaaacccagccgatcgctctcctccaattaagcagcaagggcagaagcaacaccggcgtgccacccggacgacgctgaatcctgcatgtcaacctgcccggccatcgtcattcgtcaac

SEQ ID NO: 3 - DNA, 2000 bp, Zea mays
  regulatory 1..2000: note = promoter - Promoter from ZmES4; regulatory_class = promoter
  source 1..2000: mol_type = unassigned DNA
tttccttaatctcctgggtcacttagaccccgcaaggaccaccacacacttaggtgtctcttgcttcgattacaaatcacttgagaaaagaaatgggaaaggaagaaggcaatccaagaggcaagagctcaaaagaacacaaaatctctctctcacaagtcactaagtgcttgagttgaatttgggacttggagaggctttgatcttttgaattgtgtctacgagtgaatgctagagctcttgtaatgaatgtgatactcagaaatcttggatgcttggagtggtggtggttggggggtatttatagccctcaaccaccaaacaaccgttggggagggccgctgtcgatgggcgcaccggacagtctggtacgccaccggacactgtccggtgcgccagccacgtcacgcaaccgttagggtttgggagctgtcgaccgttggaggctttgtcctctagtggcaccggacagtccggtgccgcaccggatagccactgttcagtgtccggtatgctctaactctaggctctgactcagccgcgtattgttgtgcgctgtagcgctcgtcaggagccgttgcagtcgaccattgcgctggattgtcgttactccgctggtgcaccggacagtccggtggcacaccggatagtccggtgaattgtagcggagagcgccagaagaaacccgagagtggccgtttggactctgtacggtcctggtgcaccagacactgtccggtggcacaccggaaagtccggtgcgtgaaagagaaatatggtttaactttttcctataaatgattttggtggttgaatgcccaacacaaataattgtactaactagtttgctccagattatatattctataggtgcataaaaggttcaacacaaaccaataaaagatcaaagttagggtttaaaaagaaaggaacaaagaaaccaaagtgtgccctggtctggcacaccggactgttcggtgtgccacgtcgatgtttgcacagctcgcccgaggccactcttgggcgggagaccccagggtagcctcgcgtgagccatgactgtgcctcccgtccgagtgtggcctcagacgacagacatagaaccatgatgtactgtagatatatatctatgtttatagtacatcaacagattctgaagtctatttcaggttgaattaggtttgtactcggacgagtgatatttatcgttccatatctatgttttatataaattttttactctcgtcgtaacatataggcacatacctgttcaaatatatgtgcgattgtgctcatatgggtagaagaaggaactggatgtgaagacatgtatatatagaccaaaaatgtatcttagccgatttcaaactgcctagcgaaaagatgaagctgctccgtgtttgggtgactgcagcattatgtatcttattaatcgatttcacagaagacttgtctctcatggaacaaacgaacctattagcagtctgtatataaagaagtgacttccagactgccaagtttagtgattagggctgctgtgactgaccatttaccttgcaaacaatctttcgatctactgctctgtcatgcatggtttttgcatcgtccattatgtttatttcccattgttgcagcttacgatatcgtgtatacctggcacctatgtttatgacaatgaaactcaccatctttattctattggcattcattttcattcaaattacataacgtatgctagaacgaccgaatacaaattactgctaataaacatcctgcgtacgcgcaatggtgcaccatttaccaattaatagctgtaacggtactgcaagtaatggtcgaagcgttatgttttctctcctctcttcccaccgtacaggatcatatattcatatatatatacacatgcctctggaacggctaattcgatagttccaccacgttacttccatatatttcccttggattggatcgtcggcgcccaaacgaatactaatccggca

SEQ ID NO: 4 - DNA, 2999 bp, Zea mays
  regulatory 1..2999: note = promoter - Promoter from ZmDMC1; regulatory_class = promoter
  source 1..2999: mol_type = unassigned DNA
acctaacctggctgagccaacacctgaggccctcccctaaaccagggagtccctgatgatccctactcaaaggtgataagggtgaaaacccttcattatacacattttgaaaagcattttcttttgatcccggctgggtggccataataacttgtctcaaaatcatatcatgcataaaataacaggctgagggttgtggttgaaaaatcataggtaatttatgcatcaaagggatccagtgagtttgtcgtgcttatccgacgaagggggaagaggagctcgcggaactgtcttctagctccactgcctggcgtagacttgcagatctggcctccacgagacgacatgaacgctccgataactatgcaacatgaataagcaaacatacaaaccaacaagtataccaacaaatatttagtatagtggtcagaatagcgatatatggatgggtagagtcgtgagtagaatctgtgtcatgtggtgttgagatactactagtggtggagcggaggtgcttaccaggagggtggactggaggcgaagcgacactatgcgtgtagccggcagagcggagtaactaagtgagtggggtgtttgccttggctgagggtgttgagtgtgtgtggagagggagagggtagctgggtgtatttatagctgggtgtatgtggtgtagcacaatgaagttcattgttggtgagaatagtgacatatgaatcgtctgacttagtatgaacaggagagttatatgcagaatatgggacaagagtattttgggagttttctggaatatgaaccatacttaagtgatgacatggttggatagggaataatttgataagaatttagaaacaagaattattggaatcggagtttggaagcctggttcgaaggaatctcaaagttaagcgtgctcaacttagagaaacctgggatgggtgaccagatgggaagttccctactggaaggaaaatcacagtcaccggagttcgtatgactgaaatatgggtctggctggtcttaggtggactggatagcatgatggatgagtagttgaaatttagggtgatcggatgatcgatgaatagtgtcttggaatgagaaggaagaaagcacgattgcaaaagctaagcgacaagagcgacaaataacacacagatcactctctctctcaagtcactaatcactaatgatcacttgtcttaattgtggaacttggagagattggaagctttgattgtgtcttggaatggattgctagctcttgtattgaatgtgaaggattggaatgcttgggtgtcatgaatggaggtggttggggttgtatttatagccctcaaccacttcctagccgttgctccttttctaccgaccgcggacggtccgcgcccctggtccggacagtccgcccctgcacatcaacggctgaaatcgcaacgatcagcagtaacggctatatcaacggctatatagcatttaatgtgtcgtcagatgtcagataaaagcagtcgcagacggtccggtcatgcaccccggacggtccgcgaggatgctataattcattttaccgaacccgtcaccttcgggtttttcggttcttcaccgacctgatggtccgcgcctgaggccggatggtccgcgcttggtctcggacggtgcttggctttccatcggacggtccgtagtgtagacttgtgtttttgtattggttctgtcctaggctcaccctagtttcgcggacggtccgccgcaagggcccagacggtccgcgcttatgtgattttccaaaaagcttctcctgtccagaataatctacggtattccggacagtcgactttagaatagttgtagatgaacttatgcacctgtggaatgatcaattagacaaaccggttagtccacaaggtttgtgatggtcgtcaaacaccaaaaccgattatagggaatattgaaactatttccctttcaattatatttgtttagttgaggttctaaattttttatagcaaggcccgtttggttagagagactaattttagtccctgacttttagtctcatttagtctctattttgccaaacggaaggactaaagtagggactaattggttttagggcatgtttggttcgttacctcaattgccacattttgcctaacttttctgcctaaggttagttattcaattcgaataactaaccttaggcaaagtggggcacagttagccacaaaccaaacaagcccttagtctttagtcccttacatagatgctaaaagggactaaaggggaatatttactctaattacccttgcctagaaaactagtgtgaaacaaaaaaaagagtattttgatctttatgtattacatttaatgtatttaaaatctgtttagcccctacaactaaacaatatagagactaaagtttagtttagggactaaactttagtcctaagactaatggagccaaacggggcccaaatctcgtttaaaaatttgaagcaaacacatccttaacggaccgtgggcaatgagctgagtctcccctgactctgggccgcaaagtcctggtacacttgatcatgttggtccatcctatggctgaccgcgacttccctagactgaaatcaacccattcctaggctcctagcccagcccaacggccgagcacacgatccgttcgagagcgagaaggtcctccggcctcagcaccctcaagtacacagtacaccctcgcaccggtaccgcggtccgcggcacacgacacccccactcgtctgtcgactcacgtctgatcgtctctccccaacaatctctcgagtcttgccattcgcctcagcttgcccctcctccaagcgtccaagccccacccggccattgcctcctcctcctgccgcaggtaagctagctgctccagcttctcttgccatcgcgtgtcctgcactcaccgccctcgcgcgtgtaactcctcctccgtcccccgatcgggctactagtgcaggcac

SEQ ID NO: 5 - DNA, 1979 bp, Zea mays
  regulatory 1..1979: note = promoter - Promoter from ZmMps1; regulatory_class = promoter
  source 1..1979: mol_type = unassigned DNA
tgatatggtttggttgcacttgagaaatgattagtttcctaatttgcaccattctaaattaatgcctcgtgttgctggtccttttaaggtgctaacaaagattaatgataatgcttatatccttgacctgtctgtagaatttggtgtttacactagttttaatgttccagatttgaaaccatacatgggcgaggatgacgagttaccgttgaggatgacttcacttcaagaaggggaggatgatgaggatatcaactgcaacacgagtacgatacaccagcagcaccaccacctcaggcgccacatccctcctcagctaggccaacttcaccttaggcaccacttccttcattagttgggcaaatcactcggacccgtgcaagagatcttaacttcgtcatgctactgaataatgagggaccagaagaatagacgaccggcccaattacgacccaaggtggaggacctaggatgggccgcccctagggttgcgtttttccaccttggccgccaccaggggctgtgcccctctctctatttatctagtagctgcacctccttttgttccttgagttttgttttacattagccttagctattcccaaacacgcacatattagcgctgtcttcgtgtattcagaactccaccatcgagtaatagactagattgctcgtatctttttcttgttcgttcttcgattgcgtgcaggaaatgatctttgtgatcaggctgatcttgcaccagcaaggtcggtaaccatagggagttggttcagcgattgcattggcgcctcgagcttgctcgtcgtagtcggaccgtgagggtcatcttcgccagatcgaaattatctccactcaccgaaagatcgggcacttcagccttatcagggagtggctgcggcatgggtcccttcggtcagctagtgatgaggtcggtgcagtgatttggtttgggccgaggagaagattcagcccaggtctaggtatgcttctcttctttatattttgtgccttctttcttgaatttagattctatattcaactccaaattatttttgagtttcaaactataaatatacaaacaataatgcaacaatattttattattctatttatttattattctagagaagcaattgcttctagctatgtaattcacacataaatagtttattttagagaaataatttctattgtatagctatcaagatatgatttaactcaaatttctttaaagagttatagatcaacaaagggataacttaattatcttttcatttgatcttttattagaaaaagttattcattttgtgagaaaatccttattaaaaagatatgttggttcaaatccttttggagaagcttttataggtgctagaataggattttggggttagagtgttgctctagctaattaaaatagtagaatagccgtttgtagtcgcggtcctagaataggcgggtttatgttaaattcgttgatcacggatcacggtccacatattcttgggtgggcctctcctctcgtttgcctgcgccgccgcgatcgccaatcggcgctcttcccaaaccccgcccgccaacgccgacggcgtcagcggccacgggcacctcctcgtcgcgaatagactcgccatcgctaaaggccgcgctcgcaatggctctcatccactacaaccgcctccctggcaaagccaacgccaccgcgggcacatcacccccgtctctcctccactggaagcgcaaggtcccattcccctagcagcaacaaccctcggattcaaaccctacctccgcctctgacattagatctccttctcccgcccaggccaaggaccggaagcgcgaaatcctccgcctccgcgaggagctcaaggtcctccaaggtatgtaaggccatgccaatctatccgcgctattgttcgtctggccgaattccattgcaggcgtcgtggaaattgagtttgttatcggagcag

SEQ ID NO: 6 - DNA, 1824 bp, Zea mays
  regulatory 1..1824: note = promoter - Promoter from ZmAdf1; regulatory_class = promoter
  source 1..1824: mol_type = unassigned DNA
agaccaacacgagaattttgtgcgtactttgtcctcactcatacgcaccagagaatacttttcgatcgatcactcatcctaaggttgctttaggccaaacacgtttaacctaaaggttctttcgcagcaggcttccgaaaaaaagatacaccttgttgatatggatattctattaattttattaagtcttagcgaggatgtcacaatccaaccaggctaggatatcacaatcctccctcttagaagaccgacgtcctcatcggtcaaccccaagccaaaaacctccctcttacccgtgactgtatgtctagtgtcgtcatattccatgccatttgaccactctgagcctacatacgtcgtgcaacatatacccaaaaccctagcctacacacgtccgtgaaaccacgagggtcgtctctgataccatttgtaacatctcatccactgtcggaccagtgatacttactcctgacaaccctctagaaccataaaccatccttacaaaccaacacgagtcttttgtgcgcactttatcctcactcatacgaactagggaggatttgtcggtcgggcacacatcctaagattgctctagaccaagcacacttaacctagatggttcttttaagacatacttcataatgtagttatttgttttgatttatttctattattccaacaaattatgtaacatttacatccttgcagtgaccataactatagatgcaatacaatgaactatggagttcaatgtatctataggtgttgttctgctagtgctaaactctagggaggttgcgctgtgtccaatgtcgctggccatgtcgcgtgacgtgcctcgcgacaggctactggtagttctgcgccgaacattggttgatccacgcctaagaccacgcactaagccggtctatccgtgttcaacatcgtagttctgcacctctgggatacacaaccgccttgtccaacgccatgccccaagccaccgcatacgtcggggctatcagtgtctcctttctcagcgtgctaacatgatacatgttacaatcgaagacgagctcgcatcgtggcaacaaattttggtgctccaaagctttggaaaggttatcctcaacattgttgggatccatggcggtctagaatcaaccatatgaaacactagcggctagggttcggaggagatcattagggaggagaaggagaggtcaagtctaagttgggctaggtgagaacgatcatgggtagatggatggcagtaccgggtaattttcatcaacttacgattaatagtgggtcttttattaaaaataagctgatataagcatcttttgagaagcttatagggttatcataatctcaagtactagattatataattttatcacataagttgcttcatacttagtttgtctatcactagcttatttacatagaatttagattatataataccaaaccctaaatgacctgaaccaaagatacccagattgcttcacatcatttgtgtggcactgtggctacatgaggacgtgggaatgggcgaatggcgcggaacgacccccgataggtttccgatattgccctccccacgcgcacgatttaaccgatccgcccccgtttctttctttttctccacgagaacgagattacgagacaagagacaagtagacaacccagctctccagcggtccagcggtccagcctctcctagccgaggccaaaacccccataccaacacacacttcgcttctcgctgcctccgcggcggcgcgccgctccgctccgagccctcttcctcctcctcctctgcccgttcggtcgaga

SEQ ID NO: 7 - DNA, 3681 bp, synthetic construct
  misc_feature 1..3681: note = Synthetic sequence
  source 1..3681: mol_type = other DNA
tcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 8 - DNA, 36 bp, Lycopersicum esculentum
  misc_structure 1..36: note = NLS1-Monopartite Nuclear Localization Signal (NLS) from the tomato heat stress transcription factor HSFA1
  source 1..36: mol_type = unassigned DNA
atggcgggatctaagaagagaagaattaaacaagat

SEQ ID NO: 9 - DNA, 33 bp, Lycopersicum esculentum
  misc_feature 1..33: note = NLS2-Monopartite Nuclear Localization Signal (NLS) from the tomato heat stress transcription factor HSFA1
  source 1..33: mol_type = unassigned DNA
ggatctaagaagagaagaattaaacaagattga

SEQ ID NO: 10 - DNA, 200 bp, Zea mays
  regulatory 1..200: note = promoter - Promoter from ZmU6; regulatory_class = promoter
  source 1..200: mol_type = unassigned DNA
cggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 11 - DNA, 2008 bp, Zea mays
  regulatory 1..2008: note = promoter - Promoter, 5' UTR and intron from ZmUbqM1; regulatory_class = promoter
  source 1..2008: mol_type = unassigned DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtc

SEQ ID NO: 12 - DNA, 927 bp, synthetic construct
  misc_feature 1..927: note = synthetic sequence
  source 1..927: mol_type = other DNA
tgcccggtctccattattcgcaagacccttcctctatataaggaagttcatttcatttggagaggacacgctgaaatcaccagtctctctctacaaatctatctctctctattttccggaccgaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 13 - DNA, 987 bp, synthetic construct
  misc_feature 1..987: note = synthetic sequence
  source 1..987: mol_type = other DNA
tgcccggtctccattattttgcttcttgtatgcccggtctccattattcctctcattatatgcccggtctccattattcgcaagacccttcctctatataaggaagttcatttcatttggagaggacacgctgaaatcaccagtctctctctacaaatctatctctctctattttccggaccgaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 14 - DNA, 1077 bp, synthetic construct
  misc_feature 1..1077: note = synthetic sequence
  source 1..1077: mol_type = other DNA
tgcccggtctccattattctgacgactatatgcccggtctccattatttcctgctaattatgcccggtctccattattatcctcaccatatgcccggtctccattattttgcttcttgtatgcccggtctccattattcctctcattatatgcccggtctccattattcgcaagacccttcctctatataaggaagttcatttcatttggagaggacacgctgaaatcaccagtctctctctacaaatctatctctctctattttccggaccgaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 15 - DNA, 18 bp, synthetic construct
  misc_feature 1..18: note = synthetic sequence
  source 1..18: mol_type = other DNA
tgcccggtctccattatt

SEQ ID NO: 16 - DNA, 2973 bp, synthetic construct
  misc_feature 1..2973: note = synthetic sequence
  source 1..2973: mol_type = other DNA
cctgcagctcaggtggacctccgcacccttggctactctcagcaacagcaagagaagatcaagccaaaggtgcggtcaacagtcgcacagcaccatgaggcactggtgggccacgggttcacccacgctcatattgtcgcactcagccagcatcctgcagcacttggcaccgttgcagtgacgtaccagcacatcattacagccctcccagaggcgacccatgaggacatcgtcggcgttgggaagcagtggagcggcgcaagagctctcgaggctctgctcaccgatgcaggcgagcttagggggcctcctctgcagctcgacacaggccaactggtgaagatcgcaaagagaggcggggtcaccgcgatggaggctgttcacgcaagccggaacgctctcaccggtgcaccgcttaatctcactccagctcaggtcgtggctatcgcgtccaacaatggcggcaagcaggcgctcgagactgtccagaggctcctccccgttctgtgccaggaccacgggctcaccccagatcaggtggtcgctatcgcgtcccatgacggcgggaagcaggccctggagaccgtgcagcgcctcctgccggtcctctgccaggaccacggcctgacgccagatcaggttgtggctattgccagccacgatggcgggaagcaggcgctcgagacagttcagaggctcctgccagtgctgtgccaggatcacggcctgactcctgatcaggtcgttgcgatcgcttcgcacgatggggggaagcaggctctggagaccgtccagcggctcctgccggttctgtgccaggatcatgggctcacgccagatcaggtggtcgccattgcgtctaacaatggcgggaagcaggcgctcgaaacagtgcagcgcctcctgccagtcctctgccaggaccatgggctcactcctgatcaggttgtggctatcgcctcaaacaatggcgggaagcaggccctggaaaccgttcagaggctcctgccggtgctgtgccaggaccatgggctgaccccagatcaggtcgttgcgattgcttccaatggcgggggcaagcaggcgctcgaaaccgtgcagcggctcctgccagttctgtgccaggatcacgggctgactcctgaccaggtcgtcgctatcgcgagccatgacgggggcaagcaggccctggagactgtccagcgcctcctgccggtcttatgccaggatcatgggctgactccggaccaggtcgtggctattgcctcgaacgggggggggaagcaggcgctcgaaactgtgcagaggctcctgccagtcctgtgccaggaccatggcctcaccccggaccaggtggttgcgatcgcttctcacgacggcgggaagcaggccctggaaacagtgcagcggctcctgccggtcctgtgccaggaccacgggctgaccccggaccaggtcgtggccattgcgtcacatgacggggggaagcaggcgctcgaaacggtccagcgcctcctgccagtcttatgccaggaccatggcctgaccccggaccaggtggtggctatcgcctccaacatcggggggaagcaggccctggaaactgtccagaggctcctgccggtcttgtgtcaggatcatgggctgacaccggaccaggtcgttgcgatcgcctccaatggcggcggcaagcaggcgctcgaaacagtacagcggctcctgccagtcttgtgtcaggaccatgggctgacacctgaccaggttgtcgctatcgcgtcgaacgggggtgggaagcaggccctggaaacggttcagcgcctcctgccggtcctatgccaggatcatggcctcaccccggatcaggtcgtcgctatcgccagcaatataggtgggaagcaggccctcgagacagtccagaggctcctgccagtcttatgtcaggatcatggcctcactccggaccaggttgttgcgatcgcttccaacggcggcggcaagcaggccctcgagactgtccagcggctcctgccggtgctctgccaggatcatggcctcactccagaccaggttgtggcgattgcctctaatgggggggggaagcaggctctcgagtctattgtggcgcagctctcaagaagggacccggcgctagcggctctgactaatgaccatctcgtggctctggcttgcctgggggggcggcctgctctggacgctgtgaagaaggggctcccacacgctccagagttcatccgcagggtgaacaggaggattgctgagcggacaagccacagggtcgctgactacgctcatgtggtccgcgttctggagttcttccagtgccactcgcatccggctcacgccttcgatgaggccatgacccagttcggcatgtctcggcatgggctggtccagctcttcaggcgggttggcgtgactgagttcgaggctcgctacgggaccctgccaccagcgtcccagcgctgggacaggatcctccaggcgagcggcatgaagagggctaagccaagccctacctcggctcagacgccagaccagacatctctccacgcgttcgctgattcactggagagggacctcgatgctccatccccaatgcatgagggcgaccagaccagggcgtccagccgcaagaggtcacggtccgatagggctgtgacggggccatcggctcagcaggctgtcgaggttagggtgcctgagcagagggacgctctccacctgccactctcctggagggtcaagcgccctaggacgaggatctggggcgggctgccagaccctggcacaccgattgccgcggatctcgctgcctcgtctactgttatgtgggagcaggacgctgctccattcgctggcgctgctgacgatttcccagccttcaatgaggaggagctggcttggctgatggagctgctgcctcagtcggggtcggttggcgggacaatctga

SEQ ID NO: 17 - protein, 990 aa, synthetic construct
  REGION 1..990: note = synthetic sequence
PAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALESIVAQLSRRDPALAALTNDHLVALACLGGRPALDAVKKGLPHAPEFIRRVNRRIAERTSHRVADYAHVVRVLEFFQCHSHPAHAFDEAMTQFGMSRHGLVQLFRRVGVTEFEARYGTLPPASQRWDRILQASGMKRAKPSPTSAQTPDQTSLHAFADSLERDLDAPSPMHEGDQTRASSRKRSRSDRAVTGPSAQQAVEVRVPEQRDALHLPLSWRVKRPRTRIWGGLPDPGTPIAADLAASSTVMWEQDAAPFAGAADDFPAFNEEELAWLMELLPQSGSVGGTI

SEQ ID NO: 18 - DNA, 612 bp, Cauliflower mosaic virus
  regulatory 1..612: note = promoter - 35S promoter; regulatory_class = promoter
  source 1..612: mol_type = unassigned DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 19 - DNA, 501 bp, Citrus yellow mosaic virus
  source 1..501: mol_type = unassigned DNA
ctggtggccctccaccagcacaaactcgcttacatcagaagcaaagctacaagggacaacgcatatgccgataggctacccacatgcaatcgggaccacgagcaactgtgtgaagtggtcgagctattagaaggaatctcggaaagaatcagcgatacagctgtctaggacagctggcttcaattatggagcgtgatggacccccccgcaataatccaaagtttggtgtgcttttagtagtgcgtctttatggaccactactttattgtaataatcgatgctttttgtagtgcgctcttcgtgcgctctactttatgcttttgcttttgtaagtgcgctgtaagtgcgcctgtctttcttcagatgcttatcctttaagcatcttttgctttttgcgtggcatcctttagttcacaatttaaagaatgacgatggggcccaagatgtgcacccggttctctaaattgcctatataaggatatcccatagccttgtttttgc

SEQ ID NO: 20 - DNA, 560 bp, Sorghum bicolor
  source 1..560: mol_type = unassigned DNA
agaagtaaaaaaaaagttcgtttcagaatcataaaggtaagttaaaaaaagaccatacaaaaaagaggtatttaatgataaactataatccagaatttgttaggatagtatataagaataagaccttgtttagtttcaaaaaaatttgcaaaattttccagattcctcgtcacatcaaatctttagaggtatgcatggagtattaaatatagacaagacctaaataagaaaacatgaaatgttcacgaaaaaaatcaagccaatgcatgatcgaagcaaacggtatagtaacggtgttaacctgatccattgatctttgtaatctttaacggccacctaccgcgggcagcaaacggcgtccccctcctcgatatctccgcggcggcctctggctttttccgcggaattgcgcggtggggacggattccacgagaccgcaacgcaaccgcctctcgccgctgggccccacaccgctcggtgccgtagcccgtagcctcacgggattctttctccctcctcccccgtgtataaattggcttcatcccctccctgcctcatcc

SEQ ID NO: 21 - DNA, 1998 bp, Zea mays
  source 1..1998: mol_type = unassigned DNA
tgcccgggtcggggccggaagtcgcgcgtggggcctgtccatcgaacaaaagcgagagaaaacgtgcatcggccggtggtggcccgtacggtgggctgggctcgaatcaacagcagatgaattggtagacaagacagcgcgatgcatgaatagtgcgtgattgggcttctcgagatagacgcggttaattgggccggattgcacacgcaaaaacaaaaaaaaaaaactgtcggcgtttgcagttgcagttgggcagtgtgcacacacgttaacaacagcagaagcagcaaatgacacggctcggctcatcaattagctgctgccacaaacaagggtttacctgtcggctcatcaattagctactactaataatcagcagcgctcggcactctggcagcacaagcgcaggacccaccagtactgtgtgcgtgacagcgtgagtactgagttgggaagccacaccccacaccacacggcgtattggctgtcaaccgacccggatcgatcgactagttgcggttgccgtcggttgctcggacactcagccctgtcaaggtagcagtactcctcccggccttatcactccggtgcagtgcagtggaaacaaaaggcagaaatccctaccacggcggcgcattgatgaatccccgctgctgtgactcagagcctgacgagggggtgctatgctacggcctactccgggctagggtgtggagcgagtgtctgggactgggagaatccagcgtcgtgtactgtccacgcgtacgcgggttctcggcaaggccaagtgggcaacatggcagggtggcaagacggtactgtatgtacctaccggcactacgcttcgacgcctccttttttccaaaaccttccaagccccgcgttcaaactgtgcctaggcgacggatcctcggtgctagtagatcacacacacacacacacacacgggcgaaatgtcggcgagttaatgacggtggttaaagtgaaacctggatccagcaccagcagcagtctttcaggtttcaagaacactcaggtcctgcgttactgtggactgtggtaaaagtgccggaagttcagttcgacgggggaagaaaagaagggccgcacctgcacacagcacacctactacactagtacactgctgcaatgtactacctgggccgtgctccaaaagtcgtaaccactcggagacttccctctgtcctatctccgatcactgatacatctcacaccctcaccacaccgtgacataatttgccagtagggtttacggtgcggtatcgataataaccacactacttgcacaacagctcacttcttcgttattttttttttgtcaagtggggtcgatctggcgctgcctatgattcaagtgtgagaattcagcaacagccagccagctcctgtttccgtgtacatacgctttctaaaagctggaccaggccggcccaatgctccagttgcccgtgcgcgctgcttcgccccctaccccaggcctagccgcacggaaatgaatgcgggcccactgcccggacgacttgcaactagtctccaccctgttcaattaaatccacagacgctgggaaaacaattctttttgaaaggtagaaataaataaaagattgtgggctgttgaagtgcaatctaacccctttctcagactatcttctaaataactacttttcgcagaatcaggcgtgtcatctctagacgatgagattacaccagtcagctacagatgacgaatctgattaaaaaaaatttggtgctagtatactctaatcgttttacgctagatatgttgtcattaattaattgataaaaaaaaaacaatatttgtaactgatgccatgcatgcagcaggctgcacggcacggtcgtccggaactccggatgatgatataagaccagcgtcacccccgcgcgcgcagcctgctgtgatgcgcccaagtcgatcaatcatcgtccgctctcatcatctcatcgcaaagccaa

SEQ ID NO: 22 - DNA, 1992 bp, Zea mays
  source 1..1992: mol_type = unassigned DNA
atctctcctctcctctcctctcctcccgttggtgtgactgtagtagatcctttgcccgtgtcagaacaagctgctcctcggaccgggtaatgttaaacatcggaggagcctttgcctaggatccgtaacggggaggaaagagaaaaaaaactaaggatgattatggataccgtgtaataactgctaactacagttagcccatctcagcggactctctgccctatattgtatgtcactttctattataaactacactatacaacctatgatgtaaaataatgttttgcacgttcatatataaatcagtcgaagaaagggtgcctcactacagggaatggtttctattggacaccttagcattcaatcagtcatgtccccccccccccccaaaaaaaaaatgcacccatccagtcgatttttgtcatatttgaattcggtggtgctccatgcacgcgtacctgctttgaccaatttatacgatcaatatataacttacgttcttacggttcttagactttatgagactttgcaagtatgtttggatacaaatcacactaatgtgcatctttgtaaactaaattcttttgattaaatttgtaattttaaggtttaacctgtttttgttgtgtagacgacgttaggcaccgatcgtcgcttcgctatatatctttgttgtagacgacgttagactcctagattaaataagcgaaaaccgatcgtcgcttcgctatctttgtttatttgtttgtggctgctctacgctgaagagcccacaggccacagccccacacgacacgttaggcacccccacccaccatccgcgcataatataagctactgcaaaatatatgccggcggagcccgagcgagctttgtacttgctccgccgtggcctggctccaggatgctttggatttcgtgcggcgccgtacgtccaggcaaacagacaagtggagctgcatgtcctaaaagcccggcaatcaaacacgctctagcagcagcatggatcacagatatcagtcatggggtggcgctggcgcgggtgggtggccaggtggaggtgggtgcatgtcgtcgtcgtcgtcccatacagaaattggctcacgtatgtatacgctgcgtacaggcagtagtacacaattactagcaccaatgcaatccaacggatggatcttcgcacacccgccacccggttaaattaagctactcctacctctcccagtctcccttggcctgcctctatatttttgggcagcctccaccagccgggcggatggggttggatcgtcgtatctgaggcggcgtggtcgtccaaggcgaaagcaacggcgcagggctgggaccctagtaggtgcatgaggtcgtgcatggcgcgcgagatgcatggtttgggttaggcctaggaggttctctctccatggcatgggtagctcgcgccgcttggctgccgttctcgtgtatgcgcatgcaccaggcatttgcaccgcgccgtgtatatttctggcgtgggggccggcgccgcattggagctgcagccccgtttcggcacggacacgggacacctcccgttagggtaagcccggggcagtgggtaactgcccagcgccactactccgaatttaccctccttttatttttaaagcttgggagaggggagaatggatggatggatggatgtagacgcgtgaaaaagatgcgcgagaccggcagcgtgtgctacaggggcagccaggcacacacacacacagtgaccctgccccttttcccggccgtctcgctctccaccgatattccgctcctcctgtccagtcctcctcccccgagccggctcattatatcgtccgtcgcgcagcacaacgcaagtttgctagcggccggatcagcagccacaaaacgaggagagcaaccacgctgcacacagagacgcccgtgtgtgagatatagagcaagctcgatcgaaggaaggagggaagctagagatcgtacgtcgcc

SEQ ID NO: 23 - DNA, 1998 bp, Zea mays
  source 1..1998: mol_type = unassigned DNA
atagaaaattcctcaaggaactgcacgaggaaatgatccagcaagacatatgcaaaatcaatccagcacacagaaccattccacgtgaggactgcggaacgcagtggaaactccaagttagaagttagaacagttgtgtttttgaaggaacagagaggccctttgtctggaaacgcatgagcatgcatgcatgtctcatgcatctgtgcaggatgtcctaattattatccattaccctggagcagcagatcaagttttttttggtgccctttagttcacaaaatataacgtaaataataataataatggtttacattcgagtactggtggtaataataagtttgaatagaccgatatcaatttttagtatgatatctgattatggttgaatttaagcaaacataatttaacgttatcagttacccaattaattacaaatataaaaatcaaacggcacctagggtacgcagatgtacacaggagtcaggaggcgctgaacgatctccatggtcagttcttaataacatccatgcatgcacctaagtcaaatcatacaataataccgaccctgcagttgttggatgaggcccagcattacgtacacacacgaaaatttttttgataatggaaataagtgttccggcttttcgcatcatcagatacgtacaaccattacatgtcaattattgattcaatggacagttatttagttctttatttgaaaaatgaaactaactcgacaaaaccaaataacatagctatggaaaataaacactaggcacacataagtcttgcatttgatcgccatcgatgaatcgcaaagatttccatcaccatcacttctaaagcttgtcacatatcgagaatttcttgttgtacttcctccttctgtaggagtctctaaaacctgagccagtgggtacctctgaaaatgacctacacaattgatgatattggtttttgattaaaagtcacctcgtttcgaataagccaaatagaccaaaataaagttgatattacagtaagtagtaacttttttatactagaccacaattacttgtccaagacctgataatatgattaatgttgactgtcgtttctatttctagagcaagataaattatcatccaaatatttttggcaatatagcaatcgaaaaagagttaagtacacacacgaactgatataggcaaacagtattttggccgctagctagtcttcctcatccctgttgcttcaactcgattttacacggttccatgtaaatatgctgctggtaaaacaagtaacgatgcatgtgtgcgtatgcatgcatatcgatctgaaagaagatttcagcatcggtctatccattattcatgtgagcctgcagtgactgtgaggtcggtatatcgtcgagtttcacaggcctgtttagtggatgtgggtaatgccggtgatgatgagaaacaaaaggttacatgtgtatgatgtctatagatagtctcatagaacatttggactgtgtatatgtatgtgggcatgcagcaggcacagtgtgaacatgatagaccaagcagggcctcgcgcttgtgcgaatggaccaaccttttcctagctaactctgaggactgtatgcagccttgacagcaagaggagtccgccgtttgaaactcaagaccatggaaaacattttcaaggtccattcggggtagtagtcccttaatttcctttttattaattttttttaaaaggaaaaggaccgggctacttttcaacgcttagcttgtcgacccagcagctagctagctggccgatagaacgagctagcggggcaagcgctctctctggattctataaatccagcattttgctcacattctgctctcgccacctttgaacgatagcctttctcactccgacacttgaaacacgttcatcaggagctcgtcctcaggcttgctagtagcttatattacctacctagctagctagctagtcaagactcaagatccatcc

SEQ ID NO: 24 - DNA, 1898 bp, Zea mays
  source 1..1898: mol_type = unassigned DNA
cgacgagaactgttttcggtctgatatccacggctctgagccagacctgtgtgcctcctggaaaaacctcgttacatccagtaggtttattttgtccccctaggtctacttttgatatgtttctatcacagatgagtgggtgcgctggtacccttttattttgtcgatatgagacaatctttactgttttgttatgcaaataatagaaacggatcctaccgtcgtaaaaaatgaccaaagtccataatggatcgatgaattagttgttgtttttgccatcaaacatgatatacgttaaaaaaactatatggacaaaagaagcaaacatactgctaagtcttttccttaccgaatcgaagccatgtgtgtcttgctgaatgtggattcccttgatgggcagggcattaggaaagagtggggtcaggtgatgtctccagctcagtgtcagggctcactggttcgccaagctcaccaactgccacggacgagtcacatcatcgtgatgagcgatattaggcttgccgggctggctggcgttgcgatgacgatgagcggggcgggcgacatcacatacctagaacgggcagtggtggtaatatcgagaacggggcatcgaggtggactgactagagcagcgtcaggaataataggattgaatggatagatgcgaggactaatccaaaaaaggaggataggagtcaagaggactggactgatcttacgaggaggccaagataaaacgattttagcccactaaaaaactttaattttttctatacttaaccctaaatgaacactaggtataagaggttataaaaattaagggcccactttgcaccccctgaatcagccgctagtaagtgtccactctccaatctccatggcggcaagcgacagacgacaggtctatttggttgtataactaagatgcctaaatttgacacatattttctgccacacttgactgaggttaggtattcaaattcttcgtcacatcttagcatactaagacaactttgctgtacttttacaagtcattgacgagttagacctctgtataggagaaaaattttgtcataactatggctacaaaccaaacacgtgactaagctagtcaaatatgtctaaccttaaacttggtaacagagacaacaaaccaaacatgccccccatgttttcctcattttctttttccacttttgtttcaatttttcttgggtaatatacagtgagtatattattttcttcttctttttctcatggccaaaatccacaatggatcgatgaattagctgtcgttgttgccaacaaaagaacaaaatcatgtgacgcacgagcacaatgcaagtagccaaactgagcttccgggcaccgacgaacggttgcacgccatcggcgggaaggaacaggccgggctgtcaatggacaaacgggccgccaagctggagggagtgtcatgggctttgagaaccatcgtcagggcctagttttttcttttgtttttatcaaaggcggtaaactcggggaacgaatatactaggaaaaacactagccagtcagagtcagtcaaagtggactgagttaaaattgcaacgatacatacacacgcagcagtcagggcgtcgggaatgaacaatggatgaatttattatagtctgaagaaaacgaagggacacagccacaacgaacactggggagtgaatgaatgcatgcattccactggactgttccagcgcttcgtgtgcatcgctagatgcgctgaacactcgaacgccatggacctcgctccgctctctatatatagagggaaggccttcagtctacttctcgggatataccactgaacgtcaccaagaagagtactgatcgatcagcaactcgatcgttctcagagagag

SEQ ID NO: 25 - DNA, 1118 bp, Zea mays
  source 1..1118: mol_type = unassigned DNA
agtcacacttgaacatgattaatacttattctgcaaatatgtcaaataacatttgaagtagtatttttttaatgtacataactaggaaaaggtatacctttttgacataagtatccacgtgcaactgcttgcaattgagctatgttatgcccataatatttgttcaattttcttcaaaactatagatgtgcatacagatagttaaaatgttaaactcatcattgttttccttccacatgtttccatagaaaaaatctaaggaaaaccattacaattttccttgaatgccttgtttctaataaaaagcgggaacaaaacggggcagtttgatctcaattaagaagggtctcttccttccattctttaattgtcgtgatcctactttcttatacttaaggggatattctatttcaatatgcattttttgagttgatggtggtcgttaataaattcttctaagaaaataatctttagtgactatatgatgttttcttagtattaactactaagttgtgtagttgttccataattctacaagattaggtacaataatacaagttgttaccacattattatattgttaagattttgaactgatttgatacttgcgatatttggactttatgtggtcaaatattgctcatattgtttaatatatttaatttagtaagctaaggattttatgtggttgtaatatttggattttatgtggttcaaatatatgagttaggctacggcttgttttagtgccgcactagttttgtatactttaggctggcacaatgccatcaaaaaaaaaattagttctacttaactcgaactattttaatacgaagcaaagtccggccagcactactgcctgccacggcacactactggcctctgtagctcacgacacggtctccaagaagaagctgtttcccaatgttatcatggagtccatggatcagtaaattagttcggggacagcgtggaatacgaaggcattttgcataaatcaagggacttatcatacaggtaatataaataccccctctgatcgctagtagtagttcactatcacactaaccttgcatcaaataaaccattactacttcaccttgccaggccgcctgggc

SEQ ID NO: 26 - DNA, 1898 bp, Zea mays
  source 1..1898: mol_type = unassigned DNA
ccgaaggttctcaaagaaaatgagcgaactcctagatgtcaccgccgttgacgtggtgagtgcattcacctacgacaccaccaacgagtcgtccacgagcttggacaggggtgacaaaagatggccaccgacctcttcgacatagcggtcgctgacgaggaagacgtggtgggggcaattttccgcaagagaaaaaactcgcgtgatgccgacgagcccagcggcgaaacgagggatcaacgagagcaccctggtagacgtcagataaaccactgccccacacgaaccgaggaaggagaagacactgtcgcggaccggtcgctaggggtgggcgttcgggttacccgaaattttcgggtcgggtaattcgggtttttaaaatttcgggttttgagaattgatacccgaaattacaatgggttttgcaatacccgaaaattcgggtacccggaatttcgggttcgggttcgggtattcccgaactacccgaactattatgttggcttcataaaaacacatataccctattaaattagtataaaaacatagtttgaataatgatatacatggacatataaaacataagctatctacaatcacaagttatgcacacttacacataattatagatgtacaaattaataattaatcatgacatgagtatatgacacatgaaagttcgggtaattcgggtacccgaattttccgaaataaattcgggttttgtaagttgctacccgaaattctcgaacaaaattcgggtatttcgagttcaggttcgggtattccggattcgggtttcgggttacgggttttttgctcagccttaccggccgcccacacaactggcgaagaacgacaacgaccacttatagagtggaaatacctatagagtggaaatttttgacacgaggcggtggctgtatggacaggaccggaggaggatgcgcggtacgtaggccgattgtgccagcctattagggcatgtacagtgggtgttttaagttgtgtcttacaacgtgtctagaggggtgaatgtaaaaaaacttaagacacgtatcttgacgaagacacagtgtcttagctctatgttcgagacagaagactagctgattggtcattttaatttattgaatgctctgattggtacaatgaatatcgtaagacacatgttttagatatgaccactgtattatgttgtgttttagttgtatcttgtacttggagtaccgtgcagcagtatctgggttgtacgtgcccttatgtaggggaagggagtagaaatacgatcaatgcgaacgcacgttgatgacggtgaaaacggtagaaaccggtggaagtagagattacagcaaacatggagggacggctttcgtccacccaccggtccttggcgcgcgcgcggcgagcctacgatccaaaagccgcgaccgcttggtgttttggctgacgcttgggaacctgccgcagctgcatttcttgctttgggagccatgtacggcgaactcatacttttgacgtcggttctttttcgaaaaaacttttgacggtcagttttccccactcccgctatatttaaaaccccccaccactcccccaatatttatgacttgacaacaattcccacatcgattcattgtcacttgccagaagtcgggcggccatcgatcccactcccaccgcttacagccggcccagtcctcgtcggcgccggacggggcgaaggtaacccccaagcttccagattctctgtcaacttcagttatgcgtttttcttttaagatgaacctgaaacattttagctttagatcaccggagtcatttttttttttcattaggcgattggagttaaaacaggccgagtgggagaatttcctggatcttatctgttctccatc

SEQ ID NO: 27 - DNA, 2000 bp, Zea mays
  source 1..2000: mol_type = unassigned DNA
ccgacaacatcggcatacggcggaaatactggcagatgctgctcgtctcgtcgctcgctatcttttatttacgacgatcacttgctcacagtcacagctagcgctagctagcgatcgaacaggcagcgaacgtggcagcggcagctgccgcgccggccgtgcggaggatggtaaaatacaactgcgggttggcctcgaacaccgcgcggcagatgacccaacctgcggcgccgggcgccttcatcatcatccccgccgaagcggggcccaccacgggccacaggaaatagatgccgaagacgcccatggcggcaaacgccaccgccgctcccatagcgcctatgccctcgtcgtccttcatgttgacgaatgacgatggccgggcaggttgacatgcaggattcagcgtcgtccgggtggcacgccggtgttgcttctgcccttgctgcttaattggaggagagcgatcggctgggtttggaatgaatggacactggacagcggtcgctgcgtgttgtgttaattgaacatctatttgtagtaggctgcagcgccggatcgaggttggccaggcggctgctcagcttcatcgcgtgcattgcgatgcatgccgtgcgcatttgctccccagtctcaaatcacgatgggaggcgacgacgaagagacagcttcgcaatgtgcgcgatcagtcgagcgcattgaagttgaagagaacggtagcgaaatgcagaagctgaattgttgcttggtagtagtgagaacgtgcataagaggtaattaatttcatctgctaattatgtagagtagtaattaaattaatttcattaactacgagtcacctcgaggccatgctataaaggccagcggtcgacatagcttaaatgatggtgcattggcccccgcagcccggtgccgtaaatacaagacatccaggtgaatctggggctcacaagggcgtgtggttttacctcgggtagaacgttggttttacccttgccttacggcgccttcctaaggaaccgacccctcaaggttacatgctttggcacactaggattaggtaacccctcaaggttacatgcatttgactcaaggtttaataatgcttttggtttatatacgaaattatgagtatattttccaaagcttccggataatataaatttctttttcattttaagggctgatttggtgatcagagattccaagatgattcatgggagagaaatccccttacaattcaattttgaatatcaaggggattcctctcatggatcctctcgggatcactgaccaccaaatcagccctaaaagtgaactgatggaaattaaaacacatacgtgcataagaggtgattaatttcatttgctaattatgtagagtagtaattaaatttcaccggcaaaagtagtatttttttttttgccgatactactcgtccactacccaccaatccgtcgtggcagcttggattagtattaaaaatgaagaaagtgacagaactcagggtattattttaaataacctaactatacccattagaagattggacttggacatgtaccaccatgagcctgcctttggattagtattgtcggaaaatgatcgttctcactccgaatcgacgagtcgacttctgcatttgatggggccggctacactttgcgaaatgggtgttgatcggcgcatcgggcggagcgggcagtagctgcagtacttggctatgcactcagagtcggaggcggagcaacgcctcttggagttcccaggccgattcgttttccccagcacgcgcacttcgccgtacgtgagacggcagcaattacgcacgcatgcaacgcacccccatagaaggccagcagccgcctctggccgacctacaaatagatgacttctctgtcgcagaaattccacgcggtgtctatcaatagtaccaaccctataccagagagcgagcgcgcttgacgacttgtttgattcatcactctccttcaagatc

SEQ ID NO: 28 - DNA, 2000 bp, Zea mays; identical to SEQ ID NO: 3
  source 1..2000: mol_type = unassigned DNA

SEQ ID NO: 29 - DNA, 2000 bp, Zea mays
  source 1..2000: mol_type = unassigned DNA
ccttaccggcgatgtgtgcgcatgccggagagcaaggaggagtgcaaggcctcacgacgcggcgccaggtgagggctactcatgtgcgtcacccattgctggtaagtcctcattgctcctcccttctcaagtctctgctacgcgtgtgcattagttgtagggaatacatgatgttaagtatgtactgattttctgatgcaacctcaatagatttgtaagttttggctatcttctaagaggtacatagaattagtgacaggatggactacttcctatctgatgtctcatgtaagctgggacctgggagcatgaaaatgttggtgcatgataccagcaatgtcttttttttttgcttatgtcatcttgtttgaaccaaaacaaaatacattaggtaacacgatcctatgatgtcgggtcatgctcaagagaatggtgctgtcgctgaaattttgcattaatttatcatgttcagctccttgtgcctagatacgagagatggttaactgaaactagatattttaccatgttcatctcctgatgtctggatacaaggtggttaactgaaacaagatggaatgtgtttgcaagggacaccaacaaggttactgtcatgtacatacatattgattaacgagcatgacacgaacctctttgttgaatgtgattttgacgtaagaacttttagttcatgtggtttcaaacctatttatatgatatataacaattgtagcaaaacacggacaactagctagtatatatttataacatttgacactttaatatccccttcctgtcacttctatagcatttgtctagtgttgctcattcaaaacttctcggtcgacttcatggtcttattttttgttctggtaaaattccgataaaaatcagtaaattccgattaaaatcggtaaccgaacaaaacgatcgtaaaaatgatacactgatttcgatactgatttcgtttcctaataatattgttacccgtgaatccgatcgagaaaaatcgaaaacggttttcgaaattctaagaaattccgaaactgttttcatcatacctttgcctgtctctccaatggattctggagtggcggcatcgcctatcagagtagaatttcttccccaagaacaaccaaccgctgaaccaacagcagaatgagggcttttcccttccaaaggtgaacatgtttcggagttaccctagctgtcgcagagaagatggagtacgaaaccagagatgatgacttgggggacttccaggctgctcaattgatataccgtgtatcttgtacataccgaatgttactgagacgacattttcatttttggtctatcttctagcggctctcaggtactatgattttttagatcaaatggtgtttgagaacacatacaagatcgaactctgaattttaaactattgttttgccaataccacggtattttggggttaaaaaatctttggtctagaccaaagtttttttgtttgcgcgcagctatatcatgatatacattaaactgcggtattataagatgttgttttaagaaataaagttcctaaataggccctcagtgttcctgtaagcctcctcttctcatattctatatacagtacgtactagtactacaacacactagctagctagtcagtcaaagtcgattgcacggatgcacacgcagtcacggtcacgggcttgagaatgaacgatggatgaccttagcaggcagcagctgttactctcatgttattaattatctgtgtgaggaaaacgaagggacacagccacccaccgcaacacaggggaatgaatggatgcattccacttgaccgttcgttccaatccagcgcttgacagtttgacactaggtagaacgccatcgaccacgctccgcactctataaatagagggaaggcctccactctcctctgcgggagataccagtaaacggcgacgaagacaattctctaccaagaagaggactgatcgagtgggagagat

SEQ ID NO: 30 - DNA, 2000 bp, Zea mays
  source 1..2000: mol_type = unassigned DNA
cctgccgaccccaagacgacccaaggatcttacccctcccgaggggcaagatgacttcgtctacttcacttaagatttcttcctcgaattgatctcttaattcctaggcgactctacgtcgtctggggacgccctaggtgacttgctgacccggagcaccctaaggtctcttccctagggggcgagatctaggtcctatgaggagaagaagacgaccatgcgccatcccagaccgtccagcccagagcgcagatcgtccgtacagcgtgcagggaagaagccgcttctgtcgccaggtcgcggaccatccaacctagagccacgggcagtccgcgcaatcgcagcaagcaccgccaggcaacccagcgacgcgtcgcggaccgccgcgccatccacctcgcacaaccgaacccaaggtattgctcatcacgagaagaccctagggttcgttactgtttggtcccacataggtggtggccatcatagaggtggtattacaccttcatgataaccatcttaggtacatcaagtgataatccccttgtctataaggattcctttgaagaccacattcaagataatgttatttgtcttacatcttatttgttatctccctccccatggtctcgacccaccgtgggcaaaacctagggatcatccccaaggtccgaactcactacgaggacgacctaggaatcatgtcacttttgtagcatttgtctagcgttgctcattcaaaacttccccgtcggattcatgggttttttattctggtaaaatttcgataaaaacgagaaatttcaattaaaatcggtaactgaacaaaacagtcgtaaaaatggtacaccgattttgataccgattatgtttcctaataatattgttaccggtgaatctgatcgagaaaaactaaaatgattttcaaaattctaaaaaattctgaaatcgttttcatccctaccttcacctgtctctccaacggattctggaatggcagcgtcgcctatcagagtataatttcttccccaagagcaactggccactgaacccaacagcagaacgagggctttcccttccaaaggtgaacctgtttcggagcaactagctaccgcaaagaagttggagcaaaaaaacggggatgacgacttaggggacttccaggctgcttaggtgatataccgtgtatcatgtacatacagaatgttacttagaccacattttcatttttggtctatcttcttgcgacactcagggtctgtatgggaacacaatattttcttagttataataccgtagcatttgcgcatactatagtattttagatcaaatgtagtttgataggacatacagaactttgtatttttaaactattgtttgctaataccacactatttttggggggttaaaaaatctttggtcttgaccaaagttttttgtttgcatgcagctacagttttcttttctcttacattacaactaaaatactatgtctctaaacaggtgttgaattggactgacgtaaaatatacatagttatatcacgatatacattaaactgcgcggtattataagctgcggttttaagaaacaaagttactaaataggcccacagtgttcctgtaagcttccttttcatgacctacaacacactatctagctagtcagtcaaagtcgattgcacagattcacacgatcacgggcttgggaatgaacaatggatgaatttagccgccgctgttactctcgtgttattaattatctgtgtgaagaaaacgaagggacacagccaccaaccgtggcaacacaggggaatcaatgaatgcatgcactccactggaccgttcgttccattccagcgcttgacactggctagaacgccatcgaccacgctccgcactctatatatagagggaaggcctccactctcctttgcgggagataccagtaaacggcgacgaaggcaatcctctattaagaagaggactgatcgagtgagagag

SEQ ID NO: 31 - DNA, 2000 bp, Zea mays
  source 1..2000: mol_type = unassigned DNA
ttcactgctcctcactcgcaaccttgccggcgaccttaccggcgaagtgtgcgcgtgccggagagcaaggaggagtgcaaggcctcacgacgcgccgccaggtgagggctactcatgtgcgtcacccattgctggtaagtcctcattgctcctcccttctcaagtctctgctacgcgtgtgcattagttgtagggaatacatgatgttaagtatgtactgattttctgatgcaacctcgatagatttgtaagttttggctatcttctaagaggtacatagaattagtgacaggatggactacttcctatctgatgtctcatgtaagctgggacctgggagcatgaaaatgttggtgcatgataccagcaatgtctttttttgcttatgtcgtcttgtttgaaccaaaacaaaatacattaggtaacacgatcctatgatgtcaggtcatgctcaagagaatggtggtgtcgctgaaattttgcattaatttatcctgttcagctccttgtgcctagatacgagagatggttaactgaaactagatattttatcatgttcatctcctgatgtctggatacaaggtggttaactgaaacaagatggaatgtgtttgcaagggacgccaacaaagttactgtcatgtacatacatattgattaacgagcatgacccgaacctctttgttgaatgtgattttgaggtaagaacttttagttcatgtagtttcaaacctatttatatgatatataacaattgtagcaaaacacggacaactagctagtatatatttataacatttgacactttaatatccccttcctgtcacttctatagcatttgtctagtgttgctcattcaaaacttctcggtcgacttcatggtcttattttttgttctggtaaaattccgataaaaaatcagtaaattccgattaaaatcggtaaccgaacaaaacggtcgtaaaaatgatacactgatttcgatactgattctgtttcctaataatattgttactcatgaatccgatcgagaaaaatcgaaaacggttttcgaaattctaagaaattccgaaactgttttcatcctacctttgcatgtctctccaatggattctggagtgacggcgtcgcctatcagagtagaatttcttccccaagaacaaccaaccgctgaaaccaacagcagaatgagggcttttcccttccaaaggtgaacatgttttggagttaccctagctgtcgcagagaagatggagtaggaaaccagagatgatgacttgggggacttccaggctcctcaattgatataccgtgtatcttgtacataccgaatgttactgagaagacattttcattttttgtctatcttctagcggctctcaggtactatgatattttagatcaaatggtgtttgagaacacatacaagatcgaactctgaattttaaactattgttttgccaataccacggtattttggggttaaaaaatctttggtctagaccaaagttttttgtttgcgcgcagctatatcatgacatacattaaactgtggtattataagatgtggttttaagaaataaagttcctaaataggcccttcctgtaagcttcctcttctcataacctatatacgtactacaacacactagctagctaatcagtcaaagtcgattgcacggatgcacacacagtcacggtcacgggcttgggaatgaacaatggatgaatttagccgctgttactctcgtgttattaattatctgtgtgaggaaaacgaaggggcacagccacccacaaccacagcacaggggaatgaatgcatgcactccactggaccgttcgttccatttcagcgcttgacactagctagaacgccatcgaccacgctccgcactctatatatagagggaaggcctccactctccactgcgggagataccagcaaacggcgacgaagacaatcctctaccaagaagaggactgatcgattgagacag

SEQ ID NO: 32 - DNA, 2000 bp, Zea mays
  source 1..2000: mol_type = unassigned DNA
gtagcaaaatggttcgtgccagatgcacgagggacagtggacaatgtttacaggttcgggccgcttagagatgcgtaacaccctacgtcctggtgagtatgcttggtgaatgaggttacaagagagctctctgaattgagaatgcagagaattgaggtgggtggctgagtaatagggtcgatcgttctgaaggggtgccccctaggccttatatactcgaccgtggggcaatacacgtggatatgataacaatgtgtaagaaaaagatagtgaactgtttgagtttatctccctatagttttgccgacctatcctcgcatggctccgttgcttgggggctccagcgcgggaaagtcggatctctgttgtggtcgctcgctcgatgttgtgggccgtccgggcttgcaccaatccggcctcatgtcaatctgctttgctgattgtccctcccaggcccacgaaagaatgaccttacgatttattggacccttgggcctttcgtgaggtcttttacttcttctagtggacccaagggatatctgtcccccacaccaccatactagttcctaaggtaagcattaatatatgtcatgaacctccatgttgtagacatgttgtccacacatgataggctagcggtgttgttcaccctacttcattgcacctcctagtacattgtgcacacattacaaggatcccaagatgctatatgtctgtcttagagcccccagagtgctttgtagactaggttgtgcgttcatgcacactttgtaatgccatttatcttgcattaaatgtgttgtatcgtataattatatactaagctattgttttataccactcttcgagatttcactcggacgaggcccacatattggttgtatacgcccctaacaagcccctagtgttcgtatggtccttgctgggcctgagctatttgttaaacatatggccgagacacgtgtgagtctaaggaaatatcacgcgagggatgccggggcgaggggccctgggacttgagggactttaaggtctcatctattatgcatcgcgaggtgcgagacgacctagggccttagtcaataggtgccttagggcacgaggcatctcgaggtcttgatcattaagcgccctagggcatgatggacctcaagtctttattcaataggtgcctcaaggcacgagggacctaggggctttatttgttaagtgtcctaacatgtgatgggtcccatggtcctatatgttaggtgttcagggtgcgaggtgttctgtggttttatttatttggtgtctcgatgcgtgaggggtccgaggccttatccattagacatcttgagataagaagagctctaaaggcttatttagtgaatgttccaagacatacgaagcctcgatccagctcgataattgaccagagatgtgtgagtccttgtgttaaggatccttgagcacgttacaatactatcgaggagggctatgtgctttcctagatactaacatatggtggagggctatgtcctttctcagatgctaacatatgatgcgcaaagaaaagtgttgtgtactttgtacctaaacttaggggctgtttggtttgtgcctaactatgccacattttgtttaaggttagtcgttcgaattgaaaaactaatcttaaacagaaaagttagacaaaatatgacaagttagggagagaaccaaacatacccttaatgtagggcattggttacggtatagcggtcaatttaattggcctgtttgttacgctgtagaaaaccttaagtaataacgaccgttgggggtacataatgcgttttttttgcatgttatacgcatcgttttccaataaatgatgtccataactccatcagttagcacctctggacgtaacagtgcaaaagaagagtcactgcatatttatatggagagagaggtagaagacgtgaagttggggattcacagccttctccttctccctcgctcgcgtgttagaaata

SEQ ID NO: 33 - DNA, 1595 bp, Zea mays
  source 1..1595: mol_type = unassigned DNA
tgagtacttcgatgaatcagcttgattcgtatattcatgtcttgcactccacatttattccagcacattgctagagcattgcaagcaccaaaagcctagtgaggagattagagaatcataatccgcgtttgttcctcattagcgctagcgagagccacctagagcacacaccacttgcattaggcttctcttggtcaagcgaaagtctacgacttgttactcttggtgatcggcatcacctagacggcttggtggcgttgggagctcggtgatcaccgtgaagatcttgttggtgacccgactcaagtttgtaagcggtctcgagggatccaccgcgccggagtggcaaaggatcatctcgtagtgagcacttggttcttgcgaggaccaagggggagcgatacccttgcgcgggtgctccaacgaggactaatggagagtgccgactcttcggtacctcgggaaaaattggaggagtcttctaaactttgctttacattccgcacttaattcaagcactttacattgtgtatttgtttagcaagtatttgaagtattgtcttagcattgttgcatttctagtattatattcttagtgctagttgttggggtgaagttgggctcttgcttagtctttaattagtgttgatttttagaaaagcccaattcaccccccctattgggcatcgtgatcctttcaaggttctttgcaatgccagataaaaattttcccagcacacaaattccttagtagatgatctgggaaggatttgcggcctattttagagagggcaataaaattgagaccatgttttttggtcagatccgaaacgtatctaagttttttaaatccttgaactcattgttgttctaaaagattccttttattgggaaacagttaattaataatcaagcccggcctctttttagaacacaacgaaccgtaatccgtaactatatggttttgtacatactgacttaatcagtccatcaatgccatctgtctcttcatgtataatatccgcacacaaatgatttaaaataaaataatcaaactcttcgtcctctagtactccctttcccataaaatcagataaatcagccttgttcttacctatattaatctctaatttttttaactggccaatagaggaagcaactgaactaggattagaacccgctaaaatatcaatatttttaaaagaatcaaaaatataattatcaggtaaaccaataaaaaacattattacctttagtcggctgaactagtatgaattggcatcggtaggaaatgcagttcacacgactaggaaggataaaatcgcagccctaaactgtaaattctctttgtgtaaactggaaaacgagaagtttctgaagtaaattagcttcaatgcccgactcttcgtgcgaactggaaagacgcttttgtcgtttcgttttctgatctgtgacctgtcagactccagggcgattcagcgcatgctgcacgcaccagccaccaggtattttaaggggtcacacttgcttgtttcatgtactagcacaagaagaaaccgtaccacttcatcaggccaaatcgccagcaatccacc

SEQ ID NO: 34 - DNA, 970 bp, Zea mays
  source 1..970: mol_type = unassigned DNA
agtgtacaaagtctaatccacagctgtgaccctgtgagctgggggttgggcctgcctcggaggggatcgtgtcttttctgtttgtgcaattatcagtgcctaaagaacatgtgttataacagttatacacggtcaattattcaatccatcttttaacgtgtgtttggttgtacgtataagaagggatcaaagagggcggccacattttctataacatttggatgagagtcacacgagaacgagatgaacaccgaagtaattttttatgacagcatgatccaaaaaaatcgagagaacggtgttatccccgtctcatcctattttgatctcaaaccaaacacataattagtcaatgagcactgacttgatgtagaagaacttggtacatataccaggaaatgactctgacttgctgagatgccttgtagtccctcggtcccgtaaattatacagtagtatttaaacttcctcgcacaaaggaaaactatttgtttttgcatgccttgtaaattagctatcaagtttttttttaaaaaaaacgacctttgaaaaccagtaaggaggactcgttcggtattcagttttaaagttcaaggttaaaaatcgaactgacgtgcaaaacaccctgggggttaaaatcaaaacacacaaatttggaaaatgagcaggcatgacttatgttgtgtccgttgaagtgccgtggctccatggcacggtggaactttacattagtgcggtagtgtcactgtggcagtaaagcccacttattttccattagagagagggagattaaaaacgtggctttacacattctgaaactaattgtgagtttgtgactcttggcttgcctgcagtgtccgcctgcaccttctataagtacagtcgcgagcctcagctatacaaaccaactagcgcatagtatctcctgagctccattcatcatcccaagtcttcactggtcttagatcac

SEQ ID NO: 35 - DNA, 1995 bp, Zea mays
  source 1..1995: mol_type = unassigned DNA
tctggagtcccaccggaccacaaaagcaaccacttgtttgcgcatgtcaagtaggccagggcaagcagattgtttgttgcaggtgacctaatccagcggaaatgtcttctgatggcgactgcaggagatggtgccagaccacaattcatgtgtcatgagtaattcagctcaggacatgccaacagagacagtggcctccagcgaggggcatccaacccaataaaacaacgttcaagttcctaatccgagacacacatcggtaaaagtagggggtgttgaagatctatccagttacagtgctcacgtatgcgtaaagcaagcccaagtgctaacattcaagacaaagcaaggccagtgccctctgtacattcttacatcacacatcagttgtgaccaatgggccataacctcttctccacaggataatacaacaacaattaaagacagaaatgtcagtgcgcaatcataaatgaactgcgacaatgaagaacaggtagcaagccttatttctagcaaacagtcagctcagcgctaagaaactaactactatactagatcatgtaattctaagaaaagggcatgattctgaatacacaagttttacttagcattctgggactcaattttagatgaatctaatttccgtcaaccatatttccgagagaaagctggaatcttccagttcaagcaactgccaagctaaatgctatggtgcagtgaattgcgactacagtaacgaagcgagcaagaatcatggaagggagggaacatgggtacgcgtgttacccacccgggagcagccgagttttggaggcccattttgcgcttacggatatctgttaagaggaggtccaaaagaatccgtaaaccaatctcaaaacaaatattagtagagaatagtgatcttctttgctccaaacagctcccctattctcctctccaaaatttcgtatccctcatctacctccttcgctcccgtatatttttgcatgattgatggctccacacgcgctggcgtttttgtttctcgcacatagacttgtttcgatggagacatcaaccaggaagctgtttggaggttacgaaggcctacatttccaaatattgcgaagggcccatatacatgtgcaaagtgggctgccaggagggcgttttattttaaatttgagtccagttttttggtttttcagttttgactgttggagtagacacaatatttcacccctaaaaaattttaggcccctttgaaatgcatgattgataaaatgcaggaatagaaaaaaaaaaacgtaggaatggagttgtactacaactacaatcatacaagaattaaaaatacttgaaaacatcaaatagagtgtttagatgcagcgtataaagaacaaatgagttagaggagagagatacacacaaaggaacgtttttcgaggttggacctcttgttataaatcctccaaaattttactacaatgaataattttatagggattttatagaatttgtaggaacttaatcctttattctaaaaggctatataggaaaatttcctatatgattccaatcctctaaaatcccactacttttcatttgttctaaaggaggccctagccctattccgataacatcttttctttgaaacattttaggttttttttttggagttgctctaaatggagctaggcaaaaagagcgatactcctctcaatttggaacttgggacgtatttattgcatgcatgcggaccggggaggagttgaacagacaggccggcgtcggggagggaaacgatgcaattgaaattgaatgaatagaatataatgcaaatgaaattgaaccgaaacgacagccaatccgatccgcttgccttcctgcgcgcctctaccacttccaccaactgagtgaatagtatacagtatacataccgctaggcgctagcttctccaaatctctaccacaccaacctgcctcgctctccaacaaacatagcagccagcc

SEQ ID NO: 36 - DNA, 7579 bp, Zea mays
  misc_difference 686..785: note = n is a, c, g, or t
  misc_difference 1937..2036: note = n is a, c, g, or t
  misc_difference 7521..7579: note = n is a, c, g, or t
  source 1..7579: mol_type = unassigned DNA
gcaagtgatgcacatattcagggggagttgtgctacaacttgaccctttgagactaaccgtttgcttgagtttgcttgatttagtctcaaaggaagtttgaaagggaaaaggtggacttggaccatgaaagacttccactacactccgatgagagggtaacttattccaagttcatctcatgtactcttattgcctttgtattcttatttgaagattttggtgaggcaatggggttattgggccaagattggtcccgttttggtgcttgatgccaaagggggagaaaataaaggccaaagcaataaatggatcagataccacttgagagtttttttgaaaatagtagaatagagctttttgtttgtcaaaaagcttttattgtctctcttgtcaaaagttggcttcttgtggggagaagtgttgattatgggaaaaagggggagtttttgaaatcttgaatcaatttctcttggaatgactctctttatgtcttaacaagtatgtttgacttagagatagaaatttgagtttgatttgcaaaaataaaccaagtggtggcaaagagtgatccatatatgccaaatatgaatcaaaacaattttgagttctcatttgcatccctcttccccttgttctagttgctttatgttgtgtggcataaatcaacgaattccaaaggtctagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttaaggttgttgttgggcataaaatcacccaaaaaggggggaggaattgaaaggggccatttctaagtattttggggattgaatgcaacacaagtgctttgtgtaatctatgcaaagtggtggacaaagtgcaaatcatgtcaaaggtatgtttctggacttagtacattgttttattgactaatgtattgtatctaagtgctggaaacaggaaaaattgaattggaaatgagatggctacgttcagcctaagtcagctcggtctgggtgcaccggacagtgtccggtggtgcaccggacagtgtctggtgcgccaggctggctctagcgaacaggctgctctcgggacttcgacgacggcgtacggctaaaattcaccggactgcccggtggtgcaccggactgtccggtgagccaacggtctgccggggcaacagtcggccgaggattccgcgcgtgacgcgtggccgagccaacgatcacattggagcaccggactgtccggtgtgcaccggacagtgtccggtgcgccaacggctcccaggcgccaacggtcggcttcgctaaataaggaaagagatccgcaccggacagtgtacggtggtgcaccggactgtccggtgcgccaggcggcagaaggcaagatcagccttcctggaatgctctcaacggctcctagctgcattggggctataaaagggaccctaggcacatggaggagatacccaagcatactcaaagcattcctaagcaccaagacttcttttccacgcatttgtttcttcgtgatagcatctagagctcttgttgagttgtgaactcgtcgggttgtgttgcgagctcttgttgccacttgtgtgcgtgttgacattctgacttcgtgtcttgtgtgcgttgatcatccctcccttactccgtgcttctttgtgaactttaagtgtaagggcgagagactccaagttgtggagattcctcgcggaagggattaagaaaagcaaagcaaaacaccgtggtattcaagtgggtctttggaccgcttgagaggggttgattgcaaccctcgtccatttggacgccacaacgtggagtaggcaagtgttggtattggccgaacccacgggataaccaccgtgcccacataaaaagaaggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctaaggattcaagctttgaattcgttgctacaagcagaaatggtaagttgaactgagttagaagaggactgattatataatcagcgtggataaatattgtttctaaccaagctttctatcctatatttgcatgttcttcaaagggatgtatatggaccttccaaatacactagacttttgcaagcaaccaaagatactctcagagaagaaggtttgccttgccttgaatacttcttgacttatgtcatattgagcatttgagctattgagaaacatactaatacagacattgttgatcttgcacttgcatgcctctatgtaataggctaaactaagcagcattgtataggatactggtacttttgcacttgcattatctcttcttgctaggtagagtcctaccccatagtctggaactagaagaattctgattttccttttaggaatcaatgtctatttgagctttgtttggtcagaaatgcattgtagaaaaatttagatggtgagatttttatctttgttctcatttttattcattgtagtataatttttttcagaggttgttctcatttatctataactttttattttttttacgaaaagccggtgcatgctgattatgcaccaagaaacatagttacaggcagatacttgttgtagtgctctctttacaatttatgtgtactatatgctgtattctcaggtagtctggctgaacggctgccccagtggaagagatgaccgcgcagtgcagtgcagcactgcgttggtggatgatcgatcgtccagttgtatgtgactaaaacgccatgccctgtgcattagagaagatagagaatcgccatgccctgtacaggacagtgagccaaattcttgttctttttgcactatgatacagattgattcattaggccctaggttggaggttgtgactgatgagttgggaagggattacttgattggtggtgtacaattcttggacaggatacaggttccagtttttggtccattagttcaggaattgtttcatttacatgtatattcagtacactcgtataatggaaacagacaaaatctctatctcattctttagttatcgtgttattatatggttccgttgcaacgcacgggcacccacctagtaatatattgtgcatgttgcgattgtaaaatacctatggtaccgcagttaagtaaaatacctatggccgacggagtgcttcatttgagaactggaaggtctggtagtaatttcacattgattcaaactcacgtgtgtttctaaaatacggcagttcaaacgaacacgcggctctcgacttccgttgcattgccaatgccaatgcggctgcattgcatgtggtgttggttcacattcgctcatcaatatacggcccggccagagctggttgcttggcaaagcaaccgttcaatctccctctccctctcctctgtagtatatatagtagtatacaactagcagtattcaagaagtcacatctctgagctctatggcaactcgttgtcagtctgctggtttcatcgaattcaagctcgacttcgcagcatccaaccaccttgccgtcggcgacatcgtccgctccgacgatttctcggcaggcgggcacttatggagagtaatttgctacccaaagggagacgaggttggtaatggcaattacctctctctctacctacggctcgtcagcgactccaagtccgagaaaatcaaggccatcatcgacgctttcctactcggcagaaacggcgcaccgtcatcatcttcgcatggaaagcgttgggtgcatgtgtattcttcacccgatggctcgcgatcacgtgggtttccagagttcgtgaaacggagcgtcctagatcagtctgactgcgtcacggacggcttcgtcacattcatggttgtagtcatagttcttagggatagccccatggccatacccgtaccatcttctgacatcgccgaccatcttggccgcctcctggatcacgctaatgacggttccaccgacgtgaccttcactgtcggcaccgagacgttccacgcgcacagggccgtgctcgctgcccggtcgccggtgttcaaagcgcagctcttcggctctatggcggacgccaagatcacgctgcaaggcatccgtccggaggtgttccggatcctgctgcggttcatgtacaccgatgcgtttccgggcgacgacagcgacaacgacaacgatgacgctgaagattcgttgccgagagacgacgacgatgaagacgagcaggagggttattccgacatcgacctgttccaggatttgctcgcggcagctgacatgtaccacctggaccggctgaagctcatgtgcgcccgcaagctgtgggaccgcgtatcgggagagactgtagccaagcttcttgtctgcgctgaactgcgtgactgctcggagctcaagagcgcctgcctcgacttctttcttgtcgagaaaaatttcaaggtggctgtactaacagatgggtatttgcagcttatgcagagctttccatccgtcatcgccgagataaaagcgcgggttcgtcagatctaaataagacatagcgtgcgtgcgagctatattatatagtttgcagtttctttaatatttaggccagaatgatgctttcgtcagatctgaataaagacatatatatgatgcgtccgatctacaatattataatggtatgctgtttctcagaatctgtaagaccgaatgttgctttatcagagtacattatatattatgtgtcgaggaaacatgaaattatgcctacctcataaaaaatgaccataaatcctaaacactaaacaagacaatctaaatctctactacatattaagacaataaaggtagtctgtctcccctcgtttttccgttttgccgtccatcgatctggaccgtccgtatcctatcgttcgtctgcgtctcccacgctccatccccctccaccgcacgaccgcacccaatccctaacctttccccgcacgttcctctcctctcaatgccacgttgcctgccccctgcccctgccgcgatagatgcctcgcggcccccgacaacgacaaccgcgccaccatgacctcccatcgcctccatgaaagagaaatgtgcccttgggccatttctaagtattttggtgatttagtgtccaacacaagtgcttaagtgataaacaagtgcttaagtgataaactatgtcaaatgatggataaaggtgcaaatcaatacaaagatatgattctagacttagtacattggtttttgtgtgctaacatgtttgtctaagtgttagaatcagagaaaatgcaattgaaaaagagtggcttgaagcagccaagagtctgctcagtctgggtgcaccggaccgtccggtggtgtaccggacagtgtccggtgcgccaggttggctctggcgaactggctgctctcaggacttcgacggcggcatacgactataaatcaccagactgtccggtggtgcaccgggtagaatccgcgcgcgacgcgtggcaagtgccaacggtcagatggggcaccggactgtccggtgcgccaacggctccaaagcgccaacggtcggcttcgccaaagaaggaaagaaatccgcaccggacagtgtccggtgcgccaggcgacagaaggcaagaattgccttcctggaatgcactcaacggctcctagctgccttagggctataaaagggacccctaggcgcatggaggaggaacccaaacattctctaagcattcctaagcaccaagacttcgattccgcgcatttgattctttgtgatagcaactagagctccattttagttgtgaacttgtcgggttgtgttgtgagatcttgttgtgacttgtgtgcatgttggttctctgatttcgtgtcttgtgtgcgttgctcatcccttccttactccgtgcttctttgtgaacatcaaagtgtaagggcgagaggctccaagttgtggagattcctcgcaaacgggatatagtaaagcaaagcaaaacaccgtggtattcaagtggtctttagaccgcttgagaggggttgattgcaaccctcgtccgttgggacgccacaacgtggagtaggcaagtgttgaacttggccgaaccacgggataaaccactgtgtctatctgtgattgatcttcttgtggttatcgtgtcttgcaaagactcttatctagccacttggctttattgtgctaactcctaaccaagttttgtggcattaaagtttaagttttacaggatcacctattcacccccctctaggtgctctcaattggtatcggagccgttctcttcaagaaagggactaatcgcccgaagagatggatcctaagggcaaggggatggtggtcaacgataaggagaaggagtcctttgtcaatgatccaaaggaggacaagcctactgactcgggctcgagccacaaaagaaaagatgggaggaagaagaaaacaaggcgcatcaaagagatagtctactacgacagcgacgaatcctcttcttcccaaaaggacgacgacaacgactatgagaaaaagaagacggttaattcaaacttctcttttgattattctcgtattcctcaaagtaccaatgctcatttactttccattccacttggtaaacctcctcattttgatggagagtactagggattttggagtcacaaaatgcgtagtcacttgttctctctccatcctagtatatgggagatagtagagagtggaatgaaatttgatagctcggatagtcctttgtttatcaatgaacagattcataaaaatgcacaagctactactgtgttgttagcctcattgtgcagggacgagtaccataagttgagcggcttggacaatgccaagcagatctgggacaccctcaagatctcacatgaggggaacgacgtcaccatgctcaccaagatggagttggtggaaggcgaactcgggagattcgcgatgataagaggggaggagccaacccatacatacaacaggctcaagactctcgtcaacaagataaggagctatggaagcacgcgatggacggaccacgacgtcgtccgcctaatgctaaggtcctttactgtacttgatccacatctggtgaacaatatttgtgaaaatcctaggtacaccaagatgtcgcccgaagaaattcttgggaaatttgtaaccgggcggatgatgatcaaggaggcgagatacgtggatgatgcattgaatggcccaatccacgagcctcaaaccgttgctctcaaggcgacaaggagcaaggaggcactacctagcaaggtggcgcaagttgaggcggtcgggcttaatgaggaagaaatggcccttatcatcaagcggttcaagatggcgctaaagggtcgcaaggagcaccccaacaagaacaagacgaagggaagacgaatctttagatttcccnnnnnnnnnnnnnnnnnnnnnnnnnnnnnnnnnnnnnnnnnnnnnnnnnnnnnnnnnnn

SEQ ID NO: 37 - DNA, 2000 bp, Zea mays
  source 1..2000: mol_type = unassigned DNA
ttgcaatcgtgtgtgtgtatatatatatatatatatatatatatatatatatatatatatataatctgcttttgtatttcacatttacttcacatctagtctttctaagataatgttcattgtgtatatactaaattgattcatttcagctactaactcctactgctatatattgtttaatcacactgctatgctatgctactagcattgaactatataattgtgtaaccatataaactaaaatgtgtgtcaattggtattacttgcagtgtgttaagtggaagcaacacatgaagcggacatggagaaggaagctggcatgacactaaaacgcataaagttgacatgtgacgttcatgttttagatgtcttattctactgccaaacttcataatactccatttttatactatattgtctatctgtacgtgtgaatggagatgatgatattatgtgatgtgatgatggatggattagcttcgttgaatattagatgatattatgtgatgatgatattaaacgggcattgcagtattgatacatgcttcatatttctggatttttagtgatgttttattgtttatgagttttctaaatattccttatttttgtttgattttttctaaatttttgatgtattttttgctcaagaggggtatttagcccctttcttcccaaacaacgccaggaaaatatatccgggaggggagattctctccgggaaataaggtgacatctaaaatcccctccagggttagtttccaagggatatttttctcttcactgccaaactagccctcagcgtccttctcgatctccgtggattggtaagtggctgctggcatttcatccccaatatcaacatactcatccacctgctcagccactgtagtactcttcacaggttcctcgctctcctgcaaaaataataaataaacaaactagcatcagcgcccaattcatatgtccactagctgcaaccaaaaacaaacgtgctgctcttattgtcattaccgcatcgtcgttgtcaaccaatgtaggcacatcgcccacaggtgcagtgtcatcctcagccacagcagtactggttacatcagcaacgccaccattgatcatcgcagcacgctgggacttcttgagcgccttgttgaagttagtaacgaaacgatcaagctcccactgtgtctcaacatccatcacatcaatatcaagctcgatctcatccccaagcatctccgggttgttgttcctcttgcgcacaatctgaagcacattgtgcatcttctcttcgggcaggctcaccagccccagcctgagcatgttcttctcctctaggctcatctccctcttgttaggctccctcgccttagggcttccgcatcctcacattccctgctctcgtcttagccttcgcctcgacaggctcaactgccggcggaggtggcagctcagccgccactggcctcggcggctcaaggcgcttgtgctcttcatcgaaacaagcgtaacatactgttatgcatttattaagtgaaatttaaattatagacgcatcggtcacctcagccttaaattccacctaggcgacccttcgtcttctgcacctatgcatcgcactcgcagctcaagcgtcaataaaatgcaggcggcagcgaaatcatactcacaccacagacccgctcgcggcctcaccaacgttttcgtatggctaatgtttcacaggccgtagctatgccgtcctcctcccgccgccacggtggtcgccacagagcagaacctcctcgcattcaccagcgcccagcgagccgagggactggacggcgctgcctcaggacatcctgataaccgtcttccttaagcttgggccccgcgagatcatgctgggcgcagagttcgtgtgcacgacgtggcggcgtgtagccgtcggcgaggctctgctgtggcgccgcatcgacatgggcatagacaaaatttcggtttttcaccccgctaggcgcgcg

SEQ ID NO: 38 - DNA, 1799 bp, Zea mays
  source 1..1799: mol_type = unassigned DNA
tttctccgggtgccccctgagaacttgccgtggggtggggttccctggtgccccgggacagtccggttgtcccacgggacagtccgtgctccacgcacagacagtctgtaggcaacacacttggactttcttagatctttttattgtcttcttttgagggtgttgctttcctcaattccttagtccaaagttaatcttagcatcctgtgaactacaaacacaaacacaaacactagaaaacttattagttcacgaattgtgttgatcatcaaacaccaaaactcaattagccaaatggcccggggtccattttccttacaccccacaaatgcggtttgatggcagtgcaaaagtgtttcaaagcctcatgtgccgtagattcaccagtgcgtatgtattcgtcaacggcatccgcaggaacacggtatgcaaggatacgaacaacagcaacaactttctgcagagcagatagacctagctcaccggcacagtttggtcgttgaacgaagtagggatcgacttgttgcacatcatgaaccaggtgctcaaacacatggcgacgcatgcgaaacctataatggaaaatacagtgagtacaagtgtgaagcacgtattatggacgtaagaatagatgggcgtacgaaaaaagatacctccttgggaattgagcatccatgtacaccggttgggcgcaaaagtagttcgctcggattcttgcatcgccactcatatggtcacgatggattcgaacatgaccgggaattgaaccagcataccgacgctcgttgcgtgcacgacgcctaagatgttggtacgtcgcaacctccaactcatgttcttcatcttccatgtcttgcgtcatcctaagaaaaatattgcaatctacgtaccggttcattgttgtggagcactctaagtgaagtgttgaagcgtgagtgattatttatagaacggataggcttcggagacaagccatcctcttgttgtgcagacgaagctagtcgatggcttgttcaagaagtgtcatggcttacggtagaagcatgtgaatttgcaaacatattttgatactaataaaatactatgcgacattttatcgacaacaattacacactaaataacatcgaatgttaattaatttttacaattcgaaattattaatcaagcattgttttggagattattatttcgaaaattagtaaatacattattttgtgcatcctttgtatagcgtgaaatgggaagaaaacaaagaaaatagaaaatgaaatggaaaaaagaaatctgttaacactgtagctttagatgaccgaatttagaggacgttactggagaataagaagatatagaggacataatcttttagagtgtgctgtaaagaacagataataatcttttaggggatggaatttaggggacgctactggagacaaccttattctctggttcctgaactctattccatttaacaagattagtttagtctttgcagtacacagaattggtggcactcaattaatattctttctaacgttgggactgccctttggacagcgttaattcgttgctgcaatgaaatttctctctatttgttcttgcgtgcgttagctcaaccgacgttctcgagaaacccatttcagtttagctccacccatgaatactgatcaatgataatgaatgctgctgtagtacttagtaactcccaatcctagctgttgtatataacaggcacgacacgtacggccatacgcagcatctgcacacagacaagtctcgaatcttccaatcccaacacggca

SEQ ID NO: 39 - DNA, 2000 bp, Zea mays
  source 1..2000: mol_type = unassigned DNA
cattcgtaatcttctctgtgttcgtcttctccatagcctcgtaaaacctagctagtgcagggtcaacaaccttcttctctttaaccatatagtaagaacttgtgcgaatgccgaagctcgcaaatcaaacaaaagctggcagagcaagaaaatccaagcttgaaaaatggcacaagtggttgagatggcacatcaaatgctatcaatgcgggttcctatttatatgcccagcgtatcacaacttggtggcctccacatgtcattgactttcactattctagcgaaggaaaggtgttttttaaaccttcgactaaaggcctttgttcacgtcgtagtccaaatttgttaatacaaaaacaaattaatactgtgatgggctactgttgggggccttcctcttccgaaggccctcaaaacgtaactaatgtgtttttcggtataacatagactgttacaggaggcttcggctttgagatgaaggtgtgcgtgatatgaaggtacggtctgaaggaagggtgaacgagcgccgaagctgtggctgaaggaccttcggtttagtatgacgacgaaggtggaagatgaaaccgacctaaaaggggatagactacttagcccttgatagctcgccctacgattaccagtaaatgtcagggatataaatgtaattttgtctgggctgtgtcctgtgcctataaatagatgaacagtaacaccgtacaattcacgctgcattgtaattgctcccgcgtcattcactttctcaccttttgtcaagccgaaggtatcaatgaaatataaatattgtgaacatctattcatgtttatgaaataaaataagaatgtaaacaattcattgctatattgttatcatttacattcttcttctgtatttcatttcgtgtttcctcatttatatttatttacttaccaagatgatgaagatacgtctttcattaccttcgtctaaagattattatatacaaaagaaaataatgtttcggaggacgaagatctttaatcattaaataatcgtgttgctttgttttttgtgtataacattcgagaacaaggaacatcaatagtccaagccacttttcctgccatctggcgcacattagtgatcggtccatcatttcctctgaaaatgtaaattatttcatgttttccataaattgtgtaatttcatttgcttaattatgtaattggatagatttttattttcataaattatgtgtttcatgtcaattaacagtctataaaatgaaactaacgtaatggtgagatagctgcaaactagcgctaaaggttgtacactaaaacgttaggtagataaagatagacaaaaatcaatagtacacttaataattaataatacagtcaatagctaaggccaaatataaccctaaaatatcagttcggtttttttaagtataaaagatggttaaaagattatatgatacaaccttaagactatgaatcataaatgtagctgaaagataacgtgtacggaaaattatgaacagatgttctattcacgaaaagtatgtcccttatgttttctcacttagctctttagagaccactcaagacagggtttacacatgccctaatgtgccacgatcggatggtaaacataaacatagaacatctgaatttcgagcatccttttagctagccgaactgtgatgagttctcgcagtcggtcaaaattgtcatgaaaaaccgtgaaggcgttagtatgaccacaatttcccacaaaaaattaattaccgcctcgctcaaaatgacgacgagtcgacgactcttcgggattgcgctttgacgctccgagttatcagacactaatcactcagacggatcagctcgtgcatgcgcagcacatttcacactagttatcaccagtatataaatacgagagctcaatagcatggtagtcttattcttcacaaagaagtagcctgctcttgttagttcttactagcagtccaacagcagccaca

SEQ ID NO: 40 - DNA, 2000 bp, Zea mays
  source 1..2000: mol_type = unassigned DNA
tatcttatataatacactaggtgagtgcccgtgcgtcacaggcccgagtttctcttcgatgtcatctcggacagctcgtatatgtcatcctttatctcataactatataacacatatgtgcatatataagttatcgagatattatatatccccgttgcaacgcacgggtactgacctagttaaataaagagagagagtggtgctatctataagcctagagtaagcctaaacacataaatcgagatggtgcagagagtgaattaagtattaaaaacagctatagatttttttttgattatggaaacaagagttccggctttagcatacaaatgcgtacagccattacataacgattattggaccatagagcagttgcttgaataattaaactaactcaaagaaaacaaaatgccatgactatagaaaacactagccatcttcaattcttgcattcgagcgccatccatgcattgcaaaaacttccatcgccaccacctccaaagctcgacaggcatcgagaatttgttggtgtttttcctccttctgtagaagtctccaaaatctgaaccagtgagtaaccctgaaaataacctacacaattgatggtattggtttatgattaaacaccacctcatttcgacagagccagatggaccaaaataaagctgatattccagtcagtaataatgttttgtatgccagagcattattagattcccaagacccaataatatgattaatgctaatgggcatgtctatttttaaagcaaagtaaatgattctccatatacttttggcaatgtaacagtcaaaaaagagatgatgtatactctcattgttgttgcaaaagctacaggtcaagctaccattccaacgccttctagctaaattatctttagtgaggatcgctccccgacataaattccacagtaaaacttttatttttagagggagcttaagtctccaaagcatcttactccgattgctgttaggattattcatcaaaagatggtacatcaattggacagagaaaatttcattcttatgaccatcccaaacaaaattgtccctagtcggattcagagtaaccgtagttagcaagtttaacaagttatgtcattccaccattttaattcccctaattgttcggcgaaaagataaattaagatttgaatctgaaaagacgtctacaaccaaggccgatttgttacgcactatattaaaggttcgggaattgctcactgagaggtcgactggttaaccatatatccttccagaatctggtagcatgaccattcctcacttgaaagtgacctcatttaagaaattgatccttaatattcattagacctgaccaaaagtgtgaattgtccggtcttctagatatttgagtaagagatttatgtcctaagtatttgtttttaagtagctgttgtcacctcccctcttcattgagtaatttaaatagtcatttgctgagtaagctaatattttttaaagccagattagagacacctaagcctcctaacgctttgggttgacaaacaatatagatagcatagatgttatagatatctactgtagatacatcattgcaaagatttactatagtatagtagtatatagatggcatagatgttattagagatgtgatatatctatatacctgtctcttcgattttcgttctgtaaaaaacgaaatggttactcccgtctataatctcttcgatctccgctctgtctcttttactaacaaattaatcctgccaagcaagtcccaataacaacacatcgtctctggttttgttccggagacgacggtccatctcctccgttacttcaatcgtcgttcttcgctgctcggagctacatgcgtcgttgacgtacacccagttctcgaatggccgtagtatacactagtatgtgctaggtgtctctatttatatctaccattatattctagtatctcgtccagcaagcactccagctactgtgtagac

SEQ ID NO: 41 - DNA, 2000 bp, Zea mays
  source 1..2000: mol_type = unassigned DNA
tttttaagtacatgtttttagctttctaattttttaaatgagtgaaacaaagaaatattccacatttgttttaatagagataaagacaaagatttacacttgtttgaatcaaacaagttttagcattctcataacttacacctacaacaactttttatagcttatagttatagtttttctttcaccgccacgacttaacaaaatagcccttatacaatgagcagacaaagtgctaacgccaaactcatctcaacccaatcactatatataaatatttgatatatttttagaataaatagacaaatagagcacgcttgaccagagcatggtaacatagattttccaactttgtcaaatgcaagattagattgctttgcaactattttggatcctagaaaaaaacttgatgggttacaaactgctttagaaggaattggcgacttgctagatatggactattcatatgctttcaatcatgtgaaagatgaattatttcgtgtttttggcttttattataacaaatatggtgaaagtgatgttgcaggaacaacgactgaacatgacatagaacattcagatagttctttgacagctcacttatggaagagatctaaggggaaagaatctgctaactctacaacaaatcaaaggtggaatcctaatgctgaattgaatcattatctatctactaactttgcagcgactgatcgcaccttgaagggtgataaggtaaaattacttgatggtggagggaacacaagtatagttttccagtattatctcattttgctagagatattcttcttgttcctgtttcgacggtttcctctgaagcaacatttagcacagtgggtagaattattgaagaaaggaggtcctctctttcacctgagacagtggaggccattacctgccttaaggattggaaaagagcatatgatagaaagcaacatcaacttgaagatcctgaggttgaacaagcttttgctgatttcagccatgattagtgttacttttatctagttcatgtggactgttattgtaatatggactggacattaaaattttttccgatctgtactcttttccttacagagggagccccacactgggttgtaaggtttttaacgaggcagatcgaggaataaaaaacattttccctctaaaatttgtttgtgttgtctaatttattgtaactgtttatttaaataaatgacttgtttatgtattttaactgtttttaattggactgttgtttatgttgcctgagaggtttggacttgtttctgtatctgacctagtcatatgatgattaattgcgggccgggtctgggccagcacagcccaatgtaagcccgtcgtgctttagagccgtgttgagcctacgttttaggaggtcagcacgttttggcccggctcgaaagaaatttatgctagcacgacccaaagtatttaagcctaaagcacgatggcccggcccggcctaacttgcaggactagctggacggattgagtcaacgagttgtttcctccgtagatccctactgagtgctagttggcgtcgttactcttatgaacaaaaaaagtaggaacaattggatctataccattaaaagatccaaactttagatatataccatggaccccacatgtcattgactcatgtggacccacatgtaagtgagatagtaatggtatggattcaaagtggtgatcttttaatggtatggatccaaatttcccaaaaagtaataataataaacatatgtaatgaagaaaaataagttacatgcacgtaacttcatcaactaatggatttaatttatgagacacagaaggttgaattataactcatgccgctagaaataaacaccattaatgactatagctccgttacagatatgtagctggaattaactacactttctcatataaagctagaccacccatgactgaagcaccaacacaatttctaccctcaaaactgttttggagaaaca

SEQ ID NO: 42 - DNA, 2000 bp, Zea mays
  source 1..2000: mol_type = unassigned DNA
ttgcgtgccgtgttagaattttgaaggcaggaattgactagccatgcttcggatcgcacaaagtttctgcaacttgggagcggtgcagagtcgataatggaggaacggcggtggagaggagaatattccggcgatactgccgatcgggtccaatattccttccagctgatgatcggaggtctggccactgccgatcggatcgaccacgcaggcagccgtgtggtgtggtgtggtgtgggtggcctgcgacggaacgtcgacggagcatgtgaccgtgacctctgcctctgcctgctgtgtcaggtcaggggctccgttgcctctactgtaccggggcgccagaaaaagacaggccttgactgtgtcgcgtcgcgtgtgtccatcgtccatgcttggatctgtactgtacgaacgagatgccatgtgaacgtactgtatgcgtgagaggcggatgcatagactggccagatggtaacttttttaaaaaaaaaaaatcgcttcatatcactcgttataaaaaaaatacactctccgctcaatttataatttatttaacttttttttaccaaatttgaataacacgtcttattcaaaaaaaacataattattattaagttctactatgttaccgtttagtatataatatattttaaataaagctttgaatttttcgttttctcgcaaatatttgaattagacgagccgtttgataaagaaagttaaacgaattataaaattaagatagagcgagtaaaaatttactcgtctttcattgacgtgcccttaatcctcgatgctcctcttccgatacgctggtgccaaaagcaaccgcgtcccccatcggagccatatccacgactccacgagcgccgtcctcactagcgagctctgcctcacacctcatctcgtgcgcttaacgagaggaccacacctatgctcgtgccaacgcatggttgtgcatgtgcctacaccacgccagcacgcgggggccactccatgcgcctccatagaggtcacgtccctctagagcttgttcggttgccgggagattgaaggggattaaatcctctcctattcaattttgaataggagggaatttaatccccttcaatccccgtcaaaccactaataaccgaacatgaccctaggcgtttctgagcacagggctattctaagcggcgcagcgcagtagccaagggggcgggtaggggccttggcccctctcgcgtaaatatatatagacaaatagagaacatataaattgtatagtttttgttgattaaaataaacaaattgtttaaataagtcgtcatgttttctaaaatattaaattcgccgctaataataaaattcatgaattttaaataaagttgtcatcagatagttgttgtgtatacgacgaatttgacatgtacgtgcgcaaattgacgaattttcagggatcctcgaggctaattagatttttgaattctcttaaaaaattgttagctgcctcgtgcatactagagtcattatataaaaagttaaatctcatcataatagtaaataacatccacgcacgtgccatatatgttagtttatttggccatgccatgcatctgcagaccatcgtgcaaaaaagttttctgaatagttattatttgatagctgagagaaatgcatagaaaagatatgcacgttttttatgcaactccatcaaataactcctaagttattaacctttttctgtcgattaaactcctaaggttatttggccaaattaacagggtttattaactagctagagcgtaactcctgtcataaatagaatcctcattaattaaacaaatccgctaataacgccaccgcagtttcataaatagaatctttattattttatatgaaatatttccacctataaaaaccaggtgttccttcctcgtcctagaccagtgcaagtcgttccatccactacaccttcttacacaacgcacgaccatcaccaagccgtgaagaacgaa

SEQ ID NO: 43 - DNA, 2000 bp, Zea mays
  source 1..2000: mol_type = unassigned DNA
ggaccttcgtcgggaacttctagggcacctacaagctccatggaagctcctggaccttaagaggtgcacccaaaagagcagtgagagccttcgggactacatccgaaggttctcaaagaaaatgagcgaactcctagatgtcaccgccgttgacgtggtgagtgcattcacctacgacaccaccaacgagtcgtccacgagcttggacaggggtgacaaaagatggccaccgacctcttcgacatagcggtcgctgacgaggaagacgtggtgggggcaattttccgcaagagaaaaaactcgcgtgatgccgacgagcccagcggcgaaacgagggatcaacgagagcaccctggtagacgtcagataaaccactgccccacacgaaccgaggaaggagaagacactgtcgcggaccggtcgctaggggtgggcgttcgggttacccgaaattttcgggtcgggtaattcgggtttttaaaatttcgggttttgagaattgatacccgaaattacaatgggttttgcaatacccgaaaattcgggtacccggaatttcgggttcgggttcgggtattcccgaactacccgaactattatgttggcttcataaaaacacatataccctattaaattagtataaaaacatagtttgaataatgatatacatggacatataaaacataagctatctacaatcacaagttatgcacacttacacataattatagatgtacaaattaataattaatcatgacatgagtatatgacacatgaaagttcgggtaattcgggtacccgaattttccgaaataaattcgggttttgtaagttgctacccgaaattctcgaacaaaattcgggtatttcgagttcaggttcgggtattccggattcgggtttcgggttacgggttttttgctcagccttaccggccgcccacacaactggcgaagaacgacaacgaccacttatagagtggaaatacctatagagtggaaatttttgacacgaggcggtggctgtatggacaggaccggaggaggatgcgcggtacgtaggccgattgtgccagcctattagggcatgtacagtgggtgttttaagttgtgtcttacaacgtgtctagaggggtgaatgtaaaaaaacttaagacacgtatcttgacgaagacacagtgtcttagctctatgttcgagacagaagactagctgattggtcattttaatttattgaatgctctgattggtacaatgaatatcgtaagacacatgttttagatatgaccactgtattatgttgtgttttagttgtatcttgtacttggagtaccgtgcagcagtatctgggttgtacgtgcccttatgtaggggaagggagtagaaatacgatcaatgcgaacgcacgttgatgacggtgaaaacggtagaaaccggtggaagtagagattacagcaaacatggagggacggctttcgtccacccaccggtccttggcgcgcgcgcggcgagcctacgatccaaaagccgcgaccgcttggtgttttggctgacgcttgggaacctgccgcagctgcatttcttgctttgggagccatgtacggcgaactcatacttttgacgtcggttctttttcgaaaaaacttttgacggtcagttttccccactcccgctatatttaaaaccccccaccactcccccaatatttatgacttgacaacaattcccacatcgattcattgtcacttgccagaagtcgggcggccatcgatcccactcccaccgcttacagccggcccagtcctcgtcggcgccggacggggcgaaggtaacccccaagcttccagattctctgtcaacttcagttatgcgtttttcttttaagatgaacctgaaacattttagctttagatcaccggagtcatttttttttttcattaggcgattggagttaaaacaggccgagtgggagaatttcctggatcttatctgttctccatc

SEQ ID NO: 44 - DNA, 2000 bp, Zea mays
  source 1..2000: mol_type = unassigned DNA
agtcatgacttgagctcgaaacacaacacacggagttcgcaactcaaatggagctcaaatcactagcacagagaatcaaatgtgtggagtcggagtctgggagttttaaaatgtttcttgaaagcttggtgtactcctccattcgcctaggggtcccttttatagccccaaggcagctaggagccattggagacaatcttggaaggcaattcctgccttctatcgagtggtgcaccagacagtccggtgcaccaccggacaaccacttttcatgtccggtgcacgatctccttccaaatcaggtgcatcctccgggcccattggcgcaccggacactgtccggtgcacaccagacagtccggtgtgcccaaccgaccgttggagcgagacacgcgtcgcccgcagattgcgcggctgaccgttggcgctggcgaccgttggctcaccggacagtccggtgaattatagccacatcgtcttttccgtttcccgagagcggcgagttcgtcgcagacgactcaccggacagtctggtgcaccaccggacagtccggtgaattatagtcgtacaactccatcgattcccgagaccggctacttcaccgttgaccagcctggcgcaccggacactgtccggtgtgccaggcccgagcagatacttggctgttcacagccaagccttttccaatgcgtttcttcttttgttggccctgtttctagcacttaggcaaacacattagtgtacaaaataatatactaagtctagaaacataccttgtacattgatttgcacttttcacccatttagcacataagtgctcaattagtgtgttgtgcatctaatcaccaaaatacttatagaaattgcccaggggcacatttccctttcagctgcttctgaccgtctacggactgtccgaccattatagccggaccgtctgcttctgtctcggaccgttcggccttagtacccggaccgtccggcgtacgcaggacaagtgaccgtgatcgggtgtccgatcgtgcttgcccccggtgccttcggtctttcttttgtctggagctttctgagtaactattctgcgtgacatatttggtgtgcggggatcaccaatgacgatatttttatctttgcttttatcggccgcacaaggccgaattatggcctttttgctcgttggcaaaaatgtggtgacagggacatatggcccatcaattttcacctctttttaaacctcaatgggtcttcgtttatagccaattgtatttgccgacggaagacgacacaatcattggtgttatggggaaaggagccatgtcatttgcattatacgcgccatttgcaatatacacaatcttagttgactaaaaattgctagtgaaattagcaagttaacaaatagctatctaactattagttaatttactaaaaataggtaatagctaaagtattagttgtactgtttggatgtctcgactaattttagtaactaactattagttttattgtattcaagcaccacttgagtagttaagatagctgccataacaagtgattcacgggctcttgccctgatgggcttggagggccgatttatgggcctatgttggctgaaagcctgttatggatagtatcacggactagttttttttttttttgataaaaaagtattgatttaacagtatgaattggcgtcggtaggaaatgcagttcacacgactaggaaggataaaatcgcagccctaaactgtaaattctccttgtgtaaactggaaaacgagaagtttctgaagtaaattagcttcaatgcccgactcttcgtgcgaactggaagaggcttttgtcgtttcgttttctgaattgtgacctgtcagattccagggcgattcagcgcatgctgcacgcaccagatattttaagggtcacacttgcttgtttcatgtactagcacaagaagtaaccgtaccacttcttcaagccaaatcgccagcaatccacc

SEQ ID NO: 45 - DNA, 2000 bp, Zea mays
  source 1..2000: mol_type = unassigned DNA
gaggtcttttatctctttagtggacccaggggatatctatcccccactgcacgtgaaggagaagaagatcaaaatatgaatgagttccgtgattggatcgcaaactggttgtttgcaagagcctggagtacatgttttgtaataaaaagtatttggactatatgtactttggtatttgaactacatgtactttggcatggtatgaacatgtgtgtacacaattctgtgcgtttgtttcctgtgaacctggcaccaagtttctgcgtgtccaaaattgttgtgtttggtgccaagcatgtgtacctggaatccaaattgttggcgccaagagcacccactttattattacgggtaatgcaatctttatgcattgtaattagtgcacttcagtgaaatttagtcaatagaactaaacatggtattttagtgattaaactttagtcattaaattttaatcgggtgagtaaatgaatcaaatgaagttttaataataaagtcaatcgctagctataagactgtcttcagcggttaccctaaatttctccccctatattccactcacgtgccacgtcagcgttctcttcccctatatctccaccctgtatagcggttcccctaaatccttcctctataccccactacaaccataaaatatcattttctatacctacttttcacctcctatcaatttttcatctactaataattggaagtgggcccacgtgaacagtgttagagggggagagagacgcacgccagaacgagggagagagagaagcttgctaggggaacctctgcgtagggcgcccgctagagtgaaacgttgagcgtatgtcgtgtgtactgtagccgttgtatttggcgtgtgggagcgatagcgagaaccgttgaagacggtctaaggatccttatagccacctatttaacccatctatatatcagtttaccttttaatattaatgcaagacctcacatgtctctctcacaaagcttcttaggccccgtttggtttgagggactaaaaattagttcctctgttttagtccctaaattactaaacggtagggctaaaacaagaattaaactgttttagtatctagttgctcaagaggtgactaaaagagactaaatcatataaaatttatttttttgtcatcctttatttcagttgtactaatgacaaaagaatgttaagagatgttttaatcatcttatgatttatttaatgtgttttgaatagttttagtcgttataatcaatattataatcaacatggtagagactaaactttaatttaatgactaaactttagtcaagccttagggcctgtttggttcgtggctaactgtgccacactttgcctaaggttagtcgttcgaattgaagaactaaccttaggcagaaaagttaggcaaaggggccgtaataaatagcaacaatacacaaagattaaaatgaagcaagaaatatccggctaccaataataaatgcccttcgcgacgtattctactttatctattctatttatttattatcactaaatttataaaaaatattataaatatttatatctctaaataaaattactaccgataattaatttattaataatatttaatatctactgtgaatattaatatttttatatatgtggtacaagttaaacacgtggcactggtaattatgggacgggaggtagtacgtcggaagccggaacgaaccgcctactagcctagtcccagtgccagcctaggctgcgagttcaagcagcgccgctgcgacttcctatgcacagcactcgcacagctcccggagcgcgctgctcgccccttcgccttcccaggtctcagcggctgcactcctcgggagtcgggcctcgccatataaacatgcctgggctaaaacatctcacacgtcgccgactcgcgtcaagaaccagcagccacactacaacgccaccacatagttcccagttcccaccattgtttgcacttggcaac

SEQ ID NO: 46 - DNA, 501 bp, Zea mays
  source 1..501: mol_type = unassigned DNA
atccatctagctagactgctgctgctacttcacaagcttgggacgatgtgtgatgatgcatgcttggactgtcatcagtctctatgtagcttctgaataaaataaaatgtaacgatgctcgattgggtttcacttgctcgcttgttccagccaagttattatatatatcatcaggctcgtacgccagctatatatatatgcatgcaggtgcatggattgtgcaacgcgaacgcgtgattgtgctaatccgttagttgatgcgttctgttgctttgatatgtaatccggccactcattaagcatgtcaaggtgcaatgatcagaagtcaatatgtagttactagttagtagtctctgaacaagcagtacggccagcccttttaattaaacaaccctccagaccaagaggagtcaaagaacgtactagcattttttagtatcaagatcttccacgagctagatgaaatgaagtgtttgaaagattagatatcatatttttgct

SEQ ID NO: 47 - DNA, 501 bp, Zea mays
  source 1..501: mol_type = unassigned DNA
gtatagctagcaggtgttgtgtaccggcgtcgtgtcttgcaggcaccgtcttatttcctcacccgctgttggtaaataaaagtacggagtcgtagcgtagtagctagcagctcactagttcagtaaggatttggctttgttgggaagtcaagacgatagttgtatgagatgggcgttagactttgtttgggaaagcttctaagtgctctctaaatccatcaaaataactatataccttttttccccaagcacttctctatcgaagaatctggattcttgaaaaattcaggtctaaggccacatccatggattttatagagtattgctccgaaaacacaagttttgtacttctgataaggtcttgttatccgtatcgaatgggattgaaaggaaatgaatcccattctatttctatttaattttaactaggaagggctcgttttggtagagttctgctccatgattctctagtttcgagagctctaatagagtgatttcgag

SEQ ID NO: 48 - DNA, 501 bp, Zea mays
  source 1..501: mol_type = unassigned DNA
gctcgcgatgatggttttcgttgacagtcttagtattactactatcttcctcatgcatgtaacgagttgcttcgtttctcgtattatggttatggtttcccttggatgcatgatgaactagtgcttgcagttgattttccccttattagtttgagctgcttaattcttagtagcccttttttttttcttgtgcttgtcatgcacgtatgcacacacacatgagacgggcagctagcattttctgtggttctatctcctctcggttttgtgcctgtcatttggggcttaaatgtatcagttgtcttttctcgtgcttggcatgcatagtatatatatgcccacatgagcaaactgctgctgcttcagttccaagtagtacaaaagattcaaattaaaattccatcgtatttaaccctagattttaattgaatatattagatccgcatagttataccaacatgactgattacacatgatacgtgccctttagttttatatcaa

SEQ ID NO: 49 - DNA, 500 bp, Zea mays
  source 1..500: mol_type = unassigned DNA
atccatcgagctagactgctagcaagctagctgcttgttgcatggcaatctagcgtgggatttgtgatcctgaatgaatgtatggaccgtcgtcagtctccgtagcctctaaataaaatgttaagctgctcgatcgtttctgggtatcatttgctcgcatgtttacgatagaagtttattatatgtcattaggcaggctcgtacgtcatcagctatttacccgatatatttcgatatatcctgttatatccatatatatcccaatatttgcacttttggagaatcctagggataaatctcttatctcgctatttaaaacaatggttcgattgagttagttccagaaaaaaaaatcttaccgccattgttttcttatctgataattcagctactagaagctcattgagttgggttagtggatggcctagtgcatgtacatatgtctcgtttgctcagttaatcagtttattgatgagaaaccagtgtaggagaacatgcaa

SEQ ID NO: 50 - DNA, 500 bp, Zea mays
  source 1..500: mol_type = unassigned DNA
atctatcgagctagatgctagctagctagctgcttgttgcatggcaagctagcatgggatttgtgatcctgaatgaatgtatggatcgtcgtcagtcttcttagcctctaaataaaatgttaagctgattgatcgtttctgggtatcatttgctcgcatgtttatcgatacaagtttattatatatcattatcaggccatttaaaacacttttggagaagcctaaggataaatctcttatcctgctatttaaaacactggttcattgagttagtgccagaaaaaaaatcttaccgccattgttttcttgcttgataattcagctactaggagctcattgagttgggttattggatggtctagtgcttgtacatatgtctcgtgtgctctgttaatcagtttagtgatgagaaaccagtgtaggagactagcacacctgcaagaatgttgagatctgttccattaggcagagatcaaaaaagctctcattgaccacatttt

SEQ ID NO: 51 - DNA, 500 bp, Zea mays
  source 1..500: mol_type = unassigned DNA
ataaatcttgctagacttgttagcaagctagctgcttgttgcatgacaagctagcgtgggatttgtgatcctgaatgaatgtatgggccgtcatcagtctccgtagcctctaaataaaatgttaagctgcttgatcgtttttggacatcatttgctcagatgtttacgagataagattattatatatcatcaggtaggctcgtacgtcatcagctaaatatatatagtatgggtcttttaaattggtgctcttagaccttagttttggtggtgtgctcacctatacgatgtagcgcttgtgtggtcaaccaacgcaaatgaatgattgtgttaatccgttagtagcttgatgctagcatgctgcttgtctttttcatatataacctgtctaccttgtgagagaagtaggaatttctttgagcacgagacggtggtggcaatgacaagctagcagcttcagttctgctgttgacaagaggttcgtgttcaccgattaatga

SEQ ID NO: 52 - DNA, 500 bp, Zea mays
  source 1..500: mol_type = unassigned DNA
tgaacgtgtccatgcgttgtttcgatcggacaaaaataaacagccagcgttatggtacagtgaagaaattttacatgtattttttatattttacttaaaagggtgcaggtgcagtgcagtggtgcacacagcaaagcaagcaaggtatgctggctagctagtcatgctgctgctgggattttcacttgccattgccatgtatgagagttatgattcttacctgatcttgtgttcatccttgtcatgctatcaattcatcttttttgccgtctggttcacgaaacgtaacgaaacgtaacgtaaatggtaatagtaatgattcacactcgattaccagtagttacaagtttgaatataacattttatcagtttctagtgcagcacctgattatggttgaacttaaccagacatggtttaacgttatcggttatcatttacgttacaaatatgtgaatcaaacaacaccttatatttgtgatatgtatggtttgaagtgtgc

SEQ ID NO: 53 - DNA, 500 bp, Zea mays
  source 1..500: mol_type = unassigned DNA
tagtcgtcgttgttgctttactctataattagccccaataataacaatctatgtctgtgttttcctgctgaatttgtaagttatcattttggccactggtacttggtaactactataacagttcgagcatataataaactagtgcatcaggcgcgcccatgcgcgcgtaccaagcagctgtcaattattttttatatttagttttagtaaggtaatataatggtatattctatatacacttgtagcagttattttaacataagtataagagcaaatttaaactggtagtagtgattacataaggtgcaccgagtttataattgtagtgattacaatctatattagactgaaagaaccacacatattacattgtgtttcagcaatgtgttgcatgagtgacagattacatagagcataggttaaactgacaaggtttcatcagctttgttttatgtacgcaaaagcatttgagcttcacgtcacatggtcagtggcggagc

SEQ ID NO: 54 - DNA, 500 bp, Zea mays
  source 1..500: mol_type = unassigned DNA
ctatggatcaggatgtggatgtgttcgttcgatcgtttccacgtccaatctcgctgaataaattgtctcgagaaagtttggctttgccgagataaataatagtgataattcttactcctgctacaaactgttttccttgaagcaactatgagcatcttccatagattcgcgacacgatttaaattttaaaaataacatttttaaaaaaatgtgtcacggttacacaactttttttttaacactagttgcgccccgcgtccaagcttactccctgaaattgcattcaaactctcgattcatgtctgcccttctcaccttctgtgtgcctgtgtgggctcgacgtctagtcgtcttcggctcttcgcctacatcagtcacctcctcacacgacgtgtctgcgacacaggccgcggcatagagcacgggcatgtagttttgaacaagtgcatgttgctacctcgactatgatcatgcaaaatattttttttattatttataga

SEQ ID NO: 55 - DNA, 500 bp, Zea mays
  source 1..500: mol_type = unassigned DNA
ggtctgcatgttgtgtcccgcccagagtgcagtacaaaaacctgatatttatttgcatttcctatcagctgggctctccgaacattattactaccaagtttcttttttgtttgtatggtcgttgcccaatttggttttgagtagcagggaggttttgaattgggaacgagaatacttcttctgtggtgcgtagcggcagactttaacgctgtttttgtggcggttggttttgatgatgctagtatctgttagacgatataacttttgttgaaatttggatgtggcacaaaaattccggcggagaagtgcaaaagtgtataacttgtgttcatctaattaacctggatatatgaatcgtcgggctgcagattctgtctagcttaattccgtaatgggacacaatccggcccaaccccactccaatcctataagtttaaaaagtcaacccagcctacttaaatccaaattgatttggtacagttcaatccaatccgtttttt

SEQ ID NO: 56 - DNA, 500 bp, Zea mays
  source 1..500: mol_type = unassigned DNA
ataagacatagcgtgcgtgcgagctatattatatagtttgcagtttctttaatatttaggccagaatgatgctttcgtcagatctgaataaagacatatatatgatgcgtccgatctacaatattataatggtatgctgtttctcagaatctgtaagaccgaatgttgctttatcagagtacattatatattatgtgtcgaggaaacatgaaattatgcctacctcataaaaaatgaccataaatcctaaacactaaacaagacaatctaaatctctactacatattaagacaataaaggtagtctgtctcccctcgtttttccgttttgccgtccatcgatctggaccgtccgtatcctatcgttcgtctgcgtctcccacgctccatccccctccaccgcacgaccgcacccaatccctaacctttccccgcacgttcctctcctctcaatgccacgttgcctgccccctgcccctgccgcgatagatgcctcgcggc

SEQ ID NO: 57 - DNA, 500 bp, Zea mays
  source 1..500: mol_type = unassigned DNA
agaattgatcatggtggaagagatggccttgattataatagctgggagattccttgcaactaaatggctgtgccatctgtcatcaagttttatatacatgaacttgccactacttgggatgatcttaaatgcactgtatccttttgcaaatgtatgtcgtcagttatgcatggatgttaatgctagatttaagtattttcactttttcagtgtggtgctagatgtatttatatgggttcaaatcttgttgccacaattaacatccacagcgcaagtgcttaagccgccattgcacctgcacggctgttttcgtgcctgacagcaccattgcgttttggcgcaattttgctggcgcgtaatggctgtttcccgtcctgccctcagccaatatgcagacttgctgatctgcatctgctccactctcatgctcggctgtgtatcttagtatcgtaataaaacgactcatcattcccctacctacaagttgcccaccctgatta

SEQ ID NO: 58 - DNA, 500 bp, Zea mays
  source 1..500: mol_type = unassigned DNA
attgcatgctggaacgtcttcaaccccgtgctcgtcggattgttcgtcatctggcgattatcttagcattgtcgtctgcttgcagtggctagcgcccctgtttatcttatccaaattagataattaaataaatgtatcgaactaccttttaatataattatttaaatttcgtctagtagcagataaaaactaattctgatattagatatatggcgtacagtcattctcttgctttccactgttatcgtcttgctaatattatgtacagctggttgttctactgctgtaccaagagtcgtagtggccttattattatttattaccggtagctcattttgctagtaaccatcatgaaattgatctccgtattggagtcaccgagcattccgttagattttggaaagcacacttttgatcttgatttatctttcaaacttccatgttattcattgaagagagactctagaaagtaagcttgtgacattgagtcagctactagt

SEQ ID NO: 59 - DNA, 500 bp, Zea mays
  source 1..500: mol_type = unassigned DNA
aggtcaggtcagcagcatgtatataatgcatgctagtcaatggatgtagtactagtaccagctttattcatggtattttgataagttcgacaaatgatatctgatgaagtctattggaaagtgttttgagttttctgttttcaagtgtcctactattatcttattactatcatgatttttttttctgttggtatttcgaattcttggctccataaaaaaatgctattttatcactctcagcgtgggctatctgttattatgtctccctatgtctatgacttgtgggaccattgtgtctatgatttgtaagtccgatggtcaaatagagaagcgcataactgagagtgacaaaatagcaaatgcccaaaaaaaagtgatgcaaaacatgttaatagtggcccatagagcaggtgtcggcggttcatgtgcacagacgccattccagggggtgctgagcttgggggactcctaccgagatgttgaagagcatttgttggttg

SEQ ID NO: 60 - DNA, 500 bp, Zea mays
  source 1..500: mol_type = unassigned DNA
acagacactcttttatcatatagttttgacagcgacgacgaacacggccgggtgtgtccagcatatatgaactggtgctgtatttgttatatgctgcgtcaaccatgtaagctagcgttcaaataaatgtcgagcacacgaccacacgtgatcattcaacatcaacaaaatgggactcagtattgttgtctaaaagttagacaactgtgcaggattggatttaatttacagatataaataatgagataagagattgagagtacaaccgtctcgactctcgtgaacccgattcacatacacgcgcatgagattttaagattatactgtcgggcaccggctcttgcgcatctccccttccgcctatggtttcctttactacgggaatccggcattttggtgaatgttcaacgctttgtcgagtatattttgtcggacactcgacaaagcagtctttatattactgtccggcaccggctcttgcgcatctccccttccgccta

SEQ ID NO: 61 - DNA, 500 bp, Zea mays
  source 1..500: mol_type = unassigned DNA
ctaagcgacacatctcgtggccaagtgtagtcaggcaacggtgttgtcgggaccataattaggggtaccctcaagacgcctaattctcagctggtaacccccatcagcataaagctgcaaaggcctgatgggtacgattaagtcagggatcagtccacacgagtgactcgatcacgcttcacccgagcctagcctcggccaagggcagccgacctcgagagacttccgtctcgcccgaggcccccctttttacggcggacacacctccggctcgcccgaggccttggcttcgctcagaagcaaccctgactaaatcgccacaccgactgaccaagttgcaggagcatttaacgcaaaggtggcctgacacctttatcctgacacgcgccccccggcagagccgaagtgaccgccgtcactccaccgctccactgaccagtctgacagaaggacagcgtcgcctgcgccactccgactgcagtgccactcgacagagtgag

SEQ ID NO: 62 - DNA, 500 bp, Zea mays
  source 1..500: mol_type = unassigned DNA
gctcgcgatgatggttttcgttgacagtcttagtattactactatcttcctcatgcatgtaacgagttgcttcgtttctcgtattatggttatggtttcccttggatgcatgatgaactagtgcttgcagttgattttccccttattagtttgagctgcttaattcttagtagcccttttttttttcttgtgcttgtcatgcacgtatgcacacacacatgagacgggcagctagcattttctgtggttctatctcctctcggttttgtgcctgtcatttggggcttaaatgtatcagttgtcttttctcgtgcttggcatgcatagtatatatatgcccacatgagcaaactgctgctgcttcagttccaagtagtacaaaagattcaaattaaaattccatcgtatttaaccctagattttaattgaatatattagatccgcatagttataccaacatgactgattacacatgatacgtgccctttagttttatatca

SEQ ID NO: 63 - DNA, 500 bp, Zea mays
  source 1..500: mol_type = unassigned DNA
actttgtgaggcgaatccaaaacaaaaaggtggatgcgagaaaggcgaagtatatgtaagccggaaggcgaatttcctggttactgatgacctgcgtgttcttccactctcattagcatccactgttaagattgttagtgattccaaaatccagacctcgaatcttgtggtcaaagaaatcacattaacaaagtccaaggtatctatatctgctacataactcacattatttgcattgtgctagactacctattcacttggtcttgtgtaactgaatcttggaacaggttatggagctacagagggctgtcctgctgctgagtcgcaacatactcagctccgtgcttctgcatcctaagaagaacaagaagctgcaccattaccatcgcatgtattagccgtcgttgttgctttactctatgattagccacaatgataactatgtactatgtctgtttttctgctgaatttgtaagttatcattttggccactggtattc

SEQ ID NO: 64 - DNA, 500 bp, Zea mays
  source 1..500: mol_type = unassigned DNA
gcatgtcatggtgtgctctgcgtgacattgacttttgggttgcgttcccagccgtatggactttgttaaggtttacatgctatgtttctgttttaatgcatgttactatcgtttagattgttatgttttatctcatcactgttcatggtggctacctatggttaacggtggcttgttcgcgccttggcgacccataagtgctgtgttttgttgtttgtttggtcactgttttttctttgcgagaccagtattaaagccagtaaaatgccttttttgtttctaggagtacatccctaattaaagccaataaatgagttacttttttttggaatgaaggacgtctttcgagtttcgatcggtagttttagttccaaaatcttctgacttgagcccctctggatgtccacgcctactaaagctctaattatgtaaaaaaatcctgagctcagtaacagattaggccttgaaagtgcaaaatgttcatataaattatactgcat

SEQ ID NO: 65 - DNA, 2000 bp, Glycine max
  source 1..2000: mol_type = unassigned DNA
taattaaaaagataatattttatattatgtaataatatattatttattataaaattttcataattagattttaattttaatattaagtagtaacaataaatattatattactacttatgcatatttttaaaaattagttactgtaaaatatatttcatgaaaagtaatattccttaatattattacagaattttataattaaatattaattttgataattgatattaagtaataatatgtcatttactatacaattctgataattaattttttattaaataataatattaattaaatattatgaaaataatgaaataaatatgaaatatgtaattagatatcaattaattaatactaaaatacatagataatcaaatacatttttaactattatttctcataaaaaatatttaaaataataaaatgagcagatgaaaaaattaatgtgtttttaatcttttgtgtgtaatatttttaaaatattaaaataaatattttatttattcactattaattattgttcgtgtttattaagtataatatataattaattttatgttttatttttgaaataaaaattatatttctattatgaaaacaaaaatataatattattgtatgtaattttgataattaaaaattaattttaatatttatcctaagtaatagtatactacttattatacagttttaatattctaattaaatatcaattttgataattttgatattaagtagtataattaaatattatattgttatctacgctaatttttaaaattacttattttaaaatatatttcaagaaaaataataatttaaaagtgtcaattttaaaaatacgtatttaaatttaaaataattcgtaatatataataattaagaatataaattaataaattaattattatgtatacatatcaatgtaatttattttaaatttagttatttaaataaattttgctaacagtaataattaaaagtaatcatttaaaagtgacgtgagtatttaatgaatatttgagcttttaatatgtaattttagtttttttataatttctaatcaatgattttaatttttttttttgtaatttttagtcttcttatggtaaaaattttataattttgatcaaatcctcaattttacatatattttatttctcaattaaaaattaataattatcttgatcaatttttacataattattttaatatgttattaaatattttaatttttaattttatcaatatatatatatatatatatatatatatattataaaatactacatacaaatataaaattatatttttgaatccaactcgtacatgatacacgagtttaaaatctatgagatgtaatcgtttccttccaagtatataggcagaaaaaacatgcattcgtctgttcttatactacaattgtaaatttattgcttattacgaagatgtcactaaaaacctcttggctaccttttcatcttccaacaacgtgcatatactactctcttgttacatttcaactgctttaacgctaagttttctaattaatgtgtcaataattgttttaaaactgataggtgataattattctctggtaatatctgatgggtttttttttaggtatactagtatttgttaataaaagaagtatattgtgtaagcctctgagatgttttattcatggtatagagcaaaaagtgtaacacaaaatttaaatattgataaaatcctatccttaaaacatgaaataaatcctaaaatactcacaacaaattcataatattttaaatctaaaataatatcctaaaaatttaaaacagatattttttttaaagataaacaaattatttctttctaaactaaatatatgagacaaattctttacaatatctactatgtagacctctatatataccctgccctttcagtccacattacagcacagatattttctcaaagggaaatagcaccatttggcattttacaatttccaagct

SEQ ID NO: 66 - DNA, 2000 bp, Glycine max
  source 1..2000: mol_type = unassigned DNA
ataaggagtgataagctgcaaaaaccaaccatgtgacatgaaagtatttaccccctaggattgaggatccacaaaaagacacaaaagttaaactcataattaaattcaatttcacttgttttcacattataattttaatcaaattattaaaattaatatttttgtccaacatttgaatcctatatactatctttcaatttatagctaaattcaactcactactgatattttaattctacaatcgcccacgtgcattatcacttagaatcaaaacataatatatatttcaaagatttcgaattcaatttcaaatcataatatcgtaaaattcaaaaccaatttccacggtgaacagaaacctatcttcatttgttatcacctatatctttcccatttttccattcccgaacagattcgctgacgagaaagtttctgctgtagctttaattttaatgctaatagtttggaatagaaatctctgacatctcagggagtcagaacgtgacatatttataggaaatatataatctaaataaacgtttgaatttcttccaaaaatatttgaaaatatatatcagaatattattaatgaagaaccaataaaaaattgcaatttgtatatgtttaatatttaaatacattatatttatcaaataaacataaaaaaatataatactaattatttataataaatctttcacaatcaaattgtgtgttctttagctagcctctatgtagtacaaatgtaaataactcattagaaaacatttagtaaaggaaaacaccgatcttatcttcatggaataatgaatttgtttggttgttcattgaactgaaatgttctgctagctccgggcgctctttttaataataataaaaaaagaactaaagtattttattttttttaatttaagattttgatgcattcacatgtgaggacaataatttatttaatggaaaagtacttgcttgatttatatcatgtataacattttcatggattaacaataattacacatcacaaacgaaattattttaaaatcctgtgtggtgggaactaatgatttgataataaaaaaaacaaaagttttttttttttaatgaaaatacttctttctttacttttttttattagaatctattgtgaagtgtctcagctttcattttatttaattttaaacaaaacaattactcctttcgttatcataaaaaatactccgttctaaaataagtgtaatgtttaatttttatataatattaatttttctttcagtgatactcataatatgtgtgctttaactttttaacgtgtcgctttaattttttaatataatattaatagtaaggttaattttataagattattacttttttatttatttattattattaatttctctttatctatgtaaaataacttacaacaacacttaagttaaaatatgtcatgtgaaggagtgctgccatgcacagtcattaatacataatttttattagttaaaagatattaaaaatacaaaattataagtgccaaaagtgagttagaaaagtttgcctattactaacattttcctaatccatgtataatccccatttacaccattttgtcatttctcgctgtggtaggtgtatcatttaacaatcccgcactggactcgctgaagtatgcttttttaaggatttcttgattccttattggtacgcacaataaatatttcttggagatgaaattctctttttcatgggaccagtctaaaaaatcactaggtatttttttcttctattggcattttcttctatggtcgaccaacgtaaaataaaattggtcttaatttttttccgcagcacacgtcttagtgcctctataaattcagcaatatgtagctgtagtaatccacataaaatccaattcataatacaggtttaaccatttcccactagcgatcttgttctctaagcctaatattactcccttgatcagtctttgcatatttagaaaa

SEQ ID NO: 67 - DNA, 2000 bp, Glycine max
  source 1..2000: mol_type = unassigned DNA
tagttggttcactaaaagaatctattattttatataatattacattaatttcttatatataaatttagatattaattcaatatcttattcataaaaataaatcaatatactaagaaactaaatcatcattcattgacatgattttttatattatatttacttattcttatttcgttacaaaattctcaattttttattttagattttgattttttttcttctaaaatcccagtttaattacttttaaagttttattgttatattaaaaacatatttttcatttatacattcaaaaacgttttttcgtattttctatttttctaataaattaaagttacttatatttttgacttatttatttaaattaattataaactatttttttatgaatttcaattttggtaattttattttatttaaaaatgactgacgcacaaaatacatacaagtacattctttacataacatttctttaaatgacgaacgcgtcgatttctagtcattaccctttaactaatacaaggtaaggtcgcgttaagttttcacgtaaaaaagttactatataaacacttctctaaagacccactggcatttgtttaaattaggaaccaaacacattcattttcaactcatccttgagataaaatttactttttgaagtaaatattaaaaatcgtgacgatgttcaatatcattcggaaagagacatactaatccgcattgacgcattttcatgccactattttgctttacgcatgaaacttccaaattctccgttggtcgctttcaactttgcaactttcaaacccaaatttataaagagatcaattttttcttttctttttgaaggatagagagagagatcaatttttaaatatatatagatattttatttaattgctaaacattcattagttagtaatatgtaaaatatatttgaattaaaattcataataaataaatattttataacatataagttatattttaaaattgtaataaatatttatatcatgatacaaatttatttttaactataggtaaattctttttgaaatattttaaaattcaatatgaaaataatatattagaaaagataaaaaagattaagaaaaatttctaaaaacatcatgattgaagaaaaactctcattaaaaattagattctaaaaacattctcattcaagataactattagtataatttttaacttaggaaacattgtttttgagaatatttcttacaaattttttctcatgagggaaatgttttaatgctatcaaaataccaaataacccttattaactcatttaataaaattatataaaattttgaattagtttcttactaaaagtgataaataaataaataaatttaagaataagaaatactaataaaaattcttagcatgtttcttatcgttttaagaatattattgcagaaacatcatggaaatattttttagaaaatgtaaaaaaaaaaaaatctaacatagagcaaatgataaattttgtgatgttaaaattaaatagaaatatgaaaaatcatacaaacctttttgtaatttttaaattcatgaaaaattttattttataacaaaacttatccaaattatagttgatcgaattaattttaaaatttaacttaatttacgtgcaagaaaatgttataataaattatttatgaatgttattatccaactagtaatactagataattttcattattctataaaaaaaatataatttgcattaaaaataaaaataaatttcactacaatttatttattttgaaatttaattctaataaacaatttaattcttattttttaaaaaatgggttgtcaaccgggagagagttggtgatttcaatcttagtaatgacgtgggagattccattccagtgtgtgtgctttttctctccctactactataaagccatgttagtgtctgcttcaactcaagtatctctttgtgttcatagcaattctctattctgaag

SEQ ID NO: 68 - DNA, 2000 bp, Glycine max
  source 1..2000: mol_type = unassigned DNA
gcattgcattcaaaatgtaaagtttacagaaatttaaaatggttcacaaacaaacatacattctccttgtgtactcgtttctctctgcgctgggtactttgagtgtataaggatttgtataaatgaattgcgaccacgaaatctaactaaaaactgctatatatagattttcgaaaataaactgccctaacggtcgaacgctatcctaatgccaagtgtcctgctacgtacaccttgcatgcacacataactgctccctagaacggttatccacattactcgaaatcgaaatgatttggtgaagatttgttcagaaactacgctaagtccagaactcttgctgcctcgacttcgactcgtccatacaccagttcgaattgcccaatggctcgaagtcctctttcttgtcttagctttgtacttcgtaaatacttctttgaacctgggaattccttcttaaagacttttctctcttttcgattcgagcaggtcctgaccagactcttgctctgtaatacgcttcgaacctcgagacgacatccaatgcatacttagagcatattttagctcgtcgaaaatatgggccaacagatatttagaattttttttctttcgtttaaaaataatatttaatttctcttatctttttagttgtttaaagtggaataaaaaatggaaaacaattttgggaagaacaaaaaaagttgactttccttcctcgatcaaattgttaaaaaataaagttttattaaacatttttttatttgatatgtatctcttagtgttagttagttggaaattaggttaggtagttagaactagtggtaattagttgatgttgtaataataacatcaagactttatattattgagtaattaatcttacatcttcattttgtgtctcttgatttcgatcttaagggtgaacaatcatcttcttctcttataacattattgatgtttgaattttgaaccttgtaaagtttgtgtccaggtgggtacccatgcttcttgtaacaatcatcagtatgattataaaaatcataatatgtgcaaatcttgtttccttttactcctgaatttcctttgtcgtaagtttccttttctggaagcatacttctgagaaaaaccatgcttcctatagcatctatcaatagtgtgattatctttcccacaataggtacatgaaggcctagaattcaatgaattgctgcaaatttgttttaattgtcaattcatcatcccgagtgattctcaatcttgtgaagtaatcggtaatgctttgatcaccttgtttgatagaagtcatttcttgctacaaatttaaaattctcaacagatctccttatgagtatcttaactttaagtccttccaaatgtctctagcattatccatctataagatactttgtcttatcgagggtaaaaccgagtggacaagccaagaaaccaccatattgttacatctcctccaagctgcatgaagaggatgaattgatgcacatttttgaattgatccatctataaattcaattttatttttcgcgcttaatgcagtaagcatcgaacaattccatgagttctagtttgtcggatcaagaatcgaagaaatcaatgcaattgcaagattttcacctggatgaaggtagtataggctatggacatttagggattggtcttgtgaagaaatctcattggtcataattaagaaaatgaaaaaaaagatttaggttcaagatcagggagttacgtaaaagagcactaatgccttgataccatcataaatattgtgcctaccatctttgaatgtaaaattaaccaaaaaagcttgcaagagaaactagaaagaaaactaggaagaaatgaatgaaattctatgtattgaaaattagttctaactacaaccaatttcaaccaactaaactaagttccgactaagtaacaagatcgagtatctttaatcaatttgcccattgattattcttacattttaatttgga

SEQ ID NO: 69 - DNA, 2000 bp, Glycine max
  source 1..2000: mol_type = unassigned DNA
acactttatacacataataattaatataaatttttatttatatatagagagctaactcaatttttaaaagtttggatgtgagtttctttcactcaaaactagacaacctttatagtttctagttttcgcatcgtcttacagataagtaataataataataatataaaaaaccctcatgcctgtcgtttcgaaggtgcaacaggttcaaaaaccattagtttcaatgacttttcacttattcagtatgtgtgaaaagtgaggagataaaggcaataatacatgggactcatgcaaaataatttcactactgcattattgtcgggaagaaaacttggaagagaagaacaaaataaccatttgtcaattttgttttatataaacataaaaaaatgtcatctaattaatatgattatatttttcttttaatttttttttatctatatatataacctaaaatttatttatttattttcagttttcctacttcattttatattatttcttttcctctacttctcccatgcaagcataatgttacggcttaatttgagaaaaagaagagagataacattcaaaccccagtgatctccccatgcggttacagtcgcacaatatatctcgctctgtgttgaattgaaaaggatgtgtaattaagggtctccacgcgtgaagtaaatgaagtagaacatatgcacaaatgctcttagtaattttagggcagattcctgccttaatttgtggtgaccgtgactaaaatgggaatgtcctagatacaaagttagcatgcttcttctattgaattatatatacactgggtcaatcaagaacattaatcatggtacatagcatcaccgtagcacatggactttaaatgaagatagatacatgtaaccatttcattaattaattcatgtcgttaacactcaacgaaaaatagaactaaattaaagaaaatttgttgatgtgaggaatgagtatgaaacgtattaagggggatggtagattaatgatgatattgacatacaggtgcggatatgtgcctgatagtttaatgcacaagtaagaccgtaccaaatcaaagtgcaacttgagcctttcgctgtctagtttctaaactactatcaagctggattgaaaaaatatgaatatagacaaacttgtacaacctggtcattaaattaaactactgtaaatgtaccaaatgtaggttttcatattaagagttcaatatatttatataaaacatttttacattaatctaaataagatttaattattttatcaggttattttattcattaacatgctgtgacgataaaaaatttcttgttagatgtttagaaaaatattttacatgcaaaattgcaaatcaaacctttttcaaattcatcgcgggaaaggaatcgatatttctgtggacactatatacacggtatgaccattaattggacatggaccaaacaagcaataacgtcaaaatttgtagttgtcgatgaatgaatcttgcagacaaaggtgtccaaaatcatccaaagcacgggcatatgtagctacttaatcttattgtgggggggagactagggtcccgtagaagataccttttggggatatatagataactttttgagaggtctggatataatttgaagttttaaacatccataaattatactcttcttgtttttttgacaaacataaattatactccaacactatatcaatactagtgtcttgatattcaaaaggtgttcccatgattttaaatagtgatctaccatcgcaatcgcaattatgataataatatcaaagtattttgattttccgtaatcgtacaacaatcgtaataatgactatattacattttttttcataatataaaagttttctaaatgatcaaccacaagtcaatttatgagagctagttaacgaatatcacagcctgtatatattccctatttaaacatcacatttgttctatacacattcaccttaatccgatg

SEQ ID NO: 70 - DNA, 500 bp, Glycine max
  source 1..500: mol_type = unassigned DNA
cgggaataaaccggaaaaaagatgtcttgtttagagatatttttactattttactctataggctacgtttgatttccaaagcttattactacattctactaggtgctgtaaggaataggaagtttattattcactgttgtggtctttgtatttcatgaagattagtactttgttatttcattacctaatgaaaatatactaggtttcaattataagattacttttatggctatattgttgtctctttatttcatgtttactttgtctgtatttcacgacgtgacgaaaataaacaatacaaggtttcaatcgcaaagaaattaaaataaaaaaataaaaattcaatagcaaaaaatagttctaaaatctaaatggaatggggcattccgagaattgaactcgggacctctcgcaccctaagcgagaatcataccactagaccaaatgccctttggaagtggtttgtgaaagttaactaatttaattagtaaaatcaatgc

SEQ ID NO: 71 - DNA, 500 bp, Glycine max
  source 1..500: mol_type = unassigned DNA
ttccattacaaaaagttaagtagcaagtattatatattatacatattgaataattattgaggctatgatagtggttcacaattatgaatttattaatttacaatatgtaatggatggtatgtatccttcatttacgataaattgttggtttgaaaattccttttgtattgattctaaagaggtacgtacaatttcttttttaatcctcagttcttttcatgattctttttgtagcaagaaaaatcacaactcttatatcaaatataggaatgatatatttaattaactcggaaatgtagaacatgcggagaagaacatcaaaacagtggatatatacgtgtatatatatatatatatatatatataagacaattccaatgtaaaacaataaaaaaaatggaccaaacttaactgtaaaattttatgtacatgttttttgagttattgtcgaatcaaaatggagttttacattagcaaattacgtaagagttgtatcaaat

SEQ ID NO: 72 - DNA, 500 bp, Glycine max
  source 1..500: mol_type = unassigned DNA
ttttttcttaagttgacttgacatggctgatagggctttctcataaggaaatcctagcaaaataatgccctgcaacttatgatgttatagattgtcaatgagttgttaggcaaggaatgttggatgtaaaatggttgggatagcatatataatcccatatatatggtacaacataatctatatataaactttatttttttacattttactatattttttttgtccaaactataattttgtttttcataagctacaacatacgcatttacagatgtaaacattaacattggctgaaaagggatgctctacattacgttgagatagatgaatgaatatacacaatagtatgtctatggactatggatattcataggtcgattagttgtgaaaaatgagacgaaaaaacaaaattaaacaaaagaacgtaagaaaaaatgtgattgaaataaattttaaatctaatataattaaatttaaattaaattttaaatagttataat

SEQ ID NO: 73 - DNA, 500 bp, Glycine max
  source 1..500: mol_type = unassigned DNA
cgtggaggaaaaaggaattgtttggtggggaaatgatggtgaccaatgtaagcttgccaaatgtccaacagcgaaaggtgtagttactgaagggtgtcctgtgttttgatttccatttcgacatcatatattttctggttagttaagaattaagatggtgagtgggggagtaatatttctgcaaataccagttgaattagattgttttaagcgctttaatttggtggacctttatgttgtggttgtgtgcaacttctccagaacagatcgttgttttttttttttttttttttatatagctagtcatttttaatttcattctatttcttcttcttttttttttaacagaatttcgttctatttcatcatcactatcatttagaattgacaatgttttagttgctagataaccgcgagatattgggagttccaacatactagtattatattagtatcaaactttcaagaattataaaccttttatttccgtttcgtgatag

SEQ ID NO: 74 - DNA, 500 bp, Glycine max
  source 1..500: mol_type = unassigned DNA
atcaatatgtatagtgacggagacatatatcatatatggtttcatatgaatcaagttaaaaatgcacatcaactaaacaaatcttctatatggaacataataataatttctcaccagactaacataggtttcgatgattccggaaataacgttgattctttatgctccaattatttttattatatatatatatatatatatatatatatatatatatatatatatatatttttttttttttttttctttttatcacgccagttattatatttactttttttttcttcttccaatccgatatatacataccattagaagatgtgaaaaatcgcctatgtttaatatatgaaaataattctaattgtttatggcatctctattttatacttatctgaacctcctgttgtttaatactattaattagcactcatcgtatttatcattttttgttctattttgtctgtatccgtttgttcacttgagatgtgtgcttttttaaa

SEQ ID NO: 75 - DNA, 592 bp, Arabidopsis thaliana
  source 1..592: mol_type = unassigned DNA
acaaagactggtcggtcggttttggtagacaattgaaattagatggatggtccggttcggtatactataagattaaaaacagttttaaattcagctaaaccgaactcatttgattttattaaaccggaatcatccgattcgagtttgtaaaaaataccgaaattgaaaacactaaacaaaaactgtattaaactgttactgaaataagagaatctcccaattcggtttacgtactactcttcagaaatcagaaccaaaaattcagaaatcggattgaaccaaacttaaattgacggtccggttagtcttcggctctacaaattaaaggcccaagtttctgctttaaaagaacgaaatagttaatgggctcaaaccatagaccaggtaagtcatgggcttggttagtccgggtcaacccggtagacccgattcctgaagaaaacctagtggaaggtttaaagttgtaaactttccgaccaaataaacaaaatcgttttccagcttcttccgtcgccactaaaccctgaggctaaacctagacgagtcaaagtgtaaaatcgttaaaccctaagagggagtgagagagagaaga

SEQ ID NO: 76 - DNA, 459 bp, Arabidopsis thaliana
  source 1..459: mol_type = unassigned DNA
ttatgatttcttcggtttcaagatgatcaaatagttatagatttcatgctcacacatgctcattagatgtgtacatactttacttacccaaatctattttctcgcaaagattttgatggtaaagctgatttggttctattgaactaaatcaaacgagtttcagactgagtgattctaatccggcccattagcccctaaacagacccactaattacgcagcttttaatagagtaattacacctagtttacccactaaaccactaagcactaattatctcacaatctaatgagcttccctcgtaattacttgggctttcactctaccatttatttgtaacagtcaagtctctactgtctctatataaactctctaaagttaacacacaattctcatcacaaacaaatcaaccaaagcaacttctactctttcttctttcgaccttatcaatctgttgagaa

SEQ ID NO: 77 - DNA, 667 bp, Arabidopsis thaliana
  source 1..667: mol_type = unassigned DNA
cagagaaactaggagacttgagaatatacattcttgtatatttgtattcgagattgtgaaaatttgaccataagtttaaattcttaaaaagatatatctgatctagatgatggttatagactgtaattttaccacatgtttaatgatggatagtgacacacatgacacatcgacaacactatagcatcttatttagattacaacatgaaatttttctgtaatacatgtctttgtacataatttaaaagtaattcctaagaaatatatttatacaaggagtttaaagaaaacatagcataaagttcaatgagtagtaaaaaccatatacagtatatagcataaagttcaatgagtttattacaaaagcattggttcactttctgtaacacgacgttaaaccttcgtctccaataggagcgctactgattcaacatgccaatatatactaaatacgtttctacagtcaaatgctttaacgtttcatgattaagtgactatttaccgtcaatcctttcccattcctcccactaatccaactttttaattactcttaaatcaccactaagcttcgaatccatccaaaaccacaatataaaaacagaactctcgtaactcaatcatcgcaaaacaaaacaaaacaaaacaaaaaccccaaaaagaaagaata

SEQ ID NO: 78 - DNA, 479 bp, Arabidopsis thaliana
  source 1..479: mol_type = unassigned DNA
cattatgtttgaactcttatatgggttcttcccatgttacaatacgtaccagggtgctctaaaatatccaattggtcccgatctatatatgtatgtagagactcttccttggatagatatttggtgaatccaaaaataaagttttacaagagatgacgaaacaaaggatagtaaaagaaatcagaatcttgattattgaaattttttaaagacagaatcagcctttataaagaaacaatattctttattcatttccttccaaaatctctcttctttaagatgatctacacaattaaataccgtcgatttcgaaaattgaagtttaaaatcaatatgaggatttgaaataagagcttacatgcacacacacatatctctctatatataagtgagtaggtttacatggtttaattttctataacacgacacacacgaaaaagagaagcaagcacaacacaattttttttataacaactaaa

SEQ ID NO: 79 - DNA, 2345 bp, Arabidopsis thaliana
  source 1..2345: mol_type = unassigned DNA
ctgctgtaaatgtgaaggtgttgggagtaaaccctcttgataccatttgctcaaaacagtttattgcatccctgaagagttcattgcatccataagcctctataatggcggtccatgtcagagagcccttgacagcaacagcatcgaaagagaaattagcacttctgagatctccacattttccatacatcttgatgattcttgcagaaacaaatgggatcgactcaaattcctttttcagaatgtgcccatgaagctctttccccaactttaatgctttgagatcactgcaaacagtcaaaaccctacccatcgtaacagaatctggcctgtgttttgataacagcatcaatctgaatacctcaatccctgctcttaaatcacaattttccacatagcagtcaatcatagcggtccatgctttcacattcctttgttccaacctgtcaaacagtctgataggatactctggaacaccgcactttgagtacatcaccatcaaggaagtgaccagggatacattcggcaagaagagattctttaaagcataacaatgaatctcctttccttgctttatagccctcagctctgcgcaaacaggaagaacagtagcaattgtaacaacatctggcctaaaaccttcctgctgcatccaaacaatcgacctcaaagcttgatcaaatctaccgttggctgcgtatccagacatcaatgcagtccaagatatggcattcctctgtttggatccatagaacacacgtctcccagaagccatatccccacacttgcagtataaatcaatcaaaccagagtgaacaaacggctgttccacgtaattcttcgatttcaatacatgggcatggacctccttccccagtttcagtgccttaacatccccaagtacgggaagaatcgttgtaagtataactgagtttggatatatcttttcttcactaatcatcgtcctaaacagtcccaacgcttcccactgccttttattgtgagccaatccagcaatcatcgccccccaaacaactatatctctctctacaatctcatcaaaaactcgacgtgcaagccctactttaccacacttaaaatacatatcaacaagagaagtcttaagaaagacactattaaacaaaccattcttaatcgcaagtgcatgcgtcttcaatccctgtctcaaagcagatgcaccagcaaaactcttaaacacattagataaactataaacattcaaatcaacaccaagctctctcatttctgtaaacgtagaaagaacatcttgataccgcttcttccctgatataacagtccctctaagcaacgcattccaagagtaaacatttgagctagtactttcatcgaacaccttctgtgcatctttaaccgaaccacaagcagtatacatatgaacaagcttggtccttagaaactcattactctccaacccattaatacgtatatgaacatggacttgtttaccgtgaagcaacgacttccggcgaacacacgcctctaaaagagcagagaaagttgtcgcatttacaggaatccctctctgttcgagataatcaagaatcgtgagagcgacttcgagattgttctgcctcgcaaaaatttgaatatctctgtggataatgtaggggttcttggagtggagaggaagagaagaagggaaagcatctctttccctaaaaggtttcggctttttggcgggagaagtggaaggtcgtctagttctgcttggtctaatcggagatttcgccggagcgccatctttatcggaacgatgaggacggtggttgagactcgtcgtcacggagagggacgccgggaggtctcggattccgagagacgacactacttccattgtgaccactaaccacttgattaagctaaaggtcgttagtatttatatgtagctttaaactatttttataaagtatgcaattgaaattgaaagctaatatttgggccagaaaattgaaatatgggcttagggcccccaaaacaattgtagcccatcagttcacacacggtttcgtttttaaaaaaagcatgtatttagaatttattggcttttattttggtggttgcttcaatcttcctcgtttaaaaacaattcatgtcaacttggacaagttacaaaaaaaacgtttcatatttttgtcactaaaaatattcagttaaataaagtaattgactcttcgttttcccgacgatactgtataaataaacatcatgtttctacttgttcacatatcgactctttgtttgtccgattctatctctctctttagaaactttatctagtctcttcgaacttaatcttaacgacg

SEQ ID NO: 80 - DNA, 289 bp, Arabidopsis thaliana
  source 1..289: mol_type = unassigned DNA
tcttgttatacaatcttcaaagtttctatatttatcaagtaaccattgactcttcgcttgtatctttccgttaagaccacctattcaatcattctctaagttacatgatttaagatttaagtaaaatcattaactctctgccctctcccacttcctccactaaaaaccatctttaatcataattaaacctcaaaaatcctttcataatcacagtattataaatagcagctcttaccaaatcctctaaaccatcacacaatacaacacaaaatcttcaaaagaaaacaca

SEQ ID NO: 81 - DNA, 267 bp, Arabidopsis thaliana
  source 1..267: mol_type = unassigned DNA
ttttgttgctcttgctgcatcaaatacatatttaaattaaaagtaaaacctttcgtatgcattgtctgttactcatcttcatcagaagccttaatcgcaccatccaccactaatcatttactttcacttatccctccccactaataaaccttttaattactctttaatatccactaacacaacaaatccttccacaaacacactataaataccaaaccatcacaagctagtctaatcacactaaaattccaaacaaaaaccacacca

SEQ ID NO: 82 - DNA, 287 bp, Arabidopsis thaliana
  source 1..287: mol_type = unassigned DNA
atactaaactaaacataataatttatttattttaatgggtttccataaagcccaatttagttggcccaatagcttgcaaaactgggcgtaacggataatttaataaagacacggtaaatgcttagatagaggattagagggtaattaaattaaccacgatcactgtgataattactacaacattaaacgacaaaaaaacttttcgtctccctcataatcttctactatatattcgtcacatcacactcataatctcttacaaaaaatccataacacaaaaagaagca

SEQ ID NO: 83 - DNA, 2821 bp, Arabidopsis thaliana
  source 1..2821: mol_type = unassigned DNA
tgaatcatgaatggtatgatcgatcttgtcccgttgtaaccacaagcaagatcagaagcgatgcatgcgtagcaaattttaaagagagaaaaaaacaagtttttggatgtgagtgttaaattatttagcaaatttttgtatgtaaatactactgcttgctaaacactattttatacctacataaccaaaatatctttactagataattaataataaaaaatttattggagctctttatgtttgacatgtaagataaaatactcatttcagaataatgttcttatatttttttttgttgccaaaactatcagattatgatgaccaactcattcttaaaaattagttcttcaaatgtaacagttattatcactaaaaacctagaagacggatcctattcatttatatacagtcagataaagagagcatctcctgatgataaaggtgggagtgtcattatttatattaaaaaagtaagaataatatcatatggaaaaaaaaatttggtctgttacaaaagtaaagcctatggtttataaatccttgacaaagaaaataaaaataaaaaatcaaattaggaaaaacaaaaatcggcatgatcatggctaattaggttttgatcatcttctagattcttaatagttttttttgcttgtagtagtaatgacgaacacattttgcacactgtaactctagaccaatgtcgtaaaatactttacaatttgaggtaatgatacaaatataaagattaaagcaatgctaatatattgaatcaaagcaaatattaacattaataaaatgttttgaaagataattgttttgacttttgactagtcgtaagtagcaatttactagagatgaggaattgtataagattaaggatgtatttcactatgagattttagagttagctttgaagagtgtcaaattggaatatcatgttggtggaaaattattatgctttgaaggttggaataagcattggaaagccgatgatgttgagatatacatttacaatggtctcgtaatcaggtgctaaatattgcaaggacatgcgttgtgtaattatcattgatttcacaataacccatggggtaaatgtggtaatttgcaaaagtagtaagggagtggaatttggtaattgtggatacattgctgtcaatgttgaaataatgctaactttggttggtgctgcaaaaaaaaaggtcagccaaatatgtgtagaacgaagccgcaatatctactacaagattgctactctatagatgttgaatggggttcccaaccacttataaaagtggatcaagatcaagaaaaggagaacatgaagtttaaggtttgtgtgtgtggatattccttcatttgaatgtggcttcctagatgttgagttcatgtttttgttttcttacatcattaggtagcaactgattgttttggtagcttgtttgctcatatatatcaaaattctgatagaattggaaacacatgtttgttgttatggtattagcatatgttactcattatacacacaagtacataagtttattgtgacaaaagtgctgatattatgtgaaggttatgtagagaatccgtggagggttggtgacgttaatgactgttgtggatgtgttaaatctttggtaatgtttctttccagctttaattacatcctcaaatgccaatgattcaatagtagtgtaatctcctgcacaaccgcaagacacctgctcaaaaccaaaacaaacaaaaattgttttcgaattgcttttaactttggtttctcgtacaaaaaaactaatacaaaggtgcatgagtagtagtaactgaataaataataagtgcaaatagttgacttaaaataagaagttgaaagagttgaatgatttcatctcactttttaccatttgagataaataagtttgaaaattgattcaacttattcaaccacaaaaattagaaaactaaaccaattataaaaaacgaattgttcaaccctttcatctactattttggagaggaattgattaaatgatgttcaacttttttttcaaactcaaaattttcctatcatttatatatctagcaattcatttgcaactgaaatgtttttttcgtgtcaaactctaggtttgaaacccgtatacatcaccaacaataactagagtttgaaatcaaataatattttttcatatataaaacaatttaaaatttcttttagataattaataaaaaatgtatgacacacagacacagctggttgatttgtttatttgaatttttgactaggattaagaacattaatcatcaaccgttgattatataagcaagttgaagaaaaggcacggttcagattcacctcttctcatatgatgcgttacataagatcctttccctctttccctcattttcaggttcagcgccatcgtcaaaaacttttggcggtcgatcaaaaaatttcgaaatttgaagatttcgccggcaaatacacaaacccgaaaatgaataacacttcaaaattttcattaccagaagaaagaaatcaaataccttcagatctctatcttcctcattcacacaccctctctctcttctccttttctctcttctccttttctctatctccctctttgttccgttcgcatcctctaatcatcgtcaacaagccgacgaagagagaaacgaatccaaagttcgttacttgaaagctacccagaagaattcaaatctcaggtacttttcctgtggatttgatctgggcactgcttattagggatttgattggatctacaaaattctgccttctgggtgattcaatttcacggaa

SEQ ID NO: 84 - DNA, 400 bp, Arabidopsis thaliana
  source 1..400: mol_type = unassigned DNA
tttaagcttattattatgtgtgttgcgaactctttttaactcttttgttaaattcttatgttttcttaactcgtttgttcgtcaccattgctttctcttcaaataatggctgatttttttctataattttggatttgttgaatgctttcttttaaaagaatcagattttggacttttgaccaaagaaaataataatatcagacgataaaatagacggctctcgataaaactaaccctaaaaataaggaaataagttcctctttgaaccaaattttctttctttgaccaatagatctttttgtcaacctcttaaatatattcttagtcaatcttctaataaacccattggccattaccaaaaattcctcggaaacgctgaataaaaaacattctatcatct

SEQ ID NO: 85 - DNA, 386 bp, Arabidopsis thaliana
  source 1..386: mol_type = unassigned DNA
gcaagcaatgtgatgacgtgttcatctataaaactcaaagctcgtttagttcttttgccatgagcttttgcagattaaccataaaatcgaacctttatgtttacaagatccttttgtctccaagtctactggtccgttgaagtttaaagattagatcagtagattaccctttttgcccctacgttaaagtttgtttatttaaatacaagtccttgtgatttttatcactttctcacttcttcaacgacactgattcgttttcaaagcatttgcgttcttgatctttttcacgagggcgatttctgaagaacacgagaatttgaattctgggaaaagctttctcgaactttatctgagtaaattgacagagagaatcgaaaaagaaa

SEQ ID NO: 86 - DNA, 3433 bp, Arabidopsis thaliana
  source 1..3433: mol_type = unassigned DNA
gatttatactatcatgtaactcaaatttaaatttatactataatgtgtttcacgtttttgtgttgttgacaaaacaaaatcccaaaccaatactatgattttcgtagtaaaaaaagttgagtctttctctttaagaaatatgcatgtaaaaggatgttttgatcctagcaaaaagcatattacataaattctaggacatcctaggatttaggaccggctccacctcttccaaaagtctcattgtacctttttcaccgctatgatcgattaatcgttataatattagtatttgattttcttctattcctactttagagttttagataataccttcggaagataaaagagcaaaggcaacgaacgtgcaaacaggttacgacagtacgttaatgttgtctgaatccttaaaaatagtaaacttagcatacgaagaatgcagcaaataggtacaaaaaaagtataatacgtgtataaacatagaatgtgatgttacatacatacgtgtaaaagtttggctacaagtgtcttctacacccaacgacaacacacaaaggagacaaaattgccttttacggcagttacgacttatacgtgtataaaatggtatatacgtctatcgttcagcttaaaaattcagattcacaaatgataataataatagtgcagagcaaatggattagatgaaactcatagaacaattaaaagaaatgttttgcaagaaaatcactcaatttttttattataatagaaatacagagaccactaaaattatagataaataaaactgccatcgtgatattcattaattcttatcagaattaaacgaaaacaagaagtaaaatatctgctgaacgaatcaagttggctcaaatttcactaaaacatgaaaatcaatgaagagattggagagtgttatacttatatgtctcactctttctcgaggcaaatctctgctcctatagattcgtaggtgcctaaaaggctcaatttgaagccatgcatgtcttttgctttcacaatttgtcattccccatgccaacttttcacaaatttttacagtccatatatgtatatgcatattgcacatttatatatctaaagttggtggcgtaacacgatgaattttaactaattgatagtcgcacaatggcatccttatgttagtctgaaatcagtactgtatcaaatttaattctgacttttatttgacttgtctagttttcagttgaagcgaaactataatggttaaatgatgtttttcttatgattatacaaaaaaaaaattcaaaaaaattcataatgtttgataaaacgcaataatgcaggatttgcgtttttgtctaaaaaaaagtaaaaacttattatgtaaataaatgattatggttatgttatgaagtcttattccgttagtctgcacatatatctatccgtacaatgtgattttttcggcgaatagttatctaatatttttcattctaaaacttgtattattatgtacaatagttatgcaatattttattttcccttttgatcttaaaagttcaatgttttgagttttttttttgttccacaattattgatattctacatcaacatcattattatttttattgttgaccaataaaaataagaaaaaaatatagttaatattattaaactaataaaataataatattaaaatgtaaaagttatttttttaatttgtatgaaaatttcacaacatcagacttgataaacttgatgtaatagagggaatactaaagagtcaatatgtagttagccatgagcccatgattgtgatgtaaaaatatgtgttgaaaaatataaaacaattaaacacgagggaattcaacaattaaagtagtatgtttcactatgctacattacaaattatttagagaaaattaaattaaaataagaggatgaggaaatgatgtgataaaaatgttctaaagtacgtatgtaccgtgaaaagtgcaattatatggaagaaaaacaccacaaacacgacactgaaacgttgtacatacgattgcttcatctgcaagtatagaacgaatatttgttttattttgtcttaaagtatcgattagttccaaaaaacagctttctgaaaccatttttcatttgtatggatttattatattttgtagttttacagatagatatcattcttaaatagttttaattagaaaaattatgttctatatttcaaaattatgttttattttatacctatagttccaacttccaaataaacgttttgtgcctaggttaagcatatcggtgatttgcgctcaactaacgtctgctatatttgagaaacttaaatttttactgaaaaacctacgaaaaacctacgaaaatatctcgtacggatatgatcaataagattctatctgaaagaaaatgacggtccatacttgaactcgccatttcctcttcaactaggaaaaaattggatcgacattggatttaagaaaggaaatgaaaccacgaggcgaaagaactaagccactagagttctctattttgacggtttactaaatataatcaagagtttgtttctttgctcgaatcttttgaatttttttcctgttcgttaagaaatcgaagagccaagctactggacttcatattgtcgtgttatataaatacaaatattataaacaaaatatgatagtactataatataagatatgtcaatttccgaagcaaactagatatgtaggcttctcatgacctacaaagaacacaatccaaccagctatcttttgccgccctatcatatttaacgttcgtatttatcctctctttcatttccaaaacttaatggttcttcttcattaatacacttcaaaaaaaaaacatccgtaatccccagaaagaataagaaaaactaataaagtcacaagtcacaactaacaattatacaccgtatctactggaaaataaagagagagttatatccattattcacatagaaaaaacatttttacatgttaaagacaagtattactgtgttatctctttttctatttgccgcattttgataaattaattataaaatgcaacatgctttttttttttttccgcgtatgtgccaatgcaacatgcttttattaaaaaaaaaacgaatagatccaaaatgtttttttttcctttctatttgtcttctcataaaaaagtagcctcttttttaccatctcagccgctccacgcgatgttttcagagtctccagcgcaaaaaacaacacgcatgtcactatctctctcttcatttgactctgttccaatccctcactaactcgctatataaatatgagagcctccccatgaatttcatgcatcattcaacccccataacctacatgcaaaccatcgtcttaaaaccctagttctccataaaaaaaatatccttgaacacaaataa

SEQ ID NO: 87 - DNA, 292 bp, Arabidopsis thaliana
  source 1..292: mol_type = unassigned DNA
atcttaaaacgtaaggcttaagataagtagtatatggtggatatggaacccgcgtaatcatctagaggctctacaaatatttattttgtatttccttcttattttgtatttgcctacgtggcattatacaacgtatttaacttgaaaccagatttatggcccaatgggtcgggtcgacccgaccgattttaaactgcgctcctaactaaaaaaaagtcaaaaccctttgaaaaacctaaaaacgcaatttgcttcgtcgtctctcatctctttctctttctccgtcgccacc

SEQ ID NO: 88 - DNA, 494 bp, Arabidopsis thaliana
  source 1..494: mol_type = unassigned DNA
ttgattactgtggatctagctagagagaagtaatggcacggacgaagaaagctaaaaagcagggagattgttaacttacgtttttgtccttctcttagcttaaaacggtctgataattaaacttcgaatctaaacaatttaatattttacagcataatcccagtttaactgtaatctaaacaaggccaattaataaatatgcaagcccataactacagtttggcccattagtttttccccgcgccaaatttgccctagttcctttcttctttcccgctatttccaaaatctcccaaaaggtcaagttccagcaaaatgacacaatctttgtaatcaacattgttctccgtcaagcttctccttcaaaattcgtaagctttctgttctctaactctttttggtttcagtatatgaatcaatccgactttggaatctctacttaaaaataaaaatcgatctttttgctgcccagatagtgaataaccttaaaatcg

SEQ ID NO: 89 - DNA, 297 bp, Arabidopsis thaliana
  source 1..297: mol_type = unassigned DNA
gtatttcattgggataaatgaagcgttgttatggatagttttgagttgctaaaaagccaaagatctaatcgcaagtcatatagggattacatttccttgtagctcttgaagtttttgctcccttttttgtttgttccttgtaccattttttcttaatacgagagataagaacttatcattttgaggctctgaagaatcaaagaggataacagttcccaattttttcattctctcagtaaagctttttgtttcgttttcccaaatgttgaagataagtatatgaaaaggcaaattatt

SEQ ID NO: 90 - DNA, 138 bp, Arabidopsis thaliana
  source 1..138: mol_type = unassigned DNA
gtcttagtattcatctttacactcctgctatgttttcttttgctaagagaattttaatctttatgtaactggtgacgaatgcttgaaaaatagcgtctctgttattacttttgagaataaaagatcatcattactcac

SEQ ID NO: 91 - DNA, 575 bp, Arabidopsis thaliana
  source 1..575: mol_type = unassigned DNA
tgtatgcaaactccagtaatgattttgttataatatatggtttttaaatgatataatggcatctccctttggatttttccgtgtgactaaaaccgcatttataatttatacctggacaaagaacaaaaacagtgttgccaagaaacagagcatagtaagaacagagaaagtttctattcaagtaacttgtaaaacaagctagtagattcatgtttttgcaaaaccctagaaaagtcatagtttcattgcttctaaacaattaacagaaaaaaacagggaacagaacatatagaaagaaaagacaagtaacaagtccaacaagaaaacggattcatgattttgtaaccatgctcaagaaaatctcagaatctagacgtgatcgagactccatggtccacctgctccgttaaaatctcagctgacgactcagggagtaaagagttggccacatcgatcacgaaccggtacctgacatcagatttagccaaacggtccatcgcagagttgatatcactcatctttatgagctcaatatccgaaacgattttatgcttggcacagaactcaagcatctc

SEQ ID NO: 92 - DNA, 190 bp, Arabidopsis thaliana
  source 1..190: mol_type = unassigned DNA
ttgttatttctttgactaatgtcattaattttctctagaataaaacttcgaaaattgaaatgtgatgtgcacaatcatttctgttttctttatttggttgataagcaaatttggctatttatgttttttttttaacggatgcctatatatgatactgcattgcagtgtttttggtctttccataaaaata

SEQ ID NO: 93 - DNA, 267 bp, Arabidopsis thaliana
  source 1..267: mol_type = unassigned DNA
gaacttttcgatagtgacgtgcatggcttgagtttgattttctcgtttttatttatgttataattattgtgttccatctatgaatgtaaaagatttatgaaggattcgatttttttctctttttttaaagattattataaacgtctttcataaccatctctttattcattatatgttcaggagtgaaaaaacaaaatctaaaaaatagggttaaatgtttgcttagcttctctctttatgtagatttgagaaccgatgaacaagaat

SEQ ID NO: 94 - DNA, 276 bp, Arabidopsis thaliana
  source 1..276: mol_type = unassigned DNA
taaatgttcctcgctgacgtaagaagacattagtaatggttataatatatagctttctatgaatgtatggtgagaaaatgtctgttcactgattttgagtttggaataaaagcatttgcgtttggtttatcattgcgtttatacaaggacagagatccactgagctggaatagcttaaaaccattatcagaacaaaataaaccattttttgttaagaatcagagcatagtaaacaacagaaacaacctaagagaggtaacttgtccaagaagatag

SEQ ID NO: 95 - DNA, 110 bp, Arabidopsis thaliana
  source 1..110: mol_type = unassigned DNA
tagtaactttcatatcaattaataaacgctcatgtttaaatgatcacaatccaatgattctgcatattgagattctttaaaatcataaagagtttaaagctgtttaggtt

SEQ ID NO: 96 - DNA, 135 bp, Arabidopsis thaliana
  source 1..135: mol_type = unassigned DNA
tagctttccctttatattttaagttaaataaaagttaccagtgaactatgtttcctttttatgtgtaacattttctgatttatctttgttaaattttgttaatcacggtctaaatcgatttaaaatcgttttggt

SEQ ID NO: 97 - DNA, 248 bp, Arabidopsis thaliana
  source 1..248: mol_type = unassigned DNA
catagtctcattgttcttcgattcagtgtgttttcttttatagttttcagttttatctcactgtttgcattttttacgagcctgtgtaataggcacaatctgttatcaatcatgtaacttgtttaatcaaataaccatagagttttatgtgaaaaggtcctttactcatttggtgttaactctttacctcttcaggtctttaactctgtttatataaaacatctaatgaacaatctgtgttttgaacg

SEQ ID NO: 98 - DNA, 285 bp, Arabidopsis thaliana
  source 1..285: mol_type = unassigned DNA
agagcattgtgttaatgatatgccattgttgtgttttcttctacagtttgagttttgcaggaacttttcttttgtacatagccagccaaacgtttgaatgtgttgtaataaggacttgatcattttaatatgtgcaggttttgtctttcttaacatgttgaatcgctttatgattttaccggaacgaaccggttgctatggtttcggtatgatttgatttctcgttataggccttagtcggcccaataacgaagattctctataaataaatgaacataaacaagg

SEQ ID NO: 99 - DNA, 392 bp, Arabidopsis thaliana
  source 1..392: mol_type = unassigned DNA
gatctatttgtaccggttatcaagttttttggatcatagaaattgtataggcttttgacattgaaatattttcatagacaaaacaaagaagcataggcaaatgtgattacactctggaacaaatcttaggtttgggaaagaaattcaataacaaaacacacaaagcacaaaaagagagaatcccaattcagaaaaagaggtaaaaggaaatgagtgcgacgaacttacagccttacaaaagtaataatcaatcatcttttctttaggcttcttcttcttcttcgtcctcctcgtactcctcttcaccgactgtagcatcttgatattgctgatactctgcaacaagatcattcatgttactctctgcttcagtgaactccatctcgtccatt

SEQ ID NO: 100 - DNA, 297 bp, Arabidopsis thaliana
  source 1..297: mol_type = unassigned DNA
cgaaggtttaatgaaattataaattttatgtaatcattagttgttttaaaaagtatttcaaactgtggagaaactcatgaacattttgaaacgaaacgtaactatatattttggtcgaatttaaaggttaaatcactctcattaaacaagtcaattatttcaatgaatttcaatttcatttattaattaaaaaaattcttctaaaattaaatttaagtactaattaaacttggtaatgaataaacttaattcatatagtgatacatctataggaggccgacatagacattctataaa

SEQ ID NO: 101 - DNA, 319 bp, Arabidopsis thaliana
  source 1..319: mol_type = unassigned DNA
gcaagggaagaagctaaagaagctccattttgtttcttgaggccctgcttagtaaggaactaagaatagaaatgttgaagagtgtcagaggatgaatcggccaagttgtgaggaagtatgtttccttccatatgtatgcctaaaattttgtaagagccttaaatgagttttgtttcttttctttgttttcaatacaatagtggatattgagctttgtatagctcatacctagatttatactatattaagaacttctttttgtttttgccgtttgtgttctcttcatttttatgagtcaatccattgtgtttaacttcga

SEQ ID NO: 102 - DNA, 202 bp, Arabidopsis thaliana
  source 1..202: mol_type = unassigned DNA
ccttagtaaggtttataacctgcttatttcagtcttagttttgatttcagtttggttcagtttgtaatgtacaagtttctctaagctttataatctatcagtagagtctctctatcttgttgtggtgtttcctaagaaaattatagcctttgagcgagtgattgaatatatttatatggtaatgaatcacaccgagtcaccg